SEQUENCE LISTING (34 sequences)

SEQ ID NO: 1 - protein, 1273 aa, Artificial Sequence
  S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauaaaguggaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 3 - protein, 218 aa, Artificial Sequence
  S Protein RBD Fusion
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 4 - RNA, 654 nt, Artificial Sequence
  Coding Sequence
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaa

SEQ ID NO: 5 - protein, 266 aa, Artificial Sequence
  S Protein RBD Fusion
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 6 - RNA, 798 nt, Artificial Sequence
  Coding Sequence
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 7 - protein, 1273 aa, Artificial Sequence
  S Protein Variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 9 - RNA, 3819 nt, Artificial Sequence
  Coding Sequence
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacaca

SEQ ID NO: 10 - protein, 41 aa, Artificial Sequence
  Foldon Sequence
GSGYIPEAPRDGQAYVRKDGEWVLLSTFLGRSLEVLFQGPG

SEQ ID NO: 11 - RNA, 123 nt, Artificial Sequence
  Coding Sequence
ggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 12 - RNA, 47 nt, Artificial Sequence
  5′-UTR
aacuaguauucuucugguccccacagacucagagagaacccgccacc

SEQ ID NO: 13 - RNA, 278 nt, Artificial Sequence
  3′-UTR
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 14 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 15 - RNA, 4282 nt, Artificial Sequence
  RBL063.1
gggcgaacuaguauucuucugguccccacagacucagagagaacccgccaccauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 16 - RNA, 4282 nt, Artificial Sequence
  RBL063.2
gggcgaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 17 - RNA, 1261 nt, Artificial Sequence
  RBL063.3
gggcgaacuaguauucuucugguccccacagacucagagagaacccgccaccauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 18 - protein, 250 aa, Artificial Sequence
  S Protein RBD Fusion
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 19 - RNA, 4283 nt, Artificial Sequence
  RBP020.1
agaauaaacuaguauucuucugguccccacagacucagagagaacccgccaccauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 20 - RNA, 4283 nt, Artificial Sequence
  RBP020.2
agaauaaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 21 - RNA, 1262 nt, Artificial Sequence
  RBP020.3
agaauaaacuaguauucuucugguccccacagacucagagagaacccgccaccauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 22 - protein, 1879 aa, Artificial Sequence
  Viral Protein
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQ

SEQ ID NO: 23 - protein, 613 aa, Artificial Sequence
  Viral Protein
RFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 24 - RNA, 11917 nt, Artificial Sequence
  RBS004.1
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 25 - RNA, 11917 nt, Artificial Sequence
  RBS004.2
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 26 - RNA, 8896 nt, Artificial Sequence
  RBS004.3
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 27 - RNA, 9079 nt, Artificial Sequence
  RBS004.4
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 28 - protein, 327 aa, Artificial Sequence
  Vaccine Antigen
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCCKFDEDDSEPVLKGVKLHYT

SEQ ID NO: 29 - protein, 311 aa, Artificial Sequence
  BNT162b3c
MFVFLVLLPLVSSQCVNLTVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 30 - RNA, 1397 nt, Artificial Sequence
  BNT162b3c
agaauaaacuaguauucuucugguccccacagacucagagagaacccgccaccauguuuguguuucuugugcugcugccucuugugucuucucagugugugaauuugacagugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 31 - protein, 314 aa, Artificial Sequence
  BNT162b3d
MDWIWRILFLVGAATGAHSQMQVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 32 - RNA, 1406 nt, Artificial Sequence
  BNT162b3d
agaauaaacuaguauucuucugguccccacagacucagagagaacccgccaccauggauuggauuuggagaauccuguuccucgugggagccgcuacaggagcccacucccagaugcaggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 33 - protein, 9 aa, Artificial Sequence
  Linker
GSPGSGSGS

SEQ ID NO: 34 - protein, 6 aa, Artificial Sequence
  Linker
GSGSGS

(19) United States
(12) Patent Application Publication
(10) Pub. No.: US 2023/0414747 A1
(43) Pub. Date: Dec. 28, 2023

(54) LNP COMPOSITIONS COMPRISING RNA AND METHODS FOR PREPARING, STORING AND USING THE SAME

(71) Applicant: BioNTech SE, Mainz (DE)

(72) Inventors: Steffen Panzner, Halle (DE); Ugur Sahin, Mainz (DE); Jorrit-Jan Krijger, Mainz (DE); Kaushik Thanki, Wiesbaden (DE); Bakul Subodh Bhatnagar, North Reading, MA (US); Ramin Darvari, Lexington, MA (US); Sumit Luthra, Westford, MA (US); Serguei A. Tchessalov, Billerica, MA (US)

(21) Appl. No.: 18/036,677

(22) PCT Filed: Nov. 15, 2021
(86) PCT No.: PCT/EP21/81675

§ 371 Date: May 12, 2023

Related U.S. Application Data
(60) Provisional application No. 63/149,372, filed on Feb. 15, 2021.
(60) Provisional application No. 63/135,723, filed on Jan. 10, 2021.
(60) Provisional application No. 63/115,128, filed on Nov. 18, 2020.
(60) Provisional application No. 63/115,588, filed on Nov. 18, 2020.
(60) Provisional application No. 63/114,478, filed on Nov. 16, 2020.

(30) Foreign Application Priority Data
Nov. 18, 2020 (WO) PCT/EP2020/082602
Apr. 12, 2021 (WO) PCT/EP2021/059460

(51) Int. Cl. A61K 39/215; A61K 9/51; A61P 31/14

(52) U.S. Cl. CPC A61K 39/215; A61K 9/5123; A61P 31/14; C12N 2770/20034; A61K 2039/53

(57) ABSTRACT

The present disclosure relates generally to the field of lipid nanoparticle (LNP) compositions comprising RNA, methods for preparing and storing such compositions, and the use of such compositions in therapy.

### TECHNICAL FIELD

The present disclosure relates generally to the field of lipid nanoparticle (LNP) compositions comprising RNA, methods for preparing and storing such compositions, and the use of such compositions in therapy.

### BACKGROUND

The use of a recombinant nucleic acid (such as DNA or RNA) for delivery of foreign genetic information into target cells is well known. The advantages of using RNA include transient expression and a non-transforming character. RNA does not need to enter the nucleus in order to be expressed and moreover cannot integrate into the host genome, thereby eliminating diverse risks such as oncogenesis.

A recombinant nucleic acid may be administered in naked form to a subject in need thereof; however, usually a recombinant nucleic acid is administered using a composition. For example, RNA may be delivered by so-called nanoparticle formulations containing RNA and a nanoparticle forming vehicle, e.g., a cationic lipid (such as a permanently charged cationic lipid), a mixture of a cationic lipid and one or more additional lipids, or a cationic polymer. The fate of such nanoparticle formulations is controlled by diverse key-factors (e.g., size and size distribution of the nanoparticles; etc.). These factors are, e.g., referred to in the FDA “Liposome Drug Products Guidance” from 2018 as specific attributes which should be analyzed and specified. The limitations to the clinical application of current nanoparticle formulations may lie in the lack of homogeneous, pure and well-characterized nanoparticle formulations.

LNPs comprising ionizable lipids may display advantages in terms of targeting and efficacy in comparison to other RNA nanoparticle products. However, it is challenging to obtain sufficient shelf life as required for regular pharmaceutical use. It is said that for stabilization, LNPs comprising ionizable lipids need to be frozen at much lower temperatures, such as −80° C., which poses substantial challenges on the cold chain, or they can only be stored above the freezing temperature, e.g. 5° C., where only limited stability can be obtained.

It is known that RNA in solution or in LNPs undergoes slow fragmentation. Furthermore, in the presence of phosphate buffered saline (PBS), RNA has the tendency to adopt a very stable folded form which is hardly accessible for translation. Both mechanisms, i.e., fragmentation and formation of this stable RNA fold, are temperature dependent and result in loss of intact and accessible RNA thereby limiting the stability of the liquid product; however, they are essentially absent in the frozen state.

Thus, there remains a need in the art for (i) compositions which comprise LNPs comprising ionizable lipids and RNA and which are stable and can be stored in a temperature range compliant to regular technologies in pharmaceutical practice, in particular at a temperature of about −25° C. or even in liquid form at temperatures between +2 and +20° C.; (ii) compositions which are ready to use; (iii) compositions which, preferably, can repeatably be frozen and thawed, and (iv) methods for preparing and storing such compositions. The present disclosure addresses these and other needs.

The inventors surprisingly found that the compositions and methods described herein fulfill the above-mentioned requirements. In particular, it is demonstrated that by using a specific buffer substance, in particular tris(hydroxymethyl)aminomethane (Tris) and its protonated form, in a low concentration (e.g., at most about 25 mM) and excluding inorganic phosphate anions as well as citrate anions and anions of EDTA, it is possible to prepare compositions which are stable and which can be stored at about −25° C. or even in liquid form.

### SUMMARY

In a first aspect, the present disclosure provides a composition comprising lipid nanoparticles (LNPs) dispersed in an aqueous phase, wherein the LNPs comprise a cationically ionizable lipid and RNA; the aqueous phase comprises a buffer system comprising a buffer substance selected from the group consisting of Tris and its protonated form, bis(2-hydroxyethyl)amino-tris(hydroxymethyl)methane (Bis-Tris-methane) and its protonated form, and triethanolamine (TEA) and its protonated form, and the monovalent anion being selected from the group consisting of chloride, acetate, glycolate, lactate, the anion of morpholinoethanesulfonic acid (MES), the anion of 3-(N-morpholino)propanesulfonic acid (MOPS), and the anion of 2-[4-(2-hydroxyethyl)piperazin-1-yl]ethanesulfonic acid (HEPES); the concentration of the buffer substance in the composition is at most about 25 mM; and the aqueous phase is substantially free of inorganic phosphate anions, substantially free of citrate anions, and substantially free of anions of ethylenediaminetetraacetic acid (EDTA).

As demonstrated in the present application, using a buffer system based on the particular buffer substances specified above, in particular Tris and its protonated form, instead of PBS in a composition comprising LNPs inhibits the formation of a very stable folded form of RNA. Furthermore, the present application demonstrates that, surprisingly, by simply lowering the concentration of the buffer substance in a composition comprising LNPs and a buffer system, wherein the LNPs comprise a cationically ionizable lipid and RNA, it is possible to obtain an RNA LNP composition having improved RNA integrity after a freeze-thaw-cycle compared to a composition comprising the same buffer substance in a concentration of 50 mM. Thus, the claimed composition provides improved stability, can be stored in a temperature range compliant to regular technologies in pharmaceutical practice, and provides a ready-to-use composition.

In a preferred embodiment of the first aspect, the buffer substance is Tris and its protonated form, i.e., a mixture of Tris and its protonated form.

In one embodiment, the monovalent anion is selected from the group consisting of chloride, acetate, glycolate, lactate, morpholinoethanesulfonate, and 3-(N-morpholino)propanesulfonate, or from the group consisting of chloride, acetate, glycolate, lactate, morpholinoethanesulfonate, and 2-[4-(2-hydroxyethyl)piperazin-1-yl]ethanesulfonate, preferably from the group consisting of chloride, acetate, lactate, and morpholinoethanesulfonate, more preferably from the group consisting of chloride, acetate, and morpholinoethanesulfonate, or from the group consisting of chloride, acetate, and lactate, such as chloride or acetate.

In one embodiment, the buffer substance is Tris and its protonated form and the monovalent anion is selected from the group consisting of chloride, acetate, glycolate, lactate, morpholinoethanesulfonate, and 3-(N-morpholino)propanesulfonate, or from the group consisting of chloride, acetate, glycolate, lactate, morpholinoethanesulfonate, and 2-[4-(2-hydroxyethyl)piperazin-1-yl]ethanesulfonate, preferably from the group consisting of chloride, acetate, lactate, and morpholinoethanesulfonate, more preferably from the group consisting of chloride, acetate, and morpholinoethanesulfonate, or from the group consisting of chloride, acetate, and lactate, such as chloride or acetate.

In one embodiment of the first aspect, the concentration of the buffer substance, in particular the total concentration of Tris and its protonated form, in the composition is at most about 20 mM, such as at most about 19 mM, at most about 18 mM, at most about 17 mM, at most about 16 mM, at most about 15 mM, at most about 14 mM, at most about 13 mM, at most about 12 mM, at most about 11 mM, or at most about 10 mM. In one embodiment, the lower limit of the buffer substance, in particular Tris and its protonated form, in the composition is at least about 1 mM, preferably at least about 2 mM, such as at least about 3 mM, at least about 4 mM, at least about 5 mM, at least about 6 mM, at least about 7 mM, at least about 8 mM, or at least about 9 mM. For example, the concentration of the buffer substance, in particular the total concentration of Tris and its protonated form, in the composition may be between about 1 mM and about 20 mM, such as between about 2 mM and about 15 mM, between about 5 mM and about 14 mM, between about 7 mM and about 13 mM, between about 8 mM and about 12 mM, between about 9 mM and about 11 mM, such as about 10 mM.

In one embodiment of the first aspect, the aqueous phase is substantially free of inorganic sulfate anions and/or carbonate anions and/or dibasic organic acid anions and/or polybasic organic acid anions. In a first subgroup, at least one of these criteria applies. For example, in one embodiment of this first subgroup, the aqueous phase is substantially free of inorganic sulfate anions. In a further embodiment of this first subgroup, the aqueous phase is substantially free of carbonate anions. In a further embodiment of this first subgroup, the aqueous phase is substantially free of dibasic organic acid anions.

In a further embodiment of this first subgroup, the aqueous phase is substantially free of polybasic organic acid anions.

In a second subgroup of the first aspect, at least two of the above criteria apply. For example, in one embodiment of this second subgroup, the aqueous phase is substantially free of inorganic sulfate anions and substantially free of carbonate anions. In a further embodiment of this second subgroup, the aqueous phase is substantially free of inorganic sulfate anions and substantially free of dibasic organic acid anions. In a further embodiment of this second subgroup, the aqueous phase is substantially free of inorganic sulfate anions and substantially free of polybasic organic acid anions. In a further embodiment of this second subgroup, the aqueous phase is substantially free of carbonate anions and substantially free of dibasic organic acid anions. In a further embodiment of this second subgroup, the aqueous phase is substantially free of carbonate anions and substantially free of polybasic organic acid anions. In a further embodiment of this second subgroup, the aqueous phase is substantially free of dibasic organic acid anions and substantially free of polybasic organic acid anions.

In a third subgroup of the first aspect, at least three of the above criteria apply. For example, in one embodiment of this third subgroup, the aqueous phase is substantially free of inorganic sulfate anions, substantially free of carbonate anions and substantially free of dibasic organic acid anions. In a further embodiment of this third subgroup, the aqueous phase is substantially free of inorganic sulfate anions, substantially free of carbonate anions and substantially free of polybasic organic acid anions. In a further embodiment of this third subgroup, the aqueous phase is substantially free of inorganic sulfate anions, substantially free of dibasic organic acid anions and substantially free of polybasic organic acid anions.

In a further embodiment of this third subgroup, the aqueous phase is substantially free of carbonate anions, substantially free of dibasic organic acid anions and substantially free of polybasic organic acid anions.

In a fourth subgroup of the first aspect, at least four of the above criteria apply. I.e., in this fourth subgroup, the aqueous phase is substantially free of inorganic sulfate anions, substantially free of carbonate anions, substantially free of dibasic organic acid anions and substantially free of polybasic organic acid anions.

In one embodiment of the first aspect (in particular in one embodiment of the above first, second, third, or fourth subgroup of the first aspect), the composition comprises a cryoprotectant. In an alternative embodiment of the first aspect (in particular in one embodiment of the above first, second, third, or fourth subgroup of the first aspect), the composition is substantially free of a cryoprotectant. Thus, particular examples of these embodiments are the following:
(1) the aqueous phase is substantially free of inorganic sulfate anions, and the composition comprises a cryoprotectant;
(2) the aqueous phase is substantially free of carbonate anions, and the composition comprises a cryoprotectant;
(3) the aqueous phase is substantially free of dibasic organic acid anions, and the composition comprises a cryoprotectant;
(4) the aqueous phase is substantially free of polybasic organic acid anions, and the composition comprises a cryoprotectant;
(5) the aqueous phase is substantially free of inorganic sulfate anions and substantially free of carbonate anions, and the composition comprises a cryoprotectant;
(6) the aqueous phase is substantially free of inorganic sulfate anions and substantially free of dibasic organic acid anions, and the composition comprises a cryoprotectant;
(7) the aqueous phase is substantially free of inorganic sulfate anions and substantially free of polybasic organic acid anions, and the composition comprises a cryoprotectant;
(8) the aqueous phase is substantially free of carbonate anions and substantially free of dibasic organic acid anions, and the composition comprises a cryoprotectant;
(9) the aqueous phase is substantially free of carbonate anions and substantially free of polybasic organic acid anions, and the composition comprises a cryoprotectant;
(10) the aqueous phase is substantially free of dibasic organic acid anions and substantially free of polybasic organic acid anions, and the composition comprises a cryoprotectant;
(11) the aqueous phase is substantially free of inorganic sulfate anions, substantially free of carbonate anions and substantially free of dibasic organic acid anions, and the composition comprises a cryoprotectant;
(12) the aqueous phase is substantially free of inorganic sulfate anions, substantially free of carbonate anions and substantially free of polybasic organic acid anions, and the composition comprises a cryoprotectant;
(13) the aqueous phase is substantially free of inorganic sulfate anions, substantially free of dibasic organic acid anions and substantially free of polybasic organic acid anions, and the composition comprises a cryoprotectant;
(14) the aqueous phase is substantially free of carbonate anions, substantially free of dibasic organic acid anions and substantially free of polybasic organic acid anions, and the composition comprises a cryoprotectant;
(15) the aqueous phase is substantially free of inorganic sulfate anions, substantially free of carbonate anions, substantially free of dibasic organic acid anions and substantially free of polybasic organic acid anions, and the composition comprises a cryoprotectant;
(16) the aqueous phase is substantially free of inorganic sulfate anions, and the composition is substantially free of a cryoprotectant;
(17) the aqueous phase is substantially free of carbonate anions, and the composition is substantially free of a cryoprotectant;
(18) the aqueous phase is substantially free of dibasic organic acid anions, and the composition is substantially free of a cryoprotectant;
(19) the aqueous phase is substantially free of polybasic organic acid anions, and the composition is substantially free of a cryoprotectant;
(20) the aqueous phase is substantially fie of inorganic sulfate anions and substantially free of carbonate anions, and the composition is substantially free of a cryoprotectant;
(21) the aqueous phase is substantially free of inorganic sulfate anions and substantially free of dibasic organic acid anions, and the composition is substantially free of a cryoprotectant;
(22) the aqueous phase is substantially free of inorganic sulfate anions and substantially free of polybasic organic acid anions, and the composition is substantially free of a cryoprotectant;
(23) the aqueous phase is substantially free of carbonate anions and substantially free of dibasic organic acid anions, and the composition is substantially free of a cryoprotectant;
(24) the aqueous phase is substantially free of carbonate anions and substantially free of polybasic organic acid anions, and the composition is substantially free of a cryoprotectant;
(25) the aqueous phase is substantially free of dibasic organic acid anions and substantially free of polybasic organic acid anions, and the composition is substantially free of a cryoprotectant;
(26) the aqueous phase is substantially free of inorganic sulfate anions, substantially free of carbonate anions and substantially free of dibasic organic acid anions, and the composition is substantially free of a cryoprotectant;
(27) the aqueous phase is substantially free of inorganic sulfate anions, substantially free of carbonate anions and substantially free of polybasic organic acid anions, and the composition is substantially free of a cryoprotectant;
(28) the aqueous phase is substantially free of inorganic sulfate anions, substantially free of dibasic organic acid anions and substantially free of polybasic organic acid anions, and the composition is substantially free of a cryoprotectant;
(29) the aqueous phase is substantially free of carbonate anions, substantially free of dibasic organic acid anions and substantially free of polybasic organic acid anions, and the composition is substantially free of a cryoprotectant;
(30) the aqueous phase is substantially free of inorganic sulfate anions, substantially free of carbonate anions, substantially free of dibasic organic acid anions and substantially free of polybasic organic acid anions, and the composition is substantially free of a cryoprotectant.

In one embodiment of the first aspect (in particular in one embodiment of the above first, second, third, or fourth subgroup of the first aspect, such as in any of the embodiments (1) to (30) listed above), wherein the composition comprises a cryoprotectant, said cryoprotectant comprises one or more compounds selected from the group consisting of carbohydrates and sugar alcohols. For example, the cryoprotectant may be selected from the group consisting of sucrose, glucose, glycerol, sorbitol, and a combination thereof. In a preferred embodiment of the first aspect (in particular in one embodiment of the above first, second, third, or fourth subgroup of the first aspect, such as in any of the embodiments (1) to (30) listed above), the composition comprises sucrose and/or glycerol as cryoprotectant.

In one embodiment of the first aspect (in particular in one embodiment of the above first, second, third, or fourth subgroup of the first aspect, such as in any of the embodiments (1) to (30) listed above), wherein the composition comprises a cryoprotectant, the concentration of the cryoprotectant in the composition is at least 1% w/v, such as at least 2% w/v, at least 3% w/v, at least 4% w/v, at least 5% w/v, at least 6% w/v, at least 7% w/v, at least 8% w/v, or at least 9% w/v. In one embodiment, the concentration of the cryoprotectant in the composition is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In one embodiment, the concentration of the cryoprotectant in the composition is 1% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In one embodiment of the first aspect (in particular in one embodiment of the above first, second, third, or fourth subgroup of the first aspect, such as in any of the embodiments (1) to (30) listed above), the composition comprises a cryoprotectant (in particular, sucrose and/or glycerol) in a concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v.

In one embodiment of the first aspect (in particular in one embodiment of the above first, second, third, or fourth subgroup of the first aspect, such as in any of the embodiments (1) to (30) listed above), wherein the composition comprises a cryoprotectant, the cryoprotectant is present in a concentration resulting in an osmolality of the composition in the range of from about 50×10⁻³ osmol/kg to about 400×10⁻³ osmol/kg (such as from about 50×10⁻³ osmol/kg to about 390×10⁻³ osmol/kg, from about 60×10⁻³ osmol/kg to about 380×10⁻³ osmol/kg, from about 70×10⁻³ osmol/kg to about 370×10⁻³ osmol/kg, from about 80×10⁻³ osmol/kg to about 360×10⁻³ osmol/kg, from about 90×10⁻³ osmol/kg to about 350×10⁻³ osmol/kg, from about 100×10⁻³ osmol/kg to about 340×10⁻³ osmol/kg, from about 120×10⁻³ osmol/kg to about 330×10⁻³ osmol/kg, from about 140×10⁻³ osmol/kg to about 320×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the composition.

In one embodiment of the first aspect (in particular in one embodiment of the above first, second, third, or fourth subgroup of the first aspect, such as in any of the embodiments (1) to (30) listed above), in particular in those embodiments of the first aspect, where the buffer substance is Tris and its protonated form, the monovalent anion is selected from the group consisting of chloride, acetate, glycolate, and lactate, and the concentration of the monovalent anion (in particular the total concentration of all monovalent anions) in the composition is at most equal to the concentration of the buffer substance in the composition. For example, the concentration of the monovalent anion (in particular the total concentration of all monovalent anions) in the composition may be less than the concentration of the buffer substance in the composition. Thus, in those embodiments of the first aspect, where the concentration of the buffer substance, in particular Tris and its protonated form, in the composition is at most about 20 mM, the concentration of the monovalent anion (in particular the total concentration of all monovalent anions) in the composition is at most equal to about 20 mM, e.g., less than 20 mM.

Generally, the concentration of the monovalent anion, such as chloride and/or acetate (in particular the total concentration of all monovalent anions) in the composition may be less than about 15 mM, such as less than about 14 mM, less than about 13 mM, less than about 12 mM, less than about 11 mM, less than about 10 mM, less than about 9 mM, less than about 8 mM, less than about 7 mM, less than about 6 mM, or less than about 5 mM. In one embodiment, the chloride concentration in the composition is as defined above (e.g., less than about 15 mM, etc.) and the composition does not comprise acetate. In an alternative embodiment, the acetate concentration in the composition is as defined above (e.g., less than about 15 mM, etc.) and the composition does not comprise chloride.

In one embodiment of the first aspect (in particular in one embodiment of the above first, second, third, or fourth subgroup of the first aspect, such as in any of the embodiments (1) to (30) listed above), in particular in those embodiments of the first aspect, where the buffer substance is Tris and its protonated form, the sodium concentration in the aqueous phase and/or composition is less than 20 mM, such as less than about 15 mM, e.g., less than about 14 mM, less than about 13 mM, less than about 12 mM, less than about 11 mM, less than about 10 mM, less than about 9 mM, less than about 8 mM, less than about 7 mM, less than about 6 mM, or less than about 5 mM.

In one embodiment of the first aspect (in particular in one embodiment of the above first, second, third, or fourth subgroup of the first aspect, such as in any of the embodiments (1) to (30) listed above), in particular in those embodiments of the first aspect, where the buffer substance is Tris and its protonated form, the monovalent anion is selected from the group consisting of the anions of MES, MOPS and HEPES, and the concentration of the monovalent anion (in particular the total concentration of all monovalent anions) in the composition is at least equal to the concentration of the buffer substance in the composition. For example, the concentration of the monovalent anion (in particular the total concentration of all monovalent anions) in the composition may be higher than the concentration of the buffer substance in the composition. Thus, in those embodiments of the first aspect, where the concentration of the buffer substance, in particular Tris and its protonated form, in the composition is at most about 20 mM, the concentration of the monovalent anion (in particular the total concentration of all monovalent anions) in the composition is at least equal to about 20 mM, e.g., higher than 20 mM.

Generally, the concentration of the monovalent anion (in particular the total concentration of all monovalent anions) in the composition may be higher than about 20 mM, such as higher than about 21 mM, higher than about 22 mM, higher than about 23 mM, higher than about 24 mM, higher than about 25 mM, higher than about 26 mM, higher than about 27 mM, higher than about 28 mM, higher than about 29 mM, or higher than about 30 mM, and preferably at most 50 mM, such as at most 45 mM, at most 40 mM or at most 35 mM.

In one embodiment of the first aspect (in particular in one embodiment of the above first, second, third, or fourth subgroup of the first aspect, such as in any of the embodiments (1) to (30) listed above), the pH of the composition is between about 6.5 and about 8.0. For example, the pH of the composition may be between about 6.9 and about 7.9, such as between about 7.0 and about 7.9, between about 7.1 and about 7.8, between about 7.2 and about 7.7, between about 7.3 and about 7.6, between about 7.4 and about 7.6, or about 7.5.

In one embodiment of the first aspect (in particular in one embodiment of the above first, second, third, or fourth subgroup of the first aspect, such as in any of the embodiments (1) to (30) listed above), the composition comprises water as the main component and/or the total amount of solvent(s) other than water contained in the composition is less than about 1.0% (v/v). For example, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w). In particular, if the composition comprises a cryoprotectant, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), or at least 90% (w/w). If the composition is substantially free of a cryoprotectant, the amount of water contained in the composition may be at least 95% (w/w).

Additionally or alternatively, the total amount of solvent(s) other than water contained in the composition may be less than about 1.0% (v/v), such as less than about 0.9% (v/v), less than about 0.8% (v/v), less than about 0.7% (v/v), less than about 0.6% (v/v), less than about 0.5% (v/v), less than about 0.4% (v/v), less than about 0.3% (v/v), less than about 0.2% (v/v), less than about 0.1% (v/v), less than about 0.05% (v/v), or less than about 0.01% (v/v). In this respect, a cryoprotectant which is liquid under normal conditions will not be considered as a solvent other than water but as cryoprotectant. In other words, the above optional limitation that the total amount of solvent(s) other than water contained in the composition may be less than about 1.0% (v/v) does not apply to cryoprotectants which are liquids under normal conditions.

In one embodiment of the first aspect (in particular in one embodiment of the above first, second, third, or fourth subgroup of the first aspect, such as in any of the embodiments (1) to (30) listed above), the osmolality of the composition is at most about 400×10⁻³ osmol/kg, such as at most about 390×10⁻³ osmol/kg, at most about 380×10⁻³ osmol/kg, at most about 370×10⁻³ osmol/kg, at most about 360×10⁻³ osmol/kg, at most about 350×10⁻³ osmol/kg at most about 340×10⁻³ osmol/kg, at most about 330×10⁻³ osmol/kg, at most about 320×10⁻³ osmol/kg, at most about 310×10⁻³ osmol/kg, or at most about 300×10⁻³ osmol/kg. If the composition does not comprise a cryoprotectant, the osmolality of the composition may be below 300×10⁻³ osmol/kg, such as at most about 250×10⁻³ osmol/kg, at most about 200×10⁻³ osmol/kg, at most about 150×10⁻³ osmol/kg, at most about 100×10⁻³ osmol/kg, at most about 50×10⁻³ osmol/kg, at most about 40×10⁻³ osmol/kg, or at most about 30×10⁻³ osmol/kg.

If the composition comprises a cryoprotectant, it is preferred that the main part of the osmolality of the composition is provided by the cryoprotectant. For example, the cryoprotectant may provide at least 50%, such as at least 55%, at least 60%, at least 65%, at least 70%, at least 75%, at least 80%, at least 85%, or at least 90%, of the osmolality of the composition.

In one embodiment of the first aspect (in particular in one embodiment of the above first, second, third, or fourth subgroup of the first aspect, such as in any of the embodiments (1) to (30) listed above), the concentration of the RNA in the composition is about 5 mg/l to about 150 mg/l. For example, the concentration of the RNA in the composition may be about 10 mg/l to about 140 mg/l, such as about 20 mg/l to about 130 mg/l, about 25 mg/l to about 125 mg/l, about 30 mg/l to about 120 mg/l, about 35 mg/l to about 115 mg/l, about 40 mg/l to about 110 mg/l, about 45 mg/l to about 105 mg/l, or about 50 mg/l to about 100 mg/l.

In one embodiment of the first aspect (in particular in one embodiment of the above first, second, third, or fourth subgroup of the first aspect, such as in any of the embodiments (1) to (30) listed above), the buffer substance is Tris and its protonated form, the pH of the composition is between about 6.5 and about 8.0, and the concentration of the RNA in the composition is about 5 mg/l to about 150 mg/l. In this embodiment, it is preferred that the pH of the composition is between about 6.9 and about 7.9 and the concentration of the RNA in the composition is about 25 mg/l to about 125 mg/l, such as about 30 mg/l to about 120 mg/l. In particularly preferred embodiment of the claimed composition, the buffer substance is Tris and its protonated form; the pH of the composition is between about 6.9 and about 7.9; the concentration of the RNA in the composition is about 30 mg/l to about 120 mg/l; the aqueous phase is substantially free of inorganic sulfate anions, substantially free of dibasic organic acids and substantially free of polybasic organic acids; and the composition comprises a cryoprotectant. In an alternative particularly preferred embodiment of the claimed composition, the buffer substance is Tris and its protonated form; the pH of the composition is between about 6.9 and about 7.9; the concentration of the RNA in the composition is about 30 mg/l to about 120 mg/l; the aqueous phase is substantially free of inorganic sulfate anions, substantially free of dibasic organic acids and substantially free of polybasic organic acids; and the composition is substantially free of a cryoprotectant.

In one embodiment of the first aspect (in particular in one embodiment of the above first, second, third, or fourth subgroup of the first aspect, such as in any of the embodiments (1) to (30) listed above), the cationically ionizable lipid comprises a head group which includes at least one nitrogen atom which is capable of being protonated under physiological conditions. For example, the cationically ionizable lipid may have the structure of Formula (I):

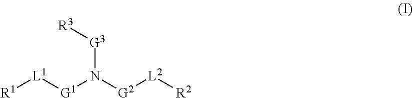

or a pharmaceutically acceptable salt, tautomer, prodrug or stereoisomer thereof, wherein L¹, L², G¹, G², G³, R¹, R², and R³ are as defined herein. Preferably, the cationically ionizable lipid is selected from the following: structures I-1 to I-36 (shown herein); and/or structures A to F (shown herein); and/or N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In a particularly preferred embodiment, the cationically ionizable lipid is the lipid having the structure 1-3.

In one embodiment of the first aspect (in particular in one embodiment of the above first, second, third, or fourth subgroup of the first aspect, such as in any of the embodiments (1) to (30) listed above), the LNPs further comprise one or more additional lipids. Preferably, the one or more additional lipids are selected from the group consisting of polymer conjugated lipids, neutral lipids, steroids, and combinations thereof. In a preferred embodiment of the first aspect (in particular in one embodiment of the above first, second, third, or fourth subgroup of the first aspect, such as in any of the embodiments (1) to (30) listed above), the LNPs comprise the cationically ionizable lipid as described herein, a polymer conjugated lipid (e.g., a pegylated lipid or a polysarcosine-lipid conjugate or a conjugate of polysarcosine and a lipid-like material), a neutral lipid (e.g., DSPC), and a steroid (e.g., cholesterol).

In one embodiment of the first aspect (in particular in one embodiment of the above first, second, third, or fourth subgroup of the first aspect, such as in any of the embodiments (1) to (30) listed above), wherein the LNPs further comprise a polymer conjugated lipid as one of the one or more additional lipids, the polymer conjugated lipid is a pegylated lipid. For example, the pegylated lipid may have the following structure:

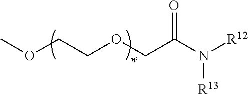

or a pharmaceutically acceptable salt, tautomer or stereoisomer thereof, wherein R¹², R¹³, and w are as defined herein.

In one embodiment of the first aspect (in particular in one embodiment of the above first, second, third, or fourth subgroup of the first aspect, such as in any of the embodiments (1) to (30) listed above), wherein the LNPs further comprise a polymer conjugated lipid as one of the one or more additional lipids, the polymer conjugated lipid is a polysarcosine-lipid conjugate or a conjugate of polysarcosine and a lipid-like material. For example, the polysarcosine-lipid conjugate or conjugate of polysarcosine and a lipid-like material may be a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

In one embodiment of the first aspect (in particular in one embodiment of the above first, second, third, or fourth subgroup of the first aspect, such as in any of the embodiments (1) to (30) listed above), wherein the LNPs further comprise a neutral lipid as one of the one or more additional lipids, the neutral lipid is a phospholipid. Such phospholipid is preferably selected from the group consisting of phosphatidylcholines, phosphatidylethanolamines, phosphatidylglycerols, phosphatidic acids, phosphatidylserines and sphingomyelins. Particular examples of phospholipids include distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In a particularly preferred embodiment, the neutral lipid is DSPC.

In one embodiment of the first aspect (in particular in one embodiment of the above first, second, third, or fourth subgroup of the first aspect, such as in any of the embodiments (1) to (30) listed above), wherein the LNPs further comprise a steroid as one of the one or more additional lipids, the steroid is a sterol such as cholesterol.

In one embodiment of the first aspect (in particular in one embodiment of the above first, second, third, or fourth subgroup of the first aspect, such as in any of the embodiments (1) to (30) listed above), the aqueous phase does not comprise a chelating agent.

In one embodiment of the first aspect (in particular in one embodiment of the above first, second, third, or fourth subgroup of the first aspect, such as in any of the embodiments (1) to (30) listed above), the LNPs comprise at least about 75% of the RNA comprised in the composition. For example, the LNPs may comprise at least about 76%, such as at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% of the RNA comprised in the composition.

In one embodiment of the first aspect (in particular in one embodiment of the above first, second, third, or fourth subgroup of the first aspect, such as in any of the embodiments (1) to (30) listed above), the RNA (such as mRNA) is encapsulated within or associated with the LNPs.

In one embodiment of the first aspect (in particular in one embodiment of the above first, second, third, or fourth subgroup of the first aspect, such as in any of the embodiments (1) to (30) listed above), the RNA (such as mRNA) comprises a modified nucleoside in place of uridine. For example, the modified nucleoside may be selected from pseudouridine (ψ), N1-methyl-pseudouridine (m1ψ), and 5-methyl-uridine (m5U).

In one embodiment of the first aspect (in particular in one embodiment of the above first, second, third, or fourth subgroup of the first aspect, such as in any of the embodiments (1) to (30) listed above), the RNA (such as mRNA) comprises one or more of the following (a) a 5′ cap, such as a cap1 or cap2 structure; (b) a 5′ UTR; (c) a 3′ UTR; and (d) a poly-A sequence, such as a poly-A sequence comprising at least 100 nucleotides, wherein the poly-A sequence preferably is an interrupted sequence of A nucleotides.

In one preferred embodiment of the first aspect (in particular in one embodiment of the above first, second, third, or fourth subgroup of the first aspect, such as in any of the embodiments (1) to (30) listed above), the RNA (such as mRNA) encodes one or more polypeptides. For example, the one or more polypeptides may comprise an epitope for inducing an immune response against an antigen in a subject.

In a preferred embodiment, the RNA (such as mRNA) comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof. In one embodiment of the first aspect (in particular in one embodiment of the above first, second, third, or fourth subgroup of the first aspect, such as in any of the embodiments (1) to (30) listed above), the RNA (such as mRNA) comprises an ORF encoding a full-length SARS-CoV2 S protein variant with proline residue substitutions at positions 986 and 987 of SEQ ID NO: 1. For example, the SARS-CoV2 S protein variant may have at least 80% identity to SEQ ID NO: 7.

In one preferred embodiment of the first aspect (in particular in one embodiment of the above first, second, third, or fourth subgroup of the first aspect, such as in any of the embodiments (1) to (30) listed above), the composition is in frozen form. Preferably, the RNA integrity after thawing the frozen composition is at least 50%, such as at least 52%, at least 54%, at least 55%, at least 56%, at least 58%, or at least 60%, e.g., after thawing the frozen composition which has been stored at −20° C. Additionally or alternatively, the size (Z_{average}) (and/or size distribution and/or polydispersity index (PDI)) of the LNPs after thawing the frozen composition is equal to the size (Z_{average}) (and/or size distribution and/or PDI) of the LNPs before the composition has been frozen. In one embodiment, the size (Z_{average}) of the LNPs after thawing the frozen composition is between about 50 nm and about 500 nm, preferably between about 40 nm and about 200 nm, more preferably between about 40 nm and about 120 nm. In one embodiment, the PDI of the LNPs after thawing the frozen composition is less than 0.3, preferably less than 0.2, more preferably less than 0.1. In one embodiment, the size (Z_{average}) of the LNPs after thawing the frozen composition is between about 50 nm and about 500 nm, preferably between about 40 nm and about 200 nm, more preferably between about 40 nm and about 120 nm, and the size (Z_{average}) (and/or size distribution and/or PDI) of the LNPs after thawing the frozen composition is equal to the size (Z_{average}) (and/or size distribution and/or PDI) of the LNPs before freezing. In one embodiment, the size (Z_{average}) of the LNPs after thawing the frozen composition is between about 50 nm and about 500 nm, preferably between about 40 nm and about 200 nm, more preferably between about 40 nm and about 120 nm, and the PDI of the LNPs after thawing the frozen composition is less than 0.3 (preferably less than 0.2, more preferably less than 0.1).

In one alternative preferred embodiment of the first aspect (in particular in one embodiment of the above first, second, third, or fourth subgroup of the first aspect, such as in any of the embodiments (1) to (30) listed above), the composition is in liquid form. Preferably, the RNA integrity of the liquid composition, when stored, e.g., at 0° C. or higher for at least one week, is sufficient to produce the desired effect, e.g., to induce an immune response. For example, the RNA integrity of the liquid composition, when stored, e.g., at 0° C. or higher for at least one week, may be at least 50%, such as at least 52%, at least 54%, at least 55%, at least 56%, at least 58%, or at least 60%. Additionally or alternatively, the size (Z_{average}) (and/or size distribution and/or polydispersity index (PDI)) of the LNPs of the liquid composition, when stored, e.g., at 0° C. or higher for at least one week, is sufficient to produce the desired effect, e.g., to induce an immune response. For example, the size (Z_{averages}) (and/or size distribution and/or polydispersity index (PDI)) of the LNPs of the liquid composition, when stored, e.g., at 0° C. or higher for at least one week, is equal to the size (Z_{average}) (and/or size distribution and/or PDI) of the LNPs of the initial composition, i.e., before storage. In one embodiment, the size (Z_{average}) of the LNPs after storage of the liquid composition e.g., at 0° C. or higher for at least one week is between about 50 nm and about 500 nm, preferably between about 40 nm and about 200 nm, more preferably between about 40 nm and about 120 nm. In one embodiment, the PDI of the LNPs after storage of the liquid composition e.g., at 0° C. or higher for at least one week is less than 0.3, preferably less than 0.2, more preferably less than 0.1. In one embodiment, the size (Z_{average}) of the LNPs after storage of the liquid composition e.g., at 0° C. or higher for at least one week is between about 50 nm and about 500 nm, preferably between about 40 nm and about 200 nm, more preferably between about 40 nm and about 120 nm, and the size (Z_{average}) (and/or size distribution and/or PDI) of the LNPs after storage of the liquid composition e.g., at 0° C. or higher for at least one week is equal to the size (Z_{average}) (and/or size distribution and/or PDI) of the LNPs before storage. In one embodiment, the size (Z_{average}) of the LNPs after storage of the liquid composition e.g., at 0° C. or higher for at least one week is between about 50 nm and about 500 nm, preferably between about 40 nm and about 200 nm, more preferably between about 40 nm and about 120 nm, and the PDI of the LNPs after storage of the liquid composition e.g., at 0° C. or higher for at least one week is less than 0.3 (preferably less than 0.2, more preferably less than 0.1).

In a second aspect, the present disclosure provides a method of preparing a composition comprising LNPs dispersed in a final aqueous phase, wherein the LNPs comprise a cationically ionizable lipid and RNA; the final aqueous phase comprises a buffer system comprising a final buffer substance and a final monovalent anion, the final buffer substance being selected from the group consisting of Tris and its protonated form, Bis-Tris-methane and its protonated form, and TEA and its protonated form, and the final monovalent anion being selected from the group consisting of chloride, acetate, glycolate, lactate, the anion of MES, the anion of MOPS, and the anion of HEPES; the concentration of the final buffer substance in the composition is at most about 25 mM; and the final aqueous phase is substantially free of inorganic phosphate anions, substantially free of citrate anions, and substantially free of anions of EDTA; wherein the method comprises:
(I) preparing a formulation comprising LNPs dispersed in the final aqueous phase, wherein the LNPs comprise the cationically ionizable lipid and RNA; and
(II) optionally freezing the formulation to about −10° C. or below,
thereby obtaining the composition,
wherein step (I) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing an intermediate formulation comprising the LNPs dispersed in an intermediate aqueous phase comprising the first buffer system; and
(d) filtrating the first intermediate formulation prepared under (c) using a final aqueous buffer solution comprising the final buffer system,
thereby preparing the formulation comprising the LNPs dispersed in the final aqueous phase.

As demonstrated in the present application, using a particular buffer system based on the above specified buffer substances, in particular Tris and its protonated form, instead of PBS in a composition comprising LNPs inhibits the formation of a very stable folded form of RNA. Furthermore, the present application demonstrates that, surprisingly, by simply lowering the concentration of the buffer substance in a composition comprising LNPs and a buffer system, wherein the LNPs comprise a cationically ionizable lipid and RNA, it is possible to obtain an LNP RNA composition having improved RNA integrity after a freeze/thaw cycle compared to a composition comprising the same buffer substance in a concentration of 50 mM. Thus, the composition prepared by the claimed method provides improved stability, can be stored in a temperature range compliant to regular technologies in pharmaceutical practice, and provides a ready-to-use preparation.

In a particularly preferred embodiment of the second aspect, the final buffer substance is Tris and its protonated form, i.e., a mixture of Tris and its protonated form.

In one embodiment of the second aspect, the final monovalent anion is selected from the group consisting of chloride, acetate, glycolate, lactate, morpholinoethanesulfonate, and 3-(N-morpholino)propanesulfonate, or from the group consisting of chloride, acetate, glycolate, lactate, morpholinoethanesulfonate, and 2-[4-(2-hydroxyethyl)piperazin-1-yl]ethanesulfonate, preferably from the group consisting of chloride, acetate, lactate, and morpholinoethanesulfonate, more preferably from the group consisting of chloride, acetate, and morpholinoethanesulfonate, or from the group consisting of chloride, acetate, and lactate, such as chloride or acetate.

In one embodiment of the second aspect, the final buffer substance is Tris and its protonated form and the final monovalent anion is selected from the group consisting of chloride, acetate, glycolate, lactate, morpholinoethanesulfonate, and 3-(N-morpholino)propanesulfonate, or from the group consisting of chloride, acetate, glycolate, lactate, morpholinoethanesulfonate, and 2-[4-(2-hydroxyethyl)piperazin-1-yl]ethanesulfonate, preferably from the group consisting of chloride, acetate, lactate, and morpholinoethanesulfonate, more preferably from the group consisting of chloride, acetate, lactate, and morpholinoethanesulfonate, more preferably from the group consisting of chloride, acetate, and morpholinoethanesulfonate, such as chloride or acetate

In one embodiment, in particular if it is desired to prepare a composition in frozen form, the method of the second aspect comprises (I) freezing the formulation to about −10° C. or below. Thus, in this embodiment, conducting the method of the second aspect results in a composition in frozen form.

In an alternative embodiment, in particular if it is desired to prepare a composition in liquid form, the method of the second aspect does not comprises step (II). Thus, in this embodiment, conducting the method of the second aspect results in a composition in liquid form.

In one embodiment of the second aspect, step (1) further comprises one or more steps selected from diluting and filtrating, such as tangential flow filtrating and diafiltrating, after step (c). For example, a diluting step may comprise adding a dilution solution to an intermediate formulation. Such dilution solution may comprise one or more additional compounds and optionally the final buffer system, wherein the one or more additional compounds may comprise a cryoprotectant. The one or more filtrating steps (including steps (d), (f), (g′), and (h′)) may be used to remove unwanted compounds (e.g., ethanol and/or one or more di- and/or polybasic organic acids) from the intermediate formulation and/or for increasing the RNA concentration of the intermediate formulation and/or for changing the pH and/or the buffer system of the intermediate formulation. To this end, an aqueous buffer solution can be used, which does not contain the unwanted compounds (such that the unwanted compounds are washed out from the intermediate formulation and into the aqueous buffer solution) and/or which is hypertonic compared to the aqueous buffer solution (such that water flows from the intermediate formulation to the aqueous buffer solution) and/or which has a pH and/or buffer system other than the pH and/or buffer system of the intermediate formulation.

In a preferred embodiment of the second aspect, step (I) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f) once or two or more times, wherein the further intermediate formulation comprising the LNPs dispersed in the further aqueous phase comprising the further buffer system obtained after step (f) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f) is absent, or the further intermediate formulation obtained in step (f), if step (f) is present and step (g′) is not present, or the further intermediate formulation obtained after step (g′), if steps (f) and (g′) are present, using a final aqueous buffer solution comprising the final buffer system and having a pH of at least 6.0; and
(i′) optionally diluting the formulation obtained in step (h′) with a dilution solution; thereby preparing the formulation comprising the LNPs dispersed in the final aqueous phase.

In one embodiment of the second aspect, the concentration of the final buffer substance, in particular the total concentration of Tris and its protonated form, in the composition is at most about 20 mM, such as at most about 19 mM, at most about 18 mM, at most about 17 mM, at most about 16 mM, at most about 15 mM, at most about 14 mM, at most about 13 mM, at most about 12 mM, at most about 11 mM, or at most about 10 mM. In one embodiment, the lower limit of the final buffer substance, in particular Tris and its protonated form, in the composition is at least about 1 mM, preferably at least about 2 mM, such as at least about 3 mM, at least about 4 mM, at least about 5 mM, at least about 6 mM, at least about 7 mM, at least about 8 mM, or at least about 9 mM. For example, the concentration of the final buffer substance, in particular the total concentration of Tris and its protonated form, in the composition may be between about 1 mM and about 20 mM, such as between about 2 mM and about 15 mM, between about 5 mM and about 14 mM, between about 7 mM and about 13 mM, between about 8 mM and about 12 mM, between about 9 mM and about 11 mM, such as about 10 mM.

In one embodiment of the second aspect, the final aqueous phase is substantially free of inorganic sulfate anions and/or carbonate anions and/or dibasic organic acid anions and/or polybasic organic acid anions. In a first subgroup of the second aspect, at least one of these criteria applies. For example, in one embodiment of this first subgroup of the second aspect, the final aqueous phase is substantially free of inorganic sulfate anions. In a further embodiment of this first subgroup of the second aspect, the final aqueous phase is substantially free of carbonate anions. In a further embodiment of this first subgroup of the second aspect, the final aqueous phase is substantially free of dibasic organic acid anions. In a further embodiment of this first subgroup of the second aspect, the final aqueous phase is substantially free of polybasic organic acid anions.

In a second subgroup of the second aspect, at least two of the above criteria apply. For example, in one embodiment of this second subgroup of the second aspect, the final aqueous phase is substantially free of inorganic sulfate anions and substantially free of carbonate anions. In a further embodiment of this second subgroup of the second aspect, the final aqueous phase is substantially free of inorganic sulfate anions and substantially free of dibasic organic acid anions. In a further embodiment of this second subgroup of the second aspect, the final aqueous phase is substantially free of inorganic sulfate anions and substantially free of polybasic organic acid anions. In a further embodiment of this second subgroup of the second aspect, the final aqueous phase is substantially free of carbonate anions and substantially free of dibasic organic acid anions. In a further embodiment of this second subgroup of the second aspect, the final aqueous phase is substantially free of carbonate anions and substantially free of polybasic organic acid anions. In a further embodiment of this second subgroup of the second aspect, the final aqueous phase is substantially free of dibasic organic acid anions and substantially free of polybasic organic acid anions.

In a third subgroup of the second aspect, at least three of the above criteria apply. For example, in one embodiment of this third subgroup of the second aspect, the final aqueous phase is substantially free of inorganic sulfate anions, substantially free of carbonate anions and substantially free of dibasic organic acid anions. In a further embodiment of this third subgroup of the second aspect, the final aqueous phase is substantially free of inorganic sulfate anions, substantially free of carbonate anions and substantially free of polybasic organic acid anions. In a further embodiment of this third subgroup of the second aspect, the final aqueous phase is substantially free of inorganic sulfate anions, substantially free of dibasic organic acid anions and substantially free of polybasic organic acid anions. In a further embodiment of this third subgroup of the second aspect, the final aqueous phase is substantially free of carbonate anions, substantially free of dibasic organic acid anions and substantially free of polybasic organic acid anions.

In a fourth subgroup of the second aspect, at least four of the above criteria apply. I.e., in this fourth subgroup of the second aspect, the final aqueous phase is substantially free of inorganic sulfate anions, substantially free of carbonate anions, substantially free of dibasic organic acid anions and substantially free of polybasic organic acid anions.

In one embodiment of the second aspect (in particular in one embodiment of the above first, second, third, or fourth subgroup of the second aspect), the formulation obtained in step (I) and/or the composition comprise(s) a cryoprotectant. In an alternative embodiment of the second aspect (in particular in one embodiment of the above first, second, third, or fourth subgroup of the second aspect), the formulation obtained in step (1) and/or the composition is substantially free of a cryoprotectant. Thus, particular examples of these embodiments are the following:
(1) the final aqueous phase is substantially free of inorganic sulfate anions, and the formulation obtained in step (1) and/or the composition comprise(s) a cryoprotectant;
(2) the final aqueous phase is substantially free of carbonate anions, and the formulation obtained in step (I) and/or the composition comprise(s) a cryoprotectant;
(3) the final aqueous phase is substantially free of dibasic organic acid anions, the formulation obtained in step (I) and/or the composition and comprise(s) a cryoprotectant;
(4) the final aqueous phase is substantially free of polybasic organic acid anions, and the formulation obtained in step (I) and/or the composition comprise(s) a cryoprotectant;
(5) the final aqueous phase is substantially free of inorganic sulfate anions and substantially free of carbonate anions, and the formulation obtained in step (I) and/or the composition comprise(s) a cryoprotectant;
(6) the final aqueous phase is substantially free of inorganic sulfate anions and substantially free of dibasic organic acid anions, and the formulation obtained in step (I) and/or the composition comprise(s) a cryoprotectant;
(7) the final aqueous phase is substantially free of inorganic sulfate anions and substantially free of polybasic organic acid anions, and the formulation obtained in step (I) and/or the composition comprise(s) a cryoprotectant;
(8) the final aqueous phase is substantially free of carbonate anions and substantially free of dibasic organic acid anions, and the formulation obtained in step (I) and/or the composition comprise(s) a cryoprotectant;
(9) the final aqueous phase is substantially free of carbonate anions and substantially free of polybasic organic acid anions, and the formulation obtained in step (I) and/or the composition comprise(s) a cryoprotectant;
(10) the final aqueous phase is substantially free of dibasic organic acid anions and substantially free of polybasic organic acid anions, and the formulation obtained in step (I) and/or the composition comprise(s) a cryoprotectant;
(11) the final aqueous phase is substantially free of inorganic sulfate anions, substantially free of carbonate anions and substantially free of dibasic organic acid anions, and the formulation obtained in step (I) and/or the composition comprise(s) a cryoprotectant;
(12) the final aqueous phase is substantially free of inorganic sulfate anions, substantially free of carbonate anions and substantially free of polybasic organic acid anions, and the formulation obtained in step (I) and/or the composition comprise(s) a cryoprotectant;
(13) the final aqueous phase is substantially free of inorganic sulfate anions, substantially free of dibasic organic acid anions and substantially free of polybasic organic acid anions, and the formulation obtained in step (I) and/or the composition comprise(s) a cryoprotectant;
(14) the final aqueous phase is substantially free of carbonate anions, substantially free of dibasic organic acid anions and substantially free of polybasic organic acid anions, and the formulation obtained in step (I) and/or the composition comprise(s) a cryoprotectant;
(15) the final aqueous phase is substantially free of inorganic sulfate anions, substantially free of carbonate anions, substantially free of dibasic organic acid anions and substantially free of polybasic organic acid anions, and the formulation obtained in step (I) and/or the composition comprise(s) a cryoprotectant;
(16) the final aqueous phase is substantially free of inorganic sulfate anions, and the formulation obtained in step (I) and/or the composition is/are substantially free of a cryoprotectant;
(17) the final aqueous phase is substantially free of carbonate anions, and the formulation obtained in step (I) and/or the composition is/are substantially free of a cryoprotectant;
(18) the final aqueous phase is substantially free of dibasic organic acid anions, and the formulation obtained in step (1) and/or the composition is/are substantially free of a cryoprotectant;
(19) the final aqueous phase is substantially free of polybasic organic acid anions, and the formulation obtained in step (I) and/or the composition is/are substantially free of a cryoprotectant;
(20) the final aqueous phase is substantially free of inorganic sulfate anions and substantially free of carbonate anions, and the formulation obtained in step (I) and/or the composition is/are substantially free of a cryoprotectant;
(21) the final aqueous phase is substantially free of inorganic sulfate anions and substantially free of dibasic organic acid anions, and the formulation obtained in step (1) and/or the composition is/are substantially free of a cryoprotectant;
(22) the final aqueous phase is substantially free of inorganic sulfate anions and substantially free of polybasic organic acid anions, and the formulation obtained in step (1) and/or the composition is/are substantially free of a cryoprotectant;
(23) the final aqueous phase is substantially free of carbonate anions and substantially free of dibasic organic acid anions, and the formulation obtained in step (1) and/or the composition is/are substantially free of a cryoprotectant;
(24) the final aqueous phase is substantially free of carbonate anions and substantially free of polybasic organic acid anions, and the formulation obtained in step (I) and/or the composition is/are substantially free of a cryoprotectant;
(25) the final aqueous phase is substantially free of dibasic organic acid anions and substantially free of polybasic organic acid anions, and the formulation obtained in step (I) and/or the composition is/are substantially free of a cryoprotectant;
(26) the final aqueous phase is substantially free of inorganic sulfate anions, substantially free of carbonate anions and substantially free of dibasic organic acid anions, and the formulation obtained in step (1) and/or the composition is/are substantially free of a cryoprotectant;
(27) the final aqueous phase is substantially free of inorganic sulfate anions, substantially free of carbonate anions and substantially free of polybasic organic acid anions, and the formulation obtained in step (I) and/or the composition is/are substantially free of a cryoprotectant;
(28) the final aqueous phase is substantially free of inorganic sulfate anions, substantially free of dibasic organic acid anions and substantially free of polybasic organic acid anions, and the formulation obtained in step (I) and/or the composition is/are substantially free of a cryoprotectant;
(29) the final aqueous phase is substantially free of carbonate anions, substantially free of dibasic organic acid anions and substantially free of polybasic organic acid anions, and the formulation obtained in step (I) and/or the composition is/are substantially free of a cryoprotectant;
(30) the final aqueous phase is substantially free of inorganic sulfate anions, substantially free of carbonate anions, substantially free of dibasic organic acid anions and substantially free of polybasic organic acid anions, and the formulation obtained in step (I) and/or the composition is/are substantially free of a cryoprotectant.

In one embodiment of the second aspect (in particular in one embodiment of the above first, second, third, or fourth subgroup of the second aspect, such as in any of the embodiments (1) to (30) listed above), wherein formulation obtained in step (I) and/or the composition comprise(s) a cryoprotectant, said cryoprotectant comprises one or more compounds selected from the group consisting of carbohydrates and sugar alcohols. For example, the cryoprotectant may be selected from the group consisting of sucrose, glucose, glycerol, sorbitol, and a combination thereof. In a preferred embodiment of the second aspect (in particular in one embodiment of the above first, second, third, or fourth subgroup of the second aspect, such as in any of the embodiments (1) to (30) listed above), the formulation obtained in step (I) and/or the composition comprise(s) sucrose and/or glycerol as cryoprotectant.

In one embodiment of the second aspect (in particular in one embodiment of the above first, second, third, or fourth subgroup of the second aspect, such as in any of the embodiments (1) to (30) listed above), wherein the formulation obtained in step (I) and/or the composition comprise(s) a cryoprotectant, the concentration of the cryoprotectant in the formulation and/or composition is at least 1% w/v, such as at least 2% w/v, at least 3% w/v, at least 4% w/v, at least 5% w/v, at least 6% w/v, at least 7% w/v, at least 8% w/v or at least 9% w/v. In one embodiment, the concentration of the cryoprotectant in the formulation and/or composition is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In one embodiment, the concentration of the cryoprotectant in the formulation and/or composition is 1% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In one embodiment of the second aspect (in particular in one embodiment of the above first, second, third, or fourth subgroup of the second aspect, such as in any of the embodiments (1) to (30) listed above), the formulation and/or composition comprise(s) a cryoprotectant (in particular, sucrose and/or glycerol) in a concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v. For example, the method of the second aspect may comprise a diluting step using a dilution solution, wherein the dilution solution comprises a sufficient amount of a cryoprotectant in order to achieve the above concentrations of cryoprotectant in the formulation obtained in step (I) and/or the composition.

In one embodiment of the second aspect (in particular in one embodiment of the above first, second, third, or fourth subgroup of the second aspect, such as in any of the embodiments (1) to (30) listed above), wherein the formulation obtained in step (I) and/or the composition comprise(s) a cryoprotectant, the cryoprotectant is present in a concentration resulting in an osmolality of the composition in the range of from about 50×10⁻³ osmol/kg to about 400×10⁻³ osmol/kg (such as from about 50×10⁻³ osmol/kg to about 390×10⁻³ osmol/kg, from about 60×10⁻³ osmol/kg to about 380×10⁻³ osmol/kg, from about 70×10⁻³ osmol/kg to about 370×10⁻³ osmol/kg, from about 80×10⁻³ osmol/kg to about 360×10⁻³ osmol/kg, from about 90×10⁻³ osmol/kg to about 350×10⁻³ osmol/kg, from about 100×10⁻³ osmol/kg to about 340×10⁻³ osmol/kg, from about 120×10⁻³ osmol/kg to about 330×10⁻³ osmol/kg, from about 140×10⁻³ osmol/kg to about 320×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the formulation/composition. For example, the method of the second aspect may comprise a diluting step using a dilution solution, wherein the dilution solution comprises a sufficient amount of a cryoprotectant in order to achieve the above osmolality values in the formulation obtained in step (I) and/or the composition.

In one embodiment of the second aspect (in particular in one embodiment of the above first, second, third, or fourth subgroup of the second aspect, such as in any of the embodiments (1) to (30) listed above), in particular in those embodiments of the second aspect, where the final buffer substance is Tris and its protonated form, the final monovalent anion is selected from the group consisting of chloride, acetate, glycolate, and lactate, and the concentration of the final monovalent anion (in particular the total concentration of all final monovalent anions) in the composition is at most equal to the concentration of the final buffer substance in the composition. For example, the concentration of the final monovalent anion (in particular the total concentration of all final monovalent anions) in the composition may be less than the concentration of the final buffer substance in the composition. Thus, in those embodiments of the second aspect, where the concentration of the final buffer substance, in particular Tris and its protonated form, in the composition is at most about 20 mM, the concentration of the final monovalent anion (in particular the total concentration of all final monovalent anions) in the composition is at most equal to about 20 mM, e.g., less than 20 mM. Generally, the concentration of the monovalent anion, such as chloride and/or acetate (in particular the total concentration of all monovalent anions) in the composition may be less than about 15 mM, such as less than about 14 mM, less than about 13 mM, less than about 12 mM, less than about 11 mM, less than about 10 mM, less than about 9 mM, less than about 8 mM, less than about 7 mM, less than about 6 mM, or less than about 5 mM. In one embodiment, the chloride concentration in the composition is as defined above (e.g., less than about 15 mM, etc.) and the composition does not comprise acetate. In an alternative embodiment, the acetate concentration in the composition is as defined above (e.g., less than about 15 mM, etc.) and the composition does not comprise chloride.

In one embodiment of the second aspect (in particular in one embodiment of the above first, second, third, or fourth subgroup of the second aspect, such as in any of the embodiments (1) to (30) listed above), in particular in those embodiments of the second aspect, where the final buffer substance is Tris and its protonated form, the sodium concentration in the aqueous phase and/or composition is less than 20 mM, such as less than about 15 mM, e.g., less than about 14 mM, less than about 13 mM, less than about 12 mM, less than about 11 mM, less than about 10 mM, less than about 9 mM, less than about 8 mM, less than about 7 mM, less than about 6 mM, or less than about 5 mM.

In one embodiment of the second aspect (in particular in one embodiment of the above first, second, third, or fourth subgroup of the second aspect, such as in any of the embodiments (1) to (30) listed above), in particular in those embodiments of the second aspect, where the final buffer substance is Tris and its protonated form, the final monovalent anion is selected from the group consisting of the anions of MES, MOPS and HEPES, and the concentration of the final monovalent anion (in particular the total concentration of all final monovalent anions) in the composition is at least equal to the concentration of the final buffer substance in the composition. For example, the concentration of the final monovalent anion (in particular the total concentration of all final monovalent anions) in the composition may be higher than the concentration of the final buffer substance in the composition. Thus, in those embodiments of the second aspect, where the concentration of the final buffer substance, in particular Tris and its protonated form, in the composition is at most about 20 mM, the concentration of the final monovalent anion (in particular the total concentration of all final monovalent anions) in the composition is at least equal to about 20 mM, e.g., higher than 20 mM. Generally, the concentration of the final monovalent anion (in particular the total concentration of all final monovalent anions) in the composition may be higher than about 20 mM, such as higher than about 21 mM, higher than about 22 mM, higher than about 23 mM, higher than about 24 mM, higher than about 25 mM, higher than about 26 mM, higher than about 27 mM, higher than about 28 mM, higher than about 29 mM, or higher than about 30 mM, and preferably at most 50 mM, such as at most 45 mM, at most 40 mM or at most 35 mM.

In one embodiment of the second aspect (in particular in one embodiment of the above first, second, third, or fourth subgroup of the second aspect, such as in any of the embodiments (1) to (30) listed above), the pH of the final buffer system (and the pH of the composition) is between about 6.5 and about 8.0. For example, the pH of the composition may be between about 6.9 and about 7.9, such as between about 7.0 and about 7.9, between about 7.1 and about 7.8, between about 7.2 and about 7.7, between about 7.3 and about 7.6, between about 7.4 and about 7.6, or about 7.5.

In one embodiment of the second aspect (in particular in one embodiment of the above first, second, third, or fourth subgroup of the second aspect, such as in any of the embodiments (1) to (30) listed above), the first buffer system (and the pH of the RNA solution obtained in step (a)) has a pH of below 6.0, preferably at most about 5.5, such as at most about 5.0, at most about 4.9, at most about 4.8, at most about 4.7, at most about 4.6, or at most about 4.5. For example, the pH of first buffer system (and the pH of the RNA solution obtained in step (a)) may be between about 3.5 and about 5.9, such as between about 4.0 and about 5.5, or between about 4.5 and about 5.0. To this end, the RNA solution obtained in step (a) may further comprises one or more di- and/or polybasic organic acids (e.g., citrate anions and/or anions of EDTA). In this embodiment, it is preferred that step (d) is conducted under conditions which remove the one or more di- and/or polybasic organic acids resulting in the formulation comprising the LNPs dispersed in final aqueous phase with the final aqueous phase being substantially free of the one or more di- and/or polybasic organic acids. For example, such conditions can include subjecting the intermediate formulation comprising the LNPs dispersed in the intermediate aqueous phase obtained in step (c) to at least one step of filtrating, such as tangential flow filtrating or diafiltrating, using a final buffer solution comprising the final buffer system (i.e., the final buffer substance and the final monovalent anion), wherein the final buffer solution does not contain the one or more di- and/or polybasic organic acids (and preferably does not contain ethanol). Alternatively, such conditions can include (i) subjecting the intermediate formulation comprising the LNPs dispersed in the intermediate aqueous phase obtained in step (c)(i.e., a first intermediate formulation) to at least one step of filtrating, such as tangential flow filtrating or diafiltrating, using a further aqueous buffer solution comprising a further buffer system, thereby preparing a further intermediate formulation comprising the LNPs dispersed in a further aqueous phase comprising the further buffer system, wherein the further buffer system of the further aqueous buffer solution may be identical to or different from the buffer system used in step (a); (ii) optionally repeating step (i) once or two or more times, wherein the further intermediate formulation comprising the LNPs dispersed in the further aqueous phase obtained after step (i) of one cycle is used as the first intermediate formulation of the next cycle, wherein in each cycle the further buffer system of the further aqueous buffer solution may be identical to or different from the first buffer system used in step (a); and (iii) subjecting the intermediate formulation obtained in step (i) (if step (ii) is not present), or the intermediate formulation obtained in step (ii) (if step (ii) is present) to at least one step of filtrating, such as tangential flow filtrating or diafiltrating, using the final aqueous buffer solution, wherein at least one of the intermediate and final aqueous buffer solutions (preferably all intermediate and final aqueous buffer solutions) does not contain the one or more di- and/or polybasic organic acids (and preferably does not contain ethanol.

Similarly, in one embodiment of the second aspect (in particular in one embodiment of the above first, second, third, or fourth subgroup of the second aspect, such as in any of the embodiments (1) to (30) listed above), where step (I) comprises steps (a′) to (e′) and (h′) (and optionally one or more of steps (f), (g′) and (i′)), the first aqueous buffer solution (and the pH of the RNA solution obtained under step (c′)) has a pH of below 6.0, preferably at most about 5.5, such as at most about 5.0, at most about 4.9, at most about 4.8, at most about 4.7, at most about 4.6, or at most about 4.5. For example, the pH of the first aqueous buffer solution (and the pH of the RNA solution obtained under step (c′)) may be between about 3.5 and about 5.9, such as between about 4.0 and about 5.5, or between about 4.5 and about 5.0. To this end, the first aqueous buffer solution provided under (b′) (and the first aqueous phase) may further comprises one or more di- and/or polybasic organic acids (e.g., citrate anions and/or anions of EDTA).

In this embodiment, it is preferred that least one of steps (f) to (h′) is conducted under conditions which remove the one or more di- and/or polybasic organic acids from the first intermediate formulation and/or from the further intermediate formulation resulting in a further inter formulation comprising the LNPs dispersed in a further aqueous phase or in the final aqueous phase with the further and/or final aqueous phase being substantially free of the one or more di- and/or polybasic organic acids. For example, such conditions can include using a further aqueous buffer solution and/or a final buffer solution, wherein at least one of the further aqueous buffer solution(s) and the final buffer solution (preferably all of the further aqueous buffer solution(s) and the final buffer solution) does not contain the one or more di- and/or polybasic organic acids (and preferably does not contain ethanol). In one embodiment, the filtrating steps can be tangential flow filtrating or diafiltrating, preferably tangential flow filtrating.

In one embodiment of the second aspect (in particular in one embodiment of the above first, second, third, or fourth subgroup of the second aspect, such as in any of the embodiments (1) to (30) listed above), the first buffer system used in step (a) comprises the final buffer substance and the final monovalent anion used in step (d), preferably the buffer system and pH of the first buffer system used in step (a) are identical to the buffer system and pH of the final aqueous buffer solution used in step (d).

For example, only one aqueous buffer solution is used in this embodiment of the second aspect.

Similarly, in one embodiment of the second aspect (in particular in one embodiment of the above first, second, third, or fourth subgroup of the second aspect, such as in any of the embodiments (1) to (30) listed above), where step (I) comprises steps (a′) to (e′) and (h′) (and optionally one or more of steps (f′), (g′) and (i′)), each of the first buffer system and every further buffer system used in steps (b′), (f) and (g′) comprises the final buffer substance and the final monovalent anion used in step (h′), preferably the buffer system and pH of each of the first aqueous buffer solution and of every further aqueous buffer solution used in steps (b′), (f′) and (g′) are identical to the buffer system and pH of the final aqueous buffer solution. For example, the aqueous buffer solutions used in steps (b′), (f), if present, (g′), if present, and (h′) of this embodiment of the second aspect are identical.

In one embodiment of the second aspect (in particular in one embodiment of the above first, second, third, or fourth subgroup of the second aspect, such as in any of the embodiments (1) to (30) listed above), the formulation and/or composition comprise(s) water as the main component and/or the total amount of solvent(s) other than water contained in the composition is less than about 1.0% (v/v). For example, the amount of water contained in the formulation and/or composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w).

In particular, if the formulation and/or composition comprise(s) a cryoprotectant, the amount of water contained in the formulation and/or composition comprise(s) may be at least 50% (w/w), such as at least at least 55% (w/w), at least 60% (w/w), at least 65% (w/w), at least 70% (w/w), at least 75% (w/w), at least 80% (w/w), at least 85% (w/w), or at least 90% (w/w). If the formulation and/or composition is/are substantially free of a cryoprotectant, the amount of water contained in the formulation and/or composition may be at least 95% (w/w). Additionally or alternatively, the total amount of solvent(s) other than water contained in the composition may be less than about 1.0% (v/v), such as less than about 0.9% (v/v), less than about 0.8% (v/v), less than about 0.7% (v/v), less than about 0.6% (v/v), less than about 0.5% (v/v), less than about 0.4% (v/v), less than about 0.3% (v/v), less than about 0.2% (v/v), less than about 0.1% (v/v), less than about 0.05% (v/v), or less than about 0.01% (v/v). In this respect, a cryoprotectant which is liquid under normal conditions will not be considered as a solvent other than water but as cryoprotectant. In other words, the above optional limitation that the total amount of solvent(s) other than water contained in the composition may be less than about 1.0% (v/v) does not apply to cryoprotectants which are liquids under normal conditions.

In one embodiment of the second aspect (in particular in one embodiment of the above first, second, third, or fourth subgroup of the second aspect, such as in any of the embodiments (1) to (30) listed above), the osmolality of the composition is at most about 400×10⁻³ osmol/kg, such as at most about 390×10⁻³ osmol/kg, at most about 380×10⁻³ osmol/kg, at most about 370×10⁻³ osmol/kg, at most about 360×10⁻³ osmol/kg, at most about 350×10⁻³ osmol/kg, at most about 340×10⁻³ osmol/kg, at most about 330×10⁻³ osmol/kg, at most about 320×10⁻³ osmol/kg, at most about 310×10⁻³ osmol/kg, or at most about 300×10⁻³ osmol/kg. If the composition does not comprise a cryoprotectant, the osmolality of the composition may be below 300×10⁻³ osmol/kg, such as at most about 250×10⁻³ osmol/kg, at most about 200×10⁻³ osmol/kg, at most about 150×10⁻³ osmol/kg, at most about 100×10⁻³ osmol/kg, at most about 50×10⁻³ osmol/kg, at most about 40×10⁻³ osmol/kg, or at most about 30×10⁻³ osmol/kg. If the composition comprises a cryoprotectant, it is preferred that the main part of the osmolality of the composition is provided by the cryoprotectant. For example, the cryoprotectant may provide at least 50%, such as at least 55%, at least 60%, at least 65%, at least 70%, at least 75%, at least 80%, at least 85%, or at least 90%, of the osmolality of the composition.

In one embodiment of the second aspect (in particular in one embodiment of the above first, second, third, or fourth subgroup of the second aspect, such as in any of the embodiments (1) to (30) listed above), the concentration of the RNA in the composition is about 5 mg/l to about 150 mg/l. For example, the concentration of the RNA in the composition may be about 10 mg/l to about 140 mg/l, such as about 20 mg/l to about 130 mg/l, about 25 mg/l to about 125 mg/l, about 30 mg/l to about 120 mg/l, about 35 mg/l to about 115 mg/l, about 40 mg/l to about 110 mg/l, about 45 mg/l to about 105 mg/l, or about 50 mg/l to about 100 mg/l.

In one embodiment of the second aspect (in particular in one embodiment of the above first, second, third, or fourth subgroup of the second aspect, such as in any of the embodiments (1) to (30) listed above), the final buffer substance is Tris and its protonated form, the pH of the composition is between about 6.5 and about 8.0, and the concentration of the RNA in the composition is about 5 mg/l to about 150 mg/l. In this embodiment, it is preferred that the pH of the composition is between about 6.9 and about 7.9 and the concentration of the RNA in the composition is about 25 mg/l to about 125 mg/l, such as about 30 mg/l to about 120 mg/l. In particularly preferred embodiment of the second aspect, the buffer substance is Tris and its protonated form; the pH of the composition is between about 6.9 and about 7.9; the concentration of the RNA in the composition is about 25 mg/l to about 125 mg/l, such as about 30 mg/l to about 120 mg/l; the final aqueous phase is substantially free of sulfate anions, substantially free of dibasic organic acids and substantially free of polybasic organic acids; and the composition comprises a cryoprotectant. In an alternative particularly preferred embodiment of the second aspect, the buffer substance is Tris and its protonated form; the pH of the composition is between about 6.9 and about 7.9; the concentration of the RNA in the composition is about 25 mg/l to about 125 mg/l, such as about 30 mg/l to about 120 mg/l; the final aqueous phase is substantially free of sulfate anions, substantially free of dibasic organic acids and substantially free of polybasic organic acids; and the composition is substantially free of a cryoprotectant.

In one embodiment of the second aspect (in particular in one embodiment of the above first, second, third, or fourth subgroup of the second aspect, such as in any of the embodiments (1) to (30) listed above), the cationically ionizable lipid comprises a head group which includes at least one nitrogen atom which is capable of being protonated under physiological conditions. For example, the cationically ionizable lipid may have the structure of Formula (I):

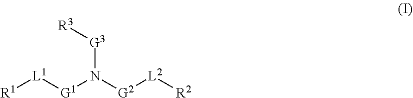

or a pharmaceutically acceptable salt, tautomer, prodrug or stereoisomer thereof, wherein L¹, L², G¹, G², G³, R¹, R², and R³ are as defined herein. Preferably, the cationically ionizable lipid is selected from the following: structures I-1 to 1-36 (shown herein); and/or structures A to F (shown herein); and/or N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In a particularly preferred embodiment, the cationically ionizable lipid is the lipid having the structure I-3.

In one embodiment of the second aspect (in particular in one embodiment of the above first, second, third, or fourth subgroup of the second aspect, such as in any of the embodiments (1) to (30) listed above), the ethanolic solution prepared in step (b) or (d′) further comprises one or more additional lipids and the LNPs further comprise the one or more additional lipids. Preferably, the one or more additional lipids are selected from the group consisting of polymer conjugated lipids, neutral lipids, steroids, and combinations thereof. In a preferred embodiment of the second aspect (in particular in one embodiment of the above first, second, third, or fourth subgroup of the second aspect, such as in any of the embodiments (1) to (30) listed above), the one or more additional lipids comprise a polymer conjugated lipid (e.g., a pegylated lipid or a polysarcosine-lipid conjugate or a conjugate of polysarcosine and a lipid-like material), a neutral lipid (e.g., DSPC), and a steroid (e.g., cholesterol), such that the LNPs comprise the cationically ionizable lipid as described herein, a polymer conjugated lipid (e.g., a pegylated lipid or a polysarcosine-lipid conjugate or a conjugate of polysarcosine and a lipid-like material), a neutral lipid (e.g., DSPC), and a steroid (e.g., cholesterol).

In one embodiment of the second aspect (in particular in one embodiment of the above first, second, third, or fourth subgroup of the second aspect, such as in any of the embodiments (1) to (30) listed above), wherein the one or more additional lipids comprise a polymer conjugated lipid, the polymer conjugated lipid is a pegylated lipid. For example, the pegylated lipid may have the following structure:

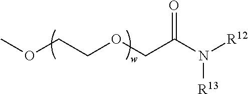

or a pharmaceutically acceptable salt, tautomer or stereoisomer thereof, wherein R¹², R¹³, and w are as defined herein.

In one embodiment of the second aspect (in particular in one embodiment of the above first, second, third, or fourth subgroup of the second aspect, such as in any of the embodiments (1) to (30) listed above), wherein the one or more additional lipids comprise a polymer conjugated lipid, the polymer conjugated lipid is a polysarcosine-lipid conjugate or a conjugate of polysarcosine and a lipid-like material. For example, the polysarcosine-lipid conjugate or conjugate of polysarcosine and a lipid-like material may be a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

In one embodiment of the second aspect (in particular in one embodiment of the above first, second, third, or fourth subgroup of the second aspect, such as in any of the embodiments (1) to (30) listed above), wherein the one or more additional lipids comprise a neutral lipid, the neutral lipid is a phospholipid. Such phospholipid is preferably selected from the group consisting of phosphatidylcholines, phosphatidylethanolamines, phosphatidylglycerols, phosphatidic acids, phosphatidylserines and sphingomyelins. Particular examples of phospholipids include distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In a particularly preferred embodiment, the neutral lipid is DSPC.

In one embodiment of the second aspect (in particular in one embodiment of the above first, second, third, or fourth subgroup of the second aspect, such as in any of the embodiments (1) to (30) listed above), wherein the one or more additional lipids comprise a steroid, the steroid is a sterol such as cholesterol.

In one embodiment of the second aspect (in particular in one embodiment of the above first, second, third, or fourth subgroup of the second aspect, such as in any of the embodiments (1) to (30) listed above), the ethanolic solution comprises the cationically ionizable lipid, the polymer conjugated lipid, the neutral lipid, and the steroid in a molar ratio of 20% to 60% of the cationically ionizable lipid, 0.5% to 15% of the polymer conjugated lipid, 5% to 25% of the neutral lipid, and 25% to 55% of the steroid, based on the total molar amount of lipids in the ethanolic solution. For example, the molar ratio may be 40% to 55% of the cationically ionizable lipid, 1.0% to 10% of the polymer conjugated lipid, 5% to 15% of the neutral lipid, and 30% to 50% of the steroid, such as 45% to 55% of the cationically ionizable lipid, 1.0% to 5% of the polymer conjugated lipid, 8% to 12% of the neutral lipid, and 35% to 45% of the steroid, based on the total molar amount of lipids in the ethanolic solution.

In one embodiment of the second aspect (in particular in one embodiment of the above first, second, third, or fourth subgroup of the first aspect, such as in any of the embodiments (1) to (30) listed above), the final aqueous phase does not comprise a chelating agent.

In one embodiment of the second aspect (in particular in one embodiment of the above first, second, third, or fourth subgroup of the second aspect, such as in any of the embodiments (1) to (30) listed above), the LNPs comprise at least about 75% of the RNA comprised in the composition. For example, the LNPs may comprise at least about 76%, such as at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% of the RNA comprised in the composition.

In one embodiment of the second aspect (in particular in one embodiment of the above first, second, third, or fourth subgroup of the second aspect, such as in any of the embodiments (1) to (30) listed above), the RNA (such as mRNA) is encapsulated within or associated with the LNPs.

In one embodiment of the second aspect (in particular in one embodiment of the above first, second, third, or fourth subgroup of the second aspect, such as in any of the embodiments (1) to (30) listed above), the RNA (such as mRNA) comprises a modified nucleoside in place of uridine. For example, the modified nucleoside may be selected from pseudouridine (ψ), N1-methyl-pseudouridine (m1ψ), and 5-methyl-uridine (m5U).

In one embodiment of the second aspect (in particular in one embodiment of the above first, second, third, or fourth subgroup of the second aspect, such as in any of the embodiments (1) to (30) listed above), the RNA (such as mRNA) comprises one or more of the following (a) a 5′ cap, such as a cap1 or cap2 structure; (b) a 5′ UTR; (c) a 3′ UTR; and (d) a poly-A sequence, such as a poly-A sequence comprising at least 100 nucleotides, wherein the poly-A sequence preferably is an interrupted sequence of A nucleotides.

In one preferred embodiment of the second aspect (in particular in one embodiment of the above first, second, third, or fourth subgroup of the second aspect, such as in any of the embodiments (1) to (30) listed above), the RNA (such as mRNA) encodes one or more polypeptides. For example, the one or more polypeptides may comprise an epitope for inducing an immune response against an antigen in a subject. In a preferred embodiment, the RNA (such as mRNA) comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

In one embodiment of the second aspect (in particular in one embodiment of the above first, second, third, or fourth subgroup of the second aspect, such as in any of the embodiments (1) to (30) listed above), the RNA (such as mRNA) comprises an ORF encoding a full-length SARS-CoV2 S protein variant with proline residue substitutions at positions 986 and 987 of SEQ ID NO:1. For example, the SARS-CoV2 S protein variant may have at least 80% identity to SEQ ID NO:7.

In a third aspect, the present disclosure provides a method of storing a composition, comprising preparing a composition according to the method of the second aspect and storing the composition at a temperature ranging from about −90° C. to about −10° C., such as from about −90° C. to about −40° C. or from about −40° C. to about −25° C. or from about −25° C. to about −10° C., or a temperature of about −20° C. In one embodiment of the third aspect, storing the frozen composition is for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. In one embodiment of the third aspect, storing the frozen composition is for at least 4 weeks, preferably at least 1 month, more preferably at least 2 months, more preferably at least 3 months, more preferably at least 6 months at −20° C. In one embodiment of the third aspect, the composition can be stored at −70° C.

In one embodiment of the third aspect, the method of storing a composition comprises preparing a composition according to the method of the second aspect comprising step (II) (i.e., freezing the formulation to about −10° C. or below); storing the frozen composition at a temperature ranging from about −90° C. to about −10° C. for a certain period of time (e.g., at least one week); and storing the frozen composition a temperature ranging from about 0° C. to about 20° C. for a certain period of time (e.g., at least one week).

It is understood that any embodiment described herein in the context of the first or second aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the third aspect.

In a fourth aspect, the present disclosure provides a method of storing a composition, comprising preparing a liquid composition according to the method of the second aspect and storing the liquid composition at a temperature ranging from about 0° C. to about 20° C., such as from about 1° C. to about 15° C., from about 2° C. to about 10° C., or from about 2° C. to about 8° C., or at a temperature of about 5° C.

In one embodiment of the fourth aspect, storing the liquid composition is for at least 1 week, such as at least 2 weeks, at least 3 weeks, at least 4 weeks, at least 1 month, at least 2 months, at least 3 months, or at least 6 months, preferably at least 4 weeks. In one embodiment of the fourth aspect, storing the liquid composition is for at least 4 weeks, preferably at least 1 month, more preferably at least 2 months, more preferably at least 3 months, more preferably at least 6 months at 5° C.

In one embodiment of the fourth aspect, the method of storing a composition comprises preparing a composition according to the method of the second aspect comprising step (I) (i.e., freezing the formulation to about −10° C. or below); and storing the frozen composition at a temperature ranging from about 0° C. to about 20° C. for a certain period of time (e.g., at least one week).

It is understood that any embodiment described herein in the context of the first, second or third aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the fourth aspect.

In a fifth aspect, the present disclosure provides a composition preparable by the method of the second, third or fourth aspect. In one embodiment of the fifth aspect, the composition can be in frozen form which, preferably, can be stored at a temperature of about −90° C. or higher, such as about −90° C. to about −10° C. For example, the frozen composition of the fifth aspect can be stored at a temperature ranging from about −90° C. to about −40° C. or from about −40° C. to about −25° C. or from about −25° C. to about −10° C., or a temperature to about −20°. In one embodiment of the fifth aspect, the composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen composition can be stored for at least 4 weeks, preferably at least 1 month, more preferably at least 2 months, more preferably at least 3 months, more preferably at least 6 months at −20° C.

In one embodiment of the fifth aspect, where the composition is in frozen form, the RNA integrity after thawing the frozen composition is at least 50%, such as at least 52%, at least 54%, at least 55%, at least 56%, at least 58%, or at least 60%, e.g., after thawing the frozen composition which has been stored at −20° C.

Additionally or alternatively, in one embodiment of the fifth aspect, where the composition is in frozen form, the size (Z_{average}) (and/or size distribution and/or polydispersity index (PDI)) of the LNPs after thawing the frozen composition is equal to the size (Z_{average}) (and/or size distribution and/or PDI) of the LNPs before the composition has been frozen. In one embodiment, the size (Z_{average}) of the LNPs after thawing the frozen composition is between about 50 nm and about 500 nm, preferably between about 40 nm and about 200 nm, more preferably between about 40 nm and about 120 nm. In one embodiment, the PDI of the LNPs after thawing the frozen composition is less than 0.3, preferably less than 0.2, more preferably less than 0.1. In one embodiment, the size (Z_{average}) of the LNPs after thawing the frozen composition is between about 50 nm and about 500 nm, preferably between about 40 nm and about 200 nm, more preferably between about 40 nm and about 120 nm, and the size (Z_{average}) (and/or size distribution and/or PDI) of the LNPs after thawing the frozen composition is equal to the size (Z_{average}) (and/or size distribution and/or PDI) of the LNPs before freezing. In one embodiment, the size (Z_{average}) of the LNPs after thawing the frozen composition is between about 50 nm and about 500 nm, preferably between about 40 nm and about 200 nm, more preferably between about 40 nm and about 120 nm, and the PDI of the LNPs after thawing the frozen composition is less than 0.3 (preferably less than 0.2, more preferably less than 0.1).

In an alternative embodiment of the fifth aspect, the composition is in liquid form.

In one embodiment of the fifth aspect, where the composition is in liquid form, the RNA integrity of the liquid composition, when stored, e.g., at 0° C. or higher for at least one week, is sufficient to produce the desired effect, e.g., to induce an immune response. For example, the RNA integrity of the liquid composition, when stored, e.g., at 0° C. or higher for at least one week, may be at least 50%, such as at least 52%, at least 54%, at least 55%, at least 56%, at least 58%, or at least 60%.

Additionally or alternatively, in one embodiment of the fifth aspect, where the composition is in liquid form, the size (Z_{average}) (and/or size distribution and/or polydispersity index (PDI)) of the LNPs of the liquid composition, when stored, e.g., at 0° C. or higher for at least one week, is sufficient to produce the desired effect, e.g., to induce an immune response. For example, the size (Z_{average}) (and/or size distribution and/or polydispersity index (PDI)) of the LNPs of the liquid composition, when stored, e.g., at 0° C. or higher for at least one week, is equal to the size (Z_{average})(and/or size distribution and/or PDI) of the LNPs of the initial composition, i.e., before storage. In one embodiment, the size (Z_{average}) of the LNPs after storage of the liquid composition e.g., at 0° C. or higher for at least one week is between about 50 nm and about 500 nm, preferably between about 40 nm and about 200 nm, more preferably between about 40 nm and about 120 nm. In one embodiment, the PDI of the LNPs after storage of the liquid composition e.g., at 0° C. or higher for at least one week is less than 0.3, preferably less than 0.2, more preferably less than 0.1. In one embodiment, the size (Z_{average}) of the LNPs after storage of the liquid composition e.g., at 0° C. or higher for at least one week is between about 50 nm and about 500 nm, preferably between about 40 am and about 200 nm, more preferably between about 40 nm and about 120 nm, and the size (Z_{average})(and/or size distribution and/or PDI) of the LNPs after storage of the liquid composition e.g., at 0° C. or higher for at least one week is equal to the size (Z_{average}) (and/or size distribution and/or PDI) of the LNPs before storage. In one embodiment, the size (of the LNPs after storage of the liquid composition e.g., at 0° C. or higher for at least one week is between about 50 nm and about 500 nm, preferably between about 40 nm and about 200 nm, more preferably between about 40 nm and about 120 nm, and the PDI of the LNPs after storage of the liquid composition e.g., at 0° C. or higher for at least one week is less than 0.3 (preferably less than 0.2, more preferably less than 0.1).

It is understood that any embodiment described herein in the context of the first, second, third, or fourth aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the fifth aspect.

In a sixth aspect, the present disclosure provides a method for preparing a ready-to-use pharmaceutical composition, the method comprising the steps of providing a frozen composition prepared by the method of the second or third aspect and thawing the frozen composition thereby obtaining the ready-to-use pharmaceutical composition.

It is understood that any embodiment described herein in the context of the first, second, third, fourth, or fifth aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the sixth aspect. In a seventh aspect, the present disclosure provides a method for preparing a ready-to-use pharmaceutical composition, the method comprising the steps of providing a liquid composition prepared by the method of the second or fourth aspect thereby obtaining the ready-to-use pharmaceutical composition.

It is understood that any embodiment described herein in the context of the first, second, third, fourth, fifth, or sixth aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the seventh aspect.

In an eighth aspect, the present disclosure provides a ready-to-use pharmaceutical composition preparable by the method of the sixth or seventh aspect.

It is understood that any embodiment described herein in the context of the first, second, third, fourth, fifth, sixth, or seventh aspect (in particular, any embodiment of the above first, second, third, or fourth subgroup of the first aspect, such as any of the embodiments (1) to (30) of the first aspect listed above or any embodiment of the above first, second, third, or fourth subgroup of the second aspect, such as any of the embodiments (1) to (30) of the second aspect listed above) may also apply to any embodiment of the eighth aspect.

In a ninth aspect, the present disclosure provides a composition of any one of the first, fifth, and eighth aspect for use in therapy.

It is understood that any embodiment described herein in the context of the first, second, third, fourth, fifth, sixth, seventh, or eighth aspect may also apply to any embodiment of the ninth aspect.

In a tenth aspect, the present disclosure provides a composition of any one of the first, fifth, and eighth aspect for use in inducing an immune response.

It is understood that any embodiment described herein in the context of the first, second, third, fourth, fifth, sixth, seventh, eighth, or ninth aspect may also apply to any embodiment of the tenth aspect.

Further itemised embodiments are as follows:

1. A composition comprising lipid nanoparticles (LNPs) dispersed in an aqueous phase, wherein the LNPs comprise a cationically ionizable lipid and RNA; the aqueous phase comprises a buffer system comprising a buffer substance and a monovalent anion, the buffer substance being selected from the group consisting of tris(hydroxymethyl)aminomethane (Tris) and its protonated form, bis(2-hydroxyethyl)amino-tris(hydroxymethyl)methane (Bis-Tris-methane) and its protonated form, and triethanolamine (TEA) and its protonated form, and the monovalent anion being selected from the group consisting of chloride, acetate, glycolate, lactate, the anion of morpholinoethanesulfonic acid (MES), the anion of 3-(N-morpholino)propanesulfonic acid (MOPS), and the anion of 2-[4-(2-hydroxyethyl)piperazin-1-yl]ethanesulfonic acid (HEPES); the concentration of the buffer substance in the composition is at most about 25 mM; and the aqueous phase is substantially free of inorganic phosphate anions, substantially free of citrate anions, and substantially free of anions of ethylenediaminetetraacetic acid (EDTA).

2. The composition of item 1, wherein the buffer substance is Tris and its protonated form.

3. The composition of item 1 or 2, wherein the concentration of the buffer substance, in particular Tris and its protonated form, in the composition is at most about 20 mM, preferably at most about 15 mM, more preferably at most about 10 mM, such as about 10 mM.

4. The composition of any one of items 1 to 3, wherein the aqueous phase is substantially free of inorganic sulfate anions and/or carbonate anions and/or dibasic organic acid anions and/or polybasic organic acid anions, in particular substantially free of inorganic sulfate anions, carbonate anions, dibasic organic acid anions and polybasic organic acid anions.

5. The composition of any one of items 1 to 4, wherein the monovalent anion is selected from the group consisting of chloride, acetate, glycolate, and lactate, and the concentration of the monovalent anion in the composition is at most equal to, preferably less than the concentration of the buffer substance in the composition, such as less than about 9 mM.

6. The composition of any one of items 1 to 4, wherein the monovalent anion is selected from the group consisting of the anions of MES, MOPS, and HEPES, and the concentration of the monovalent anion in the composition is at least equal to, preferably higher than the concentration of the buffer substance in the composition.

7. The composition of any one of items 1 to 6, wherein the pH of the composition is between about 6.5 and about 8.0, preferably between about 6.9 and about 7.9, such as between about 7.0 and about 7.8.

8. The composition of any one of items 1 to 7, wherein water is the main component in the composition and/or the total amount of solvent(s) other than water contained in the composition is less than about 0.5% (v/v).

9. The composition of any one of items 1 to 8, wherein the osmolality of the composition is at most about 400×10⁻³ osmol/kg.

10. The composition of any one of items 1 to 9, wherein the concentration of the RNA in the composition is about 5 mg/l to about 150 mg/l, preferably about 10 mg/l to about 130 mg/l, more preferably about 30 mg/l to about 120 mg/l.

11. The composition of any one of items 1 to 10, wherein the composition comprises a cryoprotectant, preferably in a concentration of at least about 1% w/v, wherein the cryoprotectant preferably comprises one or more compounds selected from the group consisting of carbohydrates and sugar alcohols, more preferably the cryoprotectant is selected from the group consisting of sucrose, glucose, glycerol, sorbitol, and a combination thereof, more preferably the cryoprotectant comprises sucrose and/or glycerol.

12. The composition of any one of items 1 to 10, wherein the composition is substantially free of a cryoprotectant.

13. The composition of any one of items 1 to 12, wherein the cationically ionizable lipid comprises a head group which includes at least one nitrogen atom which is capable of being protonated under physiological conditions.

14. The composition of any one of items 1 to 13, wherein the cationically ionizable lipid has the structure of Formula (I):

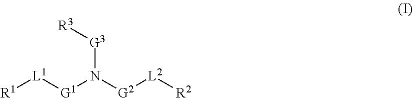

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)—, —S—S—, —C(O)S—, SC(═O)—, —NR^{a}C(═O)—, —C(═O)NR^{a}—, NR^{a}C(═O)NR^{a}—, —OC(═O)NR^{a}— or NR^{a}C(═O)O— or a direct bond;
G¹ and G² are each independently unsubstituted C₁-C₁₂ alkylene or C₂-C₁₂ alkenylene;
G³ is C₁-C₂₄ alkylene, C₂-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

15. The composition of any one of items 1 to 13, wherein:
(α) the cationically ionizable lipid is selected from the structures I-1 to 1-36 shown herein; or
(β) the cationically ionizable lipid is selected from the structures A to F shown herein; or
(γ) the cationically ionizable lipid is the lipid having the structure I-3 shown herein.

16. The composition of any one of items 1 to 15, wherein the LNPs further comprise one or more additional lipids, preferably selected from the group consisting of polymer conjugated lipids, neutral lipids, steroids, and combinations thereof, more preferably the LNPs comprise the cationically ionizable lipid, a polymer conjugated lipid, a neutral lipid, and a steroid.

17. The composition of item 16, wherein the polymer conjugated lipid comprises a pegylated lipid, wherein the pegylated lipid preferably has the following structure:

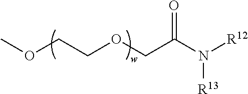

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

18. The composition of item 16, wherein the polymer conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

19. The composition of any one of items 16 to 18, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

20. The composition of any one of items 16 to 19, wherein the steroid comprises a sterol such as cholesterol.

21. The composition of any one of items 1 to 20, wherein the aqueous phase does not comprise a chelating agent.

22. The composition of any one of items 1 to 21, wherein the LNPs comprise at least about 75%, preferably at least about 80% of the RNA comprised in the composition.

23. The composition of any one of items 1 to 22, wherein the RNA is encapsulated within or associated with the LNPs.

24. The composition of any one of items 1 to 23, wherein the RNA comprises a modified nucleoside in place of uridine, wherein the modified nucleoside is preferably selected from pseudouridine (ψ), N1-methyl-pseudouridine (m1ψ), and 5-methyl-uridine (m5U).

25. The composition of any one of items 1 to 24, wherein the RNA comprises at least one of the following, preferably all of the following: a 5′ cap; a 5′ UTR; a 3′ UTR; and a poly-A sequence.

26. The composition of item 25, wherein the poly-A sequence comprises at least 100 A nucleotides, wherein the poly-A sequence preferably is an interrupted sequence of A nucleotides.

27. The composition of item 25 or 26, wherein the 5′ cap is a cap1 or cap2 structure.

28. The composition of any one of items 1 to 27, wherein the RNA encodes one or more polypeptides, wherein the one or more polypeptides preferably comprise an epitope for inducing an immune response against an antigen in a subject.

29. The composition of item 28, wherein the RNA comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

30. The composition of item 28 or 29, wherein the RNA comprises an ORF encoding a full-length SARS-CoV2 S protein variant with proline residue substitutions at positions 986 and 987 of SEQ ID NO: 1.

31. The composition of item 29 or 30, wherein the SARS-CoV2 S protein variant has at least 80% identity to SEQ ID NO: 7.

32. The composition of any one of items 1 to 31, wherein the composition is in frozen form.

33. The composition of item 32, wherein the RNA integrity after thawing the frozen composition is at least 50% compared to the RNA integrity before the composition has been frozen.

34. The composition of item 32 or 33, wherein the size (Z_{average}) and/or size distribution and/or polydispersity index (PDT) of the LNPs after thawing the frozen composition is equal to the size (Z_{average}) and/or size distribution and/or PDI of the LNPs before the composition has been frozen.

35. The composition of any one of items 1 to 31, wherein the composition is in liquid form.

36. A method of preparing a composition comprising LNPs dispersed in a final aqueous phase, wherein the LNPs comprise a cationically ionizable lipid and RNA; the final aqueous phase comprises a final buffer system comprising a final buffer substance and a final monovalent anion, the final buffer substance being selected from the group consisting of Tris and its protonated form, Bis-Tris-methane and its protonated form, and TEA and its protonated form, and the final monovalent anion being selected from the group consisting of chloride, acetate, glycolate, lactate, the anion of MES, the anion of MOPS, and the anion of HEPES; the concentration of the final buffer substance in the composition is at most about 25 mM; and the final aqueous phase is substantially free of inorganic phosphate anions, substantially free of citrate anions, and substantially free of anions of EDTA; wherein the method comprises:
(I) preparing a formulation comprising LNPs dispersed in the final aqueous phase, wherein the LNPs comprise the cationically ionizable lipid and RNA; and
(I) optionally freezing the formulation to about −10° C. or below,
thereby obtaining the composition,
wherein step (I) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing a first intermediate formulation comprising the LNPs dispersed in a first aqueous phase comprising the first buffer system; and
(d) filtrating the first intermediate formulation prepared under (c) using a final aqueous buffer solution comprising the final buffer system,
thereby preparing the formulation comprising the LNPs dispersed in the final aqueous phase.

37. The method of item 36, wherein step (I) further comprises one or more steps selected from diluting and filtrating.

38. The method of item 36 or 37, wherein step (I) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f) once or two or more times, wherein the further intermediate formulation comprising the LNPs dispersed in the further aqueous phase comprising the further buffer system obtained after step (f′) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f) is absent, or the further intermediate formulation obtained in step (f), if step (f) is present and step (g′) is not present, or the further intermediate formulation obtained after step (g′), if steps (f′) and (g′) are present, using a final aqueous buffer solution comprising the final buffer system and having a pH of at least 6.0; and
(i′) optionally diluting the formulation obtained in step (h′) with a dilution solution; thereby preparing the formulation comprising the LNPs dispersed in the final aqueous phase.

39. The method of any one of items 36 to 38, wherein filtrating is tangential flow filtrating or diafiltrating, preferably tangential flow filtrating.

40. The method of any one of items 36 to 39, which comprises (II) freezing the formulation to about −10° C. or below.

41. The method of any one of items 36 to 40, wherein the final buffer substance is Tris and its protonated form.

42. The method of any one of items 36 to 41, wherein the concentration of the final buffer substance, in particular Tris and its protonated form, in the composition is at most about 20 mM, preferably at most about 15 mM, more preferably at most about 10 mM, such as about 10 mM.

43. The method of any one of items 36 to 42, wherein the final aqueous phase is substantially free of inorganic sulfate anions and/or carbonate anions and/or dibasic organic acid anions and/or polybasic organic acid anions, in particular substantially free of inorganic sulfate anions, carbonate anions dibasic organic acid anions and polybasic organic acid anions.

44. The method of any one of items 36 to 43, wherein (i) the RNA solution prepared in step (a) further comprises one or more di- and/or polybasic organic acid anions, and step (d) is conducted under conditions which remove the one or more di- and/or polybasic organic acid anions resulting in the formulation comprising the LNPs dispersed in the final aqueous phase with the final aqueous phase being substantially free of the one or more di- and/or polybasic organic acid anions present in the RNA solution prepared in step (a); or (ii) the first aqueous buffer solution and the first aqueous phase comprise one or more di- and/or polybasic organic acid anions and least one of steps (f) to (h′) is conducted under conditions which remove the one or more di- and/or polybasic organic acid anions from the first intermediate formulation and/or from the further intermediate formulation.

45. The method of any one of items 36 to 44, wherein (i) the RNA solution obtained in step (a) has a pH of below 6.0, preferably at most about 5.0, more preferably at most about 4.5; or (ii) the first aqueous buffer solution has a pH of below 6.0, preferably at most about 5.0, more preferably at most about 4.5.

46. The method of item 44 or 45, wherein the one or more di- and/or polybasic organic acid anions comprise citrate anions and/or anions of EDTA.

47. The method of any one of items 36 to 43, wherein (i) the first buffer system used in step (a) comprises the final buffer substance and the final monovalent anion used in step (d), preferably the buffer system and pH of the first buffer system used in step (a) are identical to the buffer system and pH of the final aqueous buffer solution used in step (d); or (ii) each of the first buffer system and every further buffer system used in steps (b′), (f) and (g′) comprises the final buffer substance and the final monovalent anion used in step (h′), preferably the buffer system and pH of each of the first aqueous buffer solution and of every further aqueous buffer solution used in steps (b′), (f) and (g′) are identical to the buffer system and pH of the final aqueous buffer solution.

48. The method of any one of items 36 to 47, wherein the final monovalent anion is selected from the group consisting of chloride, acetate, glycolate, and lactate, and the concentration of the final monovalent anion in the composition is at most equal to, preferably less than the concentration of the final buffer substance in the composition, such as less than about 9 mM.

49. The method of any one of items 36 to 48, wherein the final monovalent anion is selected from the group consisting of the anions of MES, MOPS, and HEPES, and the concentration of the final monovalent anion in the composition is at least equal to, preferably higher than the concentration of the final buffer substance in the composition.

50. The method of any one of items 36 to 49, wherein the pH of the composition is between about 6.5 and about 8.0, preferably between about 6.9 and about 7.9, such as between about 7.0 and about 7.8.

51. The method of any one of items 36 to 50, wherein water is the main component in the formulation and/or composition and/or the total amount of solvent(s) other than water contained in the composition is less than about 0.5% (v/v).

52. The method of any one of items 36 to 51, wherein the osmolality of the composition is at most about 400×10¹ osmol/kg.

53. The method of any one of items 36 to 52, wherein the concentration of the RNA in the composition is about 5 mg/l to about 150 mg/l, preferably about 10 mg/l to about 130 mg/l, more preferably about 30 mg/l to about 120 mg/l.

54. The method of any one of items 36 to 53, wherein (i) step (I) further comprises diluting the formulation prepared under (d) with a dilution solution, or step (i′) is present, wherein the dilution solution comprises a cryoprotectant; and/or (ii) the formulation obtained in step (1) and the composition comprise a cryoprotectant, preferably in a concentration of at least about 1% w/v, wherein the cryoprotectant preferably comprises one or more selected from the group consisting of carbohydrates and sugar alcohols, more preferably the cryoprotectant is selected from the group consisting of sucrose, glucose, glycerol, sorbitol, and a combination thereof, more preferably the cryoprotectant comprises sucrose and/or glycerol.

55. The method of any one of items 36 to 53, wherein the formulation obtained in step (I) and the composition is substantially free of a cryoprotectant.

56. The method of any one of items 36 to 55, wherein the cationically ionizable lipid comprises a head group which includes at least one nitrogen atom which is capable of being protonated under physiological conditions.

57. The method of any one of items 36 to 56, wherein the cationically ionizable lipid has the structure of Formula (I):

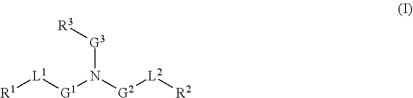

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NRC(═O)O—, and the other of L¹ or L² is —((C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond,
G¹ and G² are each independently unsubstituted C₁-C₁₂ alkylene or C₂-C₁₂ alkenylene;
G³ is C₁-C₂₄ alkylene, C₂-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

58. The method of any one of items 36 to 56, wherein:
(α) the cationically ionizable lipid is selected from the structures I-1 to I-36 shown herein; or
(β) the cationically ionizable lipid is selected from the structures A to F shown herein; or
(γ) the cationically ionizable lipid is the lipid having the structure I-3 shown herein.

59. The method of any one of items 36 to 58, wherein the ethanolic solution prepared in step (b) or (d′) further comprises one or more additional lipids and the LNPs further comprise the one or more additional lipids, wherein the one or more additional lipids are preferably selected from the group consisting of polymer conjugated lipids, neutral lipids, steroids, and combinations thereof, more preferably the one or more additional lipids comprise a polymer conjugated lipid, a neutral lipid, and a steroid.

60. The method of item 59, wherein the polymer conjugated lipid comprises a pegylated lipid, wherein the pegylated lipid preferably has the following structure:

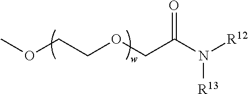

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

61. The method of item 59, wherein the polymer conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

62. The method of any one of items 59 to 61, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadeceayl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

63. The method of any one of items 59 to 62, wherein the steroid comprises a sterol such as cholesterol.

64. The method of any one of items 36 to 63, wherein the cationically ionizable lipid, the polymer conjugated lipid, the neutral lipid, and the steroid are present in the ethanolic solution in a molar ratio of 20% to 60% of the cationically ionizable lipid, 0.5% to 15% of the polymer conjugated lipid, 5% to 25% of the neutral lipid, and 25% to 55% of the steroid, preferably in a molar ratio of 45% to 55% of the cationically ionizable lipid, 1.0% to 5% of the polymer conjugated lipid, 8% to 12% of the neutral lipid, and 35% to 45% of the steroid.

65. The method of any one of items 36 to 64, wherein the final aqueous phase does not comprise a chelating agent.

66. The method of any one of items 36 to 65, wherein the LNPs comprise at least about 75%, preferably at least about 80% of the RNA comprised in the composition.

67. The method of any one of items 36 to 66, wherein the RNA is encapsulated within or associated with the LNPs.

68. The method of any one of items 36 to 67, wherein the RNA comprises a modified nucleoside in place of uridine, wherein the modified nucleoside is preferably selected from pseudouridine (ψ), N1-methyl-pseudouridine (m1ψ), and 5-methyl-uridine (m5U).

69. The method of any one of items 36 to 68, wherein the RNA comprises at least one of the following, preferably all of the following: a 5′ cap; a 5′ UTR; a 3′ UTR; and a poly-A sequence.

70. The method of item 69, wherein the poly-A sequence comprises at least 100 A nucleotides, wherein the poly-A sequence preferably is an interrupted sequence of A nucleotides.

71. The method of item 69 or 70, wherein the 5′ cap is a cap1 or cap2 structure.

72. The method of any one of items 36 to 71, wherein the RNA encodes one or more polypeptides, wherein the one or more polypeptides preferably comprise an epitope for inducing an immune response against an antigen in a subject.

73. The method of item 72, wherein the RNA comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

74. The method of item 72 or 73, wherein the RNA comprises an ORF encoding a full-length SARS-CoV2 S protein variant with proline residue substitutions at positions 986 and 987 of SEQ ID NO: 1.

75. The method of item 73 or 74, wherein the SARS-CoV2 S protein variant has at least 80% identity to SEQ ID NO: 7.

76. The method of any one of items 36 to 39 and 41 to 75, which does not comprise step (II).

77. A method of storing a composition, comprising preparing a composition according to the method of any one of items 36 to 75 and storing the composition at a temperature ranging from about −90° C. to about −10° C., such as from about −90° C. to about −40° C. or from about −25° C. to about −10° C.

78. The method of item 77, wherein storing the composition is for at least 1 week, such as at least 2 weeks, at least 3 weeks, at least 4 weeks, at least 1 month, at least 2 months, at least 3 months, at least 6 months, at least 12 months, at least 24 months, or at least 36 months.

79. A method of storing a composition, comprising preparing a composition according to the method of any one of items 36 to 78 and storing the composition at a temperature ranging from about 0° C. to about 20° C., such as from about 1° C. to about 15° C., from about 2° C. to about 10° C., or from about 2° C. to about 8° C., or at a temperature of about 5° C.

80. The method of item 79, wherein storing the composition is for at least 1 week, such as at least 2 weeks, at least 3 weeks, at least 4 weeks, at least 1 month, at least 2 months, at least 3 months, or at least 6 months.

81. A composition preparable by the method of any one of items 36 to 80.

82. The composition of item 81, which is in frozen form.

83. The composition of item 82, wherein the RNA integrity after thawing the frozen composition is at least 50% compared to the RNA integrity of the composition before the composition has been frozen.

84. The composition of item 82 or 83, wherein the size (Z_{average}) and/or size distribution and/or polydispersity index (PDI) of the LNPs after thawing the frozen composition is equal to the size (Z_{average}) and/or size distribution and/or PDI of the LNPs before the composition has been frozen.

85. The composition of item 81, which is in liquid form.

86. The composition of item 85, wherein the RNA integrity after storage of the composition for at least 1 week is at least 50% compared to the RNA integrity before storage.

87. The composition of item 85 or 86, wherein the size (Z_{average}) and/or size distribution and/or polydispersity index (PDI) of the LNPs after storage of the composition for at least one week is equal to the size (Z_{average}) and/or size distribution and/or PDI of the LNPs before storage.

88. A method for preparing a ready-to-use pharmaceutical composition, the method comprising the steps of providing a frozen composition prepared by the method of any one of items 36 to 75, 77, and 78, and thawing the frozen composition thereby obtaining the ready-to-use pharmaceutical composition.

89. A method for preparing a ready-to-use pharmaceutical composition, the method comprising the step of providing a liquid composition prepared by the method of any one of items 36 to 39, 41 to 76, 79, and 80, thereby obtaining the ready-to-use pharmaceutical composition.

90. A ready-to-use pharmaceutical composition preparable by the method of item 88 or 89.

91. A composition of any one of items 1 to 35, 81 to 87, and 90 for use in therapy.

92. A composition of any one of items 1 to 35, 81 to 87, and 90 for use in inducing an immune response in a subject.

Further aspects of the present disclosure are disclosed herein.

### BRIEF DESCRIPTION OF THE FIGURES

FIG. 1 is a schematic of in vivo assay for BNT162b1 material.

FIG. 2 shows RNA integrity determined by capillary electrophoresis. RNA LNPs were prepared by the aqueous-ethanol mixing protocol using 20 mM Tris added to the organic phase. LNPs were generated in Tris:acetate pH 4, pH 5.5 or pH 6.8 and the resulting primary LNPs were split: one portion was subjected to dialysis against PBS (A); the other portion was subjected to dialysis against Tris:acetate pH 7.4 (B). For comparison, the organic phase did not receive Tris, LNP were generated in Na-acetate buffer pH 5.5 and the material was dialysed against Tris:acetate pH 7.4. All samples were stored for 50 h at room temperature.

FIG. 3 shows the morphology of selected RNA LNP compositions. Vitrified samples were analyzed by cryo electron microscopy. For the d028 sample a 2.5× higher magnification was used.

FIG. 4 shows mouse immunogenicity of RNA LNP compositions. 1 μg of RNA LNP composition D028 (LNP A), D029 (LNP B) and D030 (LNP C) were injected i.m. into mice, a reference composition (ATM) and saline were used as controls. Expression of the S1 protein (left panels) and generation of S1 IgG (right panels) was followed for 28 days. All RNA LNP compositions have comparable bioactivity amongst each other and in relation to the reference composition.

FIG. 5 shows the stability of the RNA LNP composition D028 (A) and of the RNA LNP compositions D029 (B) and D030 (C). Squares: room temperature, diamonds: 5° C., triangles: −20° C., circles −70° C. Solid lines: particle size, RNA integrity or RNA content; dotted lines: PDI, LMS (denotes the stable folded RNA) or RNA encapsulation.

FIG. 6 shows the colloidal stability RNA LNP compositions having a buffer strength of 10 mM or 50 mM. Squares: room temperature, diamonds: 5° C., triangles: −20° C. Solid lines represent particle size and dotted lines represent PDI.

FIG. 7 shows the stability of the RNA in relation to the strength of the Tris buffer. Results represent % of the RNA modality being present in samples after certain times and conditions. RNA denotes the full-length RNA; LMS denotes the highly stable folded form of RNA; and Frag denotes the RNA fragments of the sample.

### DESCRIPTION OF THE SEQUENCES

The following table provides a listing of certain sequences referenced herein.

TABLE 1
DESCRIPTION OF THE SEQUENCES

| | | |
|---|---|---|
| SEQ | | |
| ID | | |
| NO: | Description | SEQUENCE |

Antigenic S protein sequences

| | | |
|---|---|---|
| 1 | S protein | MFVFLVLLPLVSSQCVNLTTRTQLP |
| | (aa) | PAYTNSFTRGVYYPDKVFRSSVLHS |
| | | TQDLFLPFFSNVTWFHAIHVSGTNG |
| | | TKRFDNPVLPFNDGVYFASTEKSNI |
| | | IRGWIFGTTLDSKTQSLLIVNNATN |
| | | VVIKVCEFQFCNDPFLGVYYHKNNK |
| | | SWMESEFRVYSSANNCTFEYVSQPF |
| | | LMDLEGKQGNFKNLREFVFKNIDGY |
| | | FKIYSKHTPINLVRDLPQGFSALEP |
| | | LVDLPIGINITRFQTLLALHRSYLT |
| | | PGDSSSGWTAGAAAYYVGYLQPRTF |
| | | LLKYNENGTITDAVDCALDPLSETK |
| | | CTLKSFTVEKGIYQTSNFRVQPTES |
| | | IVRFPNITNLCPFGEVFNATRFASV |
| | | YAWNRKRISNCVADYSVLYNSASFS |
| | | TFKCYGVSPTKLNDLCFTNVYADSF |
| | | VIRGDEVRQIAPGQTGKIADYNYKL |
| | | PDDFTGCVIAWNSNNLDSKVGGNYN |
| | | YLYRLFRKSNLKPFERDISTEIYQA |
| | | GSTPCNGVEGFNCYFPLQSYGFQPT |
| | | NGVGYQPYRVVVLSFELLHAPATVC |
| | | GPKKSTNLVKNKCVNFNFNGLTGTG |
| | | VLTESNKKFLPFQQFGRDIADTTDA |
| | | VRDPQTLEILDITPCSFGGVSVITP |
| | | GTNTSNQVAVLYQDVNCTEVPVAIH |
| | | ADQLTPTWRVYSTGSNVFQTRAGCL |
| | | IGAEHVNNSYECDIPIGAGICASYQ |
| | | TQTNSPRRARSVASQSIIAYTMSLG |
| | | AENSVAYSNNSIAIPTNFTISVTTE |
| | | ILPVSMTKTSVDCTMYICGDSTECS |
| | | NLLLQYGSFCTQLNRALTGIAVEQD |
| | | KNTQEVFAQVKQIYKTPPIKDFGGF |
| | | NFSQILPDPSKPSKRSFIEDLLFNK |
| | | VTLADAGFIKQYGDCLGDIAARDLI |
| | | CAQKFNGLTVLPPLLTDEMIAQYTS |
| | | ALLAGTITSGWTFGAGAALQIPFAM |
| | | QMAYRFNGIGVTQNVLYENQKLIAN |
| | | QFNSAIGKIQDSLSSTASALGKLQD |
| | | VVNQNAQALNTLVKQLSSNFGAISS |
| | | VLNDILSRLDKVEAEVQIDRLITGR |
| | | LQSLQTYVTQQLIRAAEIRASANLA |
| | | ATKMSECVLGQSKRVDFCGKGYHLM |
| | | SFPQSAPHGVVFLHVTYVPAQEKNF |
| | | TTAPAICHDGKAHFPREGVFVSNGT |
| | | HWFVTQRNFYEPQIITTDNTFVSGN |
| | | CDVVIGIVNNTVYDPLQPELDSFKE |
| | | ELDKYFKNHTSPDVDLGDISGINAS |
| | | VVNIQKEIDRLNEVAKNLNESLIDL |
| | | QELGKYEQYIKWPWYIWLGFIAGLI |
| | | AIVMVTIMLCCMTSCCSCLKGCCSC |
| | | GSCCKFDEDDSEPVLKGVKLHYT |
| 2 | S protein | auguuuguguuucuugugcugcugc |
| | (CDS) | cucuugugucuucucagugugugaa |
| | | uuugacaacaagaacacagcugcca |
| | | ccagcuuauacaaauuuuuuaccag |
| | | aggaguguauuauccugauaaagug |
| | | uuuagaucuucugugcugcacagca |
| | | cacaggaccuguuucugccauuuuu |
| | | uagcaaugugacaugguuucaugca |
| | | auucaugugucuggaacaaauggaa |
| | | caaaaagauuugauaauccugugcu |
| | | gccuuuuaaugauggaguguauuuu |
| | | gcuucaacagaaaagucaaauauua |
| | | uuagaggauggauuuuuggaacaac |
| | | acuggauucuaaaacacagucucug |
| | | cugauugugaauaaugcaacaaaug |
| | | uggugauuaaagugugugaauuuca |
| | | guuuuguaaugauccuuuucuggga |
| | | guguauuaucacaaaaauaauaaau |
| | | cuuggauggaaucugaauuuagagu |
| | | guauuccucugcaaauaauuguaca |
| | | uuugaauaugugucucagccuuuuc |
| | | ugauggaucuggaaggaaaacaggg |
| | | caauuuuaaaaaucugagagaauuu |
| | | guguuuaaaaauauugauggauauu |
| | | uuaaaauuuauucuaaacacacacc |
| | | aauuaauuuagugagagaucugccu |
| | | cagggauuuucugcucuggaaccuc |
| | | ugguggaucugccaauuggcauuaa |
| | | uauuacaagauuucagacacugcug |
| | | gcucugcacagaucuuaucugacac |
| | | cuggagauucuucuucuggauggac |
| | | agccggagcugcagcuuauuaugug |
| | | ggcuaucugcagccaagaacauuuc |
| | | ugcugaaauauaaugaaaauggaac |
| | | aauuacagaugcuguggauugugcu |
| | | cuggauccucugucugaaaaaaaug |
| | | uacauuaaaaucuuuuacaguggaa |
| | | aaaggcauuuaucagacaucuaauu |
| | | uuagagugcagccaacagaaucuau |
| | | ugugagauuuccaaauauuacaaau |
| | | cuguguccauuuggagaaguguuua |
| | | augcaacaagauuugcaucugugua |
| | | ugcauggaauagaaaaagaauuucu |
| | | aauuguguggcugauuauucugugc |
| | | uguauaauagugcuucuuuuuccac |
| | | auuuaaauguuauggagugucucca |
| | | acaaaauuaaaugauuuauguuuua |
| | | caaauguguaugcugauucuuuugu |
| | | gaucagaggugaugaagugagacag |
| | | auugcccccggacagacaggaaaaa |
| | | uugcugauuacaauuacaaacugcc |
| | | ugaugauuuuacaggaugugugauu |
| | | gcuuggaauucuaauaauuuagauu |
| | | cuaaagugggaggaaauuacaauua |
| | | ucuguacagacuguuuagaaaauca |
| | | aaucugaaaccuuuugaaagagaua |
| | | uuucaacagaaauuuaucaggcugg |
| | | aucaacaccuuguaauggaguggaa |
| | | ggauuuaauuguuauuuuccauuac |
| | | agagcuauggauuucagccaaccaa |
| | | uggugugggauaucagccauauaga |
| | | gugguggugcugucuuuugaacugc |
| | | ugcaugcaccugcaacagugugugg |
| | | accuaaaaaaucuacaaauuuagug |
| | | aaaaauaaaugugugaauuuuaauu |
| | | uuaauggauuaacaggaacaggagu |
| | | gcugacagaaucuaauaaaaaauuu |
| | | cugccuuuucagcaguuuggcagag |
| | | auauugcagauaccacagaugcagu |
| | | gagagauccucagacauuagaaauu |
| | | cuggauauuacaccuuguuuuuugg |
| | | gggugugucugugauuacaccugga |
| | | acaaauacaucuaaucagguggcug |
| | | ugcuguaucaggaugugaauuguac |
| | | agaagugccaguggcaauucaugca |
| | | gaucagcugacaccaacauggagag |
| | | uguauucuacaggaucuaauguguu |
| | | ucagacaagagcaggaugucugauu |
| | | ggagcagaacaugugaauaauucuu |
| | | augaaugugauauuccaauuggagc |
| | | aggcauuugugcaucuuaucagaca |
| | | cagacaaauuccccaaggagagcaa |
| | | gaucuguggcaucucagucuauuau |
| | | ugcauacaccaugucucugggagca |
| | | gaaaauucuguggcauauucuaaua |
| | | auucuauugcuauuccaacaaauuu |
| | | uaccauuucugugacaacagaaauu |
| | | uuaccugugucuaugacaaaaacau |
| | | cuguggauuguaccauguacauuug |
| | | uggagauucuacagaauguucuaau |
| | | cugcugcugcaguauggaucuuuuu |
| | | guacacagcugaauagagcuuuaac |
| | | aggaauugcuguggaacaggauaaa |
| | | aauacacaggaaguguuugcucagg |
| | | ugaaacagauuuacaaaacaccacc |
| | | aauuaaagauuuuggaggauuuaau |
| | | uuuagccagauucugccugauccuu |
| | | cuaaaccuucuaaaagaucuuuuau |
| | | ugaagaucugcuguuuaauaaagug |
| | | acacuggcagaugcaggauuuauua |
| | | aacaguauggagauugccuggguga |
| | | uauugcugcaagagaucugauuugu |
| | | gcucagaaauuuaauggacugacag |
| | | ugcugccuccucugcugacagauga |
| | | aaugauugcucaguacacaucugcu |
| | | uuacuggcuggaacaauuacaagcg |
| | | gauggacauuuggagcuggagcugc |
| | | ucugcagauuccuuuugcaaugcag |
| | | auggcuuacagauuuaauggaauug |
| | | gagugacacagaauguguuauauga |
| | | aaaucagaaacugauugcaaaucag |
| | | uuuaauucugcaauuggcaaaauuc |
| | | aggauucucugucuucuacagcuuc |
| | | ugcucugggaaaacugcaggaugug |
| | | gugaaucagaaugcacaggcacuga |
| | | auacucuggugaaacagcugucuag |
| | | caauuuuggggcaauuucuucugug |
| | | cugaaugauauucugucuagacugg |
| | | auaaaguggaagcugaagugcagau |
| | | ugauagacugaucacaggaagacug |
| | | cagucucugcagacuuaugugacac |
| | | agcagcugauuagagcugcugaaau |
| | | uagagcuucugcuaaucuggcugcu |
| | | acaaaaaugucugaaugugugcugg |
| | | gacagucaaaaagaguggauuuuug |
| | | uggaaaaggauaucaucugaugucu |
| | | uuuccacagucugcuccacauggag |
| | | ugguguuuuuacaugugacauaugu |
| | | gccagcacaggaaaagaauuuuacc |
| | | acagcaccagcaauuugucaugaug |
| | | gaaaagcacauuuuccaagagaagg |
| | | aguguuugugucuaauggaacacau |
| | | ugguuugugacacagagaaauuuuu |
| | | augaaccucagauuauuacaacaga |
| | | uaauacauuugugucaggaaauugu |
| | | gauguggugauuggaauugugaaua |
| | | auacaguguaugauccacugcagcc |
| | | agaacuggauucuuuuaaagaagaa |
| | | cuggauaaauauuuuaaaaaucaca |
| | | caucuccugauguggauuuaggaga |
| | | uauuucuggaaucaaugcaucugug |
| | | gugaauauucagaaagaaauugaua |
| | | gacugaaugaaguggccaaaaaucu |
| | | gaaugaaucucugauugaucugcag |
| | | gaacuuggaaaauaugaacaguaca |
| | | uuaaauggccuugguacauuuggcu |
| | | uggauuuauugcaggauuaauugca |
| | | auugugauggugacaauuauguuau |
| | | guuguaugacaucauguuguucuug |
| | | uuuaaaaggauguuguucuugugga |
| | | agcuguuguaaauuugaugaagaug |
| | | auucugaaccuguguuaaaaggagu |
| | | gaaauugcauuacaca |
| 3 | S protein RBD | MFVFLVLLPLVSSQCVVRFPNITNL |
| | (amino | CPFGEVFNATRFASVYAWNRKRISN |
| | acid)(V05) | CVADYSVLYNSASFSTFKCYGVSPT |
| | | KLNDLCFTNVYADSFVIRGDEVRQI |
| | | APGQTGKIADYNYKLPDDFTGCVIA |
| | | WNSNNLDSKVGGNYNYLYRLFRKSN |
| | | LKPFERDISTEIYQAGSTPCNGVEG |
| | | FNCYFPLQSYGFQPTNGVGYQPYRV |
| | | VVLSFELLHAPATVCGPK |
| 4 | S protein RBD | auguuuguguuucuugugcugcugc |
| | (CDS) (V05) | cucuugugucuucucaguguguggu |
| | | gagauuuccaaauauuacaaaucug |
| | | uguccauuuggagaaguguuuaaug |
| | | caacaagauuugcaucuguguaugc |
| | | auggaauagaaaaagaauuucuaau |
| | | uguguggcugauuauucugugcugu |
| | | auaauagugcuucuuuuuccacauu |
| | | uaaauguuauggagugucuccaaca |
| | | aaauuaaaugauuuauguuuuacaa |
| | | auguguaugcugauucuuuugugau |
| | | cagaggugaugaagugagacagauu |
| | | gcccccggacagacaggaaaaauug |
| | | cugauuacaauuacaaacugccuga |
| | | ugauuuuacaggaugugugauugcu |
| | | uggaauucuaauaauuuagauucua |
| | | aagugggaggaaauuacaauuaucu |
| | | guacagacuguuuagaaaaucaaau |
| | | cugaaaccuuuugaaagagauauuu |
| | | caacagaaauuuaucaggcuggauc |
| | | aacaccuuguaauggaguggaagga |
| | | uuuaauuguuauuuuccauuacaga |
| | | gcuauggauuucagccaaccaaugg |
| | | ugugggauaucagccauauagagug |
| | | guggugcugucuuuugaacugcugc |
| | | augcaccugcaacaguguguggacc |
| | | uaaa |
| 5 | S protein RBD/ | MFVFLVLLPLVSSQCVVRFPNITNL |
| | acid) (V05) | CPFGEVFNATRFASVYAWNRKRISN |
| | Fibritin (amino | CVADYSVLYNSASFSTFKCYGVSPT |
| | | KLNDLCFTNVYADSFVIRGDEVRQI |
| | | APGQTGKIADYNYKLPDDFTGCVIA |
| | | WNSNNLDSKVGGNYNYLYRLFRKSN |
| | | LKPFERDISTEIYQAGSTPCNGVEG |
| | | FNCYFPLQSYGFQPTNGVGYQPYRV |
| | | VVLSFELLHAPATVCGPKGSPGSGS |
| | | GSGYIPEAPRDGQAYVRKDGEWVLL |
| | | STFLGRSLEVLFQGPG |
| 6 | S protein RBD/ | auguuuguguuucuugugcugcugc |
| | Fibritin (CDS) | cucuugugucuucucaguguguggu |
| | (V05) | gagauuuccaaauauuacaaaucug |
| | | uguccauuuggagaaguguuuaaug |
| | | caacaagauuugcaucuguguaugc |
| | | auggaauagaaaaagaauuucuaau |
| | | uguguggcugauuauucugugcugu |
| | | auaauagugcuucuuuuuccacauu |
| | | uaaauguuauggagugucuccaaca |
| | | aaauuaaaugauuuauguuuuacaa |
| | | auguguaugcugauucuuuugugau |
| | | cagaggugaugaagugagacagauu |
| | | gcccccggacagacaggaaaaauug |
| | | cugauuacaauuacaaacugccuga |
| | | ugauuuuacaggaugugugauugcu |
| | | uggaauucuaauaauuuagauucua |
| | | aagugggaggaaauuacaauuaucu |
| | | guacagacuguuuagaaaaucaaau |
| | | cugaaaccuuuugaaagagauauuu |
| | | caacagaaauuuaucaggcuggauc |
| | | aacaccuuguaauggaguggaagga |
| | | uuuaauuguuauuuuccauuacaga |
| | | gcuauggauuucagccaaccaaugg |
| | | ugugggauaucagccauauagagug |
| | | guggugcugucuuuugaacugcugc |
| | | augcaccugcaacaguguguggacc |
| | | uaaaggcucccccggcuccggcucc |
| | | ggaucugguuauauuccugaagcuc |
| | | caagagaugggcaagcuuacguucg |
| | | uaaagauggcgaauggguauuacuu |
| | | ucuaccuuuuuaggccggucccugg |
| | | aggugcuguuccagggccccggc |
| 7 | S protein PP | MFVFLVLLPLVSSQCVNLTTRTQLP |
| | (amino acid) | PAYTNSFTRGVYYPDKVFRSSVLHS |
| | (V08/V09) | TQDLFLPFFSNVTWFHAIHVSGTNG |
| | | TKRFDNPVLPFNDGVYFASTEKSNI |
| | | IRGWIFGTTLDSKTQSLLIVNNATN |
| | | VVIKVCEFQFCNDPFLGVYYHKNNK |
| | | SWMESEFRVYSSANNCTFEYVSQPF |
| | | LMDLEGKQGNFKNLREFVFKNIDGY |
| | | FKIYSKHTPINLVRDLPQGFSALEP |
| | | LVDLPIGINITRFQTLLALHRSYLT |
| | | PGDSSSGWTAGAAAYYVGYLQPRTF |
| | | LLKYNENGTITDAVDCALDPLSETK |
| | | CTLKSFTVEKGIYQTSNFRVQPTES |
| | | IVRFPNITNLCPFGEVFNATRFASV |
| | | YAWNRKRISNCVADYSVLYNSASFS |
| | | TFKCYGVSPTKLNDLCFTNVYADSF |
| | | VIRGDEVRQIAPGQTGKIADYNYKL |
| | | PDDFTGCVIAWNSNNLDSKVGGNYN |
| | | YLYRLFRKSNLKPFERDISTEIYQA |
| | | GSTPCNGVEGFNCYFPLQSYGFQPT |
| | | NGVGYQPYRVVVLSFELLHAPATVC |
| | | GPKKSTNLVKNKCVNFNFNGLTGTG |
| | | VLTESNKKFLPFQQFGRDIADTTDA |
| | | VRDPQTLEILDITPCSFGGVSVITP |
| | | GTNTSNQVAVLYQDVNCTEVPVAIH |
| | | ADQLTPTWRVYSTGSNVFQTRAGCL |
| | | IGAEHVNNSYECDIPIGAGICASYQ |
| | | TQTNSPRRARSVASQSIIAYTMSLG |
| | | AENSVAYSNNSIAIPTNFTISVTTE |
| | | ILPVSMTKTSVDCTMYICGDSTECS |
| | | NLLLQYGSFCTQLNRALTGIAVEQD |
| | | KNTQEVFAQVKQIYKTPPIKDFGGF |
| | | NFSQILPDPSKPSKRSFIEDLLFNK |
| | | VTLADAGFIKQYGDCLGDIAARDLI |
| | | CAQKFNGLTVLPPLLTDEMIAQYTS |
| | | ALLAGTITSGWTFGAGAALQIPFAM |
| | | QMAYRFNGIGVTQNVLYENQKLIAN |
| | | QFNSAIGKIQDSLSSTASALGKLQD |
| | | VVNQNAQALNTLVKQLSSNFGAISS |
| | | VLNDILSRLDPPEAEVQIDRLITGR |
| | | LQSLQTYVTQQLIRAAEIRASANLA |
| | | ATKMSECVLGQSKRVDFCGKGYHLM |
| | | SFPQSAPHGVVFLHVTYVPAQEKNF |
| | | TTAPAICHDGKAHFPREGVFVSNGT |
| | | HWFVTQRNFYEPQIITTDNTFVSGN |
| | | CDVVIGIVNNTVYDPLQPELDSFKE |
| | | ELDKYFKNHTSPDVDLGDISGINAS |
| | | VVNIQKEIDRLNEVAKNLNESLIDL |
| | | QELGKYEQYIKWPWYIWLGFIAGLI |
| | | AIVMVTIMLCCMTSCCSCLKGCCSC |
| | | GSCCKFDEDDSEPVLKGVKLHYT |
| 8 | S protein PP | auguuuguguuucuugugcugcugc |
| | (CDS) (V08) | cucuugugucuucucagugugugaa |
| | | uuugacaacaagaacacagcugcca |
| | | ccagcuuauacaaauuuuuuaccag |
| | | aggaguguauuauccugauaaagug |
| | | uuuagaucuucugugcugcacagca |
| | | cacaggaccuguuucugccauuuuu |
| | | uagcaaugugacaugguuucaugca |
| | | auucaugugucuggaacaaauggaa |
| | | caaaaagauuugauaauccugugcu |
| | | gccuuuuaaugauggaguguauuuu |
| | | gcuucaacagaaaagucaaauauua |
| | | uuagaggauggauuuuuggaacaac |
| | | acuggauucuaaaacacagucucug |
| | | cugauugugaauaaugcaacaaaug |
| | | uggugauuaaagugugugaauuuca |
| | | guuuuguaaugauccuuuucuggga |
| | | guguauuaucacaaaaauaauaaau |
| | | cuuggauggaaucugaauuuagagu |
| | | guauuccucugcaaauaauuguaca |
| | | uuugaauaugugucucagccuuuuc |
| | | ugauggaucuggaaggaaaacaggg |
| | | caauuuuaaaaaucugagagaauuu |
| | | guguuuaaaaauauugauggauauu |
| | | uuaaaauuuauucuaaacacacacc |
| | | aauuaauuuagugagagaucugccu |
| | | cagggauuuucugcucuggaaccuc |
| | | ugguggaucugccaauuggcauuaa |
| | | uauuacaagauuucagacacugcug |
| | | gcucugcacagaucuuaucugacac |
| | | cuggagauucuucuucuggauggac |
| | | agccggagcugcagcuuauuaugug |
| | | ggcuaucugcagccaagaacauuuc |
| | | ugcugaaauauaaugaaaauggaac |
| | | aauuacagaugcuguggauugugcu |
| | | cuggauccucugucugaaacaaaau |
| | | guacauuaaaaucuuuuacagugga |
| | | aaaaggcauuuaucagacaucuaau |
| | | uuuagagugcagccaacagaaucua |
| | | uugugagauuuccaaauauuacaaa |
| | | ucuguguccauuuggagaaguguuu |
| | | aaugcaacaagauuugcaucugugu |
| | | augcauggaauagaaaaagaauuuc |
| | | uaauuguguggcugauuauucugug |
| | | cuguauaauagugcuuuuuuuccac |
| | | auuuaaauguuauggagugucucca |
| | | acaaaauuaaaugauuuauguuuua |
| | | caaauguguaugcugauucuuuugu |
| | | gaucagaggugaugaagugagacag |
| | | auugcccccggacagacaggaaaaa |
| | | uugcugauuacaauuacaaacugcc |
| | | ugaugauluuuacaggaugugugau |
| | | ugcuuggaauucuaauaauuuagau |
| | | ucuaaagugggaggaaauuacaauu |
| | | aucuguacagacuguuuagaaaauc |
| | | aaaucugaaaccuuuugaaagagau |
| | | auuucaacagaaauuuaucaggcug |
| | | gaucaacaccuuguaauggagugga |
| | | aggauuuaauuguuauuuuccauua |
| | | cagagcuauggauuucagccaacca |
| | | auggugugggauaucagccauauag |
| | | agugguggugcugucuuuugaacug |
| | | cugcaugcaccugcaacagugugug |
| | | gaccuaaaaaaucuacaaauuuagu |
| | | gaaaaauaaaugugugaauuuuaau |
| | | uuuaauggauuaacaggaacaggag |
| | | ugcugacagaaucuaauaaaaaauu |
| | | ucugccuuuucagcaguuuggcaga |
| | | gauauugcagauaccacagaugcag |
| | | ugagagauccucagacauuagaaau |
| | | ucuggauauuacaccuuguuuuuug |
| | | ggggugugucugugauuacaccugg |
| | | aacaaauacaucuaaucagguggcu |
| | | gugcuguaucaggaugugaauugua |
| | | cagaagugccaguggcaauucaugc |
| | | agaucagcugacaccaacauggaga |
| | | guguauucuacaggaucuaaugugu |
| | | uucagacaagagcaggaugucugau |
| | | uggagcagaacaugugaauaauucu |
| | | uaugaaugugauauuccaauuggag |
| | | caggcauuugugcaucuuaucagac |
| | | acagacaaauuccccaaggagagca |
| | | agaucuguggcaucucagucuauua |
| | | uugcauacaccaugucucugggagc |
| | | agaaaauucuguggcauauucuaau |
| | | aauucuauugcuauuccaacaaauu |
| | | uuaccauuucugugacaacagaaau |
| | | uuuaccugugucuaugacaaaaaca |
| | | ucuguggauuguaccauguacauuu |
| | | guggagauucuacagaauguucuaa |
| | | ucugcugcugcaguauggaucuuuu |
| | | uguacacagcugaauagagcuuuaa |
| | | caggaauugcuguggaacaggauaa |
| | | aaauacacaggaaguguuugcucag |
| | | gugaaacagauuuacaaaacaccac |
| | | caauuaaagauuuuggaggauuuaa |
| | | uuuuagccagauucugccugauccu |
| | | ucuaaaccuucuaaaagaucuuuua |
| | | uugaagaucugcuguuuaauaaagu |
| | | gacacuggcagaugcaggauuuauu |
| | | aaacaguauggagauugccugggug |
| | | auauugcugcaagagaucugauuug |
| | | ugcucagaaauuuaauggacugaca |
| | | gugcugccuccucugcugacagaug |
| | | aaaugauugcucaguacacaucugc |
| | | uuuacuggcuggaacaauuacaagc |
| | | ggauggacauuuggagcuggagcug |
| | | cucugcagauuccuuuugcaaugca |
| | | gauggcuuacagauuuaauggaauu |
| | | ggagugacacagaauguguuauaug |
| | | aaaaucagaaacugauugcaaauca |
| | | guuuaauucugcaauuggcaaaauu |
| | | caggauucucugucuucuacagcuu |
| | | cugcucugggaaaacugcaggaugu |
| | | ggugaaucagaaugcacaggcacug |
| | | aauacucuggugaaacagcugucua |
| | | gcaauuuuggggcaauuucuucugu |
| | | gcugaaugauauucugucuagacug |
| | | gauccuccugaagcugaagugcaga |
| | | uugauagacugaucacaggaagacu |
| | | gcagucucugcagacuuaugugaca |
| | | cagcagcugauuagagcugcugaaa |
| | | uuagagcuucugcuaaucuggcugc |
| | | uacaaaaaugucugaaugugugcug |
| | | ggacagucaaaaagaguggauuuuu |
| | | guggaaaaggauaucaucugauguc |
| | | uuuuccacagucugcuccacaugga |
| | | gugguguuuuuacaugugacauaug |
| | | ugccagcacaggaaaagaauuuuac |
| | | cacagcaccagcaauuugucaugau |
| | | ggaaaagcacauuuuccaagagaag |
| | | gaguguuugugucuaauggaacaca |
| | | uugguuugugacacagagaaauuuu |
| | | uaugaaccucagauuauuacaacag |
| | | auaauacauuugugucaggaaauug |
| | | ugauguggugauuggaauugugaau |
| | | aauacaguguaugauccacugcagc |
| | | cagaacuggauuuuuuaaagaagaa |
| | | cuggauaaauauuuuaaaaaucaca |
| | | caucuccugauguggauuuaggaga |
| | | uauuucuggaaucaaugcaucugug |
| | | gugaauauucagaaagaaauugaua |
| | | gacugaaugaaguggccaaaaaucu |
| | | gaaugaaucucugauugaucugcag |
| | | gaacuuggaaaauaugaacaguaca |
| | | uuaaauggccuugguacauuuggcu |
| | | uggauuuauugcaggauuaauugca |
| | | auugugauggugacaauuauguuau |
| | | guuguaugacaucauguuguucuug |
| | | uuuaaaaggauguuguucuugugga |
| | | agcuguuguaaauuugaugaagaug |
| | | auucugaaccuguguuaaaaggagu |
| | | gaaauugcauuacaca |
| 9 | S protein PP | auguucguguuccuggugcugcugc |
| | (CDS) (V09) | cucugguguccagccagugugugaa |
| | | ccugaccaccagaacacagcugccu |
| | | ccagccuacaccaacagcuuuacca |
| | | gaggcguguacuaccccgacaaggu |
| | | guucagauccagcgugcugcacucu |
| | | acccaggaccuguuccugccuuucu |
| | | ucagcaacgugaccugguuccacgc |
| | | cauccacguguccggcaccaauggc |
| | | accaagagauucgacaaccccgugc |
| | | ugcccuucaacgacgggguguacuu |
| | | ugccagcaccgagaaguccaacauc |
| | | aucagaggcuggaucuucggcacca |
| | | cacuggacagcaagacccagagccu |
| | | gcugaucgugaacaacgccaccaac |
| | | guggucaucaaagugugcgaguucc |
| | | aguucugcaacgaccccuuccuggg |
| | | cgucuacuaccacaagaacaacaag |
| | | agcuggauggaaagcgaguuccggg |
| | | uguacagcagcgccaacaacugcac |
| | | cuucgaguacgugucccagccuuuc |
| | | cugauggaccuggaaggcaagcagg |
| | | gcaacuucaagaaccugcgcgaguu |
| | | cguguuuaagaacaucgacggcuac |
| | | uucaagaucuacagcaagcacaccc |
| | | cuaucaaccucgugcgggaucugcc |
| | | ucagggcuucucugcucuggaaccc |
| | | cugguggaucugcccaucggcauca |
| | | acaucacccgguuucagacacugcu |
| | | ggcccugcacagaagcuaccugaca |
| | | ccuggogauagcagcagcggaugga |
| | | cagcuggugccgccgcuuacuaugu |
| | | gggcuaccugcagccuagaaccuuc |
| | | cugcugaaguacaacgagaacggca |
| | | ccaucaccgacgccguggauugugc |
| | | ucuggauccucugagcgagacaaag |
| | | ugcacccugaaguccuucaccgugg |
| | | aaaagggcaucuaccagaccagcaa |
| | | cuuccgggugcagcccaccgaaucc |
| | | aucgugcgguuccccaauaucacca |
| | | aucugugccccuucggcgagguguu |
| | | caaugccaccagauucgccucugug |
| | | uacgccuggaaccggaagcggauca |
| | | gcaauugcguggccgacuacuccgu |
| | | gcuguacaacuccgccagcuucagc |
| | | accuucaagugcuacggcguguccc |
| | | cuaccaagcugaacgaccugugcuu |
| | | cacaaacguguacgccgacagcuuc |
| | | gugauccggggagaugaagugcggc |
| | | agauugccccuggacagacaggcaa |
| | | gaucgccgacuacaacuacaagcug |
| | | cccgacgacuucaccggcuguguga |
| | | uugccuggaacagcaacaaccugga |
| | | cuccaaagucggcggcaacuacaau |
| | | uaccuguaccggcuguuccggaagu |
| | | ccaaucugaagcocuucgaggggac |
| | | aucuccaccgagaucuaucaggccg |
| | | gcagcaccccuuguaacggcgugga |
| | | aggcuucaacugcuacuucccacug |
| | | caguccuacggcuuucagcccacaa |
| | | auggcgugggcuaucagcccuacag |
| | | agugguggugcugagcuucgaacug |
| | | cugcaugccccugccacagugugcg |
| | | gcccuaagaaaagcaccaaucucgu |
| | | gaagaacaaaugcgugaacuucaac |
| | | uucaacggccugaccggcaccggcg |
| | | ugcugacagagagcaacaagaaguu |
| | | ccugccauuccagcaguuuggccgg |
| | | gauaucgccgauaccacagacgccg |
| | | uuagagauccccagacacuggaaau |
| | | ccuggacaucaccccuugcagcuuc |
| | | ggeggagugucugugaucaccccug |
| | | gcaccaacaccagcaaucagguggc |
| | | agugcuguaccaggacgugaacugu |
| | | accgaagugcccguggccauucacg |
| | | cogaucagcugacaccuacauggcg |
| | | gguguacuccaccggcagcaaugug |
| | | uuucagaccagagccggcugucuga |
| | | ucggagocgagcacgugaacaauag |
| | | cuacgagugcgacauccccaucggc |
| | | gcuggaaucugcgocagcuaccaga |
| | | cacagacaaacagcccucggagagc |
| | | cagaagcguggccagccagagcauc |
| | | auugccuacacaaugucucugggcg |
| | | cogagaacagcguggccuacuccaa |
| | | caacucuaucgcuauccccaccaac |
| | | uucaccaucagcgugaccacagaga |
| | | uccugccuguguccaugaccaagac |
| | | cagcguggacugcaccauguacauc |
| | | ugcggcgauuccaccgagugcucca |
| | | accugcugcugcaguacggcagcuu |
| | | cugcacccagcugaauagagcccug |
| | | acagggaucgccguggaacaggaca |
| | | agaacacccaagagguguucgccca |
| | | agugaagcagaucuacaagaccccu |
| | | ccuaucaaggacuucggcggcuuca |
| | | auuucagccagauucugcccgaucc |
| | | uagcaagcccagcaagcggagcuuc |
| | | aucgaggaccugcuguucaacaaag |
| | | ugacacuggccgacgccggcuucau |
| | | caagcaguauggcgauugucugggc |
| | | gacauugccgccagggaucugauuu |
| | | gcgcccagaaguuuaacggacugac |
| | | agugcugccuccucugcugaccgau |
| | | gagaugaucgcccaguacacaucug |
| | | cccugcuggccggcacaaucacaag |
| | | cggcuggacauuuggagcaggcgcc |
| | | gcucugcagauccccuuugcuaugc |
| | | agauggccuaccgguucaacggcau |
| | | cggagugacccagaaugugcuguac |
| | | gagaaccagaagcugaucgccaacc |
| | | aguucaacagcgccaucggcaagau |
| | | ccaggacagccugagcagcacagca |
| | | agcgcccugggaaagcugcaggacg |
| | | uggucaaccagaaugcccaggcacu |
| | | gaacacccuggucaagcagcugucc |
| | | uccaacuucggcgccaucagcucug |
| | | ugcugaacgauauccugagcagacu |
| | | ggacccuccugaggccgaggugcag |
| | | aucgacagacugaucacaggcagac |
| | | ugcagagccuccagacauacgugac |
| | | ccagcagcugaucagagccgccgag |
| | | auuagagccucugccaaucuggccg |
| | | ccaccaagaugucugagugugugcu |
| | | gggccagagcaagagaguggacuuu |
| | | ugcggcaagggcuaccaccugauga |
| | | gcuucccucagucugccccucacgg |
| | | cgugguguuucugcacgugacauau |
| | | gugcccgcucaagagaagaauuuca |
| | | ccaccgcuccagccaucugccacga |
| | | cggcaaagcccacuuuccuagagaa |
| | | ggcguguucgugudcaacggcaccc |
| | | auugguucgugacacagcggaacuu |
| | | cuacgagccccagaucaucaccacc |
| | | gacaacaccuucgugucuggcaacu |
| | | gcgacgucgugaucggcauugugaa |
| | | caauaccguguacgacccucugcag |
| | | cccgagcuggacagcuucaaagagg |
| | | aacuggacaaguacuuuaagaacca |
| | | cacaagccccgacguggaccugggc |
| | | gauaucagcggaaucaaugccagcg |
| | | ucgugaacauccagaaagagaucga |
| | | ccggcugaacgagguggccaagaau |
| | | cugaacgagagccugaucgaccugc |
| | | aagaacuggggaaguacgagcagua |
| | | caucaaguggcccugguacaucugg |
| | | cugggcuuuaucgccggacugauug |
| | | ccaucgugauggucacaaucaugcu |
| | | guguugcaugaccagcugcuguagc |
| | | ugccugaagggcuguuguagcugug |
| | | gcagcugcugcaaguucgacgagga |
| | | cgauucugagcccgugcugaagggc |
| | | gugaaacugcacuacaca |

Foldon

| | | |
|---|---|---|
| 10 | Foldon (aa) | GSGYIPEAPRDGQAYVRKDGEWVLL |
| | | STFLGRSLEVLFQGPG |
| 11 | Foldon (CDS) | ggaucugguuauauuccugaagcuc |
| | | caagagaugggcaagcuuacguucg |
| | | uaaagauggcgaauggguauuacuu |
| | | ucuaccuuuuuaggccggucccugg |
| | | aggugcuguuccagggccccggc |

5′-UTR (hAg-Kozak)

| | | |
|---|---|---|
| 12 | 5′-UTR | AACUAGUAUUCUUCUGGUCCCCACA |
| | | GACUCAGAGAGAACCCGCCACC |

3′-UTR (FI element)

| | | |
|---|---|---|
| 13 | 3′-UTR | CUGGUACUGCAUGCACGCAAUGCUA |
| | | GCUGCCCCUUUCCCGUCCUGGGUAC |
| | | CCCGAGUCUCCCCCGACCUCGGGUC |
| | | CCAGGUAUGCUCCCACCUCCACCUG |
| | | CCCCACUCACCACCUCUGCUAGUUC |
| | | CAGACACCUCCCAAGCACGCAGCAA |
| | | UGCAGCUCAAAACGCUUAGCCUAGC |
| | | CACACCCCCACGGGAAACAGCAGUG |
| | | AUUAACCUUUAGCAAUAAACGAAAG |
| | | UUUAACUAAGCUAUACUAACCCCAG |
| | | GGUUGGUCAAUUUCGUGCCAGCCAC |
| | | ACC |

A30L70

| | |
|---|---|
| 14 | AAAAAAAAAAAAAAAAAAAAAAAAA |
| | AAAAAGCAUAUGACUAAAAAAAAAA |
| | AAAAAAAAAAAAAAAAAAAAAAAAA |
| | AAAAAAAAAAAAAAAAAAAAAAAAA |
| | AAAAAAAAAA |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in a preferred embodiment the composition (or formulation) comprises a cryoprotectant and in another preferred embodiment the cationically ionizable lipid has the structure I-3, then in a further preferred embodiment the composition (or formulation) comprises a cryoprotectant and the cationically ionizable lipid having the structure I-3.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Römpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%.

“Physiological pH” as used herein refers to a pH of about 7.5.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “% (w/v)” (or “% w/v”) refers to weight by volume percent, which is a unit of concentration measuring the amount of solute in grams (g) expressed as a percent of the total volume of solution in milliliters (ml).

As used in the present disclosure, “% by weight” or “% (w/w)” (or “% w/w”) refers to weight percent, which is a unit of concentration measuring the amount of a substance in grams (g) expressed as a percent of the total weight of the total composition in grams (g).

Regarding the presence of divalent inorganic ions, in particular divalent inorganic cations, their concentration or effective concentration (presence of free ions) due to the presence of chelating agents is in one embodiment sufficiently low so as to prevent degradation of the RNA. In one embodiment, the concentration or effective concentration of divalent inorganic ions is below the catalytic level for hydrolysis of the phosphodiester bonds between RNA nucleotides. In one embodiment, the concentration of free divalent inorganic ions is 20 μM or less. In one embodiment, there are no or essentially no free divalent inorganic ions.

“Osmolality” refers to the concentration of a particular solute expressed as the number of osmoles of solute per kilogram of solvent.

The term “freezing” relates to the solidification of a liquid, usually with the removal of heat.

The term “aqueous phase” as used herein in relation to a composition/formulation comprising particles, in particular LNPs, means the mobile or liquid phase, i.e., the continuous water phase including all components dissolved therein but (formally) excluding the particles. Thus, if particles, such as LNPs, are dispersed in an aqueous phase and the aqueous phase is to be substantially free of compound X, the aqueous phase is free of X is such manner as it is practically and realistically feasible, e.g., the concentration of compound X in the aqueous composition is less than 1% by weight. However, it is possible that, at the same time, the particles dispersed in the aqueous phase may comprise compound X in an amount of more than 1% by weight.

The expression “protonated form” as used herein in relation with a base (e.g., an organic primary amine such as Tris) means the conjugate acid of the base, wherein the conjugate acid contains a proton which is removable by deprotonation resulting in the base. For example, the protonated form of Tris has the formula [H₃N(CH₂CH₂OH)₃]⁺. A “buffer substance” as used herein refers to a mixture of the base and its protonated form (e.g., a mixture of Tris and [H₃N(CH₂CH₂OH)₃]⁺). Consequently, the amount of a buffer substance contained in a composition is sum of the amounts of both the base and the conjugate acid in the composition.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., preferably from about 15° C. to about 35° C., from about 15° C. to about 30° C., from about 15° C. to about 25° C., or from about 17° C. to about 22° C.

Such temperatures will include 15° C., 16° C., 17° C., 18° C., 19° C., 20° C., 21° C. and 22° C.

The term “alkyl” refers to a monoradical of a saturated straight or branched hydrocarbon. Preferably, the alkyl group comprises from 1 to 12 (such as 1 to 10) carbon atoms, i.e., 1, 2, 3, 4, 5, 6, 7, 8, 9, 10, 11, or 12 carbon atoms, abbreviated as C₁₋₁₂ alkyl, (such as 1, 2, 3, 4, 5, 6, 7, 8, 9, or 10 carbon atoms, abbreviated as C₁₋₁₀ alkyl), more preferably 1 to 8 carbon atoms, such as 1 to 6 or 1 to 4 carbon atoms.

Exemplary alkyl groups include methyl, ethyl, propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent, as specified herein. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2)hydrogen atoms of the alkylene group are replaced with a substituent other than hydrogen (when more than one hydrogen atom is replaced the substituents may be the same or different). Preferably, the substituent other than hydrogen is a 1^{st} level substituent, as specified herein.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenyl group comprises from 2 to 12, abbreviated as C₂₋₁₂ alkenyl, (e.g., 2 to 10) carbon atoms and 1, 2, 3, 4, 5, or 6 (e.g., 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, I-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. If an alkenyl group is attached to a nitrogen atom, the double bond cannot be alpha to the nitrogen atom. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). Preferably, the substituent other than hydrogen is a 1^{st} level substituent as specified herein.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom. A “substituted alkenylene” means that one or more (such as 1 to the maximum number of hydrogen atoms bound to an alkenylene group, e.g., 1, 2, 3, 4, 5, 6, 7, 8, 9, or up to 10, such as between 1 to 5, 1 to 4, or 1 to 3, or 1 or 2) hydrogen atoms of the alkenylene group are replaced with a substituent other than hydrogen (when more than one hydrogen atom is replaced the substituents may be the same or different). Preferably, the substituent other than hydrogen is a 1^{st} level substituent as specified herein.

The term “cycloalkylene” represents cyclic non-aromatic versions of “alkylene” and is a geminal, vicinal or isolated diradical. In certain embodiments, the cycloalkylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkylene. Generally, instead of using the ending “ylene” for cycloalkylene moieties as specified above, one can also use the ending “diyl” (e.g., 1,2-cyclopropylene can also be called cyclopropan-1,2-diyl) Exemplary cycloalkylene groups include cyclohexylene, cycloheptylene, cyclopropylene, cyclobutylene, cyclopentylene, cyclooctylene, bicyclo[3.2.1]octylene, bicyclo[3.2.2]nonylene, and adamantanylene (e.g., tricyclo[3.3.1.1^{3.7}]decan-2,2-diyl). A “substituted cycloalkylene” means that one or more (such as 1 to the maximum number of hydrogen atoms bound to an cycloalkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1′ level substituent as specified herein.

The term “cycloalkenylene” represents cyclic non-aromatic versions of “alkenylene” and is a geminal, vicinal or isolated diradical. Generally, the maximal number of carbon-carbon double bonds in the cycloalkenylene group can be equal to the integer which is calculated by dividing the number of carbon atoms in the cycloalkenylene group by 2 and, if the number of carbon atoms in the cycloalkenylene group is uneven, rounding the result of the division down to the next integer. For example, for an cycloalkenylene group having 9 carbon atoms, the maximum number of carbon-carbon double bonds is 4. Preferably, the cycloalkenylene group has 1 to 6 (such as 1 to 4), i.e., 1, 2, 3, 4, 5, or 6, carbon-carbon double bonds. In certain embodiments, the cycloalkenylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkenylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkenylene. Exemplary cycloalkenylene groups include cyclohexenylene, cycloheptenylene, cyclopropenylene, cyclobutenylene, cyclopentenylene, and cyclooctenylene. A “substituted cycloalkenylene” means that one or more (such as 1 to the maximum number of hydrogen atoms bound to an cycloalkenylene group, e.g., 1, 2, 3, 4, 5, 6, 7, 8, 9, or up to 10, such as between 1 to 5, 1 to 4, or 1 to 3, or 1 or 2) hydrogen atoms of the cycloalkenylene group are replaced with a substituent other than hydrogen (when more than one hydrogen atom is replaced the substituents may be the same or different). Preferably, the substituent other than hydrogen is a 1V level substituent as specified herein.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure.

Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic).

Typical 1^{st} level substituents are preferably selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. Particularly preferred 1^{st} level substituents are selected from the group consisting of methyl, ethyl, propyl, isopropyl, halogen (such as F, Cl, or Br), and —CF₃, such as halogen (e.g., F, Cl, or Br), and —CF₃.

The expression “after thawing the frozen composition”, as used herein in context with a frozen composition, means that the frozen composition has to be thawed before the characteristics (such as RNA integrity and/or size (Z_{average}) and/or size distribution and/or the PDI of the LNPs contained in the composition) can be measured.

A “monovalent” compound relates to a compound having only one functional group of interest. For example, a monovalent anion relates to a compound having only one negatively charged group, preferably under physiological conditions.

A “divalent” or “dibasic” compound relates to a compound having two functional groups of interest. For example, a dibasic organic acid has two acid groups.

A “polyvalent” or “polybasic” compound relates to a compound having three or more functional groups of interest. For example, a polybasic organic acid has three or more acid groups.

The expression “RNA integrity” means the percentage of the full-length (i.e., non-fragmented) RNA to the total amount of RNA (i.e., non-fragmented plus fragmented RNA) contained in a sample. The RNA integrity may be determined by chromatographically separating the RNA (e.g., using capillary electrophoresis), determining the peak area of the main RNA peak (i.e., the peak area of the full-length (i.e., non-fragmented) RNA), determining the peak area of the total RNA, and dividing the peak area of the main RNA peak by the peak area of the total RNA.

The term “cryoprotectant” relates to a substance that is added to a preparation (e.g., formulation or composition) in order to protect the active ingredients of the preparation during the freezing stages.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and immunostimulants such as cytokines.

According to the present disclosure, it is preferred that a nucleic acid such as RNA (e.g., mRNA) encoding a peptide or protein once taken up by or introduced, i.e. transfected or transduced, into a cell which cell may be present in vitro or in a subject results in expression of said peptide or protein. The cell may express the encoded peptide or protein intracellularly (e.g. in the cytoplasm and/or in the nucleus), may secrete the encoded peptide or protein, or may express it on the surface.

According to the present disclosure, terms such as “nucleic acid expressing” and “nucleic acid encoding” or similar terms are used interchangeably herein and with respect to a particular peptide or polypeptide mean that the nucleic acid, if present in the appropriate environment, preferably within a cell, can be expressed to produce said peptide or polypeptide.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

According to the present disclosure, a part or fragment of a peptide or protein preferably has at least one functional property of the peptide or protein from which it has been derived. Such functional properties comprise a pharmacological activity, the interaction with other peptides or proteins, an enzymatic activity, the interaction with antibodies, and the selective binding of nucleic acids. E.g., a pharmacological active fragment of a peptide or protein has at least one of the pharmacological activities of the peptide or protein from which the fragment has been derived. A part or fragment of a peptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains.

Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence.

In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In a preferred embodiment, the RNA (such as mRNA) used in the present disclosure is in substantially purified form. In one embodiment, a solution (preferably an aqueous solution) of RNA (such as mRNA) in substantially purified form contains a first buffer system.

The term “genetic modification” or simply “modification” includes the transfection of cells with nucleic acid. The term “transfection” relates to the introduction of nucleic acids, in particular RNA, into a cell.

For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection.

Generally, nucleic acid encoding antigen is transiently transfected into cells. RNA can be transfected into cells to transiently express its coded protein.

According to the present disclosure, an analog of a peptide or protein is a modified form of said peptide or protein from which it has been derived and has at least one functional property of said peptide or protein. E.g., a pharmacological active analog of a peptide or protein has at least one of the pharmacological activities of the peptide or protein from which the analog has been derived. Such modifications include any chemical modification and comprise single or multiple substitutions, deletions and/or additions of any molecules associated with the protein or peptide, such as carbohydrates, lipids and/or proteins or peptides. In one embodiment, “analogs” of proteins or peptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said proteins and peptides.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied.

In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be both a humoral as well as a cellular immune response. In the context of some embodiments of the present disclosure, the antigen is preferably presented by a cell, preferably by an antigen presenting cell, in the context of MHC molecules, which results in an immune response against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In a preferred embodiment, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4+ and CD8+ lymphocytes, in particular via the modulation of the activity of a T-cell receptor.

The term “tumor antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to the present disclosure, a tumor antigen preferably comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

Terms such as “epitope”, “fragment of an antigen”, “immunogenic peptide” and “antigen peptide” are used interchangeably herein and preferably relate to an incomplete representation of an antigen which is preferably capable of eliciting an immune response against the antigen or a cell expressing or comprising and preferably presenting the antigen. Preferably, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class 1 and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In one embodiment, vaccine antigen, i.e., an antigen whose inoculation into a subject induces an immune response, is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In one embodiment, an antigen is presented by a diseased cell (such as tumor cell or an infected cell). In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

In one embodiment, an antigen receptor is an antibody or B cell receptor which binds to an epitope in an antigen. In one embodiment, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In certain embodiments of the present disclosure, the RNA (in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In one embodiment, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells.

By “antigen-responsive CTL” is meant a CD8⁺ T-cell that is responsive to an antigen or a peptide derived from said antigen, which is presented with class I MHC on the surface of antigen presenting cells.

According to the disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and preferably refers to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, preferably CD4⁺ and CD8⁺ T-lymphocytes, more preferably CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, preferably the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. Preferably, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and the first component (C1q) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the V_{L} (variable light chain) domain, C_{L}(constant light chain) domain, V_{H} (variable heavy chain) domain, and the CH (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class I molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide or protein.

With respect to RNA, the term “expression” or “translation” relates to the process in the ribosomes of a cell by which a strand of mRNA directs the assembly of a sequence of amino acids to make a peptide or protein.

The term “optional” or “optionally” as used herein means that the subsequently described event, circumstance or condition may or may not occur, and that the description includes instances where said event, circumstance, or condition occurs and instances in which it does not occur.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (t). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero)cyclic rings, such as chair, half-chair, boat, and twist-boat forms of cyclohexane.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses m, (i=1, 2, 3, . . . , n), located at fixed distances s, from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors \vec{q} the structure function S is defined as follows:

$$S ( \vec{q} ) \approx N \cdot ( 1 - \frac{{q}^{2} - {R}_{g}^{2}}{3} )$$

wherein N is the number of components (Guinier's law).

The “D10 value”, in particular regarding a quantitative size distribution of particles, is the diameter at which 10% of the particles have a diameter less than this value. The D10 value is a means to describe the proportion of the smallest particles within a population of particles (such as within a particle peak obtained from a field-flow fractionation).

“D50 value”, in particular regarding a quantitative size distribution of particles, is the diameter at which 50% of the particles have a diameter less than this value. The D50 value is a means to describe the mean particle size of a population of particles (such as within a particle peak obtained from a field-flow fractionation).

The “D90 value”, in particular regarding a quantitative size distribution of particles, is the diameter at which 90% of the particles have a diameter less than this value. The “D95”, “D99”, and “D100” values have corresponding meanings. The D90, D95, D99, and D100 values are means to describe the proportion of the larger particles within a population of particles (such as within a particle peak obtained from a field-flow fractionation).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; Tis the temperature; η is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles such as LNPs contained in a formulation or composition as disclosed herein or the hydrodynamic radius of a particle peak obtained from subjecting such a formulation or composition to field-flow fractionation) is to measure the DLS signal of said particle or population of particles (such as DLS signal of particles such as LNPs contained in a formulation or composition as disclosed herein or the DLS signal of a particle peak obtained from subjecting such a formulation or composition to field-flow fractionation).

The term “aggregate” as used herein relates to a cluster of particles, wherein the particles are identical or very similar and adhere to each other in a non-covalently manner (e.g., via ionic interactions, H bridge interactions, dipole interactions, and/or van der Waals interactions).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In one preferred embodiment, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{{K}^{*} c} = {M}_{w} P ( θ ) - 2 {A}_{2} {M}_{w}^{2} {P}^{2} ( θ )$$

wherein c is the mass concentration of the particles in the solvent (g/mL); A₂ is the second virial coefficient (mol·mL/g²); P(θ) is a form factor relating to the dependence of scattered light intensity on angle; R_{θ} is the excess Rayleigh ratio (cm⁻¹); and K* is an optical constant that is equal to 4π²ηₒ (dn/dc)²λ₀⁻⁴N_{A}⁻¹, where ηₒ is the refractive index of the solvent at the incident radiation (vacuum) wavelength, λ₀ is the incident radiation (vacuum) wavelength (nm), N_{A} is Avogadro's number (mol⁻¹), and dn/dc is the differential refractive index increment (mL/g) (cf., e.g., Buchholz et al. (Electrophoresis 22(2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term:

$$\sqrt{\frac{{R}_{θ}}{{K}^{*} c}}$$

wherein c, R_{θ} and K* are as defined above. Preferably, the Debye plot is calculated the following term:

$$\frac{{K}^{*} c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is preferably modified by one or more alkyl groups, more preferably one or more C₁₋₄ alkyl groups, even more preferably one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N⁶—C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

Herein, the term “DNA” relates to a nucleic acid molecule which includes deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99/), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

RNA

According to the present disclosure, the term “RNA” means a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA).

In a preferred embodiment, the RNA comprises an open reading frame (ORF) encoding a peptide or protein.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

According to the present disclosure, the term “mRNA” means “messenger-RNA” and relates to a “transcript” which may be generated by using a DNA template and may encode a peptide or protein. Typically, an mRNA comprises a 5′-UTR, a peptide/protein coding region, and a 3′-UTR. In the context of the present disclosure, mRNA is preferably generated by in vitro transcription (IVT) from a DNA template. As set forth above, the in vitro transcription methodology is known to the skilled person, and a variety of in vitro transcription kits is commercially available.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide or protein.

In one embodiment, the RNA which preferably encodes a peptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

In one embodiment, the RNA (such as mRNA) contains a 5′ untranslated region (5′-UTR), a peptide coding region and a 3′ untranslated region (3′-UTR). In some embodiments, the RNA (such as mRNA) is produced by in vitro transcription or chemical synthesis. In one embodiment, the RNA (such as mRNA) is produced by in vitro transcription using a DNA template. The in vitro transcription methodology is known to the skilled person; cf., e.g., Molecular Cloning: A Laboratory Manual, 2^{nd} Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In one embodiment, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter.

A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). In one particularly preferred embodiment, the replicon or self-replicating RNA (such as self-replicating mRNA) is derived from or comprises elements derived from an ssRNA virus, in particular a positive-stranded ssRNA virus such as an alphavirus. Alphaviruses are typical representatives of positive-stranded RNA viruses.

Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment of the present disclosure, the RNA (such as mRNA) contains one or more modifications, e.g., in order to increase its stability and/or increase translation efficiency and/or decrease immunogenicity and/or decrease cytotoxicity. For example, in order to increase expression of the RNA (such as mRNA), it may be modified within the coding region, i.e., the sequence encoding the expressed peptide or protein, preferably without altering the sequence of the expressed peptide or protein. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA). The term “modification” in the context of modified mRNA according to the present disclosure preferably relates to any modification of an mRNA which is not naturally present in said RNA (such as mRNA).

In some embodiments, the RNA (such as mRNA) comprises a 5′-cap structure. In one embodiment, the mRNA does not have uncapped 5′-triphosphates. In one embodiment, the RNA (such as mRNA) may comprise a conventional 5′-cap and/or a 5′-cap analog. The term “conventional 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′triphosphate linkage to the rest of the mRNA). The guanosine may be methylated at position N⁷ (resulting in the cap structure m⁷Gppp). The term “5′-cap analog” refers to a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)).

Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2′O}G(5′)ppSp(5′)G (referred to as beta-S-ARCA or β-S-ARCA)), as described in PCT/EP2019/056502. Providing an RNA (such as mRNA) with a 5′-cap structure as described herein may be achieved by in vitro transcription of a DNA template in presence of a corresponding 5′-cap compound, wherein said 5′-cap structure is co-transcriptionally incorporated into the generated RNA (such as mRNA) strand, or the RNA (such as mRNA) may be generated, for example, by in vitro transcription, and the 5′-cap structure may be attached to the mRNA post-transcriptionally using capping enzymes, for example, capping enzymes of vaccinia virus.

In some embodiments, the RNA (such as mRNA) comprises a 5′-cap structure selected from the group consisting of m₂^{7,2′O}G(5′)ppSp(5′)G (in particular its D1 diastereomer), m₂^{7,3′O}G(5′)ppp(5′)G, and m₂^{7,3′-O}Gppp(m₁^{2′-O})ApG.

In some embodiments, the RNA (such as mRNA) comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The D1 diastereomer of beta-S-ARCA (β-S-ARCA) has the following structure:

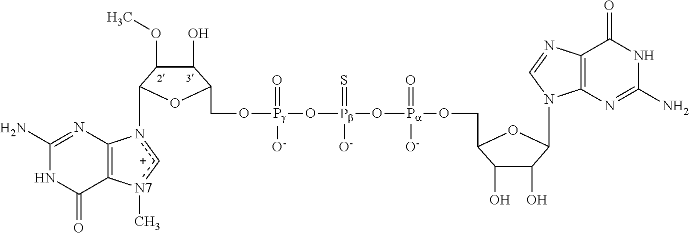

The “D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In one embodiment, a Supelcosil LC-18-T RP column, preferably of the format: 5 μm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp(m₁^{2′-O})ApG (also referred to as m₂^{7,3′O}G(5′)ppp(5′)m^{2′-O}ApG) which is a building block of a cap1 has the following structure.

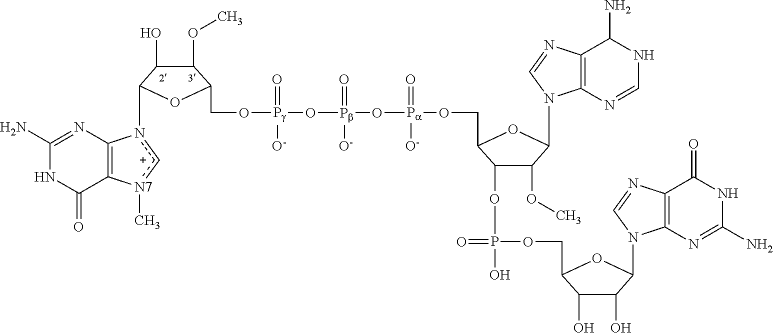

An exemplary cap0 miRNA comprising β-S-ARCA and mRNA has the following structure:

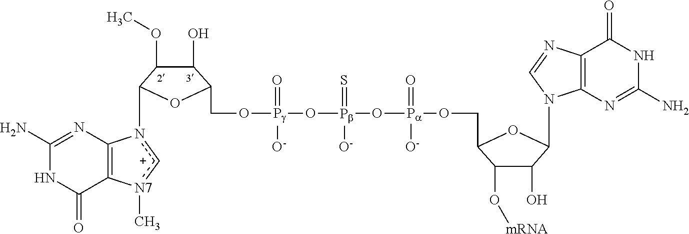

An exemplary cap0 mRNA comprising m₂^{7,3′O}G(5′)ppp(5′)G and mRNA has the following structure:

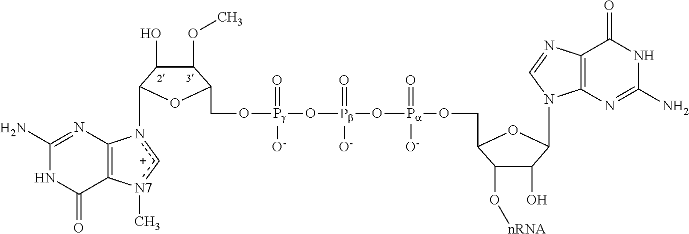

An exemplary cap1 mRNA comprising m₂^{7,3′-O}Gppp(m₁^{2′-O})ApG and mRNA has the following structure:

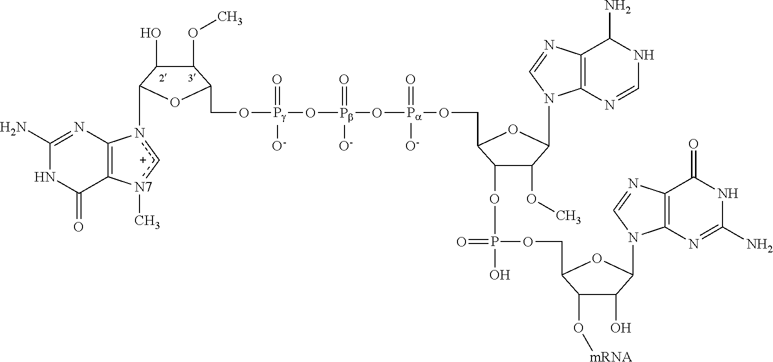

As used herein, the term “poly-A tail” or “poly-A sequence” refers to an uninterrupted or interrupted sequence of adenylate residues which is typically located at the 3′-end of an RNA (such as mRNA) molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs (such as mRNAs) disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of mRNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail am A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and d).

Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an mRNA molecule described herein essentially consists of A nucleotides, but is interrupted by a random sequence of the four nucleotides (A, C, G, U). Such random sequence may be 5 to 50, 10 to 30, or 10 to 20 nucleotides in length. In some embodiments, no nucleotides other than A nucleotides flank a poly-A tail at its 3′-end, i.e., the poly-A tail is not masked or followed at its 3′-end by a nucleotide other than A.

In one embodiment, the poly-A sequence comprises at least 100 nucleotides. In one embodiment, the poly-A sequence comprises or consists of the nucleotide sequence of SEQ ID NO: 14. In one embodiment, the poly-A sequence has a nucleotide sequence having at least 99%, 98%, 97%, 96%, 95%, 90%, 85%, or 80% identity to the nucleotide sequence of SEQ ID NO: 14.

In some embodiments, RNA (such as mRNA) used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (preferably mRNA) into which they are introduced. In one particular embodiment the 3′-UTR is derived from a globin gene or mRNA, such as a gene or mRNA of alpha2-globin, alpha1-globin, or beta-globin, preferably beta-globin, more preferably human beta-globin. For example, the RNA (preferably mRNA) may be modified by the replacement of the existing 3′-UTR with or the insertion of one or more, preferably two copies of a 3-UTR derived from a globin gene, such as alpha2-globin, alpha1-globin, beta-globin, preferably beta-globin, more preferably human beta-globin.

In some embodiments, the RNA (such as mRNA) used in present disclosure comprises a 5′ UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, the RNA (such as mRNA) used in present disclosure comprises a 3′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

The RNA (such as mRNA) may have modified ribonucleotides in order to increase its stability and/or decrease immunogenicity and/or decrease cytotoxicity. For example, in one embodiment, uridine in the RNA (such as mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N1-methyl-pseudouridine (m1ψ), 5-methyl-uridine (m5U), and combinations thereof.

In some embodiments, the modified nucleoside replacing (partially or completely, preferably completely) uridine in the RNA (such as mRNA) may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-undine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (um5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-cramoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

An RNA (preferably mRNA) which is modified by pseudouridine (replacing partially or completely, preferably completely, uridine) is referred to herein as “T-modified”, whereas the term “m1Ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such Ψ- or m1Ψ- or m5U-modified RNAs usually exhibit decreased immunogenicity compared to their unmodified forms and, thus, are preferred in applications where the induction of an immune response is to be avoided or minimized.

The codons of the RNA (preferably mRNA) used in the present disclosure may further be optimized, e.g., to increase the GC content of the RNA and/or to replace codons which are rare in the cell (or subject) in which the peptide or protein of interest is to be expressed by codons which are synonymous frequent codons in said cell (or subject). In some embodiments, the amino acid sequence encoded by the RNA used in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using the RNA (preferably mRNA) described herein. Codon-optimization is based on the finding that the translation efficiency is also determined by a different frequency in the occurrence of tRNAs in cells. Thus, the sequence of RNA (preferably mRNA) may be modified such that codons for which frequently occurring tRNAs are available are inserted in place of “rare codons”.

In some embodiments, the guanosine/cytosine (G/C) content of the coding region of the RNA (preferably mRNA) described herein is increased compared to the G/C content of the corresponding coding sequence of the wild type RNA, wherein the amino acid sequence encoded by the RNA (preferably mRNA) is preferably not modified compared to the amino acid sequence encoded by the wild type RNA. This modification of the RNA sequence is based on the fact that the sequence of any RNA region to be translated is important for efficient translation of that RNA (preferably mRNA). Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA (preferably mRNA), there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the mRNA described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine), and codon optimization, has a synergistic influence on the stability of RNA (preferably mRNA) and increase in translation efficiency.

Thus, in a preferred embodiment, the RNA (preferably mRNA) used in the present disclosure, in particular an RNA (preferably mRNA) encoding an antigen or epitope for inducing an immune response disclosed herein, contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine), and (v) codon optimization. In one embodiment, the RNA comprises a cap1 or cap2, preferably a cap1 structure. In one embodiment, the poly-A sequence comprises at least 100 nucleotides. In one embodiment, the poly-A sequence comprises or consists of the nucleotide sequence of SEQ ID NO: 14. In one embodiment, the a 5′ UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12. In one embodiment, the 3′ UTR comprising the nucleotide sequence of SEQ ID NO: 13, or a nucleotide sequence having at least 99%, 98%, 97%, 96%, 95%, 90%, 85%, or 80% h identity to the nucleotide sequence of SEQ ID NO: 13.

Some aspects of the disclosure involve the targeted delivery of the RNA (preferably mRNA) disclosed herein to certain cells or tissues. In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA (preferably mRNA) administered is RNA (preferably mRNA) encoding an antigen or epitope for inducing an immune response. In one embodiment, the target cell is a spleen cell. In one embodiment, the target cell is an antigen presenting cell such as a professional antigen presenting cell in the spleen. In one embodiment, the target cell is a dendritic cell in the spleen. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

Lipid-based RNA (such as mRNA) delivery systems have an inherent preference to the liver. Liver accumulation is caused by the discontinuous nature of the hepatic vasculature or the lipid metabolism (liposomes and lipid or cholesterol conjugates). In one embodiment, the target organ is liver and the target tissue is liver tissue. The delivery to such target tissue is preferred, in particular, if presence of mRNA or of the encoded peptide or protein in this organ or tissue is desired and/or if it is desired to express large amounts of the encoded peptide or protein and/or if systemic presence of the encoded peptide or protein, in particular in significant amounts, is desired or required.

In one embodiment, after administration of the RNA LNP compositions described herein, at least a portion of the RNA is delivered to a target cell or target organ. In one embodiment, at least a portion of the RNA is delivered to the cytosol of the target cell. In one embodiment, the RNA is RNA (preferably mRNA) encoding a peptide or protein and the RNA is translated by the target cell to produce the peptide or protein. In one embodiment, the target cell is a cell in the liver. In one embodiment, the target cell is a muscle cell. In one embodiment, the target cell is an endothelial cell. In one embodiment the target cell is a tumor cell or a cell in the tumor microenvironment. In one embodiment, the target cell is a blood cell. In one embodiment, the target cell is a cell in the lymph nodes. In one embodiment, the target cell is a cell in the lung. In one embodiment, the target cell is a blood cell. In one embodiment, the target cell is a cell in the skin. In one embodiment, the target cell is a spleen cell. In one embodiment, the target cell is an antigen presenting cell such as a professional antigen presenting cell in the spleen. In one embodiment, the target cell is a dendritic cell in the spleen. In one embodiment, the target cell is a T cell. In one embodiment, the target cell is a B cell. In one embodiment, the target cell is a NK cell. In one embodiment, the target cell is a monocyte. Thus, RNA LNP compositions described herein may be used for delivering RNA (preferably mRNA) to such target cell. Accordingly, the present disclosure also relates to a method for delivering RNA (preferably mRNA) to a target cell in a subject comprising the administration of the RNA LNP compositions described herein to the subject. In one embodiment, the RNA is delivered to the cytosol of the target cell. In one embodiment, the RNA is RNA (preferably mRNA) encoding a peptide or protein and the RNA is translated by the target cell to produce the peptide or protein.

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Both the coding strand, the nucleotide sequence of which is identical to the RNA sequence and is usually provided in sequence listings, and the non-coding strand, used as the template for transcription of a gene or cDNA, can be referred to as encoding the protein or other product of that gene or cDNA.

In one embodiment, RNA (preferably mRNA) used in the present disclosure comprises a nucleic acid sequence (e.g., an ORF) encoding one or more polypeptides, e.g., a peptide or protein, preferably a pharmaceutically active peptide or protein.

In a preferred embodiment, RNA (preferably mRNA) used in the present disclosure comprises a nucleic acid sequence (e.g., an ORF) encoding a peptide or protein, preferably a pharmaceutically active peptide or protein, and is capable of expressing said peptide or protein, in particular if transferred into a cell or subject. Thus, the RNA (preferably mRNA) used in the present disclosure preferably contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such RNA (preferably mRNA) encoding a pharmaceutically active peptide or protein is also referred to herein as “pharmaceutically active RNA” (or “pharmaceutically active mRNA”).

According to the present disclosure, the term “pharmaceutically active peptide or protein” means a peptide or protein that can be used in the treatment of an individual where the expression of a peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. Preferably, a pharmaceutically active peptide or protein has a positive or advantageous effect on the condition or disease state of an individual when administered to the individual in a therapeutically effective amount. A pharmaceutically active peptide or protein may have prophylactic properties and may be used to delay the onset of a disease or disorder or to lessen the severity of such disease or disorder. The term “pharmaceutically active peptide or protein” includes entire proteins or polypeptides, and can also refer to pharmaceutically active fragments thereof. It can also include pharmaceutically active analogs of a peptide or protein.

Specific examples of pharmaceutically active peptides and proteins include, but are not limited to, cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

The term “cytokines” relates to proteins which have a molecular weight of about 5 to 20 kDa and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. IFN-gamma is the sole type II interferon; all others are type I interferons. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFγ), interleukin 2 (IL-2), interleukin 4 (IL-4), interleukin 10 (IL-10), interleukin 11 (IL-11), interleukin 12 (IL-12), and interleukin 21 (IL-21).

In one embodiment, a pharmaceutically active peptide or protein comprises a replacement protein. In this embodiment, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In one embodiment, a “hormone” is a peptide or protein hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., P-selectin), and addressins.

Integrins are also involved in signal transduction. In particular, upon ligand binding, integrins modulate cell signaling pathways, e.g., pathways of transmembrane protein kinases such as receptor tyrosine kinases (RTK). Such regulation can lead to cellular growth, division, survival, or differentiation or to apoptosis. Particular examples of integrins include: α₁β₁, α₂β₁, α₃β₁, α₄β₁, α₅β₁, α₆β₁, α₇β₁, α_{L}β₂, α_{M}β₂, α_{IIb}β₃, α_{V}β₁, α_{V}β₃, α_{V}β₅, α_{V}β₆, α_{V}β₈, and α₆β₄.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86), and other.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants. Particular examples of immunologically active compounds include interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, and antigens, in particular tumor-associated antigens, pathogen-associated antigens (such as bacterial, parasitic, or viral antigens), allergens, and autoantigens. A preferred immunologically active compound is a vaccine antigen, i.e., an antigen whose inoculation into a subject induces an immune response.

The term “autoantigen” or “self-antigen” refers to an antigen which originates from within the body of a subject (i.e., the autoantigen can also be called “autologous antigen”) and which produces an abnormally vigorous immune response against this normal part of the body. Such vigorous immune reactions against autoantigens may be the cause of “autoimmune diseases”.

The term “allergen” refers to a kind of antigen which originates from outside the body of a subject (i.e., the allergen can also be called “heterologous antigen”) and which produces an abnormally vigorous immune response in which the immune system of the subject fights off a perceived threat that would otherwise be harmless to the subject. “Allergies” are the diseases caused by such vigorous immune reactions against allergens. An allergen usually is an antigen which is able to stimulate a type-I hypersensitivity reaction in atopic individuals through immunoglobulin E (IgE) responses. Particular examples of allergens include allergens derived from peanut proteins (e.g., Ara h 2.02), ovalbumin, grass pollen proteins (e.g., Phl p 5), and proteins of dust mites (e.g., Der p 2).

The term “growth factors” refers to molecules which are able to stimulate cellular growth, proliferation, healing, and/or cellular differentiation. Typically, growth factors act as signaling molecules between cells. The term “growth factors” include particular cytokines and hormones which bind to specific receptors on the surface of their target cells. Examples of growth factors include bone morphogenetic proteins (BMPs), fibroblast growth factors (FGFs), vascular endothelial growth factors (VEGFs), such as VEGFA, epidermal growth factor (EGF), insulin-like growth factor, ephrins, macrophage colony-stimulating factor, granulocyte colony-stimulating factor, granulocyte macrophage colony-stimulating factor, neuregulins, neurotrophins (e.g., brain-derived neurotrophic factor (BDNF), nerve growth factor (NGF)), placental growth factor (PGF), platelet-derived growth factor (PDGF), renalase (RNLS) (anti-apoptotic survival factor), T-cell growth factor (TCGF), thrombopoietin (TPO), transforming growth factors (transforming growth factor alpha (TGF-α), transforming growth factor beta (TGF-β)), and tumor necrosis factor-alpha (TNF-α). In one embodiment, a “growth factor” is a peptide or protein growth factor.

The term “protease inhibitors” refers to molecules, in particular peptides or proteins, which inhibit the function of proteases. Protease inhibitors can be classified by the protease which is inhibited (e.g., aspartic protease inhibitors) or by their mechanism of action (e.g., suicide inhibitors, such as serpins). Particular examples of protease inhibitors include serpins, such as alpha 1-antitrypsin, aprotinin, and bestatin.

The term “enzymes” refers to macromolecular biological catalysts which accelerate chemical reactions.

Like any catalyst, enzymes are not consumed in the reaction they catalyze and do not alter the equilibrium of said reaction. Unlike many other catalysts, enzymes are much more specific. In one embodiment, an enzyme is essential for homeostasis of a subject, e.g., any malfunction (in particular, decreased activity which may be caused by any of mutation, deletion or decreased production) of the enzyme results in a disease. Examples of enzymes include herpes simplex virus type 1 thymidine kinase (HSV1-TK), hexosaminidase, phenylalanine hydroxylase, pseudocholinesterase, and lactase.

The term “receptors” refers to protein molecules which receive signals (in particular chemical signals called ligands) from outside a cell. The binding of a signal (e.g., ligand) to a receptor causes some kind of response of the cell, e.g., the intracellular activation of a kinase. Receptors include transmembrane receptors (such as ion channel-linked (ionotropic) receptors, G protein-linked (metabotropic) receptors, and enzyme-linked receptors) and intracellular receptors (such as cytoplasmic receptors and nuclear receptors). Particular examples of receptors include steroid hormone receptors, growth factor receptors, and peptide receptors (i.e., receptors whose ligands are peptides), such as P-selectin glycoprotein ligand-1 (PSGL-1). The term “growth factor receptors” refers to receptors which bind to growth factors.

The term “apoptosis regulators” refers to molecules, in particular peptides or proteins, which modulate apoptosis, i.e., which either activate or inhibit apoptosis. Apoptosis regulators can be grouped into two broad classes: those which modulate mitochondrial function and those which regulate caspases. The first class includes proteins (e.g., BCL-2, BCL-xL) which act to preserve mitochondrial integrity by preventing loss of mitochondrial membrane potential and/or release of pro-apoptotic proteins such as cytochrome C into the cytosol. Also to this first class belong proapoptotic proteins (e.g., BAX, BAK, BIM) which promote release of cytochrome C. The second class includes proteins such as the inhibitors of apoptosis proteins (e.g., XIAP) or FLIP which block the activation of caspases.

The term “transcription factors” relates to proteins which regulate the rate of transcription of genetic information from DNA to messenger RNA, in particular by binding to a specific DNA sequence.

Transcription factors may regulate cell division, cell growth, and cell death throughout life; cell migration and organization during embryonic development; and/or in response to signals from outside the cell, such as a hormone. Transcription factors contain at least one DNA-binding domain which binds to a specific DNA sequence, usually adjacent to the genes which are regulated by the transcription factors. Particular examples of transcription factors include MECP2, FOXP2, FOXP3, the STAT protein family, and the HOX protein family.

The term “tumor suppressor proteins” relates to molecules, in particular peptides or proteins, which protect a cell from one step on the path to cancer. Tumor-suppressor proteins (usually encoded by corresponding tumor-suppressor genes) exhibit a weakening or repressive effect on the regulation of the cell cycle and/or promote apoptosis. Their functions may be one or more of the following: repression of genes essential for the continuing of the cell cycle; coupling the cell cycle to DNA damage (as long as damaged DNA is present in a cell, no cell division should take place); initiation of apoptosis, if the damaged DNA cannot be repaired; metastasis suppression (e.g., preventing tumor cells from dispersing, blocking loss of contact inhibition, and inhibiting metastasis); and DNA repair. Particular examples of tumor-suppressor proteins include p53, phosphatase and tensin homolog (PTEN), SWI/SNF (SWItch/Sucrose Non-Fermentable), von Hippel-Lindau tumor suppressor (pVHL), adenomatous polyposis coli (APC), CD95, suppression of tumorigenicity 5 (ST5), suppression of tumorigenicity 5 (ST5), suppression of tumorigenicity 14 (ST14), and Yippee-like 3 (YPEL3).

The term “structural proteins” refers to proteins which confer stiffness and rigidity to otherwise-fluid biological components. Structural proteins are mostly fibrous (such as collagen and elastin) but may also be globular (such as actin and tubulin). Usually, globular proteins are soluble as monomers, but polymerize to form long, fibers which, for example, may make up the cytoskeleton. Other structural proteins are motor proteins (such as myosin, kinesin, and dynein) which are capable of generating mechanical forces, and surfactant proteins. Particular examples of structural proteins include collagen, surfactant protein A, surfactant protein B, surfactant protein C, surfactant protein D, elastin, tubulin, actin, and myosin.

The term “reprogramming factors” or “reprogramming transcription factors” relates to molecules, in particular peptides or proteins, which, when expressed in somatic cells optionally together with further agents such as further reprogramming factors, lead to reprogramming or de-differentiation of said somatic cells to cells having stem cell characteristics, in particular pluripotency. Particular examples of reprogramming factors include OCT4, SOX2, c-MYC, KLF4, LIN28, and NANOG.

The term “genomic engineering proteins” relates to proteins which are able to insert, delete or replace DNA in the genome of a subject. Particular examples of genomic engineering proteins include meganucleases, zinc finger nucleases (ZFNs), transcription activator-like effector nucleases (TALENs), and clustered regularly spaced short palindromic repeat-CRISPR-associated protein 9 (CRISPR-Cas9).

The term “blood proteins” relates to peptides or proteins which are present in blood plasma of a subject, in particular blood plasma of a healthy subject. Blood proteins have diverse functions such as transport (e.g., albumin, transferrin), enzymatic activity (e.g., thrombin or ceruloplasmin), blood clotting (e.g., fibrinogen), defense against pathogens (e.g., complement components and immunoglobulins), protease inhibitors (e.g., alpha 1-antitrypsin), etc. Particular examples of blood proteins include thrombin, serum albumin, Factor VII, Factor VIII, insulin, Factor IX, Factor X, tissue plasminogen activator, protein C, von Willebrand factor, antithrombin III, glucocerebrosidase, erythropoietin, granulocyte colony stimulating factor (G-CSF), modified Factor VIII, and anticoagulants.

Thus, in one embodiment, the pharmaceutically active peptide or protein is (i) a cytokine, preferably selected from the group consisting of erythropoietin (EPO), interleukin 4 (IL-2), and interleukin 10 (IL-11), more preferably EPO; (ii) an adhesion molecule, in particular an integrin; (iii) an immunoglobulin, in particular an antibody; (iv) an immunologically active compound, in particular an antigen; (v) a hormone, in particular vasopressin, insulin or growth hormone; (vi) a growth factor, in particular VEGFA; (vii) a protease inhibitor, in particular alpha 1-antitrypsin; (viii) an enzyme, preferably selected from the group consisting of herpes simplex virus type 1 thymidine kinase (HSV1-TK), hexosaminidase, phenylalanine hydroxylase, pseudocholinesterase, pancreatic enzymes, and lactase; (ix) a receptor, in particular growth factor receptors; (x) an apoptosis regulator, in particular BAX; (xi) a transcription factor, in particular FOXP3; (xii) a tumor suppressor protein, in particular p53; (xiii) a structural protein, in particular surfactant protein B; (xiv) a reprogramming factor, e.g., selected from the group consisting of OCT4, SOX2, c-MYC, KLF4, LIN28 and NANOG; (xv) a genomic engineering protein, in particular clustered regularly spaced short palindromic repeat-CRISPR-associated protein 9 (CRISPR-Cas9); and
(xvi) a blood protein, in particular fibrinogen.

In one embodiment, a pharmaceutically active peptide or protein comprises one or more antigens or one or more epitopes, i.e., administration of the peptide or protein to a subject elicits an immune response against the one or more antigens or one or more epitopes in a subject which may be therapeutic or partially or fully protective.

In certain embodiments, the RNA (preferably mRNA) encodes at least one epitope.

In certain embodiments, the epitope is derived from a tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUD IN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 1 1, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies.

The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by mRNA described herein, e.g., as a single polypeptide wherein the epitopes are optionally separated by linkers. In certain embodiments of the present disclosure, the mRNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include mRNA that encodes at least five epitopes (termed a “pentatope”) and mRNA that encodes at least ten epitopes (termed a “decatope”).

In certain embodiments, the epitope is derived from a pathogen-associated antigen, in particular from a viral antigen. In one embodiment, the epitope is derived from a SARS-CoV-2 S protein, an immunogenic variant thereof, or an immunogenic fragment of the SARS-CoV-2 S protein or the immunogenic variant thereof. Thus, in one embodiment, the RNA (preferably mRNA) used in the present disclosure encodes an amino acid sequence comprising a SARS-CoV-2 S protein, an immunogenic variant thereof, or an immunogenic fragment of the SARS-CoV-2 S protein or the immunogenic variant thereof. In one embodiment, the RNA comprises an ORF encoding a full-length SARS-CoV2 S protein variant with proline residue substitutions at positions 986 and 987 of SEQ ID NO:1. In one embodiment, the SARS-CoV2 S protein variant has at least 80% identity (such as at least 85% identity, at least 90% identity, at least 91% identity, at least 92% identity, at least 93% identity, at least 94% identity, at least 95% identity, at least 96% identity, at least 97% identity, at least 98% identity, or at least 99% identity) to SEQ ID NO:7.

In one embodiment, an immunogenic fragment of the SARS-CoV-2 S protein comprises the S1 subunit of the SARS-CoV-2 S protein, or the receptor binding domain (RBD) of the S1 subunit of the SARS-CoV-2 S protein. In some embodiments, the RNA (e.g., mRNA) used in the present disclosure comprises an open reading frame encoding a polypeptide that comprises a receptor-binding portion of a SARS-CoV-2 S protein, which RNA is suitable for intracellular expression of the polypeptide. In some embodiments, such an encoded polypeptide does not comprise the complete S protein. In some embodiments, the encoded polypeptide comprises the receptor binding domain (RBD), for example, as shown in SEQ ID NO: 5. In some embodiments, the encoded polypeptide comprises the peptide according to SEQ ID NO: 29 or 31.

In one embodiment, the amino acid sequence comprising a SARS-CoV-2 S protein, an immunogenic variant thereof, or an immunogenic fragment of the SARS-CoV-2 S protein or the immunogenic variant thereof is able to form a multimeric complex, in particular a trimeric complex. To this end, the amino acid sequence comprising a SARS-CoV-2 S protein, an immunogenic variant thereof, or an immunogenic fragment of the SARS-CoV-2 S protein or the immunogenic variant thereof may comprise a domain allowing the formation of a multimeric complex, in particular a trimeric complex of the amino acid sequence comprising a SARS-CoV-2 S protein, an immunogenic variant thereof, or an immunogenic fragment of the SARS-CoV-2 S protein or the immunogenic variant thereof. In one embodiment, the domain allowing the formation of a multimeric complex comprises a trimerization domain, for example, a trimerization domain as described herein.

In one embodiment, the trimerization domain as defined herein includes, without being limited thereto, a sequence comprising the amino acid sequence of amino acids 3 to 29 of SEQ ID NO: 10 or a functional variant thereof. In one embodiment, the trimerization domain as defined herein includes, without being limited thereto, a sequence comprising the amino acid sequence of SEQ ID NO: 10 or a functional variant thereof.

In one embodiment, a trimerization domain comprises the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In one embodiment, a trimerization domain comprises the amino acid sequence of amino acids 3 to 29 of SEQ ID NO: 10.

In one embodiment, RNA encoding a trimerization domain (i) comprises the nucleotide sequence of nucleotides 7 to 87 of SEQ ID NO: 11, a nucleotide sequence having at least 99%, 98%, 97%, 96%, 95%, 90%, 85%, or 80% identity to the nucleotide sequence of nucleotides 7 to 87 of SEQ ID NO: 11, or a fragment of the nucleotide sequence of nucleotides 7 to 87 of SEQ ID NO: 11, or the nucleotide sequence having at least 99%, 98%, 97%, 96%, 95%, 90%, 85%, or 80% identity to the nucleotide sequence of nucleotides 7 to 87 of SEQ ID NO: 11; and/or (ii) encodes an amino acid sequence comprising the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In one embodiment, RNA encoding a trimerization domain (i) comprises the nucleotide sequence of nucleotides 7 to 87 of SEQ ID NO: 11; and/or (ii) encodes an amino acid sequence comprising the amino acid sequence of amino acids 3 to 29 of SEQ ID NO: 10.

In some embodiments, the RBD antigen expressed by an RNA encoding a SARS-CoV-2 S protein, an immunogenic variant thereof, or an immunogenic fragment of the SARS-CoV-2 S protein or the immunogenic variant thereof (e.g., as described herein) can be modified by addition of a T4-fibritin-derived “foldon” trimerization domain, for example, to increase its immunogenicity.

In one embodiment, the amino acid sequence comprising a SARS-CoV-2 S protein, an immunogenic variant thereof, or an immunogenic fragment of the SARS-CoV-2 S protein or the immunogenic variant thereof is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1.

In one embodiment, (i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7.

In one embodiment, the amino acid sequence comprising a SARS-CoV-2 S protein, an immunogenic variant thereof, or an immunogenic fragment of the SARS-CoV-2 S protein or the immunogenic variant thereof comprises a secretory signal peptide.

In one embodiment, the secretory signal peptide is fused, preferably N-terminally, to a SARS-CoV-2 S protein, an immunogenic variant thereof, or an immunogenic fragment of the SARS-CoV-2 S protein or the immunogenic variant thereof.

In one embodiment,
(i) the RNA encoding the secretory signal peptide comprises the nucleotide sequence of nucleotides 1 to 48 of SEQ ID NO: 2, 8 or 9, a nucleotide sequence having at least 99%, 98%, 97%, 96%, 95%, 90%, 85%, or 80% identity to the nucleotide sequence of nucleotides 1 to 48 of SEQ ID NO: 2, 8 or 9, or a fragment of the nucleotide sequence of nucleotides 1 to 48 of SEQ ID NO: 2, 8 or 9, or the nucleotide sequence having at least 99%, 98%, 97%, 96%, 95%, 90%, 85%, or 80% identity to the nucleotide sequence of nucleotides 1 to 48 of SEQ ID NO: 2, 8 or 9; and/or
(ii) the secretory signal peptide comprises the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or
(ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 4, a nucleotide sequence having at least 99%, 98%, 97%, 96%, 95%, 90%, 85%, or 80% identity to the nucleotide sequence of SEQ ID NO: 4, or a fragment of the nucleotide sequence of SEQ ID NO: 4, or the nucleotide sequence having at least 99%, 98%, 97%, 96%, 95%, 90%, 85%, or 80% identity to the nucleotide sequence of SEQ ID NO: 4; and/or
(ii) encodes an amino acid sequence comprising the amino acid sequence of SEQ ID NO: 3, an amino acid sequence having at least 99%, 98%, 97%, 96%, 95%, 90%, 85%, or 80% identity to the amino acid sequence of SEQ ID NO: 3, or an immunogenic fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 4; and/or (ii) encodes an amino acid sequence comprising the amino acid sequence of SEQ ID NO: 3.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 716 of SEQ ID NO: 30, a nucleotide sequence having at least 99%, 98%, 977%, 96%, 95%, 90%, 85%, or 80% identity to the nucleotide sequence of nucleotides 54 to 716 of SEQ ID NO: 30, or a fragment of the nucleotide sequence of nucleotides 54 to 716 of SEQ ID NO: 30, or the nucleotide sequence having at least 99%, 987%, 97%, 96%, 95%, 90%, 85%, or 80% identity to the nucleotide sequence of nucleotides 54 to 716 of SEQ ID NO: 30; and/or
(ii) encodes an amino acid sequence comprising the amino acid sequence of amino acids 1 to 221 of SEQ ID NO: 29, an amino acid sequence having at least 99%, 98%, 97%, 96%, 95%, 90%, 85%, or 80% identity to the amino acid sequence of amino acids 1 to 221 of SEQ ID NO: 29, or an immunogenic fragment of the amino acid sequence of amino acids 1 to 221 of SEQ ID NO: 29, or the amino acid sequence having at least 99%, 98%, 97%, 96%, 95%, 90%, 85%, or 80% identity to the amino acid sequence of amino acids 1 to 221 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 716 of SEQ ID NO: 30; and/or (ii) encodes an amino acid sequence comprising the amino acid sequence of amino acids 1 to 221 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 725 of SEQ ID NO: 32, a nucleotide sequence having at least 99%, 98%, 97%, 96%, 95%, 90%, 85%, or 80% identity to the nucleotide sequence of nucleotides 54 to 725 of SEQ ID NO: 32, or a fragment of the nucleotide sequence of nucleotides 54 to 725 of SEQ ID NO: 32, or the nucleotide sequence having at least 99%, 98%, 97%, 96%, 95%, 90%, 85%, or 80% identity to the nucleotide sequence of nucleotides 54 to 725 of SEQ ID NO: 32; and/or
(ii) encodes an amino acid sequence comprising the amino acid sequence of amino acids 1 to 224 of SEQ ID NO: 31, an amino acid sequence having at least 99%, 98%, 97%, 96%, 95%, 90%, 85%, or 80% identity to the amino acid sequence of amino acids 1 to 224 of SEQ ID NO: 31, or an immunogenic fragment of the amino acid sequence of amino acids 1 to 224 of SEQ ID NO: 31, or the amino acid sequence having at least 99%, 98%, 97%, 96%, 95%, 90%, 85%, or 80% identity to the amino acid sequence of amino acids 1 to 224 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 17, 21, or 26, a nucleotide sequence having at least 99%, 98%, 97%, 96%, 95%, 90%, 85%, or 80% identity to the nucleotide sequence of SEQ ID NO: 17, 21, or 26, or a fragment of the nucleotide sequence of SEQ ID NO: 17, 21, or 26, or the nucleotide sequence having at least 99%, 98%, 97%, 96%, 95%, 90%, 85%, or 80% identity to the nucleotide sequence of SEQ ID NO: 17, 21, or 26; and/or
(ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 17, 21, or 26; and/or (ii) encodes an amino acid sequence comprising the amino acid sequence of SEQ ID NO: 5.

In one embodiment, a vaccine antigen comprises the amino acid sequence of SEQ ID NO: 18, an amino acid sequence having at least 99%, 98%, 97%, 96%, 95%, 90%, 85%, or 80% identity to the amino acid sequence of SEQ ID NO: 18, or an immunogenic fragment of the amino acid sequence of SEQ ID NO: 18, or the amino acid sequence having at least 99%, 98%, 97%, 96%, 95%, 90%, 85%, or 80% identity to the amino acid sequence of SEQ ID NO: 18. In one embodiment, a vaccine antigen comprises the amino acid sequence of SEQ ID NO: 18.

In one embodiment, a vaccine antigen comprises the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29. In one embodiment, a vaccine antigen comprises the amino acid sequence of amino acids 1 to 257 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 824 of SEQ ID NO: 30, a nucleotide sequence having at least 99%, 98%, 97%, 96%, 95%, 90%, 85%, or 80% identity to the nucleotide sequence of nucleotides 54 to 824 of SEQ ID NO: 30, or a fragment of the nucleotide sequence of nucleotides 54 to 824 of SEQ ID NO: 30, or the nucleotide sequence having at least 99%, 98%, 97%, 96%, 95%, 90%, 85%, or 80% identity to the nucleotide sequence of nucleotides 54 to 824 of SEQ ID NO: 30; and/or
(ii) encodes an amino acid sequence comprising the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 824 of SEQ ID NO: 30; and/or (ii) encodes an amino acid sequence comprising the amino acid sequence of amino acids 1 to 257 of SEQ ID NO: 29.

In one embodiment, a vaccine antigen comprises the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31. In one embodiment, a vaccine antigen comprises the amino acid sequence of amino acids 1 to 260 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 833 of SEQ ID NO: 32, a nucleotide sequence having at least 99%, 98%, 97%, 96%, 95%, 90%, 85%, or 80% identity to the nucleotide sequence of nucleotides 54 to 833 of SEQ ID NO: 32, or a fragment of the nucleotide sequence of nucleotides 54 to 833 of SEQ ID NO: 32, or the nucleotide sequence having at least 99%, 98%, 97%, 96%, 95%, 90%, 85%, or 80% identity to the nucleotide sequence of nucleotides 54 to 833 of SEQ ID NO: 32; and/or
(ii) encodes an amino acid sequence comprising the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 833 of SEQ ID NO: 32; and/or (ii) encodes an amino acid sequence comprising the amino acid sequence of amino acids 1 to 260 of SEQ ID NO: 31.

In one embodiment, a vaccine antigen comprises the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, a vaccine antigen comprises the amino acid sequence of amino acids 20 to 257 of SEQ ID NO: 29.

In one embodiment, the RNA encoding a SARS-CoV-2 S protein, an immunogenic variant thereof, or an immunogenic figment of the SARS-CoV-2 S protein or the immunogenic variant thereof
(i) comprises the nucleotide sequence of nucleotides 111 to 824 of SEQ ID NO: 30, a nucleotide sequence having at least 99%, 98%, 97%, 96%, 95%, 90%, 85%, or 80% identity to the nucleotide sequence of nucleotides 111 to 824 of SEQ ID NO: 30, or a fragment of the nucleotide sequence of nucleotides 111 to 824 of SEQ ID NO: 30, or the nucleotide sequence having at least 99%, 98%, 97%, 96%, 95%, 90%, 85%, or 80% identity to the nucleotide sequence of nucleotides 111 to 824 of SEQ ID NO: 30; and/or
(ii) encodes an amino acid sequence comprising the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, RNA encoding a vaccine antigen (i) comprises the nucleotide sequence of nucleotides 111 to 824 of SEQ ID NO: 30; and/or (ii) encodes an amino acid sequence comprising the amino acid sequence of amino acids 20 to 257 of SEQ ID NO: 29.

In one embodiment, a vaccine antigen comprises the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31. In one embodiment, a vaccine antigen comprises the amino acid sequence of amino acids 23 to 260 of SEQ ID NO: 31.

In one embodiment, the RNA encoding a SARS-CoV-2 S protein, an immunogenic variant thereof or an immunogenic fragment of the SARS-CoV-2 S protein or the immunogenic variant thereof
(i) comprises the nucleotide sequence of nucleotides 120 to 833 of SEQ ID NO: 32, a nucleotide sequence having at least 99%, 98%, 97%, 96%, 95%, 90%, 85%, or 80% identity to the nucleotide sequence of nucleotides 120 to 833 of SEQ ID NO: 32, or a fragment of the nucleotide sequence of nucleotides 120 to 833 of SEQ ID NO: 32, or the nucleotide sequence having at least 99%, 98%, 97%, 96%, 95%, 90%, 85%, or 80% identity to the nucleotide sequence of nucleotides 120 to 833 of SEQ ID NO: 32; and/or
(ii) encodes an amino acid sequence comprising the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 120 to 833 of SEQ ID NO: 32; and/or (ii) encodes an amino acid sequence comprising the amino acid sequence of amino acids 23 to 260 of SEQ ID NO: 31.

According to certain embodiments, a transmembrane domain is fused, either directly or through a linker, e.g., a glycine/serine linker, to a SARS-CoV-2 S protein, a variant thereof, or a fragment thereof, i.e., the antigenic peptide or protein. Accordingly, in one embodiment, a transmembrane domain is fused to the above described amino acid sequences derived from SARS-CoV-2 S protein or immunogenic fragments thereof (antigenic peptides or proteins) comprised by the vaccine antigens described above (which may optionally be fused to a signal peptide and/or trimerization domain as described above). Such transmembrane domains are preferably located at the C-terminus of the antigenic peptide or protein, without being limited thereto. Preferably, such transmembrane domains are located at the C-terminus of the trimerization domain, if present, without being limited thereto. In one embodiment, a trimerization domain is present between the SARS-CoV-2 S protein, a variant thereof, or a fragment thereof, i.e., the antigenic peptide or protein, and the transmembrane domain. Transmembrane domains as defined herein preferably allow the anchoring into a cellular membrane of the antigenic peptide or protein as encoded by the RNA.

In one embodiment, the transmembrane domain sequence as defined herein includes, without being limited thereto, the transmembrane domain sequence of SARS-CoV-2 S protein, in particular a sequence comprising the amino acid sequence of amino acids 1207 to 1254 of SEQ ID NO: 1 or a functional variant thereof.

In one embodiment, a transmembrane domain sequence comprises the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In one embodiment, a transmembrane domain sequence comprises the amino acid sequence of amino acids 1207 to 1254 of SEQ ID NO: 1.

In one embodiment, RNA encoding a transmembrane domain sequence (i) comprises the nucleotide sequence of nucleotides 3619 to 3762 of SEQ ID NO: 2, 8 or 9, a nucleotide sequence having at least 99%, 98%, 97%, 96%, 95%, 90%, 85%, or 80% identity to the nucleotide sequence of nucleotides 3619 to 3762 of SEQ ID NO: 2, 8 or 9, or a fragment of the nucleotide sequence of nucleotides 3619 to 3762 of SEQ ID NO: 2, 8 or 9, or the nucleotide sequence having at least 99%, 98%, 97%, 96%, 95%, 90%, 85%, or 80% identity to the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In one embodiment, RNA encoding a transmembrane domain sequence (i) comprises the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1.

In one embodiment, a vaccine antigen comprises the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29. In one embodiment, a vaccine antigen comprises the amino acid sequence of amino acids 1 to 311 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 986 of SEQ ID NO: 30, a nucleotide sequence having at least 99%, 98%, 97%, 96%, 95%, 90%, 85%, or 80% identity to the nucleotide sequence of nucleotides 54 to 986 of SEQ ID NO: 30, or a fragment of the nucleotide sequence of nucleotides 54 to 986 of SEQ ID NO: 30, or the nucleotide sequence having at least 99%, 98%, 97%, 96%, 95%, 90%, 85%, or 80% identity to the nucleotide sequence of nucleotides 54 to 986 of SEQ ID NO: 30; and/or
(ii) encodes an amino acid sequence comprising the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 986 of SEQ ID NO: 30; and/or (ii) encodes an amino acid sequence comprising the amino acid sequence of amino acids 1 to 311 of SEQ ID NO: 29.

In one embodiment, a vaccine antigen comprises the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31. In one embodiment, a vaccine antigen comprises the amino acid sequence of amino acids 1 to 314 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 995 of SEQ ID NO: 32, a nucleotide sequence having at least 99%, 98%, 97%, 96%, 95%, 90%6, 85%, or 80% identity to the nucleotide sequence of nucleotides 54 to 995 of SEQ ID NO: 32, or a fragment of the nucleotide sequence of nucleotides 54 to 995 of SEQ ID NO: 32, or the nucleotide sequence having at least 99%, 98%, 97%, 96%, 95%, 90%, 85%, or 80% identity to the nucleotide sequence of nucleotides 54 to 995 of SEQ ID NO: 32; and/or
(ii) encodes an amino acid sequence comprising the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 995 of SEQ ID NO: 32; and/or (ii) encodes an amino acid sequence comprising the amino acid sequence of amino acids 1 to 314 of SEQ ID NO: 31. In one embodiment, a vaccine antigen comprises the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29. In one embodiment, a vaccine antigen comprises the amino acid sequence of amino acids 20 to 311 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 111 to 986 of SEQ ID NO: 30, a nucleotide sequence having at least 99%, 98%, 97%, 96%, 95%, 90%, 85%, or 80% identity to the nucleotide sequence of nucleotides 111 to 986 of SEQ ID NO: 30, or a fragment of the nucleotide sequence of nucleotides 111 to 986 of SEQ ID NO: 30, or the nucleotide sequence having at least 99%, 98%, 97%, 96%, 95%, 90%, 85%, or 80% identity to the nucleotide sequence of nucleotides 111 to 986 of SEQ ID NO: 30; and/or
(ii) encodes an amino acid sequence comprising the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 111 to 986 of SEQ ID NO: 30; and/or (ii) encodes an amino acid sequence comprising the amino acid sequence of amino acids 20 to 311 of SEQ ID NO: 29.

In one embodiment, a vaccine antigen comprises the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95/c, 90%, 85%, or 80% identity to the amino acid sequence of amino acids 23 to 314 of SEQ ID NO: 31. In one embodiment, a vaccine antigen comprises the amino acid sequence of amino acids 23 to 314 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 120 to 995 of SEQ ID NO: 32, a nucleotide sequence having at least 99%, 98%, 97%, 96%, 95%, 90%, 85%, or 80% identity to the nucleotide sequence of nucleotides 120 to 995 of SEQ ID NO: 32, or a fragment of the nucleotide sequence of nucleotides 120 to 995 of SEQ ID NO: 32, or the nucleotide sequence having at least 99%, 98%, 97%, 96%, 95%, 90%, 85%, or 80% identity to the nucleotide sequence of nucleotides 120 to 995 of SEQ ID NO: 32; and/or
(ii) encodes an amino acid sequence comprising the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95%, 90%, 85%, or 80% identity to the amino acid sequence of amino acids 23 to 314 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 120 to 995 of SEQ ID NO: 32; and/or (ii) encodes an amino acid sequence comprising the amino acid sequence of amino acids 23 to 314 of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 30, a nucleotide sequence having at least 99%, 98%, 97%, 96%, 95%, 90%, 85%, or 80% identity to the nucleotide sequence of SEQ ID NO: 30, or a fragment of the nucleotide sequence of SEQ ID NO: 30, or the nucleotide sequence having at least 99%, 98%, 97%, 96%, 95%, 90%, 85%, or 80% identity to the nucleotide sequence of SEQ ID NO: 30; and/or
(ii) encodes an amino acid sequence comprising the amino acid sequence of SEQ ID NO: 29, an amino acid sequence having at least 99%, 98%, 97%, 96%, 95%, 90%, 85%, or 80% identity to the amino acid sequence of SEQ ID NO: 29, or an immunogenic fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29.

In one embodiment, RNA encoding a vaccine antigen (i) comprises the nucleotide sequence of SEQ ID NO: 30; and/or (ii) encodes an amino acid sequence comprising the amino acid sequence of SEQ ID NO: 29.

In one embodiment, the RNA encoding a SARS-CoV-2 S protein, an immunogenic variant thereof or an immunogenic fragment of the SARS-CoV-2 S protein or the immunogenic variant thereof
(i) comprises the nucleotide sequence of SEQ ID NO: 32, a nucleotide sequence having at least 99%, 98%, 97%, 96%, 95%, 90%, 85%, or 80% identity to the nucleotide sequence of SEQ ID NO: 32, or a fragment of the nucleotide sequence of SEQ ID NO: 32, or the nucleotide sequence having at least 99%, 98%, 97%, 96%, 95%, 90%, 85%, or 80% identity to the nucleotide sequence of SEQ ID NO: 32; and/or
(ii) encodes an amino acid sequence comprising the amino acid sequence of SEQ ID NO: 31, an amino acid sequence having at least 99%, 98%, 97%, 96%, 95%, 90%, 85%, or 80% identity to the amino acid sequence of SEQ ID NO: 31, or an immunogenic fragment of the amino acid sequence of SEQ ID NO: 31, or the amino acid sequence having at least 99%, 98%, 97%, 96%, 95%, 90%, 85%, or 80% identity to the amino acid sequence of SEQ ID NO: 31.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 32; and/or (ii) encodes an amino acid sequence comprising the amino acid sequence of SEQ ID NO: 31.

In one embodiment, a vaccine antigen comprises the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28. In one embodiment, a vaccine antigen comprises the amino acid sequence of SEQ ID NO: 28.

In one embodiment, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 27, a nucleotide sequence having at least 99%, 98%, 97%, 96%, 95%, 90%, 85%, or 80% identity to the nucleotide sequence of SEQ ID NO: 27, or a fragment of the nucleotide sequence of SEQ ID NO: 27, or the nucleotide sequence having at least 99%, 98%, 97%, 96%, 95%, 90%, 85%, or 80% identity to the nucleotide sequence of SEQ ID NO: 27; and/or
(ii) encodes an amino acid sequence comprising the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 27; and/or (ii) encodes an amino acid sequence comprising the amino acid sequence of SEQ ID NO: 28.

In one embodiment, the vaccine antigens described above comprise a contiguous sequence of SARS-CoV-2 coronavirus spike (S) protein that consists of or essentially consists of the above described amino acid sequences derived from SARS-CoV-2 S protein or immunogenic fragments thereof (antigenic peptides or proteins) comprised by the vaccine antigens described above. In one embodiment, the vaccine antigens described above comprise a contiguous sequence of SARS-CoV-2 coronavirus spike (S) protein of no more than 220 amino acids, 215 amino acids, 210 amino acids, or 205 amino acids.

In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) described herein as BNT162b1 (RBP020.3), BNT162b2 (RBP020.1 or RBP020.2). In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) described herein as RBP020.2.

In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 21, a nucleotide sequence having at least 99%, 98%, 97%, 96%, 95%, 90%, 85%, or 80% identity to the nucleotide sequence of SEQ ID NO: 21, and/or (ii) encodes an amino acid sequence comprising the amino acid sequence of SEQ ID NO: 5, or an amino acid sequence having at least 99%, 98%, 97%, 96%, 95%, 90%, 85%, or 80% identity to the amino acid sequence of SEQ ID NO: 5. In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 21; and/or (ii) encodes an amino acid sequence comprising the amino acid sequence of SEQ ID NO: 5.

In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 19, or 20, a nucleotide sequence having at least 99%, 98%, 97%, 96%, 95%, 90%, 85%, or 80% identity to the nucleotide sequence of SEQ ID NO: 19, or 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 19, or 20; and/or (ii) encodes an amino acid sequence comprising the amino acid sequence of SEQ ID NO: 7.

In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 20, a nucleotide sequence having at least 99%, 98%, 97%, 96%, 95%, 90%, 85%, or 80% identity to the nucleotide sequence of SEQ ID NO: 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 20; and/or (ii) encodes an amino acid sequence comprising the amino acid sequence of SEQ ID NO: 7.

In one embodiment, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 725 of SEQ ID NO: 32; and/or (ii) encodes an amino acid sequence comprising the amino acid sequence of amino acids 1 to 224 of SEQ ID NO: 31.

In one embodiment of the RNA encoding a SARS-CoV-2 S protein, an immunogenic variant thereof, or an immunogenic fragment of the SARS-CoV-2 S protein or the immunogenic variant thereof, said RNA contains one or more of the above described RNA modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine), and codon optimization. In one embodiment, said RNA contains a combination of the above described modifications, preferably a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (f) or N(I)-methylpseudouridine (m1Ψ) or 5-methyluridine (m5U) for uridine), and (v) codon optimization.

In one embodiment of the RNA encoding a SARS-CoV-2 S protein, an immunogenic variant thereof, or an immunogenic fragment of the SARS-CoV-2 S protein or the immunogenic variant thereof, said RNA is a modified RNA, in particular a stabilized mRNA. In one embodiment, said RNA comprises a modified nucleoside in place of at least one uridine. In one embodiment, said RNA comprises a modified nucleoside in place of uridine, such as in place of each uridine. In one embodiment, the modified nucleoside is independently selected from pseudouridine (ψ), N1-methyl-pseudouridine (m1ψ), and 5-methyl-uridine (m5U). In one embodiment, said RNA comprises a 5′ cap, preferably a cap1 or cap2 structure, more preferably a cap1 structure. In one embodiment, said RNA comprises a 5′ UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12. In one embodiment, said RNA comprises a 3′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13. In one embodiment, said RNA comprises a poly-A sequence. In one embodiment, the poly-A sequence comprises at least 100 nucleotides. In one embodiment, the poly-A sequence comprises or consists of the nucleotide sequence of SEQ ID NO: 14.

In one embodiment of the RNA encoding a SARS-CoV-2 S protein, an immunogenic variant thereof, or an immunogenic fragment of the SARS-CoV-2 S protein or the immunogenic variant thereof, said variants include mutations in RBD (e.g., but not limited to Q321L, V341I, A348T, N354D, S359N, V367F, K378R, R408I, Q409E, A435S, N439K, K458R, I472V, G476S, S477N, V483A, Y508H, H519P, etc., as compared to SEQ ID NO: 1), and/or mutations in spike protein (e.g., but not limited to D614G, etc., as compared to SEQ ID NO: 1). Those skilled in the art are aware of various spike variants, and/or resources that document them (e.g., the Table of mutating sites in Spike maintained by the COVID-19 Viral Genome Analysis Pipeline and found at https://cov.lanl.gov/components/sequence/COV/int_sites_tbls.comp) (last accessed 24 Aug. 2020), and, reading the present specification, will appreciate that RNA compositions and/or methods described herein can be characterized for their ability to induce sera in vaccinated subject that display neutralizing activity with respect to any or all of such variants and/or combinations thereof.

In one embodiment of the RNA encoding a SARS-CoV-2 S protein, an immunogenic variant thereof, or an immunogenic fragment of the SARS-CoV-2 S protein or the immunogenic variant thereof, said variants include a mutation at position 501 in spike protein as compared to SEQ ID NO: 1 and optionally may include one or more further mutations as compared to SEQ ID NO: 1 (e.g., but not limited to H69/V70 deletion, Y144 deletion, A570D, D614G, P681H, T716I, S982A, D1118H, D80A, D215G, E484K, A701V, L18F, R246I, K417N, L242/A243/L244 deletion etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include “Variant of Concern 202012/01” (VOC-202012/01; also known as lineage B.1.1.7). The variant had previously been named the first Variant Under Investigation in December 2020 (VUI—202012/01) by Public Health England, but was reclassified to a Variant of Concern (VOC-202012/01). VOC-202012/01 is a variant of SARS-CoV-2 which was first detected in October 2020 during the COVID-19 pandemic in the United Kingdom from a sample taken the previous month, and it quickly began to spread by mid-December. It is correlated with a significant increase in the rate of COVID-19 infection in United Kingdom; this increase is thought to be at least partly because of change N501Y inside the spike glycoprotein's receptor-binding domain, which is needed for binding to ACE2 in human cells. The VOC-202012/01 variant is defined by 23 mutations: 13 non-synonymous mutations, 4 deletions, and 6 synonymous mutations (i.e., them are 17 mutations that change proteins and six that do not). The spike protein changes in VOC 202012/01 include deletion 69-70, deletion 144, N501Y, A570D, D614G, P681H, 17161, S982A, and D1I18H. One of the most important changes in VOC-202012/01 seems to be N501Y, a change from asparagine (N) to tyrosine (Y) at amino-acid site 501. This mutation alone or in combination with the deletion at positions 69/70 in the N terminal domain (NTD) may enhance the transmissibility of the virus.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: deletion 69-70, deletion 144, N501Y, A570D, D614G, P681H, 1716I, S982A, and D1118H as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include variant “501.V2”. This variant was first observed in samples from October 2020, and since then more than 300 cases with the 501.V2 variant have been confirmed by whole genome sequencing (WGS) in South Africa, where in December 2020 it was the dominant form of the virus. Preliminary results indicate that this variant may have an increased transmissibility. The 501.V2 variant is defined by multiple spike protein changes including: D80A, D215G, E484K, N501Y and A701V, and more recently collected viruses have additional changes: L18F, R246I, K417N, and deletion 242-244.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a H69/V70 deletion in spike protein as compared to SEQ ID NO: 1. Said SARs-CoV-2 spike variant may also include one or more further mutations as compared to SEQ ID NO: 1 (e.g., but not limited to Y144 deletion, N501Y, A570D, D614G, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1). In particular embodiments, said SARs-CoV-2 spike variant includes the following mutations: deletion 69-70, deletion 144, N501Y, A570D, D614G, P681H, T716I, S982A, and D1118H as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include variant “Cluster 5”, also referred to as ΔFVI-spike by the Danish State Serum Institute (SSI). It was discovered in North Jutland, Denmark, and is believed to have been spread from minks to humans via mink farms. In cluster 5, several different mutations in the spike protein of the virus have been confirmed. The specific mutations include 69-70deltaHV (a deletion of the histidine and valine residues at the 69th and 70th position in the protein), Y453F (a change from tyrosine to phenylalanine at position 453), I692V (isoleucine to valine at position 692), M1229I (methionine to isoleucine at position 1229), and optionally S1147L (serine to leucine at position 1147).

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: deletion 69-70, Y453F, 1692V, M12291, and optionally S1147L, as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 614 in spike protein as compared to SEQ ID NO: 1, such as a D614G mutation in spike protein as compared to SEQ ID NO: 1.

Said SARs-CoV-2 spike variants including a mutation at position 614 in spike protein as compared to SEQ ID NO: 1 may also include one or more further mutations as compared to SEQ ID NO: 1 (e.g., but not limited to H69N70 deletion, Y144 deletion, N501Y, A570D, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: deletion 69-70, deletion 144, N501Y, A570D, D614G, P681H, T716I, S982A, and D1118H as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501 and 614 in spike protein as compared to SEQ ID NO: 1. In some embodiments, said SARs-CoV-2 spike variants include a N501Y mutation and a D614G mutation in spike protein as compared to SEQ ID NO: 1. In some embodiments, said SARs-CoV-2 spike variants include one or more further mutations as compared to SEQ ID NO: 1 (e.g., but not limited to H69N70 deletion, Y144 deletion, A570D, P681H, T716I, S982A, D1118H, D80A, D215G, E484K, A701V, L18F, R2461, K417N, L242/A243/L244 deletion, Y453F, I692V, S1147L, M1229I etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: deletion 69-70, deletion 144, N501Y, A570D, D614G, P681H, T716I, S982A, and D1118H as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 484 in spike protein as compared to SEQ ID NO: 1, such as a E484K mutation in spike protein as compared to SEQ ID NO: 1.

In some embodiments, said SARs-CoV-2 spike variants may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, A701V, L18F, R246I, K417N, L242/A243/L244 deletion, Y453F, I692V, S1147L, M1229L, T20N, P26S, D138Y, R190S, K417T, H655Y, T1027I, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, and A701V, as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include variant lineage B.1.1.248, known as the Brazil(ian) variant. This variant of SARS-CoV-2 has been named P.1 lineage and has 17 unique amino acid changes, 10 of which in its spike protein, including N501Y and E484K. B.1.1.248 originated from B.1.1.28. E484K is present in both B.1.1.28 and B.1.1.248. B.1.1.248 has a number of S-protein polymorphisms [L18F, T20N, P26S, D138Y, R190S, K417T, E484K, N501Y, H655Y, T1027I, V1176F] and is similar in certain key RBD positions (K417, E484, N501) to variant described from South Africa.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: L18F, T20N, P26S, D138Y, R190S, K417T, E484K, N501Y, H655Y, T1027I, and V1176F as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501 and 484 in spike protein as compared to SEQ ID NO: 1, such as a N501Y mutation and a E484K mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, A570D, D614G, P681, T716I, S982A, D1118H, D80A, D215G, A701V, L18F, R246I, K417N, L242/A243/L244 deletion, Y453F, I692V, S1147L, M12291, T20N, P26S, D138Y, R190S, K417T, H655Y, T1027I, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: L18F, T20N, P26S, D138Y, R190S, K417T, E484K, N501Y, H655Y, T1027I, and V1176F as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501, 484 and 614 in spike protein as compared to SEQ ID NO: 1, such as a N501Y mutation, a E484K mutation and a D614G mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, A570D, P681H, 1716I, S982A, D1118H, D80A, D215G, A701V, L18F, R246I, K417N, L242/A243/L244 deletion, Y453F, I692V, S1147L, M1229I, T20N, P26S, D138Y, R190S, K417T, H655Y, T10271, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a L242/A243/L244 deletion in spike protein as compared to SEQ ID NO: 1. In some embodiments, said SARs-CoV-2 spike variants may include one or more further mutations as compared to SEQ ID NO: 1 (e.g., but not limited to H69N70 deletion, Y144 deletion, N501Y, A570D, D614G, P681H, T716I, S982A, D1118H, D80A, D215G, E484K, A701V, L18F, R246I, K417N, Y453F, 1692V, S1147L, M12291, T20N, P26S, D138Y, R190S, K417T, H655Y, T1027I, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V and deletion 242-244 as compared to SEQ ID NO: 1, and optionally: L18F, R246, and K417N, as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 417 in spike protein as compared to SEQ ID NO: 1, such as a K417N or K417T mutation in spike protein as compared to SEQ ID NO: 1. In some embodiments, said SARs-CoV-2 spike variants may include one or more further mutations as compared to SEQ ID NO: 1 (e.g., but not limited to H69N70 deletion, Y144 deletion, N501Y, A570D, D614G, P681H, 17161, S982A, D1118H, D80A, D215G, E484K, A701V, L18F, R246I, L242/A243/L244 deletion, Y453F, I692V, S1147L, M1229L, T20N, P26S, D138Y, R190S, H655Y, T1027I, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: L18F, T20N, P26S, D138Y, R190S, K417T, E484K, N501 Y, H655Y, T1027L, and V176F as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 417 and 484 and/or 501 in spike protein as compared to SEQ ID NO: 1, such as a K417N or K417T mutation and a E484K and/or N501Y mutation in spike protein as compared to SEQ ID NO: 1. In some embodiments, said SARs-CoV-2 spike variants may include one or more further mutations as compared to SEQ ID NO: 1 (e.g., but not limited to H69N70 deletion, Y144 deletion, A570D, D614G, P681H, 1716I, S982A, D1118H, D80A, D215G, A701V, L18F, R246I, L242/A243/L244 deletion, Y453F, I692V, S1147L, M1229I, T20N, P26S, D138Y, R190S, H655Y, T1027I, V1176F etc., as compared to SEQ ID NO: 1).

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In one embodiment of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including following mutations: L18F, T20N, P26S, D138Y, R190S, K417T, E484K, N501Y, H655Y, T1027I, and V1176F as compared to SEQ ID NO: 1.

The SARs-CoV-2 spike variants described herein may or may not include a D614G mutation as compared to SEQ ID NO: 1.

In one embodiment of the present disclosure, the antigen (such as a tumor antigen or vaccine antigen) is preferably administered as single-stranded, 5′ capped RNA (preferably mRNA) that is translated into the respective protein upon entering cells of a subject being administered the RNA. Preferably, the RNA contains structural elements optimized for maximal efficacy of the RNA with respect to stability and translational efficiency (5′ cap, 5′ UTR, 3′ UTR, poly(A) sequence).

In one embodiment, beta-S-ARCA(D1) is utilized as specific capping structure at the 5′-end of the RNA. In one embodiment, m₂^{7,3′-O}Gppp(m₁^{2′-O})ApG is utilized as specific capping structure at the 5′-end of the RNA. In one embodiment, the 5′-UTR sequence is derived from the human alpha-globin mRNA and optionally has an optimized ‘Kozak sequence’ to increase translational efficiency. In one embodiment, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. In one embodiment, two re-iterated 3′-UTRs derived from the human beta-globin mRNA are placed between the coding sequence and the poly(A) sequence to assure higher maximum protein levels and prolonged persistence of the mRNA. In one embodiment, a poly(A) sequence measuring 110 nucleotides in length, consisting of a stretch of 30 adenosine residues, followed by a 10 nucleotide linker sequence and another 70 adenosine residues is used. This poly(A) sequence was designed to enhance RNA stability and translational efficiency.

In the following, embodiments of three different RNA platforms are described each of which encodes a SARS-CoV-2 S protein, an immunogenic variant thereof, or an immunogenic fragment of the SARS-CoV-2 S protein or the immunogenic variant thereof.

In general, vaccine RNA described herein may comprise, from 5′ to 3′, one of the following structures:
Cap-5′-UTR-Vaccine Antigen-Encoding Sequence-3′-UTR-Poly(A)
   or
beta-S-ARCA(D1)-hAg-Kozak-Vaccine Antigen-Encoding Sequence-FI-A30L70.

In general, a vaccine antigen described herein may comprise, from N-terminus to C-terminus, one of the following structures:
Signal Sequence-RBD-Trimerization Domain
   or
Signal Sequence-RBD-Trimerization Domain-Transmembrane Domain.

RBD and Trimerization Domain may be separated by a linker, in particular a GS linker such as a linker having the amino acid sequence GSPGSGSGS (SEQ ID NO: 33). Trimerization Domain and Transmembrane Domain may be separated by a linker, in particular a GS linker such as a linker having the amino acid sequence GSGSGS (SEQ ID NO: 34).

Signal Sequence may be a signal sequence as described herein. RBD may be a RBD domain as described herein. Trimerization Domain may be a trimerization domain as described herein. Transmembrane Domain may be a transmembrane domain as described herein.

In one embodiment,
Signal sequence comprises the amino acid sequence of amino acids 1 to 16 or 1 to 19 of SEQ ID NO: 1 or the amino acid sequence of amino acids 1 to 22 of SEQ ID NO: 31, or an amino acid sequence having at least 99%6, 98%, 97%, 96%, 95%, 90%, 85%, or 80% identity to this amino acid sequence,
RBD comprises the amino acid sequence of amino acids 327 to 528 of SEQ ID NO: 1, or an amino acid sequence having at least 99%, 98%, 97%, 96%, 95%, 90%, 85%, or 80% identity to this amino acid sequence,
Trimerization Domain comprises the amino acid sequence of amino acids 3 to 29 of SEQ ID NO: 10 or the amino acid sequence of SEQ ID NO: 10, or an amino acid sequence having at least 99%, 98%, 97%, 96%, 95%, 90%, 85%, or 80% identity to this amino acid sequence; and
Transmembrane Domain comprises the amino acid sequence of amino acids 1207 to 1254 of SEQ ID NO: 1, or an amino acid sequence having at least 99%, 98%, 97%, 96%, 95%, 90%, 85%, or 80% identity to this amino acid sequence.

In one embodiment,
Signal sequence comprises the amino acid sequence of amino acids 1 to 16 or 1 to 19 of SEQ ID NO: 1 or the amino acid sequence of amino acids 1 to 22 of SEQ ID NO: 31,
RBD comprises the amino acid sequence of amino acids 327 to 528 of SEQ ID NO: 1, Trimerization Domain comprises the amino acid sequence of amino acids 3 to 29 of SEQ ID NO: 10 or the amino acid sequence of SEQ ID NO: 10; and
Transmembrane Domain comprises the amino acid sequence of amino acids 1207 to 1254 of SEQ ID NO: 1.

The above described RNA or RNA encoding the above described vaccine antigen may be non-modified uridine containing mRNA (uRNA), nucleoside modified mRNA (modRNA) or self-amplifying RNA (saRNA). In one embodiment, the above described RNA or RNA encoding the above described vaccine antigen is nucleoside modified mRNA (modRNA).

Non-Modified Uridine Messenger RNA (uRNA)

The active principle of the non-modified messenger RNA (uRNA) is a single-stranded mRNA that is translated upon entering a cell. In addition to the sequence encoding the coronavirus vaccine antigen (i.e. open reading frame), each uRNA preferably contains common structural elements optimized for maximal efficacy of the RNA with respect to stability and translational efficiency (5′-cap, 5′-UTR, 3′-UTR, poly(A)-tail). The preferred 5′ cap structure is beta-S-ARCA(D1)(m₂^{7,2′-O}GppSpG). The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14.

Different embodiments of this platform are as follows:

RBL063.1 (SEQ ID NO: 15; SEQ ID NO: 7)

| | |
|---|---|
| Structure | beta-S-ARCA(D1)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

RBL063.2 (SEQ ID NO: 16; SEQ ID NO: 7)

| | |
|---|---|
| Structure | beta-S-ARCA(D1)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

BNT162a1; RBL063.3 (SEQ ID NO: 17; SEQ ID NO: 5)

| | |
|---|---|
| Structure | beta-S-ARCA(D1)-hAg-Kozak-RBD-GS-Fibritin-FI-A30L70 |
| Encoded antigen | Viral spike protein (S protein) of the SARS-CoV-2 (partial sequence, Receptor |
| | Binding Domain (RBD) of S1S2 protein) |

In this respect, “hAg-Kozak” mean the 5′-UTR sequence of the human alpha-globin mRNA with an optimized ‘Kozak sequence’ to increase translational efficiency; “S1S2 protein”/“S1S2 RBD” means the sequences encoding the respective antigen of SARS-CoV-2; “FI element” means that the 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression; “A30L70” means a poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells; “GS” means a glycine-serine linker, i.e., sequences coding for short linker peptides predominantly consisting of the amino acids glycine (G) and serine (S), as commonly used for fusion proteins.

Nucleoside Modified Messenger RNA (modRNA)

The active principle of the nucleoside modified messenger RNA (modRNA) drug substance is as well a single-stranded mRNA that is translated upon entering a cell. In addition to the sequence encoding the coronavirus vaccine antigen (i.e., open reading frame), each modRNA contains common structural elements optimized for maximal efficacy of the RNA as the uRNA (5′-cap, 5′-UTR, 3′-UTR, poly(A)-tail). Compared to the uRNA, modRNA contains 1-methyl-pseudouridine instead of uridine. The preferred 5′ cap structure is m₂^{7,3′-O}Gppp(m₁^{2′-O})ApG. The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14. An additional purification step is applied for modRNA to reduce dsRNA contaminants generated during the in vitro transcription reaction.

Different embodiments of this platform are as follows:

BNT162b2; RBP020.1 (SEQ ID NO: 19; SEQ ID NO: 7)

| | |
|---|---|
| Structure | m₂^{7, 3′-O}Gppp(m₁^{2′-O})ApG)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

BNT162b2; RBP020.2 (SEQ ID NO: 20; SEQ ID NO: 7)

| | |
|---|---|
| Structure | m₂^{7, 3′-O}Gppp(m₁^{2′-O})ApG)-hAg-Kozak-S1S2-PP-FI-A30L70 |
| Encoded antigen | Viral spike protein (S1S2 protein) of the SARS-CoV-2 (S1S2 full-length |
| | protein, sequence variant) |

BNT162b1; RBP020.3 (SEQ ID NO: 21; SEQ ID NO: 5)

| | |
|---|---|
| Structure | m₂^{7, 3′-O}Gppp(m₁^{2′-O})ApG)-hAg-Kozak-RBD-GS-Fibritin-FI-A30L70 |
| Encoded antigen | Viral spike protein (S1S2 protein) of the SARS-CoV-2 (partial sequence, |
| | Receptor Binding Domain (RBD) of S1S2 protein fused to fibritin) |

BNT162b3c (SEQ ID NO: 29; SEQ ID NO: 30)

| | |
|---|---|
| Structure | m₂^{7, 3′-O}Gppp(m₁^{2′-O})ApG-hAg-Kozak-RBD-GS-Fibritin-GS-TM-FI-A30L70 |
| Encoded antigen | Viral spike protein (S1S2 protein) of the SARS-CoV-2 (partial sequence, |
| | Receptor Binding Domain (RBD) of S1S2 protein fused to Fibritin fused to |
| | Transmembrane Domain (TM) of S1S2 protein); intrinsic S1S2 protein |
| | secretory signal peptide (aa 1-19) at the N-terminus of the antigen sequence |

BNT162b3d (SEQ ID NO: 31; SEQ ID NO: 32)

| | |
|---|---|
| Structure | m₂^{7, 3′-O}Gppp(m₁^{2′-O})ApG-hAg-Kozak-RBD-GS-Fibritin-GS-TM-FI-A30L70 |
| Encoded antigen | Viral spike protein (S1S2 protein) of the SARS-CoV-2 (partial sequence, |
| | Receptor Binding Domain (RBD) of S1S2 protein fused to Fibritin fused to |
| | Transmembrane Domain (TM) of S1S2 protein); immunoglobulin secretory |
| | signal peptide (aa 1-22) at the N-terminus of the antigen sequence |

Self-amplifying RNA (saRNA)

The active principle of the self-amplifying mRNA (saRNA) drug substance is a single-stranded RNA, which self-amplifies upon entering a cell, and the coronavirus vaccine antigen is translated thereafter. In contrast to uRNA and modRNA that preferably code for a single protein, the coding region of saRNA contains two open reading frames (ORFs). The 5′-ORF encodes the RNA-dependent RNA polymerase such as Venezuelan equine encephalitis virus (VEEV) RNA-dependent RNA polymerase (replicase). The replicase ORF is followed 3′ by a subgenomic promoter and a second ORF encoding the antigen. Furthermore, saRNA UTRs contain 5′ and 3′ conserved sequence elements (CSEs) required for self-amplification. The saRNA contains common structural elements optimized for maximal efficacy of the RNA as the uRNA (5′-cap, 5′-UTR, 3′-UTR, poly(A)-tail). The saRNA preferably contains uridine. The preferred 5′ cap structure is beta-S-ARCA(D1) (m₂^{7,2′-O}GppSpG).

Cytoplasmic delivery of saRNA initiates an alphavirus-like life cycle. However, the saRNA does not encode for alphaviral structural proteins that are required for genome packaging or cell entry, therefore generation of replication competent viral particles is very unlikely to not possible. Replication does not involve any intermediate steps that generate DNA. The use/uptake of saRNA therefore poses no risk of genomic integration or other permanent genetic modification within the target cell. Furthermore, the saRNA itself prevents its persistent replication by effectively activating innate immune response via recognition of dsRNA intermediates.

Different embodiments of this platform are as follows:

RBS004.1 (SEQ ID NO: 24; SEQ ID NO: 7)

| | |
|---|---|
| Structure | beta-S-ARCA(D1)-replicase-S1S2-PP-FI-A30L70 |
| Encoded antigen | Viral spike protein (S protein) of the SARS-CoV-2 (S1S2 full-length protein, |
| | sequence variant) |

RBS004.2 (SEQ ID NO: 25; SEQ ID NO: 7)

| | |
|---|---|
| Structure | beta-S-ARCA(D1)-replicase-S1S2-PP-FI-A30L70 |
| Encoded antigen | Viral spike protein (S protein) of the SARS-CoV-2 (S1S2 full-length protein, |
| | sequence variant) |

BNT162c1; RBS004.3 (SEQ ID NO: 26; SEQ ID NO: 5)

| | |
|---|---|
| Structure | beta-S-ARCA(D1)-replicase-RBD-GS-Fibritin-FI-A30L70 |
| Encoded antigen | Viral spike protein (S protein) of the SARS-CoV-2 (partial sequence, |
| | Receptor Binding Domain (RBD) of S1S2 protein) |

RBS004.4 (SEQ ID NO: 27; SEQ ID NO: 28)

| | |
|---|---|
| Structure | beta-S-ARCA(D1)-replicase-RBD-GS-Fibritin-TM-FI-A30L70 |
| Encoded antigen | Viral spike protein (S protein) of the SARS-CoV-2 (partial sequence, |
| | Receptor Binding Domain (RBD) of S1S2 protein) |

Furthermore, a secretory signal peptide (sec) may be fused to the antigen-encoding regions preferably in a way that the sec is translated as N terminal tag. In one embodiment, sec corresponds to the secretory signal peptide of the S protein. Sequences coding for short linker peptides predominantly consisting of the amino acids glycine (G) and serine (S), as commonly used for fusion proteins may be used as GS/Linkers.

In one embodiment, RNA (preferably mRNA) encoding an antigen (such as a tumor antigen or a vaccine antigen) is expressed in cells of the subject treated to provide the antigen. In one embodiment, the RNA is transiently expressed in cells of the subject. In one embodiment, the RNA is in vitro transcribed. In one embodiment, expression of the antigen is at the cell surface. In one embodiment, the antigen is expressed and presented in the context of MHC. In one embodiment, expression of the antigen is into the extracellular space, i.e., the antigen is secreted.

The antigen molecule or a procession product thereof, e.g., a fragment thereof, may bind to an antigen receptor such as a BCR or TCR carried by immune effector cells, or to antibodies.

A peptide and protein antigen which is provided to a subject according to the present disclosure by administering RNA (such as mRNA) encoding a peptide and protein antigen, wherein the antigen is a vaccine antigen, preferably results in the induction of an immune response, e.g., a humoral and/or cellular immune response in the subject being provided the peptide or protein antigen. Said immune response is preferably directed against a target antigen. Thus, a vaccine antigen may comprise the target antigen, a variant thereof, or a fragment thereof. In one embodiment, such fragment or variant is immunologically equivalent to the target antigen. In the context of the present disclosure, the term “fragment of an antigen” or “variant of an antigen” means an agent which results in the induction of an immune response which immune response targets the antigen, i.e. a target antigen. Thus, the vaccine antigen may correspond to or may comprise the target antigen, may correspond to or may comprise a fragment of the target antigen or may correspond to or may comprise an antigen which is homologous to the target antigen or a fragment thereof. Thus, according to the present disclosure, a vaccine antigen may comprise an immunogenic fragment of a target antigen or an amino acid sequence being homologous to an immunogenic fragment of a target antigen. An “immunogenic fragment of an antigen” according to the disclosure preferably relates to a fragment of an antigen which is capable of inducing an immune response against the target antigen. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence.

In one embodiment, the RNA (preferably mRNA) used in the present disclosure is non-immunogenic. RNA encoding an immunostimulant may be administered according to the present disclosure to provide an adjuvant effect. The RNA encoding an immunostimulant may be standard RNA or non-immunogenic RNA.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In one embodiment, the modified nucleosides comprise a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-undine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(tm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoymethyl-2′-O-methyl-uridine (ncm⁵Um), 5-caboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In one embodiment, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of RNA (preferably mRNA) by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseI that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In one embodiment, the removal of dsRNA (especially mRNA) from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA (especially mRNA) is free or essentially free of dsRNA. In some embodiments, the non-immunogenic RNA (especially mRNA) composition comprises a purified preparation of single-stranded nucleoside modified RNA. For example, in some embodiments, the purified preparation of single-stranded nucleoside modified RNA (especially mRNA) is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In one embodiment, the non-immunogenic RNA (especially mRNA) is translated in a cell more efficiently than standard RNA with the same sequence. In one embodiment, translation is enhanced by a factor of 2-fold relative to its unmodified counterpart. In one embodiment, translation is enhanced by a 3-fold factor. In one embodiment, translation is enhanced by a 4-fold factor. In one embodiment, translation is enhanced by a 5-fold factor. In one embodiment, translation is enhanced by a 6-fold factor.

In one embodiment, translation is enhanced by a 7-fold factor. In one embodiment, translation is enhanced by an 8-fold factor. In one embodiment, translation is enhanced by a 9-fold factor. In one embodiment, translation is enhanced by a 10-fold factor. In one embodiment, translation is enhanced by a 15-fold factor. In one embodiment, translation is enhanced by a 20-fold factor. In one embodiment, translation is enhanced by a 50-fold factor. In one embodiment, translation is enhanced by a 100-fold factor. In one embodiment, translation is enhanced by a 200-fold factor. In one embodiment, translation is enhanced by a 500-fold factor. In one embodiment, translation is enhanced by a 1000-fold factor. In one embodiment, translation is enhanced by a 2000-fold factor. In one embodiment, the factor is 10-1000-fold. In one embodiment, the factor is 10-100-fold. In one embodiment, the factor is 10-200-fold.

In one embodiment, the factor is 10-300-fold. In one embodiment, the factor is 10-500-fold. In one embodiment, the factor is 20-1000-fold. In one embodiment, the factor is 30-1000-fold. In one embodiment, the factor is 50-1000-fold. In one embodiment, the factor is 100-1000-fold. In one embodiment, the factor is 200-1000-fold. In one embodiment, translation is enhanced by any other significant amount or range of amounts.

In one embodiment, the non-immunogenic RNA (especially mRNA) exhibits significantly less innate immunogenicity than standard RNA with the same sequence. In one embodiment, the non-immunogenic RNA (especially mRNA) exhibits an innate immune response that is 2-fold less than its unmodified counterpart. In one embodiment, innate immunogenicity is reduced by a 3-fold factor. In one embodiment, innate immunogenicity is reduced by a 4-fold factor. In one embodiment, innate immunogenicity is reduced by a 5-fold factor. In one embodiment, innate immunogenicity is reduced by a 6-fold factor. In one embodiment, innate immunogenicity is reduced by a 7-fold factor. In one embodiment, innate immunogenicity is reduced by a 8-fold factor. In one embodiment, innate immunogenicity is reduced by a 9-fold factor. In one embodiment, innate immunogenicity is reduced by a 10-fold factor. In one embodiment, innate immunogenicity is reduced by a 15-fold factor. In one embodiment, innate immunogenicity is reduced by a 20-fold factor. In one embodiment, innate immunogenicity is reduced by a 50-fold factor. In one embodiment, innate immunogenicity is reduced by a 100-fold factor. In one embodiment, innate immunogenicity is reduced by a 200-fold factor. In one embodiment, innate immunogenicity is reduced by a 500-fold factor. In one embodiment, innate immunogenicity is reduced by a 1000-fold factor. In one embodiment, innate immunogenicity is reduced by a 2000-fold factor.

The term “exhibits significantly less innate immunogenicity” refers to a detectable decrease in innate immunogenicity. In one embodiment, the term refers to a decrease such that an effective amount of the non-immunogenic RNA (especially mRNA) can be administered without triggering a detectable innate immune response. In one embodiment, the term refers to a decrease such that the non-immunogenic RNA (especially mRNA) can be repeatedly administered without eliciting an innate immune response sufficient to detectably reduce production of the protein encoded by the non-immunogenic RNA. In one embodiment, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked”, “fused”, or “fusion” are used interchangeably. These terms refer to the joining together of two or more elements or components or domains.

Lipid Nano Particles

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

Electrostatic interactions between positively charged molecules such as polymers and lipids and negatively charged nucleic acid are involved in particle formation. This results in complexation and spontaneous formation of nucleic acid particles.

In the context of the present disclosure, the term “particle” relates to a structured entity formed by molecules or molecule complexes, in particular particle forming compounds. Preferably, the particle contains an envelope (e.g., one or more layers or lamellas) made of one or more types of amphiphilic substances (e.g., amphiphilic lipids, amphiphilic polymers, and/or amphiphilic proteins/polypeptides). In this context, the expression “amphiphilic substance” means that the substance possesses both hydrophilic and lipophilic properties. The envelope may also comprise additional substances (e.g., additional lipids and/or additional polymers) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids, amphiphilic polymers, and/or amphiphilic proteins/polypeptides) optionally in combination with additional substances (e.g., additional lipids and/or additional polymers) which do not have to be amphiphilic. In one embodiment, the term “particle” relates to a micro- or nano-sized structure, such as a micro- or nano-sized compact structure. In this respect, the term “micro-sized” means that all three external dimensions of the particle are in the microscale, i.e., between 1 and 5 μm. According to the present disclosure, the term “particle” includes lipoplex particles (LPXs), lipid nanoparticles (LNPs), polyplex particles, lipopolyplex particles, virus-like particles (VLPs), and mixtures thereof (e.g., a mixture of two or more of particle types, such as a mixture of LPXs and VLPs or a mixture of LNPs and VLPs).

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In one embodiment, particles described herein further comprise at least one lipid or lipid-like material other than a cationically ionizable lipid.

In some embodiments, nucleic acid particles (especially RNA particles such as RNA LNPs (e.g., mRNA particles such as mRNA LNPs)) comprise more than one type of nucleic acid molecules, where the molecular parameters of the nucleic acid molecules may be similar or different from each other, like with respect to molar mass or fundamental structural elements such as molecular architecture, capping, coding regions or other features,

As used in the present disclosure, “nanoparticle” refers to a particle comprising nucleic acid (especially mRNA) as described herein and at least one cationic lipid, wherein all three external dimensions of the particle are in the nanoscale, i.e., at least about 1 nm and below about 1000 nm (preferably, between 10 and 990 nm, such as between 15 and 900 nm, between 20 and 800 nm, between 30 and 700 nm, between 40 and 600 nm, or between 50 and 500 nm). Preferably, the longest and shortest axes do not differ significantly. Preferably, the size of a particle is its diameter.

Nucleic acid particles described herein (especially RNA LNPs) may exhibit a polydispersity index (PDI) less than about 0.5, less than about 0.4, less than about 0.3, less than about 0.2, less than about 0.1, or less than about 0.05. By way of example, the nucleic acid particles can exhibit a polydispersity index in a range of about 0.01 to about 0.4 or about 0.1 to about 0.3.

In the context of the present disclosure, the term “lipoplex particle” relates to a particle that contains an amphiphilic lipid, in particular cationic amphiphilic lipid, and nucleic acid (especially RNA such as mRNA) as described herein. Electrostatic interactions between positively charged liposomes (made 35 from one or more amphiphilic lipids, in particular cationic amphiphilic lipids) and negatively charged nucleic acid (especially RNA such as mRNA) results in complexation and spontaneous formation of nucleic acid lipoplex particles. Positively charged liposomes may be generally synthesized using a cationic amphiphilic lipid, such as DOTMA, and additional lipids, such as DOPE. In one embodiment, a nucleic acid (especially RNA such as mRNA) lipoplex particle is a nanoparticle.

The term “lipid nanoparticle” relates to a nano-sized lipid containing particle.

In the context of the present disclosure, the term “polyplex particle” relates to a particle that contains an amphiphilic polymer, in particular a cationic amphiphilic polymer, and nucleic acid (especially RNA such as mRNA) as described herein. Electrostatic interactions between positively charged cationic amphiphilic polymers and negatively charged nucleic acid (especially RNA such as mRNA) results in complexation and spontaneous formation of nucleic acid polyplex particles. Positively charged amphiphilic polymers suitable for the preparation of polyplex particle include protamine, polyethyleneimine, poly-L-lysine, poly-L-arginine and histone. In one embodiment, a nucleic acid (especially RNA such as mRNA) polyplex particle is a nanoparticle.

The term “lipopolyplex particle” relates to particle that contains amphiphilic lipid (in particular cationic amphiphilic lipid) as described herein, amphiphilic polymer (in particular cationic amphiphilic polymer) as described herein, and nucleic acid (especially RNA such as mRNA) as described herein. In one embodiment, a nucleic acid (especially RNA such as mRNA) lipopolyplex particle is a nanoparticle.

The term “virus-like particle” (abbreviated herein as VLP) refers to a molecule that closely resembles a virus, but which does not contain any genetic material of said virus and, thus, is non-infectious. Preferably, VLPs contain nucleic acid (preferably RNA) as described herein, said nucleic acid (preferably RNA) being heterologous to the virus(es) from which the VLPs are derived. VLPs can be synthesized through the individual expression of viral structural proteins, which can then self-assemble into the virus-like structure. In one embodiment, combinations of structural capsid proteins from different viruses can be used to create recombinant VLPs. VLPs can be produced from components of a wide variety of virus families including Hepatitis B virus (HBV) (small HBV derived surface antigen (HBsAg)), Parvoviridae (e.g., adeno-associated virus), Papillomaviridae (e.g., HPV), Retroviridae (e.g., HIV), Flaviviridae (e.g., Hepatitis C virus) and bacteriophages (e.g. Qβ, AP205).

The term “nucleic acid containing particle” relates to a particle as described herein to which nucleic acid (especially RNA such as mRNA) is bound. In this respect, the nucleic acid (especially RNA such as mRNA) may be adhered to the outer surface of the particle (surface nucleic acid (especially surface RNA such as surface mRNA)) and/or may be contained in the particle (encapsulated nucleic acid (especially encapsulated RNA such as encapsulated mRNA)).

In one embodiment, the particles utilized in the methods and uses of the present disclosure have a size (preferably a diameter, i.e., double the radius such as double the radius of gyration (R_{g}) value or double the hydrodynamic radius) in the range of about 10 to about 2000 nm, such as at least about 15 nm (preferably at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most 1900 nm (preferably at most about 1900 nm, at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), preferably in the range of about 20 to about 1500 nm, such as about 30 to about 1200 nm, about 40 to about 1100 nm, about 50 to about 1000 nm, about 60 to about 900 nm, about 70 to 800 nm, about 80 to 700 nm, about 90 to 600 nm, or about 50 to 500 nm or about 100 to 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, or 70 to 150 nm.

With respect to RNA lipid particles (especially RNA LNPs such as mRNA LNPs), the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles (especially RNA LNPs such as mRNA LNPs) described herein can be prepared using a wide range of methods that may involve obtaining a colloid from at least one cationic or cationically ionizable lipid and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the nucleic acid (especially RNA such as mRNA) lipoplex particles described herein are obtainable by adding nucleic acid (especially RNA such as mRNA) to a colloidal liposome dispersion. Using the ethanol injection technique, such colloidal liposome dispersion is, in one embodiment, formed as follows: an ethanol solution comprising lipids, such as cationically ionizable lipids and additional lipids, is injected into an aqueous solution under stirring. In one embodiment, the nucleic acid (especially RNA such as mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid. Each component is responsible for payload protection, and enables effective intracellular delivery. LNPs may be prepared by mixing lipids dissolved in ethanol rapidly with nucleic acid in an aqueous buffer.

Different types of nucleic acid containing particles have been described previously to be suitable for delivery of nucleic acid in particulate form (cf., e.g., Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

In one preferred embodiment, the LNPs comprising RNA and at least one cationically ionizable lipid described herein further comprise one or more additional lipids.

In one embodiment, the LNPs comprising RNA and at least one cationically ionizable lipid described herein are prepared by (a) preparing an RNA solution containing water and a first buffer system; (b) preparing an ethanolic solution comprising the cationically ionizable lipid and, if present, one or more additional lipids; (c) mixing the RNA solution prepared under (a) with the ethanolic solution prepared under (b), thereby preparing a first intermediate formulation comprising the LNPs dispersed in a first aqueous phase comprising the first buffer system; and (d) filtrating the first intermediate formulation prepared under (c) using a final aqueous buffer solution comprising the final buffer system, thereby preparing the formulation comprising LNPs dispersed in a final aqueous phase comprising the final buffer system. After step (c) one or more steps selected from diluting and filtrating, such as tangential flow filtrating or diafiltrating, can follow. In one embodiment, the first buffer system differs from the final buffer system. In an alternative embodiment, the first buffer system and the final buffer system are the same.

In an alternative embodiment, the LNPs comprising RNA and at least one cationically ionizable lipid described herein are prepared by (a′) preparing liposomes or a colloidal preparation of the cationically ionizable lipid and, if present, one or more additional lipids in an aqueous phase; (b′) preparing an RNA solution containing water and a buffering system; and (c′) mixing the liposomes or colloidal preparation prepared under (a′) with the mRNA solution prepared under (b′). After step (c′) one or more steps selected from diluting and filtrating, such as tangential flow filtrating, can follow.

The present disclosure describes compositions which comprise particles comprising RNA (especially LNPs comprising RNA) and at least one cationically ionizable lipid which associates with the RNA to form nucleic acid particles. The RNA particles may comprise RNA which is complexed in different forms by non-covalent interactions to the particle. The particles described herein are not viral particles, in particular infectious viral particles, i.e., they are not able to virally infect cells.

Suitable cationically ionizable lipids are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

Cationically Ionizable Lipids

The nucleic acid particles (especially RNA LNPs) described herein comprise at least one cationically ionizable lipid as particle forming agent. Cationically ionizable lipids contemplated for use herein include any cationically ionizable lipids or lipid-like materials which are able to electrostatically bind nucleic acid. In one embodiment, cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., a lipid which is not permanently cationic. Thus, depending on the pH of the composition in which the cationically ionizable lipid is solved, the cationically ionizable lipid is either positively charged or neutral.

In one embodiment, the cationically ionizable lipid comprises a head group which includes at least one nitrogen atom (N) which is positive charged or capable of being protonated, preferably under physiological conditions.

Examples of cationically ionizable lipids are disclosed, for example, in WO 2016/176330 and WO 2018/078053. In some embodiments, the cationically ionizable lipid has the structure of Formula (I):

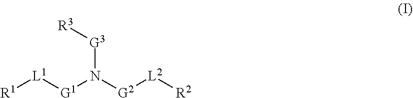

or a pharmaceutically acceptable salt, tautomer, prodrug or stereoisomer thereof, wherein: one of L¹ and L² is —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ and L² is —O(C═O)—, —(C═O)O—, —C(═O)—, —O—, —S(O)—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂-C₁₂ alkenylene;
G³ is C₁-C₂₄ alkylene, C₂-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (I), the lipid has one of the following structures (IA) or (IB):

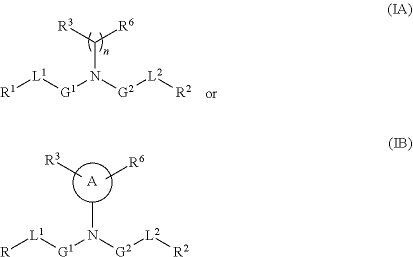

wherein:
A is a 3 to 8-membered cycloalkyl or cycloalkylene group;
R⁶ is, at each occurrence, independently H, OH or C₁-C₂₄ alkyl;
n is an integer ranging from 1 to 15.

In some of the foregoing embodiments of Formula (I), the lipid has structure (IA), and in other embodiments, the lipid has structure (IB).

In other embodiments of Formula (I), the lipid has one of the following structures (IC) or (ID):

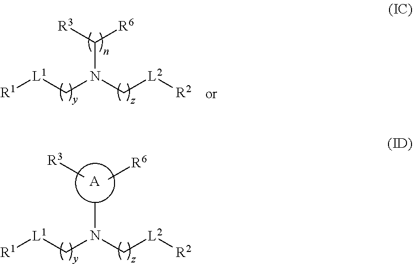

wherein y and z are each independently integers ranging from 1 to 12.

In any of the foregoing embodiments of Formula (I), one of L¹ and L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (I), the lipid has one of the following structures (IE) or (IF):

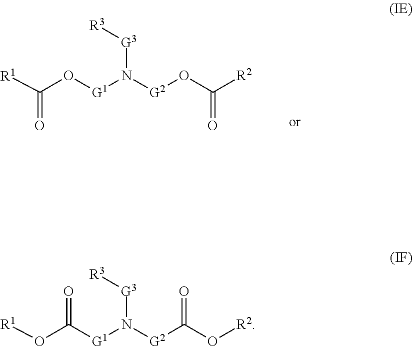

In some of the foregoing embodiments of Formula (I), the lipid has one of the following structures (IG), (IH), (IJ), or (IK):

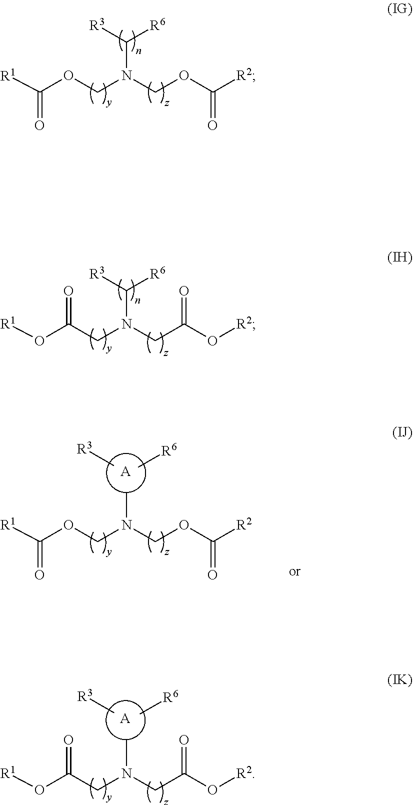

In some of the foregoing embodiments of Formula (I), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (I), y and z are each independently an integer ranging from 2 to 10. For example, in some embodiments, y and z are each independently an integer ranging from 4 to 9 or from 4 to 6.

In some of the foregoing embodiments of Formula (I), R⁶ is H. In other of the foregoing embodiments, R⁶ is C₁-C₂₄ alkyl. In other embodiments, R⁶ is OH.

In some embodiments of Formula (I), G³ is unsubstituted. In other embodiments, G³ is substituted. In various different embodiments, G³ is linear C₁-C₂₄ alkylene or linear C₂-C₂₄ alkenylene.

In some other foregoing embodiments of Formula (I), R¹ or R², or both, is C₆-C₂₄ alkenyl. For example, in some embodiments, R¹ and R² each, independently have the following structure:

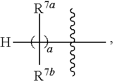

wherein:
R^{7a} and R^{7b} are, at each occurrence, independently H or C₁-C₁₂ alkyl; and a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (I), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (I), R¹ or R², or both, has one of the following structures:

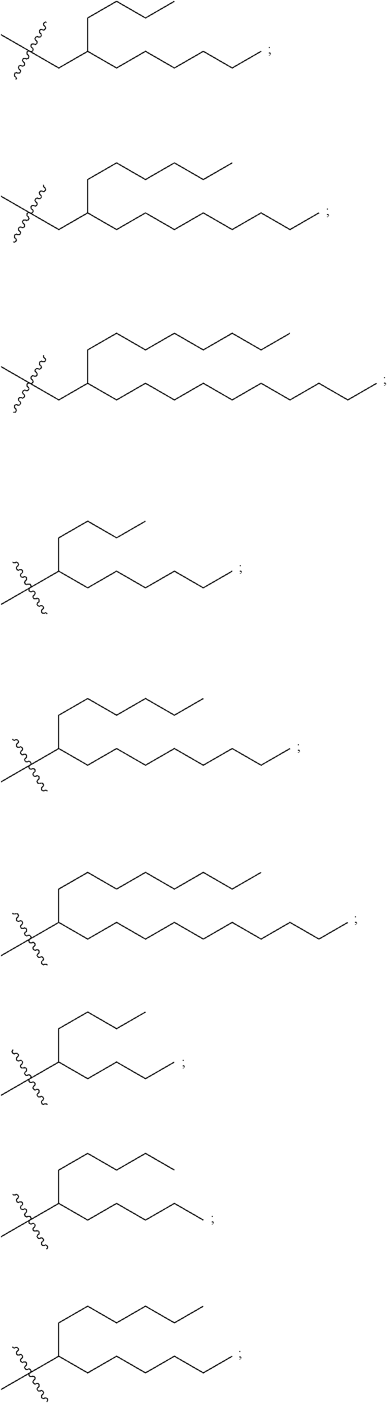

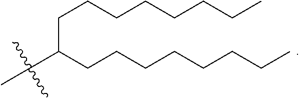 [SMILES: *C(CCCCCCCC)CCCCCCCC]

In some of the foregoing embodiments of Formula (I), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (I) has one of the structures set forth below.

Representative Compounds of Formula (I).

| No. | Structure |
|---|---|
| I-1 | 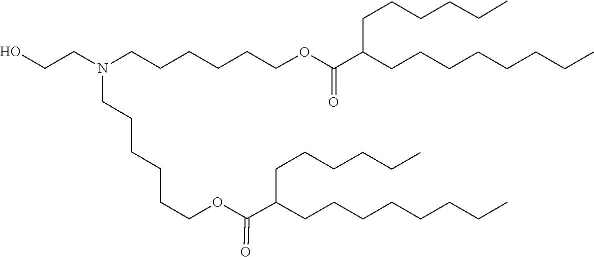 [SMILES: CCCCCCCCC(CCCCCC)C(=O)OCCCCCCN(CCO)CCCCCCOC(=O)C(CCCCCC)CCCCCCCC] |
| I-2 | 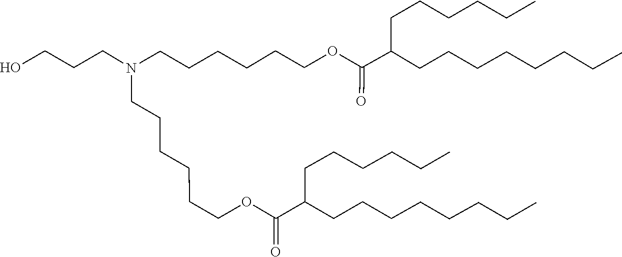 [SMILES: CCCCCCCCC(CCCCCC)C(=O)OCCCCCCN(CCCO)CCCCCCOC(=O)C(CCCCCC)CCCCCCCC] |
| I-3 | 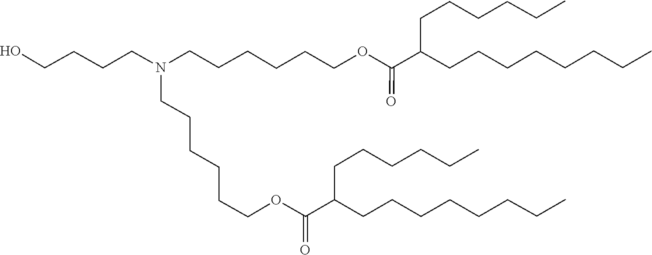 [SMILES: CCCCCCCCC(CCCCCC)C(=O)OCCCCCCN(CCCCO)CCCCCCOC(=O)C(CCCCCC)CCCCCCCC] |
| I-4 | 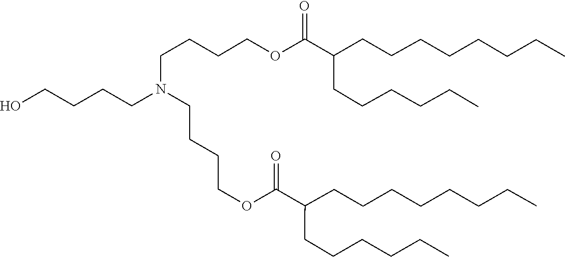 [SMILES: CCCCCCCCC(CCCCCC)C(=O)OCCCCN(CCCCO)CCCCOC(=O)C(CCCCCC)CCCCCCCC] |
| I-5 | 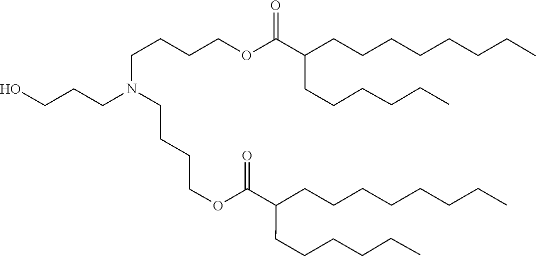 [SMILES: CCCCCCCCC(CCCCCC)C(=O)OCCCCN(CCCO)CCCCOC(=O)C(CCCCCC)CCCCCCCC] |
| I-6 | 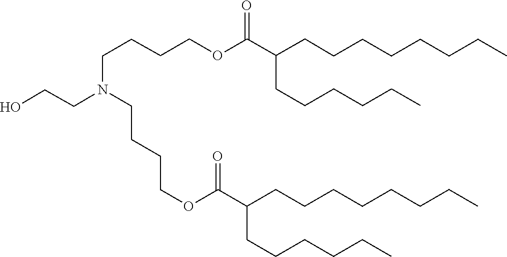 [SMILES: CCCCCCCCC(CCCCCC)C(=O)OCCCCN(CCO)CCCCOC(=O)C(CCCCCC)CCCCCCCC] |
| I-7 | 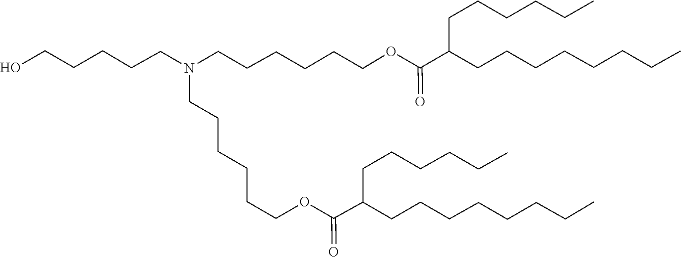 [SMILES: CCCCCCCCC(CCCCCC)C(=O)OCCCCCCN(CCCCCO)CCCCCCOC(=O)C(CCCCCC)CCCCCCCC] |
| I-8 | 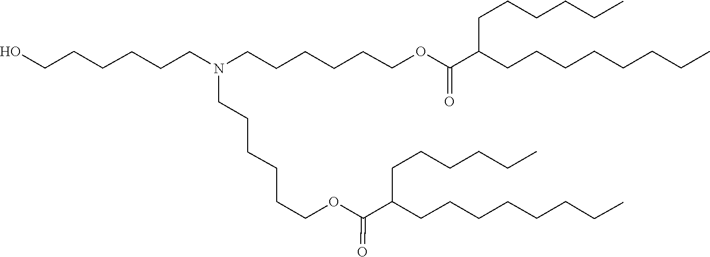 [SMILES: CCCCCCCCC(CCCCCC)C(=O)OCCCCCCN(CCCCCCO)CCCCCCOC(=O)C(CCCCCC)CCCCCCCC] |
| I-9 | 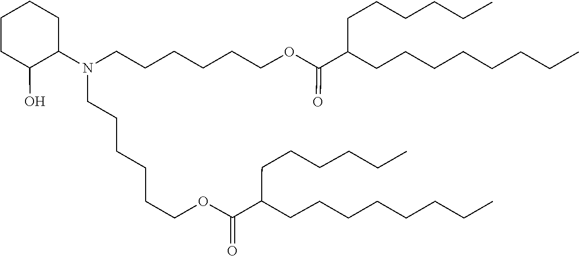 [SMILES: CCCCCCCCC(CCCCCC)C(=O)OCCCCCCN(CCCCCCOC(=O)C(CCCCCC)CCCCCCCC)C1CCCCC1O] |
| I-10 | 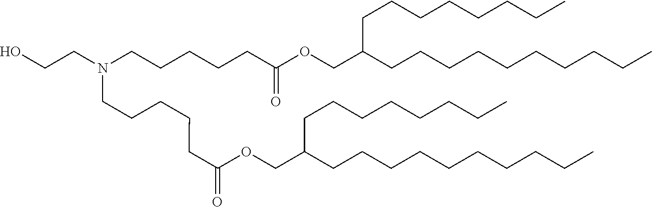 [SMILES: CCCCCCCCCCC(CCCCCCCC)COC(=O)CCCCCN(CCO)CCCCCC(=O)OCC(CCCCCCCC)CCCCCCCCCC] |
| I-11 | 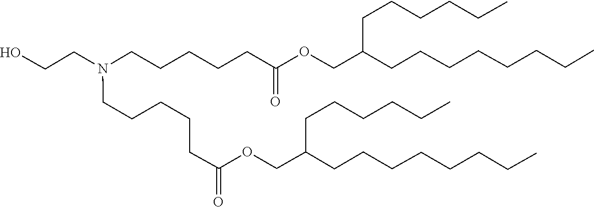 [SMILES: CCCCCCCCC(CCCCCC)COC(=O)CCCCCN(CCO)CCCCCC(=O)OCC(CCCCCC)CCCCCCCC] |
| I-12 | 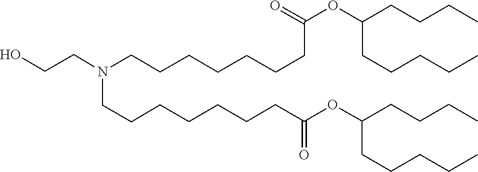 [SMILES: CCCCCC(CCCC)OC(=O)CCCCCCCN(CCO)CCCCCCCC(=O)OC(CCCC)CCCCC] |
| I-13 | 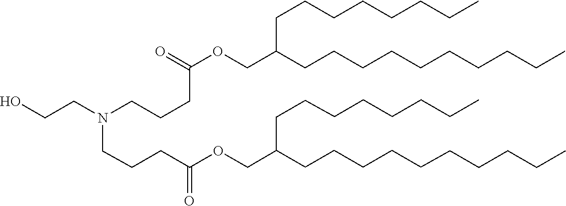 [SMILES: CCCCCCCCCCC(CCCCCCCC)COC(=O)CCCN(CCO)CCCC(=O)OCC(CCCCCCCC)CCCCCCCCCC] |
| I-14 | 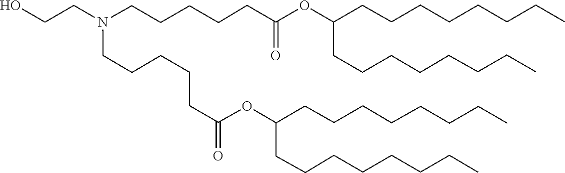 [SMILES: CCCCCCCCC(CCCCCCCC)OC(=O)CCCCCN(CCO)CCCCCC(=O)OC(CCCCCCCC)CCCCCCCC] |
| I-15 | 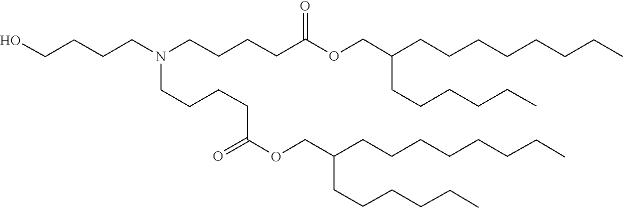 [SMILES: CCCCCCCCC(CCCCCC)COC(=O)CCCCN(CCCCO)CCCCC(=O)OCC(CCCCCC)CCCCCCCC] |
| I-16 | 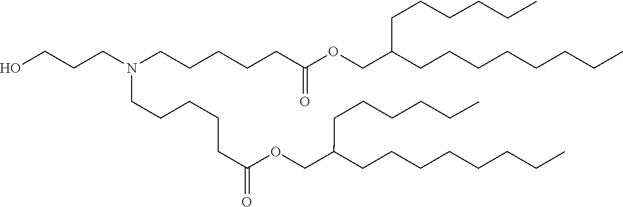 [SMILES: CCCCCCCCC(CCCCCC)COC(=O)CCCCCN(CCCO)CCCCCC(=O)OCC(CCCCCC)CCCCCCCC] |
| I-17 | 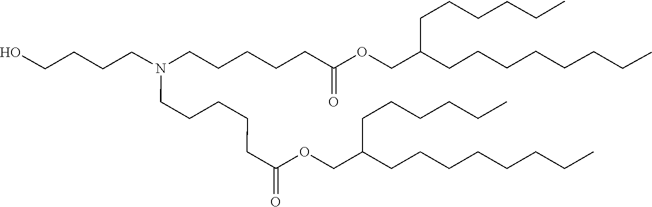 [SMILES: CCCCCCCCC(CCCCCC)COC(=O)CCCCCN(CCCCO)CCCCCC(=O)OCC(CCCCCC)CCCCCCCC] |
| I-18 | 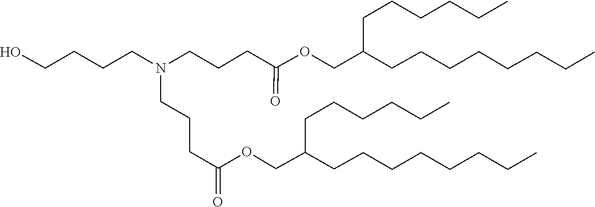 [SMILES: CCCCCCCCC(CCCCCC)COC(=O)CCCN(CCCCO)CCCC(=O)OCC(CCCCCC)CCCCCCCC] |
| I-19 | 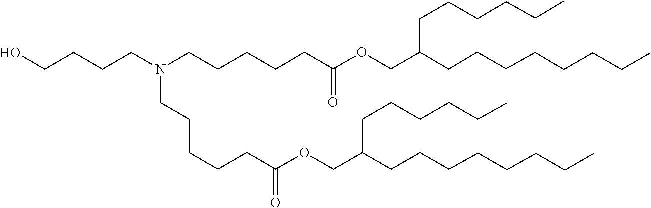 [SMILES: CCCCCCCCC(CCCCCC)COC(=O)CCCCCN(CCCCO)CCCCCC(=O)OCC(CCCCCC)CCCCCCCC] |
| I-20 | 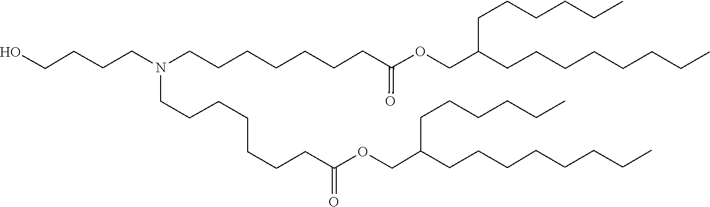 [SMILES: CCCCCCCCC(CCCCCC)COC(=O)CCCCCCCN(CCCCO)CCCCCCCC(=O)OCC(CCCCCC)CCCCCCCC] |
| I-21 | 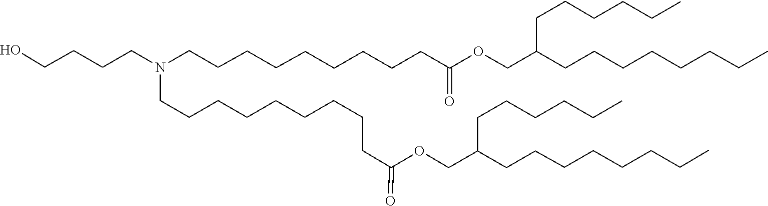 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 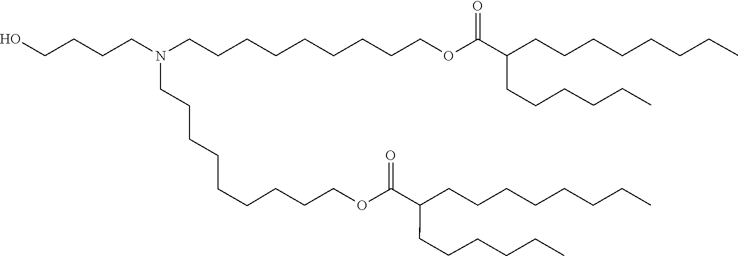 [SMILES: CCCCCCCCC(CCCCCC)C(=O)OCCCCCCCCCN(CCCCO)CCCCCCCCCOC(=O)C(CCCCCC)CCCCCCCC] |
| I-23 | 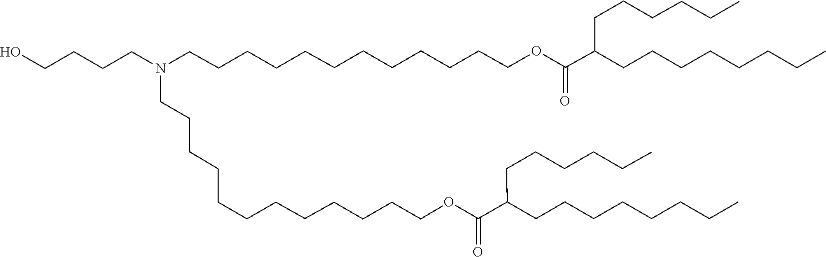 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 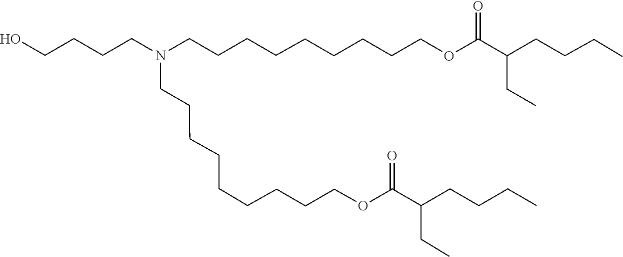 [SMILES: CCCCC(CC)C(=O)OCCCCCCCCCN(CCCCO)CCCCCCCCCOC(=O)C(CC)CCCC] |
| I-25 | 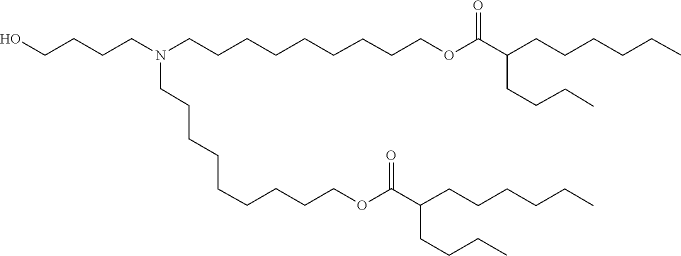 [SMILES: CCCCCCC(CCCC)C(=O)OCCCCCCCCCN(CCCCO)CCCCCCCCCOC(=O)C(CCCC)CCCCCC] |
| I-26 | 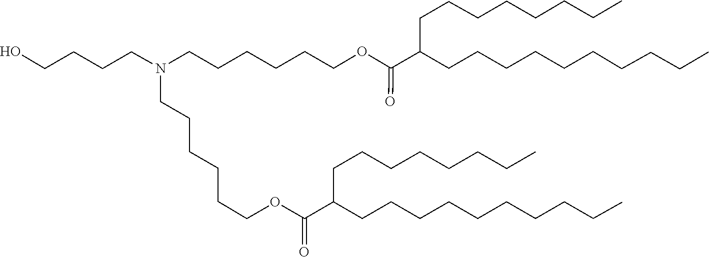 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 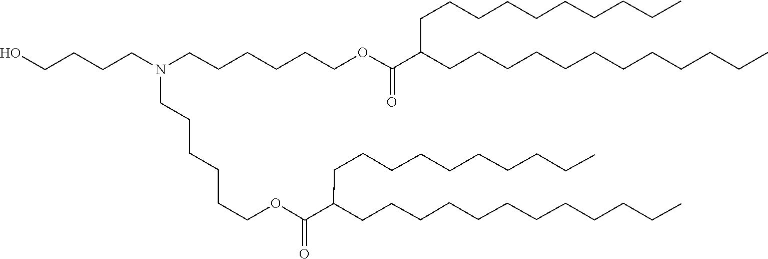 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 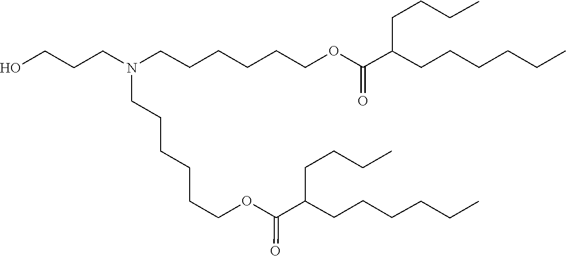 [SMILES: CCCCCCC(CCCC)C(=O)OCCCCCCN(CCCO)CCCCCCOC(=O)C(CCCC)CCCCCC] |
| I-29 | 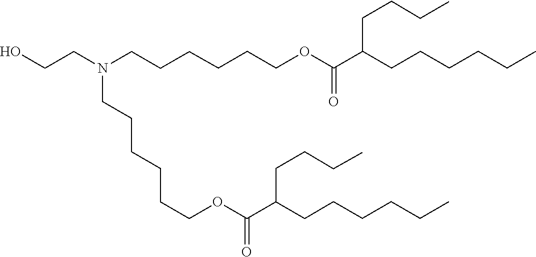 [SMILES: CCCCCCC(CCCC)C(=O)OCCCCCCN(CCO)CCCCCCOC(=O)C(CCCC)CCCCCC] |
| I-30 | 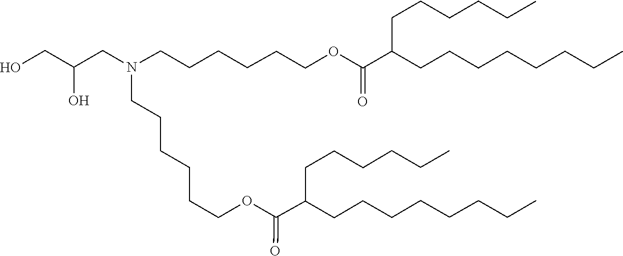 [SMILES: CCCCCCCCC(CCCCCC)C(=O)OCCCCCCN(CCCCCCOC(=O)C(CCCCCC)CCCCCCCC)CC(O)CO] |
| I-31 | 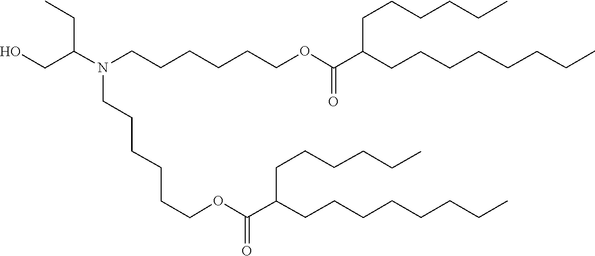 [SMILES: CCCCCCCCC(CCCCCC)C(=O)OCCCCCCN(CCCCCCOC(=O)C(CCCCCC)CCCCCCCC)C(CC)CO] |
| I-32 | 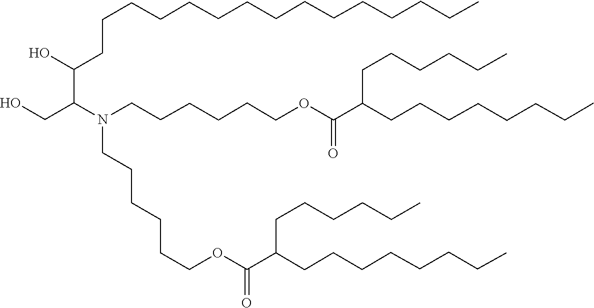 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 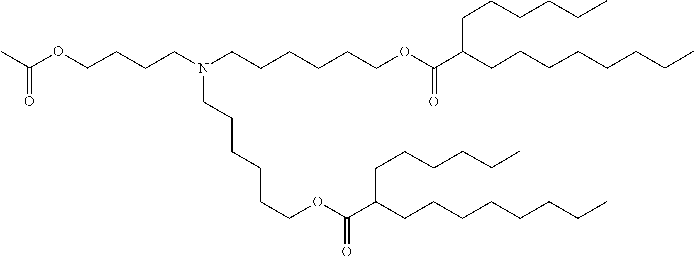 [SMILES: CCCCCCCCC(CCCCCC)C(=O)OCCCCCCN(CCCCCCOC(=O)C(CCCCCC)CCCCCCCC)CCCCOC(C)=O] |
| I-34 | 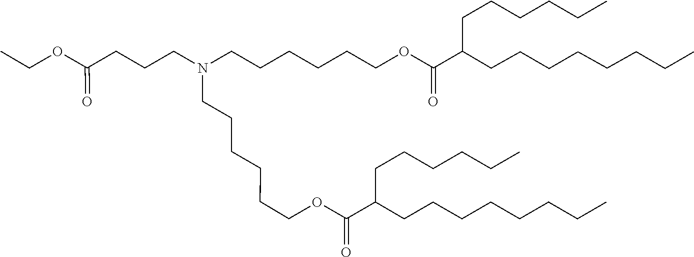 [SMILES: CCCCCCCCC(CCCCCC)C(=O)OCCCCCCN(CCCCCCOC(=O)C(CCCCCC)CCCCCCCC)CCCC(=O)OCC] |
| I-35 | 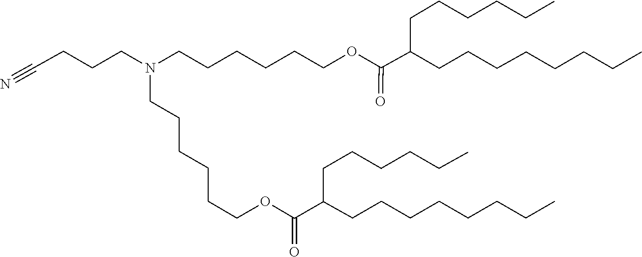 [SMILES: CCCCCCCCC(CCCCCC)C(=O)OCCCCCCN(CCCC#N)CCCCCCOC(=O)C(CCCCCC)CCCCCCCC] |
| I-36 | 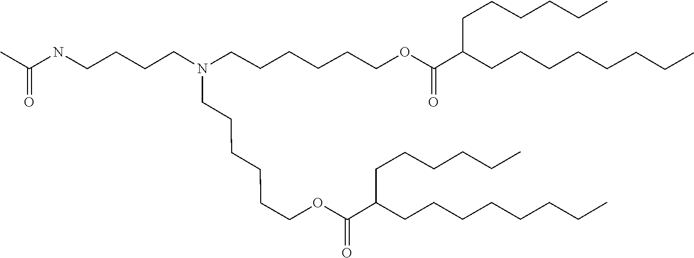 |

In various different embodiments, the cationically ionizable lipid has one of the structures set forth in the table below.

| No. | Structure |
|---|---|
| A | 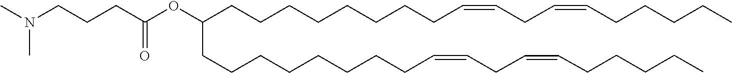 [SMILES: CCCCC/C=C\C/C=C\CCCCCCCCC(CCCCCCCC/C=C\C/C=C\CCCCC)OC(=O)CCCN(C)C] |
| B | 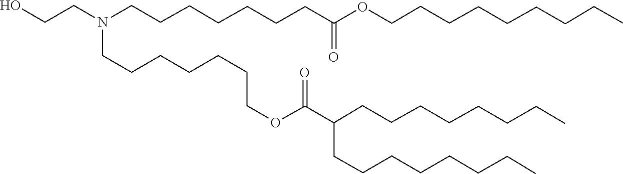 [SMILES: CCCCCCCCCOC(=O)CCCCCCCN(CCO)CCCCCCCOC(=O)C(CCCCCCCC)CCCCCCCC] |
| C | 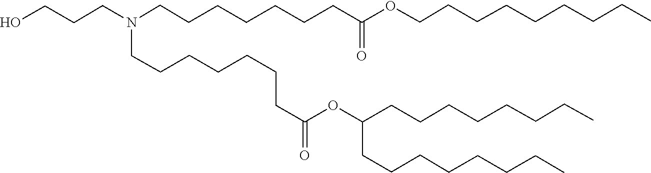 [SMILES: CCCCCCCCCOC(=O)CCCCCCCN(CCCO)CCCCCCCC(=O)OC(CCCCCCCC)CCCCCCCC] |
| D | 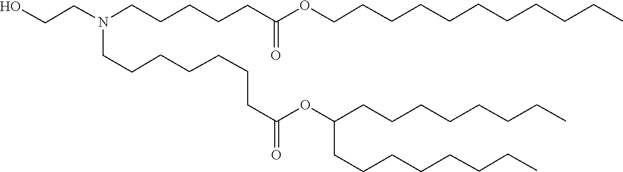 [SMILES: CCCCCCCCCCCOC(=O)CCCCCN(CCO)CCCCCCCC(=O)OC(CCCCCCCC)CCCCCCCC] |
| E | 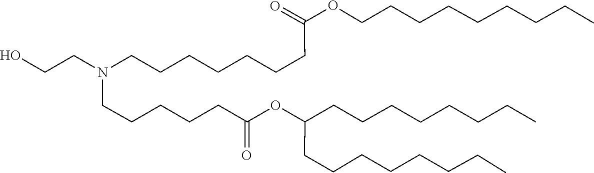 [SMILES: CCCCCCCCCOC(=O)CCCCCCCN(CCO)CCCCCC(=O)OC(CCCCCCCC)CCCCCCCC] |
| F | 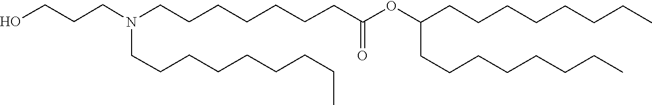 [SMILES: CCCCCCCCCN(CCCO)CCCCCCCC(=O)OC(CCCCCCCC)CCCCCCCC] |

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14).

Further examples of cationically ionizable lipids include, but are not limited to, 3-(N—(N′,N′-dimethylaminoethane)-carbamoyl)cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), di((Z)-non-2-en-1-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In one preferred embodiment, the cationically ionizable lipid has the structure I-3.

In some embodiments, the cationically ionizable lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

In one embodiment, wherein the particles (in particular the RNA LNPs) described herein comprise a cationically ionizable lipid and one or more additional lipids, the cationically ionizable lipid comprises from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipid present in the particles.

In one embodiment, the particles (in particular the RNA LNPs) described herein comprise from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationically ionizable lipid. In one embodiment, the particles (in particular the RNA LNPs) comprise about 47.0, 47.1, 47.2, 47.3, 47.4, 47.5, 47.6, 47.7, 47.8, 47.9 or 48.0 mol percent of the cationically ionizable lipid.

Additional Lipids

Particles (in particular RNA LNPs) described herein may also comprise lipids or lipid-like materials other than cationically ionizable lipids, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to a cationically ionizable lipid may enhance particle stability and efficacy of nucleic acid delivery.

The terms “lipid” and “lipid-like material” are broadly defined herein as molecules which comprise one or more hydrophobic moieties or groups and optionally also one or more hydrophilic moieties or groups.

Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s).

The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge.

Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits).

Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine.

Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

Cationic or cationically ionizable lipids and lipid-like materials may be used to electrostatically bind RNA. Cationically ionizable lipids and lipid-like materials are materials that are preferably positively charged only at acidic pH. This ionizable behavior is thought to enhance efficacy through helping with endosomal escape and reducing toxicity as compared with particles that remain cationic at physiological pH. The particles may also comprise non-cationic lipids or lipid-like materials. Collectively, anionic and neutral lipids or lipid-like materials are referred to herein as non-cationic lipids or lipid-like materials.

Optimizing the formulation of RNA particles by addition of other hydrophobic moieties, such as cholesterol and lipids, in addition to an ionizable/cationic lipid or lipid-like material enhances particle stability and can significantly enhance efficacy of RNA delivery.

One or more additional lipids may be incorporated which may or may not affect the overall charge of the nucleic acid particles. In certain embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In certain embodiments, the nucleic acid particles (especially the RNA LNPs) described herein comprise a cationically ionizable lipid and one or more additional lipids.

Without wishing to be bound by theory, the amount of the cationically ionizable lipid compared to the amount of the one or more additional lipids may affect important nucleic acid particle characteristics, such as charge, particle size, stability, tissue selectivity, and bioactivity of the nucleic acid. Accordingly, in some embodiments, the molar ratio of the cationically ionizable lipid to the one or more additional lipids is from about 10:0 to about 1:9, about 4:1 to about 1:2, or about 3:1 to about 1:1.

In one embodiment, the one or more additional lipids comprised in the nucleic acid particles (especially in the RNA LNPs) described herein comprise one or more of the following: neutral lipids, steroids, polymer conjugated lipids, and combinations thereof.

In one embodiment, the one or more additional lipids comprise a neutral lipid which is a phospholipid.

Preferably, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecxnoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In one embodiment, the neutral lipid is selected from the group consisting of DSPC, DOPC, DMPC, DPPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

Thus, in one embodiment, the nucleic acid particles (especially the RNA LNPs) described herein comprise a cationically ionizable lipid and DSPC.

In one embodiment, the neutral lipid is present in the particles (in particular the RNA LNPs) described herein in a concentration ranging from 5 to 15 mol percent, from 7 to 13 mol percent, or from 9 to 11 mol percent. In one embodiment, the neutral lipid is present in a concentration of about 9.5, 10 or 10.5 mol percent of the total lipids present in the particles (especially the RNA LNPs) described herein.

In one embodiment, the steroid is cholesterol. Thus, in one embodiment, the nucleic acid particles (especially the RNA LNPs) comprise a cationically ionizable lipid and cholesterol.

In one embodiment, the steroid is present in the particles (in particular the RNA LNPs) in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent of the total lipids present in the particles (especially the RNA LNPs) described herein.

In certain preferred embodiments, the nucleic acid particles (especially the RNA LNPs) described herein comprise DSPC and cholesterol, preferably in the concentrations given above.

In some embodiments, the combined concentration of the neutral lipid (in particular, one or more phospholipids) and steroid (in particular, cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, such as from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipids present in the nucleic acid particles (especially the RNA LNPs) described herein.

In one embodiment, a polymer conjugated lipid is a pegylated lipid or a polysarcosine-lipid conjugate or a conjugate of polysarcosine and a lipid-like material.

The term “pegylated lipid” refers to a molecule comprising both a lipid portion and a polyethylene glycol portion. Pegylated lipids are known in the art. In one embodiment, the polymer conjugated lipid is a pegylated lipid. In one embodiment, the pegylated lipid has the following structure:

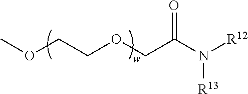

or a pharmaceutically acceptable salt, tautomer or stereoisomer thereof, wherein R¹² and R¹³ are each independently a straight or branched, alkyl or alkenyl chain containing from 10 to 30 carbon atoms, wherein the alkyl or alkenyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is 2-[(polyethylene glycol)-2000]-N,N-ditetradecylacetamide/2-[2-(w-methoxy (polyethyleneglycol2000) ethoxy]-N,N-ditetradecylacetamide, e.g., having the following structure:

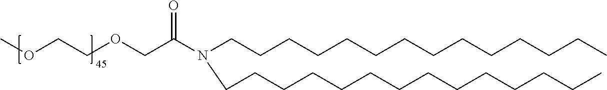

In one embodiment, the nucleic acid particles (especially the RNA LNPs) described herein comprise a cationically ionizable lipid and a pegylated lipid, e.g., a pegylated lipid as defined above.

In one embodiment, the pegylated lipid is present in the particles (in particular the RNA LNPs) in a concentration ranging from 1 to 10 mol percent, from 1 to 5 mol percent, or from 1 to 2.5 mol percent of the total lipids present in the particles (especially the RNA LNPs) described herein.

In one embodiment, the polymer conjugated lipid is a polysarcosine-lipid conjugate or a conjugate of polysarcosine and a lipid-like material, i.e., a lipid or lipid-like material which comprises polysarcosine (poly(N-methylglycine)). The polysarcosine may comprise acetylated (neutral end group) or other functionalized end groups. In the case of RNA-lipid particles, the polysarcosine in one embodiment is conjugated to, preferably covalently bound to a non-cationic lipid or lipid-like material comprised in the particles.

In certain embodiments, the end groups of the polysarcosine may be functionalized with one or more molecular moieties conferring certain properties, such as positive or negative charge, or a targeting agent that will direct the particle to a particular cell type, collection of cells, or tissue.

A variety of suitable targeting agents are known in the art. Non-limiting examples of targeting agents include a peptide, a protein, an enzyme, a nucleic acid, a fatty acid, a hormone, an antibody, a carbohydrate, mono-, oligo- or polysaccharides, a peptidoglycan, a glycopeptide, or the like. For example, any of a number of different materials that bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab′, F(ab′)2 or scFv fragments or single-domain antibodies (e.g. camelids VHH fragments). Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

In certain embodiments, the polysarcosine comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 sarcosine units.

In certain embodiments, the polysarcosine comprises the following general formula (II):

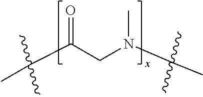

wherein x refers to the number of sarcosine units. The polysarcosine through one of the bonds may be linked to a particle-forming component or a hydrophobic component. The polysarcosine through the other bond may be linked to H, a hydrophilic group, an ionizable group, or to a linker to a functional moiety such as a targeting moiety.

The polysarcosine may be conjugated, in particular covalently bound to or linked to, any particle forming component such as a lipid or lipid-like material. The polysarcosine-lipid conjugate is a molecule wherein polysarcosine is conjugated to a lipid as described herein such as a cationic lipid or cationically ionizable lipid or an additional lipid. Alternatively, polysarcosine is conjugated to a lipid or lipid-like material which is different from the cationically ionizable lipid or the one or more additional lipids.

In certain embodiments, the polysarcosine-lipid conjugate or a conjugate of polysarcosine and a lipid-like material comprises the following general formula (Ha):

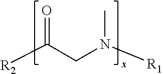

wherein one of R₁ and R₂ comprises a hydrophobic group and the other is H, a hydrophilic group, an ionizable group or a functional group optionally comprising a targeting moiety. In one embodiment, the hydrophobic group comprises a linear or branched alkyl group or aryl group, preferably comprising from 10 to 50, 10 to 40, or 12 to 20 carbon atoms. In one embodiment, R₁ or R₂ which comprises a hydrophobic group comprises a moiety such as a heteroatom, in particular N, linked to one or more linear or branched alkyl groups.

In certain embodiments, a polysarcosine-lipid conjugate or a conjugate of polysarcosine and a lipid-like material comprises the following general formula (IIb):

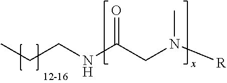

wherein R is H, a hydrophilic group, an ionizable group or a functional group optionally comprising a targeting moiety.

The symbol “x” in the general formulas (IIa) and (IIb) refers to the number of sarcosine units and may be a number as defined herein.

In certain embodiments, the polysarcosine-lipid conjugate or a conjugate of polysarcosine and a lipid-like material is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

Typically, the polysarcosine moiety has between 2 and 200, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, or between 10 and 80 sarcosine units.

Thus, in one embodiment, the nucleic acid particles (especially the RNA LNPs) described herein comprise a cationically ionizable lipid and a polysarcosine-lipid conjugate or a conjugate of polysarcosine and a lipid-like material, e.g., a polysarcosine-lipid conjugate or a conjugate of polysarcosine and a lipid-like material as defined above.

In certain instances, the polysarcosine-lipid conjugate may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the nucleic acid particles (especially the RNA LNPs) described herein.

In some embodiments, the one or more additional lipids comprise one of the following components: (1) a neutral lipid; (2) a steroid; (3) a polymer conjugated lipid; (4) a mixture of a neutral lipid and a steroid; (5) a mixture of a neutral lipid and a polymer conjugated lipid; (6) a mixture of a steroid and a polymer conjugated lipid; or (7) a mixture of a neutral lipid, a steroid, and a polymer conjugated lipid, preferably each in the concentration given above. In some embodiments, the one or more additional lipids comprise one of the following components: (1) a phospholipid; (2) cholesterol; (3) a pegylated lipid; (4) a mixture of a phospholipid and cholesterol; (5) a mixture of a phospholipid and a pegylated lipid; (6) a mixture of cholesterol and a pegylated lipid; or (7) a mixture of a phospholipid, cholesterol, and a pegylated lipid, preferably each in the concentration given above.

Thus, in preferred embodiments, the nucleic acid particles (especially the RNA LNPs) described herein comprise a cationically ionizable lipid and one of the following lipids or lipid mixtures: (1) a neutral lipid; (2) a steroid; (3) a polymer conjugated lipid; (4) a mixture of a neutral lipid and a steroid; (5) a mixture of a neutral lipid and a polymer conjugated lipid; (6) a mixture of a steroid and a polymer conjugated lipid; or (7) a mixture of a neutral lipid, a steroid, and a polymer conjugated lipid, preferably each in the concentration given above. In one specific embodiment, the cationically ionizable lipid is present in a concentration of from 40 to 50 mol percent; the neutral lipid is present in a concentration of from 5 to 15 mol percent; the steroid is present in a concentration of from 35 to 45 mol; and the polymer conjugated lipid is present in a concentration of from 1 to 10 mol percent, wherein the RNA is encapsulated within or associated with the LNPs.

In more preferred embodiments, the nucleic acid particles (especially the RNA LNPs) described herein comprise a cationically ionizable lipid and one of the following lipids or lipid mixtures: (1) a phospholipid; (2) cholesterol; (3) a pegylated lipid; (4) a mixture of a phospholipid and cholesterol; (5) a mixture of a phospholipid and a pegylated lipid; (6) a mixture of cholesterol and a pegylated lipid; or (7) a mixture of a phospholipid, cholesterol, and a pegylated lipid, preferably each in the concentration given above. In one specific embodiment, the cationically ionizable lipid is present in a concentration of from 40 to 50 mol percent; the phospholipid is present in a concentration of from 5 to 15 mol percent; the cholesterol is present in a concentration of from 35 to 45 mol; and the pegylated lipid is present in a concentration of from 1 to 10 mol percent, wherein the RNA is encapsulated within or associated with the LNPs.

The N/P value is preferably at least about 4. In some embodiments, the N/P value ranges from 4 to 20, 4 to 12, 4 to 10, 4 to 8, or 5 to 7. In one embodiment, the N/P value is about 6.

LNPs described herein may have an average diameter that in one embodiment ranges from about 30 nm to about 200 nm, or from about 60 nm to about 120 nm.

Generally, the LNPs comprising RNA (or “RNA LNPs”) described herein are “RNA-lipid particles” that can be used to deliver RNA to a target site of interest (e.g., cell, tissue, organ, and the like). An RNA-lipid particle is typically formed from a cationically ionizable lipid (such as the lipid having the structure I-3) and one or more additional lipids, such as a phospholipid (e.g., DSPC), a steroid (e.g., cholesterol or analogues thereof), and a polymer conjugated lipid (e.g., a pegylated lipid or a polysarcosine-lipid conjugate or a conjugate of polysarcosine and a lipid-like material).

Without intending to be bound by any theory, it is believed that the cationically ionizable lipid and the one or more additional lipids combine together with the RNA to form colloidally stable particles, wherein the nucleic acid is bound to the lipid matrix.

In some embodiments, RNA-lipid particles comprise more than one type of RNA molecules, where the molecular parameters of the RNA molecules may be similar or different from each other, like with respect to molar mass or fundamental structural elements such as molecular architecture, capping, coding regions or other features.

In some embodiments, the RNA-lipid LNPs (such as mRNA-lipid LNPs) in addition to RNA comprise (i) a cationically ionizable lipid which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipids present in the particle, (ii) a neutral lipid and/or a steroid, (e.g., one or more phospholipids and/or cholesterol) which may comprise from about 0 mol % to about 90 mol %, from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipids present in the particle, and (iii) a polymer conjugated lipid (e.g., a pegylated lipid which may comprise from 1 mol % to 10 mol %, from 1 mol % to 5 mol %, or from 1 mol % to 2.5 mol % of the total lipids present in the particle; or a polysarcosine-lipid conjugate which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the particle).

In certain preferred embodiments, the neutral lipid comprises a phospholipid of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle.

In certain preferred embodiments, the steroid comprises cholesterol or a derivative thereof of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle.

In certain preferred embodiments, the neutral lipid and the steroid comprises a mixture of: (i) a phospholipid such as DSPC of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle; and (ii) cholesterol or a derivative thereof such as cholesterol of from about 10 mot % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle. As a non-limiting example, an mRNA LNP comprising a mixture of a phospholipid and cholesterol may comprise DSPC of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle and cholesterol of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle.

In some embodiments, the RNA-lipid particles in addition to RNA comprise (i) a cationically ionizable lipid (such as the lipid having the structure I-3) which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipids present in the particle, (ii) DSPC which may comprise from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle, (iii) cholesterol which may comprise from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle and (iv) a pegylated lipid which may comprise from 1 mol % to 10 mol %, from 1 mol % to 5 mol %, or from 1 mol % to 2.5 mol % of the total lipids present in the particle; or (iv′) a polysarcosine-lipid conjugate which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the particle.

RNA LNPs described herein have an average diameter that in one embodiment ranges from about 30 nm to about 1000 nm, from about 30 nm to about 800 nm, from about 30 nm to about 700 nm, from about 30 nm to about 600 nm, from about 30 nm to about 500 nm, from about 30 nm to about 450 nm, from about 30 nm to about 400 nm, from about 30 nm to about 350 nm, from about 30 nm to about 300 nm, from about 30 nm to about 250 nm, from about 30 nm to about 200 nm, from about 30 nm to about 190 nm, from about 30 nm to about 180 nm, from about 30 nm to about 170 nm, from about 30 nm to about 160 nm, from about 30 nm to about 150 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 50 am to about 190 am, from about 50 nm to about 180 nm, from about 50 nm to about 170 nm, from about 50 nm to about 160 nm, or from about 50 nm to about 150 nm.

In certain embodiments, RNA LNPs described herein have an average diameter that ranges from about 40 nm to about 800 nm, from about 50 nm to about 700 nm, from about 60 nm to about 600 nm, from about 70 nm to about 500 m, from about 80 am to about 400 am, from about 150 nm to about 800 am, from about 150 nm to about 700 am, from about 150 nm to about 600 nm, from about 200 nm to about 600 am, from about 200 nm to about 500 am, or from about 200 nm to about 400 nm.

RNA LNPs described herein, e.g. prepared by the methods described herein, exhibit a polydispersity index less than about 0.5, less than about 0.4, less than about 0.3, less than about 0.2, less than about 0.1 or about 0.05 or less. By way of example, the RNA LNPs can exhibit a polydispersity index in a range of about 0.05 to about 0.2, such as about 0.05 to about 0.1.

In certain embodiments of the present disclosure, the RNA in the RNA LNPs described herein is at a concentration from about 2 mg/l to about 5 g/l, from about 2 mg/l to about 2 g/l, from about 5 mg/l to about 2 g/l, from about 10 mg/l to about 1 g/1, from about 50 mg/l to about 0.5 g/l or from about 100 mg/l to about 0.5 g/l. In specific embodiments, the RNA is at a concentration from about 5 mg/l to about 150 mg/l, from about 0.005 mg/mL to about 0.09 mg/mL, from about 0.005 mg/mL to about 0.08 mg/mL, from about 0.005 mg/mL to about 0.07 mg/mL, from about 0.005 mg/mL to about 0.06 mg/mL, or from about 0.005 mg/mL to about 0.05 mg/mL.

Compositions/Formulations Comprising-RNA Particles

The compositions/formulations described herein comprise RNA LNPs, preferably a plurality of RNA LNPs. The term “plurality of RNA LNPs” or “plurality of RNA-lipid particles” refers to a population of a certain number of particles. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles.

In one embodiment, the compositions/formulations described herein comprise particles with a size of at least 10 μm in an amount of less than 4000/ml, preferably at most 3500/ml, such as at most 3400/ml, at most 3300/ml, at most 3200/ml, at most 3100/ml, or at most 3000/ml.

It will be apparent to those of skill in the art that the plurality of particles can include any fraction of the foregoing ranges or any range therein.

In some embodiments, the composition described herein is a liquid or a solid, with a solid referring to a frozen form.

The present inventors have surprisingly found that using a buffer based on Tris, Bis-Tris-methane or TEA, in particular Tris, instead of PBS in a composition comprising LNPs inhibits the formation of a very stable folded form of RNA.

Furthermore, the present application demonstrates that subjecting a composition comprising (i) a buffer system at a concentration of 50 mM and (ii) LNPs comprising a cationically ionizable lipid and RNA to a freeze-thaw-cycle results in a significant loss of RNA integrity, whereas, surprisingly, by simply lowering the concentration of the buffer substance in the composition, it is possible to obtain an RNA LNP composition having improved RNA integrity after a freeze-thaw-cycle. Thus, the claimed composition provides improved stability, can be stored in a temperature range compliant to regular technologies in pharmaceutical practice, and provides a ready-to-use formulation.

In addition, it has been surprisingly found that the presence of certain polyvalent anions (in particular inorganic phosphate anions, citrate anions, and anions of EDTA, and optionally inorganic sulfate anions, carbonate anions, dibasic organic acid anions and/or polybasic organic acid anions) in the aqueous phase of an RNA LNP composition may result in an increase of the particle size when the composition is frozen and then thawed (i.e., when the composition is subjected to at least one freeze-thaw-cycle), and that RNA compositions which comprise a buffer based on Tris, Bis-Tris-methane or TEA as disclosed herein and whose aqueous phase is substantially free of such di- and/or polyvalent anions can be frozen and thawed without increasing the particle size.

Thus, according to the present disclosure, the aqueous phase of compositions described herein is substantially free of inorganic phosphate anions, substantially free of citrate anions, and substantially free of anions of EDTA, and preferably is substantially free of sulfate anions and/or carbonate anions and/or dibasic organic acid anions and/or polybasic organic acid anions. In one embodiment, the aqueous phase of compositions described herein is preferably substantially free of inorganic phosphate anions, citrate anions, anions of EDTA, inorganic sulfate anions, carbonate anions, dibasic organic acid anions and polybasic organic acid anions.

The expression “substantially free of X”, as used herein, means that a mixture (such as an aqueous phase of a composition or formulation described herein) is free of X is such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the mixture.

Thus, if the aqueous phase of an RNA LNP composition described herein is to be substantially free of inorganic phosphate anions, it is preferred that the amount of inorganic phosphate anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of citrate anions, it is preferred that the amount of citrate anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of anions of EDTA, it is preferred that the amount of anions of EDTA in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of inorganic sulfate anions, it is preferred that the amount of inorganic sulfate anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of carbonate anions, it is preferred that the amount of carbonate anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of dibasic organic acid anions, it is preferred that the amount of dibasic organic acid anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If the aqueous phase of an RNA LNP composition described herein is to be substantially free of polybasic organic acid anions, it is preferred that the amount of polybasic organic acid anions in the aqueous phase of the RNA LNP composition is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

The expression “inorganic phosphate anion”, as used herein, means any compound which contains an inorganic phosphate anion and which when solved in an aqueous medium releases the inorganic phosphate anion. Examples of compounds which contain an inorganic phosphate anion and which when solved in an aqueous medium release the inorganic phosphate anion, include phosphoric acid and salts of phosphoric acid, conjugates of phosphoric acid, and salts of such conjugates, such as diphosphates, triphosphates, etc. Preferably, the expression “inorganic phosphate anion” does not include esters of phosphoric acid with one or more organic alcohols. Thus, preferably, the expression “inorganic phosphate anion” does not encompass nucleotides, oligonucleotides or polynucleotides.

The expression “citrate anion”, as used herein, means any compound which contains a citrate anion and which when solved in an aqueous medium releases the citrate anion. Examples of compounds which contain a citrate anion and which release the citrate anion when solved in an aqueous medium, include citric acid and salts of citric acid.

The expression “anion of EDTA”, as used herein, means any compound which contains an anion of EDTA and which when solved in an aqueous medium releases the anion of EDTA. Examples of compounds which contain an anion of EDTA and which release an anion when solved in an aqueous medium, include ethylenediaminetetraacetic acid (EDTA) and salts of EDTA.

The expression “inorganic sulfate anion”, as used herein, means any compound which contains an inorganic sulfate anion and which when solved in an aqueous medium releases the inorganic sulfate anion. Examples of compounds which contain an inorganic sulfate anion and which when solved in an aqueous medium release the inorganic sulfate anion, include sulfuric acid and salts of sulfuric acid.

Preferably, the expression “inorganic sulfate anion” does not include esters of sulfuric acid with one or more organic alcohols.

The expression “carbonate anion”, as used herein, means any compound which contains a carbonate anion (i.e., HCO₃⁻ and CO₃²⁻) and which when solved in an aqueous medium releases the carbonate anion. Examples of compounds which contain a carbonate anion and which when solved in an aqueous medium release the carbonate anion, include aqueous solutions of carbon dioxide, and carbonate salts.

Preferably, the expression “carbonate anion” does not include carbonate esters with one or more organic alcohols.

The expression “dibasic organic acid anions”, as used herein, means any organic compound containing two acid groups which are in free form (i.e., protonated), anhydride form or salt form. In this respect, the term “acid group” refers to a carboxylic acid or sulfate group. Preferably, the expression “dibasic organic acids” does not include esters of a carboxylic or sulfate group with one or more organic alcohols. Examples of dibasic organic acids include oxalic acid, malic acid, and tartaric acid.

The expression “polybasic organic acid anions”, as used herein, means any organic compound containing three or more acid groups which are in free form (i.e., protonated), anhydride form or salt form. In this respect, the term “acid group” refers to a carboxylic acid or sulfate group. Preferably, the expression “polybasic organic acids” does not include esters of a carboxylic or sulfate group with one or more organic alcohols. One example of a polybasic organic acid includes citric acid.

The expression “equal to”, as used herein with respect to the size (Z_{average}) of particles (such as LNPs), means that the Z_{average} value of the particles contained in a composition after a processing step (e.g., after a freeze/thaw cycle) corresponds to the Z_{average} value of the particles before the processing step (e.g., before the freeze/thaw cycle)±30% (preferably, ±25%, more preferably 24%, such as ±20%, ±15%, ±10%, ±5%, or ±1%). For example, if the size (Z_{average}) value of particles (such as LNPs) contained in a composition not yet subjected to a freeze/thaw cycle is 90 nm, and the size (Z_{average}) value of particles (such as LNPs) contained in the composition subjected to a freeze/thaw cycle is 115 nm, then the size (Z_{average}) of particles after the freeze/thaw cycle, i.e., after thawing the frozen composition, is considered being equal to the size (Z_{average}) of particles before the freeze/thaw cycle, i.e., before freezing the composition. The expression “equal to”, as used herein with respect to the size distribution or PDI of particles (such as LNPs), is to be interpreted accordingly. For example, if the PDI value of particles (such as LNPs) contained in a composition not yet subjected to a freeze/thaw cycle is 0.30, and the PDI value of particles (such as LNPs) contained in the composition subjected to a freeze/thaw cycle is 0.38, then the PDI of particles after the freeze/thaw cycle, i.e., after thawing the frozen composition, is considered being equal to the PDI of particles before the freeze/thaw cycle, i.e., before freezing the composition.

Compositions described herein may also comprise a cyroprotectant and/or a surfactant as stabilizer to avoid substantial loss of the product quality and, in particular, substantial loss of RNA activity during storage and/or freezing, for example to reduce or prevent aggregation, particle collapse, RNA degradation and/or other types of damage.

In an embodiment, the cryoprotectant is a carbohydrate. The term “carbohydrate”, as used herein, refers to and encompasses monosaccharides, disaccharides, trisaccharides, oligosaccharides and polysaccharides.

In an embodiment, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In an embodiment, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like.

The term “trisaccharide” means three sugars linked together to form one molecule. Examples of a trisaccharides include raffinose and melezitose.

In an embodiment, the cryoprotectant is an oligosaccharide. The term “oligosaccharide”, as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In an embodiment, the cryoprotectant is a cyclic oligosaccharide. The term “cyclic oligosaccharide”, as used herein refers to a compound or a chemical moiety formed by 3 to about 15, preferably 6, 7, 8, 9, or monosaccharide units that are bonded together through glycosidic linkages, for example through 1-4 linkages or 1-6 linkages, to forma cyclic structure. Exemplary cyclic oligosaccharide cryoprotectants include cyclic oligosaccharides that are discrete compounds, such as a cyclodextrin, β cyclodextrin, or γ cyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an α, β, or γcyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-o-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In an embodiment, the cryoprotectant is a sugar alcohol. The term “sugar alcohol”, as used herein, refers to organic compounds containing at least two carbon atoms and one hydroxyl group attached to each carbon atom. Typically, sugar alcohols are derived from sugars (e.g., by hydrogenation of sugars) and are water-soluble solids. The term “sugar”, as used herein, refers sweet-tasting, soluble carbohydrates.

Examples of sugar alcohols include ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. In one embodiment, the sugar alcohol has the formula HOCH₂(CHOH)ₙCH₂OH, wherein n is 0 to 22 (e.g., 0, 1, 2, 3, or 4), or a cyclic variant thereof (which can formally be derived by dehydration of the sugar alcohol to give cyclic ethers; e.g. isosorbide is the cyclic dehydrated variant of sorbitol).

In an embodiment, the cryoprotectant is glycerol and/or sorbitol.

In one embodiment, RNA LNP compositions may include sucrose as cryoprotectant. Without wishing to be bound by theory, sucrose functions to promote cryoprotection of the compositions, thereby preventing nucleic acid (especially RNA) particle aggregation and maintaining chemical and physical stability of the composition. Certain embodiments contemplate alternative cryoprotectants to sucrose in the present disclosure. Alternative stabilizers include, without limitation, glucose, glycerol, and sorbitol.

A preferred cryoprotectant is selected from the group consisting of sucrose, glucose, glycerol, sorbitol, and a combination thereof. In a preferred embodiment, the cryoprotectant comprises sucrose and/or glycerol. In a more preferred embodiment, the cryoprotectant is sucrose.

In one embodiment, the RNA LNP composition described herein comprises the cryoprotectant in a concentration of at least 1% w/v, such as at least 2% w/v, at least 3% w/v, at least 4% w/v, at least 5% w/v, at least 6% w/v, at least 7% w/v, at least 8% w/v or at least 9% w/v. In one embodiment, the concentration of the cryoprotectant in the composition is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In one embodiment, the concentration of the cryoprotectant in the composition is 1% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In one embodiment, the RNA LNP composition described herein comprises a cryoprotectant (in particular, sucrose and/or glycerol) in a (total) concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v.

Preferably, the RNA LNP composition described herein comprises the cryoprotectant in a concentration resulting in an osmolality of the composition in the range of from about 50×10⁻³ osmol/kg to about 1 osmol/kg (such as from about 100×10⁻³ osmol/kg to about 900×10⁻³ osmol/kg, from about 120×10⁻³ osmol/kg to about 800×10⁻³ osmol/kg, from about 140×10⁻³ osmol/kg to about 700×10⁻³ osmol/kg, from about 160×10⁻³ osmol/kg to about 600×10⁻³ osmol/kg, from about 180×10⁻³ osmol/kg to about 500×10⁻³ osmol/kg, or from about 200×10⁻³ osmol/kg to about 400×10⁻³ osmol/kg), for example, from about 50×10⁻³ osmol/kg to about 400×10⁻³ osmol/kg (such as from about 50×10⁻³ osmol/kg to about 390×10⁻³ osmol/kg, from about 60×10⁻³ osmol/kg to about 380×10⁻³ osmol/kg, from about 70×10⁻³ osmol/kg to about 370×10⁻³ osmol/kg, from about 80×10⁻³ osmol/kg to about 360×10⁻³ osmol/kg, from about 90×10⁻³ osmol/kg to about 350×10⁻³ osmol/kg, from about 100×10⁻³ osmol/kg to about 340×10⁻³ osmol/kg, from about 120×10⁻³ osmol/kg to about 330×10⁻³ osmol/kg, from about 140×10⁻³ osmol/kg to about 320×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the composition.

In one preferred embodiment, RNA LNP compositions/formulations comprise sucrose as cryoprotectant and Tris as buffer substance, preferably in the amounts/concentrations specified herein.

In one alternative preferred embodiment, RNA LNP compositions/formulations are substantially free of a cryoprotectant, for example they do not contain any cryoprotectant.

Certain embodiments of the present disclosure contemplate the use of a chelating agent in an RNA LNP composition/formulation described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In certain embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In a preferred alternative embodiment, the aqueous phase of RNA LNP compositions/formulations described herein do not comprise a chelating agent. For example, it is preferred that if RNA LNP compositions/formulations described herein comprise a chelating agent, said chelating agent is only present in the LNPs.

Pharmaceutical Compositions

The RNA LNP compositions described herein are useful as or for preparing pharmaceutical compositions or medicaments for therapeutic or prophylactic treatments.

The RNA LNPs described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In the context of the present disclosure, the pharmaceutical composition comprises RNA LNPs as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in in a frozen form or in a “ready-to-use form” (i.e., in a form, in particular a liquid form, which can be immediately administered to a subject, e.g., without any processing such as thawing, reconstituting or diluting). Thus, prior to administration of a storable form of a pharmaceutical composition, this storable form has to be processed or transferred into a ready-to-use or administrable form. E.g., a frozen pharmaceutical composition has to be thawed.

Ready to use injectables can be presented in containers such as vials, ampoules or syringes wherein the container may contain one or more doses.

In one embodiment, the pharmaceutical compositions is in frozen form and can be stored at a temperature of about −90° C. or higher, such as about −90° C. to about −10° C. For example, the frozen pharmaceutical compositions described herein (such as the frozen compositions prepared by the methods of the second, third or sixth aspect, or the frozen compositions of the fifth, eighth, ninth, or tenth aspect) can be stored at a temperature ranging from about −90° C. to about −10° C., such as from about −905° C. to about −40° C. or from about −40° C. to about −25° C., or from about −25° C. to about −10° C., or a temperature of about −20° C.

In one embodiment of the pharmaceutical compositions in frozen form, the pharmaceutical composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen pharmaceutical composition can be stored for at least 4 weeks, preferably at least 1 month, more preferably at least 2 months, more preferably at least 3 months, more preferably at least 6 months at −20° C.

In one embodiment of the pharmaceutical compositions in frozen form, the RNA integrity after thawing the frozen pharmaceutical composition is at least 500%, such as at least 52%, at least 54%, at least 55%, at least 56%, at least 58%, or at least 60%, e.g., after thawing the frozen composition which has been stored at −20° C.

In one embodiment of the pharmaceutical compositions in frozen form, the size (Z_{average}) and/or size distribution and/or PDI of the LNPs after thawing the frozen pharmaceutical composition is equal to the size (Z_{average}) and/or size distribution and/or PDI of the LNPs before freezing. For example, if a ready-to-use pharmaceutical composition is prepared from a frozen pharmaceutical composition as described herein, it is preferred that the size (Z_{average}) and/or size distribution and/or PDI of the LNPs contained in the ready-to-use pharmaceutical composition is equal to the size (Z_{average}) and/or size distribution and/or PDI of the LNPs contained in the frozen pharmaceutical composition before freezing (such as contained in the formulation prepared in step (1) of the method of the second aspect).

In one embodiment, the pharmaceutical compositions is in liquid form and can be stored at a temperature ranging from about 0° C. to about 20° C. For example, the liquid pharmaceutical compositions described herein (such as the liquid compositions prepared by the methods of the second, fourth or seventh aspect, or the liquid compositions of the fifth, eighth, ninth, or tenth aspect) can be stored at a temperature ranging from about 1° C. to about 15° C., such as from about 2° C. to about 10° C., or from about 2° C. to about 8° C., or at a temperature of about 5° C.

In one embodiment of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, or at least 6 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In one embodiment of the pharmaceutical composition in liquid form, the RNA integrity of the liquid composition, when stored, e.g., at 0° C. or higher for at least one week, is sufficient to produce the desired effect, e.g., to induce an immune response. For example, the RNA integrity of the liquid composition, when stored, e.g., at 0° C. or higher for at least one week, may be at least 50%, such as at least 52%, at least 54%, at least 55%, at least 56%, at least 58%, or at least 60%, compared to the RNA integrity of the initial composition, i.e., the RNA integrity before the composition has been stored.

In one embodiment of the pharmaceutical composition in liquid form, the size (Z_{average}) (and/or size distribution and/or polydispersity index (PDI)) of the LNPs of the pharmaceutical composition, when stored, e.g., at 0° C. or higher for at least one week, is sufficient to produce the desired effect, e.g., to induce an immune response. For example, the size (Z_{average}) (and/or size distribution and/or polydispersity index (PDI)) of the LNPs of the pharmaceutical composition, when stored, e.g., at 0° C. or higher for at least one week, is equal to the size (Z_{average}) (and/or size distribution and/or PDI) of the LNPs of the initial pharmaceutical composition, i.e., before storage. In one embodiment, the size (Z_{average}) of the LNPs after storage of the pharmaceutical composition e.g., at 0° C. or higher for at least one week is between about 50 nm and about 500 nm, preferably between about 40 nm and about 200 nm, more preferably between about 40 nm and about 120 nm. In one embodiment, the PDI of the LNPs after storage of the pharmaceutical composition e.g., at 0° C. or higher for at least one week is less than 0.3, preferably less than 0.2, more preferably less than 0.1. In one embodiment, the size (Z_{average}) of the LNPs after storage of the pharmaceutical composition e.g., at 0° C. or higher for at least one week is between about 50 nm and about 500 nm, preferably between about 40 nm and about 200 nm, more preferably between about 40 nm and about 120 nm, and the size (Z_{average})(and/or size distribution and/or PDI) of the LNPs after storage of the pharmaceutical composition e.g., at 0° C. or higher for at least one week is equal to the size (Z_{average}) (and/or size distribution and/or PDI) of the LNPs before storage. In one embodiment, the size (Z_{average}) of the LNPs after storage of the pharmaceutical composition e.g., at 0° C. or higher for at least one week is between about 50 nm and about 500 nm, preferably between about 40 nm and about 200 nm, more preferably between about 40 nm and about 120 nm, and the PDI of the LNPs after storage of the pharmaceutical composition e.g., at 0° C. or higher for at least one week is less than 0.3 (preferably less than 0.2, more preferably less than 0.1).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease.

The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical compositions which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the RNA LNP compositions/formulations with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

“Pharmaceutically acceptable salts” comprise, for example, acid addition salts which may, for example, be formed by using a pharmaceutically acceptable acid such as hydrochloric acid, acetic acid, lactic acid, 2-(N-morpholino)ethanesulfonic acid (MES), 3-(N-morpholino)propanesulfonic acid (MOPS), 2-[4-(2-hydroxyethyl)piperazin-1-yl]ethanesulfonic acid (HEPES) or benzoic acid. Furthermore, suitable pharmaceutically acceptable salts may include alkali metal salts (e.g., sodium or potassium salts); alkaline earth metal salts (e.g., calcium or magnesium salts); ammonium (NH₄); and salts formed with suitable organic ligands (e.g., quaternary ammonium and amine cations). Illustrative examples of pharmaceutically acceptable salts can be found in the prior art; see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

Routes of Administration of Pharmaceutical Compostions

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumorally.

In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

Use of Pharmaceutical Compositions

RNA particles described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder.

The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide protection against an infectious disease by vaccination.

In one embodiment of the disclosure, the aim is to provide secreted therapeutic proteins, such as antibodies, bispecific antibodies, cytokines, cytokine fusion proteins, enzymes, to a subject, in particular a subject in need thereof.

In one embodiment of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In one embodiment of the disclosure, the aim is to modulate/reprogram immune cells in the blood.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated.

Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

Methods

Manufacturing of the RNA LNPs

Manufacturing protocols are described here with taking lipid I-3 as an example for the cationically ionizable lipid. The same protocols apply as well for other cationically ionizable lipids. Accordingly, also other formulations with ratios between cationically ionizable lipid and RNA (N/P ratio), e.g., higher or lower N/P ratios, including those with negative charge excess, can be manufactured and stabilized as described. In addition, other lipid ratios (phospholipid, cholesterol, polymer conjugated lipid), as well as other types of polymer conjugated lipids (e.g., polysarcosine lipids) can be used. Protocols also apply for products without any polymer conjugated lipid.

RNA LNPs were prepared by an aqueous-ethanol mixing protocol. Briefly, RNA (such as BNT162b2 encoding an amino acid sequence comprising a SARS-CoV-2 S protein) in aqueous buffer conditions (e.g., 50 mM citrate, pH 4.0) is mixed with ethanolic lipid mix comprising of lipid I-3, DSPC, cholesterol and 2-[(polyethylene glycol)-2000]-N,N-ditetradecylacetamide in molar ratio of 47.5:10:40.7:1.8, respectively in the volume ratio of 3 parts of RNA and 1 part of lipid mix. The mixing is achieved using standard pump based set-up using T mixing element. The lipid nanoparticle raw colloid is further diluted with 2 parts of buffer (e.g., with citrate buffer 50 mM, pH 4.0). The total flow is between 400 and 2000 mL/min, e.g. 720 ml/min. The primary LNP product thus obtained is further subjected to tangential flow filtration against a buffer (such as PBS buffer pH 7.4 (control) or Tris buffer 10 or 50 mM pH 7.4 or a different buffer) for buffer exchange and removal of ethanol. After completion of the diafiltration, the formulation is concentrated. Subsequently, the buffer exchanged RNA LNP formulation is diluted, e.g., with PBS supplemented with sucrose (control) or with Tris buffer 10 or 50 mM pH 7.4 supplemented with sucrose so that final RNA concentration in LNP formulations is 0.1 to 0.5 mg/ml and the sucrose content is 10% w/v. Samples of the RNA LNP formulations were either stored at 5° C. or room temperature or were frozen at stored at different temperatures (e.g., −5° C., −20° C., −70° C. and/or −80° C.).

LNP Size and Polydispersity

Mean particle size and size distribution of LNPs in an RNA LNP formulation/composition (or a sample thereof) is evaluated by dynamic light scattering (DLS). The method employs a particle sizer that uses back-scatter at 173° to determine particle size. The results are reported as the Z_{average} size of the particles and the polydispersity index. The polydispersity values are used to describe the width of fitted log-normal distribution around the measured Z_{average} size and are generated using proprietary mathematical calculations within the particle sizing software. Results for size and polydispersity are reported as nm and polydispersity index value, respectively.

RNA Integrity

RNA integrity is determined by capillary electrophoresis. RNA-LNPs treated with Triton™ X-100/ethanol are applied to a gel matrix contained in a capillary. The RNA and its derivates, degradants and impurities are separated according to their sizes. The gel matrix contains a fluorescence dye which binds specifically to the RNA components which allows detection by blue LED-induced fluorescence, detected by a CCD detector. The excitation wavelength is 470 nm. The integrity of the RNA is determined by comparing the peak area of the main RNA peak to the total detected peak area.

RNA Content and Encapsulation

The RNA content is determined by disrupting the LNPs with the detergent Triton™ X-100 and subsequently measuring the total RNA content based on the signal of the RNA-binding fluorescent dye RiboGreen® using a spectrofluorophotometer. RNA encapsulation is calculated by comparing the RiboGreen® signals of LNP samples in the absence (free RNA) and presence (total RNA) of Triton™ X-100. Results for RNA content and encapsulation are reported as mg/mL and percentage, respectively.

Lipid Identity and Lipid Content

An HPLC-CAD assay determines identity and concentration of lipids in the aliquot using a method that resolves all four lipids (I-3, DSPC, cholesterol, and 2-[(polyethylene glycol)-2000]-N,N-ditetradecylacetamide). Individual lipid identities are determined by comparison of retention times with those of the reference standards. Concentration of each individual lipid is determined by sample area response against the respective five-point calibration curve generated from the reference standards, with peak detection performed using a charged aerosol detector (CAD). Results for lipid identity and lipid content are reported as relative retention time compared to reference standard and as mg/mL, respectively.

Electron Microscopy

Fully processed and frozen (−80° C.) samples were brought to RT. 5 μl of each sample was applied to a gold grid (ULTRAuFoil 2/1, Quantifoil Micro Tools, Jena, Germany) and excess of liquid as blotted automatically onto paper. Samples were plunge-frozen in liquid ethane at −180° C. in a cryobox (Carl Zeiss NTS GmbH, Oberkochen, Germany). Excess ethane was removed and the samples were transferred immediately with a Gatan 626 cryo-transfer holder (Gatan Pleasanton, USA) into the pre-cooled cryo-electron microscope Philips CM120, Eindhoven, Netherlands) operated at 120 kV and viewed under low dose conditions. Images were recorded using a 2k CMO Camera (F216 TVIPS, Gauting, Germany). Four images were averaged per frame for noise reduction.

In vitro expression (IVE)

The protein expression (e.g., the spike protein expression) of LNP samples is measured using a characterization assay currently undergoing additional evaluation to increase day to day robustness. First, the LNP samples are added to HEK-293T cells at the RNA level indicated (non-saturating concentration). Protein expression is measured using an anti-protein monoclonal antibody (e.g., an anti-spike protein receptor binding domain (RBD) rabbit monoclonal antibody). Expression is measured by quantifying the number of cells that have a positive signal for bound anti-protein antibody (e.g., bound anti-RBD antibody).

Mouse Immunogenicity

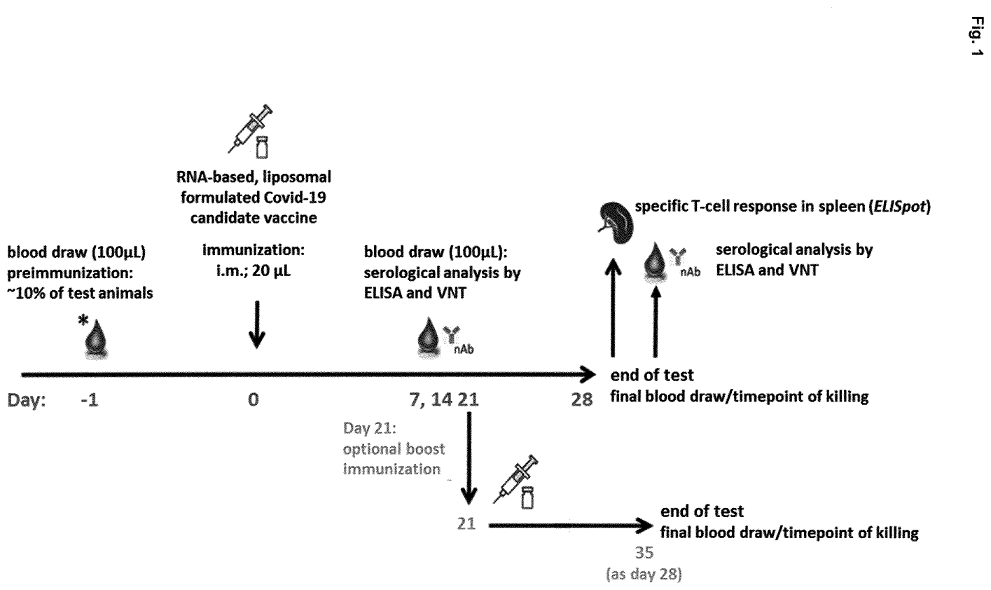

5 groups of 5 female BALB/c mice are immunized once (on day 0) with the formulated drug product at a 1 μg dose level, or with the buffer alone (control group) immunizations are given intramuscularly (i.m.) in a dose volume of 20 μL. Blood is collected once weekly for three weeks (days 7, 14, and 21) to analyze the antibody immune response by ELISA and pseudovirus-based neutralization assay (pVNT). At the end of the study (on day 28), blood is collected and animals are then euthanized for spleen collection and additional analysis of the T-cell response in splenocytes by ELISpot and intracellular cytokine staining (ICS); see FIG. 1.

### Example 1

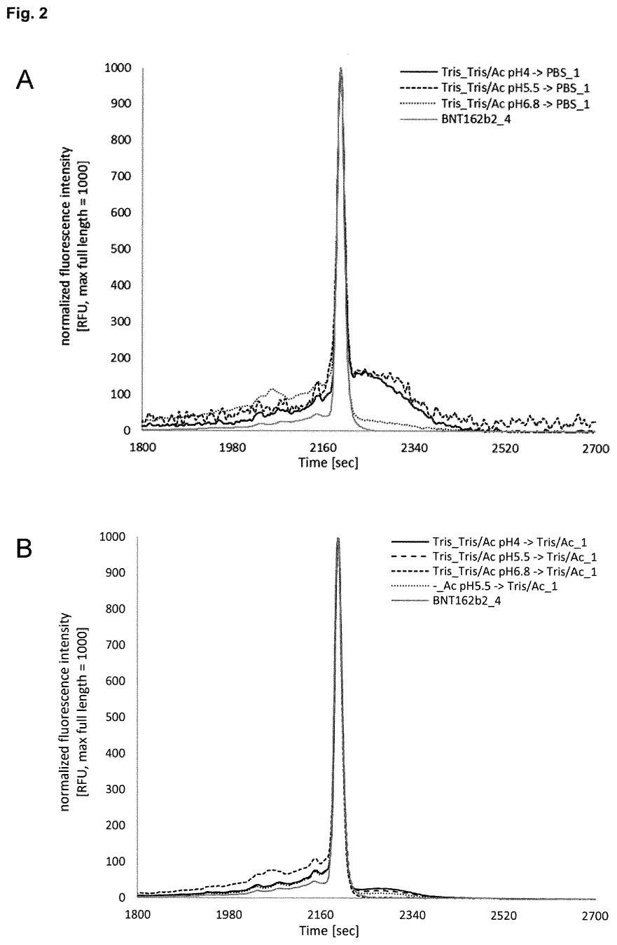

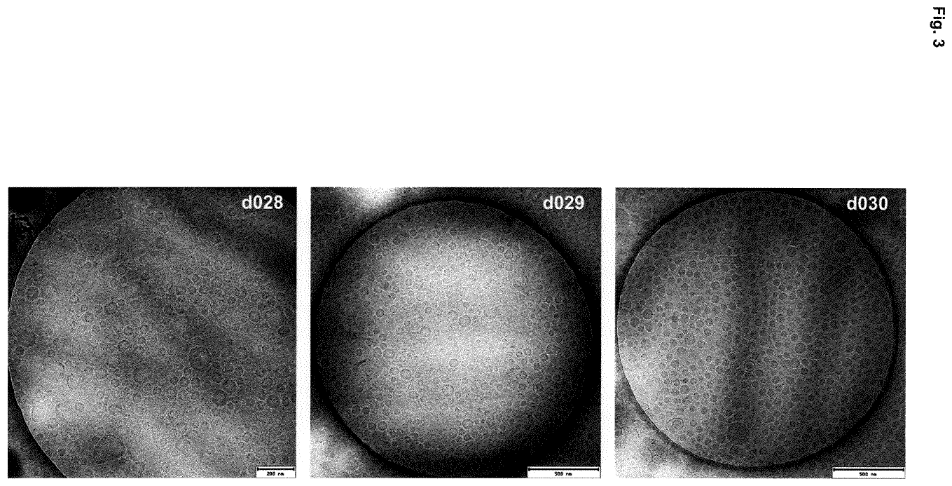

RNA LNPs were prepared by the aqueous-ethanol mixing protocol using 20 mM Tris added to the organic phase. LNPs were generated in 50 mM Tris:acetate pH 4, pH 5.5 or pH 6.8 and the resulting primary LNPs were split: one portion was subjected to dialysis against PBS (A); the other portion was subjected to dialysis against 50 mM Tris:acetate pH 7.4 (B). For comparison, the organic phase did not receive Tris, LNP were generated in 50 mM Na-acetate buffer pH 5.5 and the material was dialysed against 50 mM Tris:acetate pH 7.4. All samples were stored for 50 h at room temperature. The RNA integrity was measured as described above using capillary electrophoresis. The results are shown in FIGS. 2A and B.

RNA LNP compositions containing a cationically ionizable lipid, in particular lipid I-3, and PBS adopt a highly stable folded form of RNA (detectable as tailing of the main peak at about 2190 sec). This is also true if the LNPs were prepared in buffer other than PBS (such as Tris), i.e., in the absence of PBS, and during the dialysis the buffer was exchanged to PBS; cf., FIG. 2A. In all these samples, the amount of this highly stable folded form of RNA was between 18% and 21%.

However, using the monovalent buffer substance Tris (instead of the polyvalent PBS) in the composition (i.e., in the preparation in which the drug product is stored, shipped and administered, when formulated as ready-to to-use composition) inhibits the formation of the highly stable folded form of RNA; cf., FIG. 2B.

Thus, from these results, one can conclude that it is sufficient to add Tris during dialysis in order to inhibit the formation of the highly stable folded form of RNA. In contrast, the addition of Tris only in the upstream parts of the LNP preparation process does not protect from the formation of the highly stable folded form of RNA when the primary LNP formulation is subjected to dialysis against PBS.

### Example 2

After having identified Tris as a preferred monovalent buffer substance inhibiting the formation of the highly stable folded form of RNA, we set out to optimize the composition components with respect to colloidal stability, in particular during freeze-thaw-cycles.

Compounds comprising (i) monovalent anions (acetate, glycolate or lactate), (ii) divalent or partially divalent anions (tartrate, phosphate, carbonate) or (iii) zwitterions (HEPES and MES) were combined with Tris as the buffer substance and the colloidal stability of the LNPs was determined over time or during freeze-thaw-cycles at −20° C. The results are shown in Table 2.

TABLE 2
Colloidal Stability of LNP in buffers comprising Tris and selected anions

| | | | 25 C. | | | | 5 C. | | |
|---|---|---|---|---|---|---|---|---|---|
| | | 18 Dec. 2020 | 26 Dec. 2020 | 4 Jan. 2021 | 21 Jan. 2021 | 18 Dec. 2020 | 26 Dec. 2020 | 4 Jan. 2021 | 22 Jan. 2021 |
| | | 0 | 8 | 17 | 33 | 0 | 8 | 17 | 33 |
| Suc 0 | T50 Hac | 100% | 96% | 94% | 100% | 100% | 100% | 104% | 99% |
| Suc 120 | T45 Hac | 100% | 100% | 93% | 98% | 100% | 101% | 99% | 96% |
| Suc 240 | T40 Hac | 100% | 102% | 98% | 101% | 100% | 101% | 104% | 97% |
| Suc 360 | T35 Hac | 100% | 101% | 98% | 101% | 100% | 102% | 104% | 99% |
| Suc 480 | T30 Hac | 100% | 103% | 98% | 103% | 100% | 101% | 106% | 99% |
| Suc 600 | T25 Hac | 100% | 100% | 98% | 101% | 100% | 99% | 107% | 97% |
| Suc 0 | T50 Lac | 100% | 96% | 96% | 99% | 100% | 102% | 105% | 95% |
| Suc 120 | T45 Lac | 100% | 99% | 101% | 100% | 100% | 102% | 100% | 102% |
| Suc 240 | T40 Lac | 100% | 101% | 97% | 103% | 100% | 99% | 103% | 99% |
| Suc 360 | T35 Lac | 100% | 101% | 99% | 103% | 100% | 100% | 103% | 100% |
| Suc 480 | T30 Lac | 100% | 106% | 101% | 106% | 100% | 104% | 100% | 100% |
| Suc 600 | T25 Lac | 100% | 101% | 100% | 103% | 100% | 99% | 103% | 98% |
| Suc 0 | T50 Gly | 100% | 91% | 98% | 75% | 100% | 95% | 98% | 75% |
| Suc 120 | T45 Gly | 100% | 105% | 102% | 78% | 100% | 101% | 106% | 77% |
| Suc 240 | T40 Gly | 100% | 103% | 102% | 79% | 100% | 104% | 101% | 77% |
| Suc 360 | T35 Gly | 100% | 102% | 99% | 77% | 100% | 102% | 100% | 78% |
| Suc 480 | T30 Gly | 100% | 103% | 103% | 80% | 100% | 102% | 101% | 80% |
| Suc 600 | T25 Gly | 100% | 102% | 101% | 80% | 100% | 100% | 97% | 76% |
| Suc 0 | T50 Pi | 100% | 98% | 97% | 99% | 100% | 96% | 101% | 101% |
| Suc 120 | T45 Pi | 100% | 98% | 96% | 98% | 100% | 98% | 103% | 100% |
| Suc 240 | T40 Pi | 100% | 107% | 102% | 100% | 100% | 100% | 101% | 102% |
| Suc 360 | T35 Pi | 100% | 102% | 103% | 103% | 100% | 100% | 103% | 101% |
| Suc 480 | T30 Pi | 100% | 105% | 105% | 103% | 100% | 102% | 101% | 99% |
| Suc 600 | T25 Pi | 100% | 103% | 99% | 103% | 100% | 103% | 100% | 100% |
| Suc 0 | HEPES 50 T | 100% | 104% | 100% | 103% | 100% | 102% | 102% | 101% |
| Suc 120 | HEPES 45 T | 100% | 103% | 101% | 100% | 100% | 99% | 100% | 99% |
| Suc 240 | HEPES 40 T | 100% | 103% | 100% | 102% | 100% | 99% | 101% | 100% |
| Suc 360 | HEPES 35T | 100% | 101% | 100% | 100% | 100% | 97% | 100% | 95% |
| Suc 480 | HEPES 30 T | 100% | 101% | 97% | 102% | 100% | 97% | 100% | 94% |
| Suc 600 | HEPES 25T | 100% | 105% | 100% | 103% | 100% | 97% | 97% | 97% |
| Suc 0 | MES 50 T | 100% | 102% | 101% | 97% | 100% | 100% | 100% | 97% |
| Suc 120 | MES 45 T | 100% | 100% | 100% | 100% | 100% | 99% | 100% | 99% |
| Suc 240 | MES 40 T | 100% | 102% | 102% | 100% | 100% | 106% | 102% | 101% |
| Suc 360 | MES 35T | 100% | 100% | 101% | 101% | 100% | 101% | 99% | 97% |
| Suc 480 | MES 30 T | 100% | 106% | 99% | 103% | 100% | 100% | 101% | 95% |
| Suc 600 | MES 25T | 100% | 109% | 102% | 111% | 100% | 102% | 103% | 100% |
| Suc 0 | 50 HCO3 | 100% | 101% | 104% | 101% | 100% | 102% | 102% | 102% |
| Suc 120 | 45 HCO3 | 100% | 98% | 98% | 101% | 100% | 99% | 98% | 99% |
| Suc 240 | 40 HCO3 | 100% | 100% | 102% | 100% | 100% | 102% | 102% | 102% |
| Suc 360 | 35 HCO3 | 100% | 104% | 101% | 101% | 100% | 102% | 99% | 103% |
| Suc 480 | 30 HCO3 | 100% | 100% | 98% | 105% | 100% | 101% | 99% | 113% |
| Suc 600 | 25 HCO3 | 100% | 102% | 102% | 106% | 100% | 101% | 99% | 98% |
| Suc 0 | 50 Tart | 100% | 99% | 99% | 99% | 100% | 99% | 101% | 97% |
| Suc 120 | 45 Tart | 100% | 101% | 99% | 101% | 100% | 100% | 100% | 99% |
| Suc 240 | 40 Tart | 100% | 102% | 97% | 101% | 100% | 99% | 98% | 98% |
| Suc 360 | 35 Tart | 100% | 105% | 103% | 105% | 100% | 102% | 102% | 101% |
| Suc 480 | 30 Tart | 100% | 105% | 99% | 107% | 100% | 103% | 101% | 103% |
| Suc 600 | 25 Tart | 100% | 104% | 104% | 110% | 100% | 101% | 99% | 102% |

| | | | −20 C. | | | | | −70 C. | |
|---|---|---|---|---|---|---|---|---|---|
| | | 18 Dec. 2020 | 4 Jan. 2021 | 5 Jan. 2021 | 6 Jan. 2021 | 22 Jan. 2021 | 18 Dec. 2020 | 22 Jan. 2021 | |
| | | 0 | FT1 | FT2 | FT3 | 33 | 0 | 33 | |
| Suc 0 | T50 Hac | 100% | 101% | 96% | 104% | 100% | 100% | 96% | Hac |
| Suc 120 | T45 Hac | 100% | 104% | 104% | 107% | 101% | 100% | 96% | |
| Suc 240 | T40 Hac | 100% | 101% | 107% | 103% | 98% | 100% | 97% | |
| Suc 360 | T35 Hac | 100% | 105% | 112% | 108% | 98% | 100% | 95% | |
| Suc 480 | T30 Hac | 100% | 103% | 108% | 105% | 99% | 100% | 95% | |
| Suc 600 | T25 Hac | 100% | 99% | 104% | 99% | 95% | 100% | 89% | |
| Suc 0 | T50 Lac | 100% | 98% | 103% | 108% | 97% | 100% | 98% | Lac |
| Suc 120 | T45 Lac | 100% | 106% | 108% | 108% | 101% | 100% | 102% | |
| Suc 240 | T40 Lac | 100% | 105% | 108% | 114% | 100% | 100% | 99% | |
| Suc 360 | T35 Lac | 100% | 102% | 110% | 108% | 104% | 100% | 97% | |
| Suc 480 | T30 Lac | 100% | 103% | 108% | 110% | 104% | 100% | 98% | |
| Suc 600 | T25 Lac | 100% | 99% | 103% | 100% | 98% | 100% | 92% | |
| Suc 0 | T50 Gly | 100% | 124% | 131% | 104% | 100% | 100% | 122% | Gly |
| Suc 120 | T45 Gly | 100% | 113% | 117% | 92% | 87% | 100% | 80% | |
| Suc 240 | T40 Gly | 100% | 107% | 113% | 89% | 82% | 100% | 81% | |
| Suc 360 | T35 Gly | 100% | 107% | 111% | 85% | 82% | 100% | 77% | |
| Suc 480 | T30 Gly | 100% | 106% | 114% | 87% | 81% | 100% | 76% | |
| Suc 600 | T25 Gly | 100% | 101% | 107% | 84% | 85% | 100% | 71% | |
| Suc 0 | T50 Pi | 100% | ND | ND | ND | ND | 100% | 333% | Pi |
| Suc 120 | T45 Pi | 100% | ND | ND | ND | 146% | 100% | 141% | |
| Suc 240 | T40 Pi | 100% | ND | 128% | 142% | 139% | 100% | 111% | |
| Suc 360 | T35 Pi | 100% | 110% | 114% | 122% | 120% | 100% | 101% | |
| Suc 480 | T30 Pi | 100% | 109% | 117% | 116% | 116% | 100% | 101% | |
| Suc 600 | T25 Pi | 100% | 109% | 115% | 113% | 105% | 100% | 98% | |
| Suc 0 | HEPES 50 T | 100% | 113% | 125% | 131% | 115% | 100% | 140% | HEPES |
| Suc 120 | HEPES 45 T | 100% | 109% | 119% | 129% | 108% | 100% | 104% | |
| Suc 240 | HEPES 40 T | 100% | 112% | 118% | 124% | 115% | 100% | 101% | |
| Suc 360 | HEPES 35T | 100% | 103% | 115% | 123% | 107% | 100% | 101% | |
| Suc 480 | HEPES 30 T | 100% | 105% | 113% | 118% | 107% | 100% | 102% | |
| Suc 600 | HEPES 25T | 100% | 112% | 115% | 122% | 113% | 100% | 98% | |
| Suc 0 | MES 50 T | 100% | 103% | 106% | 102% | 99% | 100% | 100% | MES |
| Suc 120 | MES 45 T | 100% | 101% | 104% | 103% | 100% | 100% | 98% | |
| Suc 240 | MES 40 T | 100% | 106% | 108% | 107% | 104% | 100% | 99% | |
| Suc 360 | MES 35T | 100% | 102% | 104% | 101% | 106% | 100% | 99% | |
| Suc 480 | MES 30 T | 100% | 104% | 102% | 105% | 99% | 100% | 97% | |
| Suc 600 | MES 25T | 100% | 109% | 109% | 108% | 105% | 100% | 97% | |
| Suc 0 | 50 HCO3 | 100% | 132% | ND | 141% | ND | 100% | 169% | HCO3 |
| Suc 120 | 45 HCO3 | 100% | ND | 137% | 150% | 136% | 100% | 109% | |
| Suc 240 | 40 HCO3 | 100% | 114% | 124% | 136% | 122% | 100% | 102% | |
| Suc 360 | 35 HCO3 | 100% | 109% | 110% | 115% | 114% | 100% | 103% | |
| Suc 480 | 30 HCO3 | 100% | 108% | 110% | 111% | 104% | 100% | 98% | |
| Suc 600 | 25 HCO3 | 100% | 110% | 108% | 108% | 108% | 100% | 103% | |
| Suc 0 | 50 Tart | 100% | 138% | 140% | 142% | 132% | 100% | 165% | Tart |
| Suc 120 | 45 Tart | 100% | 154% | 153% | 163% | 139% | 100% | 112% | |
| Suc 240 | 40 Tart | 100% | 115% | 125% | 130% | 122% | 100% | 112% | |
| Suc 360 | 35 Tart | 100% | 109% | 117% | 119% | 114% | 100% | 104% | |
| Suc 480 | 30 Tart | 100% | 109% | 118% | 119% | 106% | 100% | 101% | |
| Suc 600 | 25 Tart | 100% | 113% | 114% | 114% | 114% | 100% | 105% | |

LNPs (formed in 50 mM Tris:Hac pH 5.5, TFF in 50 mM Tris:Hac pH 7.4) were diluted 10-fold into the matrix listed on the left column. Materials were incubated at the temperatures and for the time indicated on the top. Particle size of LNP is expressed as relative to the original size. Values between 90% and 110% represent material that is considered stable. Suc = concentration of sucrose in mM, T = Tris, Hac = acetic acid, Lac = lactic acid, Gly = glycolic acid, Pi = inorganic phosphate, HEPES = hydroxyethylpiperazine ethanesulfonic acid, MES = morpholinoethanesulfonic acid, Tart = tartaric acid.

As evident from the data presented in Table 2, the presence of the monovalent anions acetate, lactate and MES facilitate freezing of the RNA LNP compositions without a collapse of the colloid. Of note, the colloidal stability extends for up to 3 freeze-thaw-cycles plus a follow-up period of 89 days at −20° C. or for the same time at 5° C.

However, the presence of partially or fully divalent ions carbonate, phosphate and tartrate results in RNA LNP composition which lack colloidal stability during freezing.

In summary, it can be concluded that LNPs are colloidal stable at −20° C. in buffers comprising Tris as cation and monovalent anions selected from acetate, lactate or MES, but are not stable in the presence of di- and/or polybasic organic acids. The colloidal stability includes repeated freeze-thaw-cycles and extended periods of storage or combinations of both factors.

### Example 3

The following three RNA LNP compositions were prepared
D028
LNP were formed in 50 mM citrate pH 4.0 and processed as set forth above. 50 mM Tris:acetate pH 7.4 was introduced during TFF and the formulation was diluted to give an RNA concentration of 0.5 mg/mL or 0.1 mg/mL in the same buffer further comprising 300 mM sucrose.
D029
For D029, a single buffer of 50 mM Tris:aceate pH 6.9 was used for LNP formation, TFF and dilution. 300 mM sucrose was added as above.
D030
LNP were formed in 50 mM Tris:acetate pH 5.5 and processed as set forth above using the same buffer. TFF and dilution were performed as has been done for D028.

The characteristics of the resulting RNA LNP compositions are summarized in Table 3.

TABLE 3
Characteristics of the RNA LNP compositions D028, D029 and D030

| | D028 | | D029 | | D030 | |
|---|---|---|---|---|---|---|
| | RNA conc. [mg/mL] | | RNA conc. [mg/mL] | | RNA conc. [mg/mL] | |
| | 0.1 | 0.5 | 0.1 | 0.5 | 0.1 | 0.5 |
| Size [nm] | 76 | 75 | 67 | 66 | 70 | 69 |
| PDI | 0.11 | 0.11 | 0.11 | 0.14 | 0.07 | 0.07 |
| Encapsulation [%] | 96 | 94 | 96 | 96 | 95 | 96 |
| RNA Integrity [%] | 68 | 68 | 67 | 70 | 66 | 70 |
| RNA content [mg/mL] | 0.11 | 0.49 | 0.09 | 0.51 | 0.10 | 0.50 |

As evident from Table 3, the RNA LNP compositions are comparable amongst each other.

The RNA LNP compositions were further characterized using cryo electron microscopy. The results thereof are shown in FIG. 3. As can be seen from FIG. 3, all RNA LNP compositions share a common morphology described as predominantly filled, spherical vesicles of 30 to 110 nm. An outer bilayer is frequently observed.

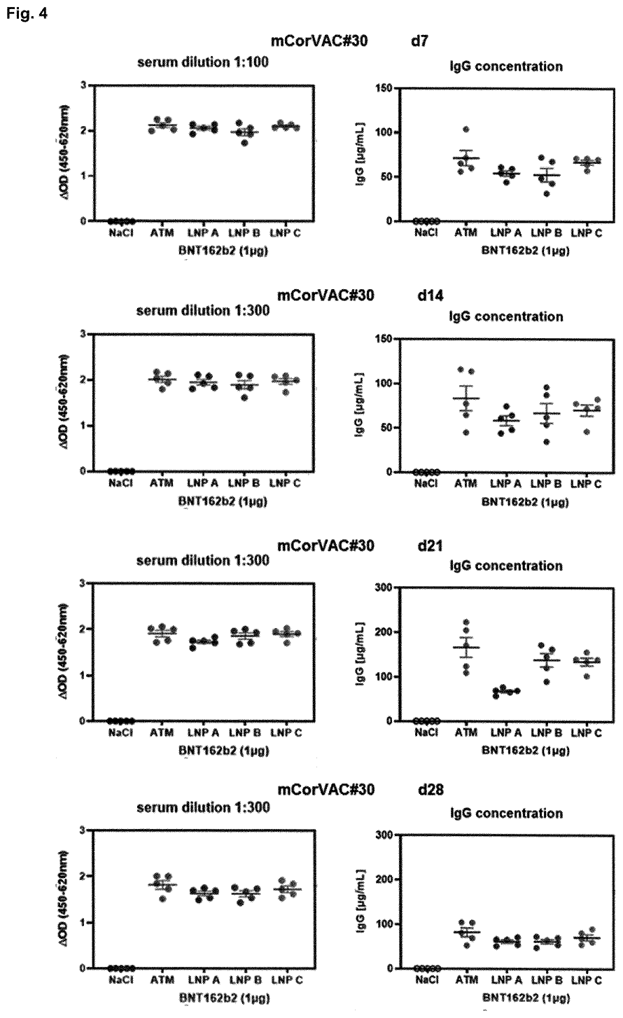

All RNA LNP compositions are also comparable to a reference and amongst each other in terms of biological activity when tested in mice. The amount of Si protein expressed and the IgG concentrations for S1 specific antibodies are comparable as shown in FIG. 4. The lower levels of S1 specific antibodies for D028 at day 21 are considered an outlier when viewed in perspective to the day 14 and day 28 titers as well as in light of the S1 expression.

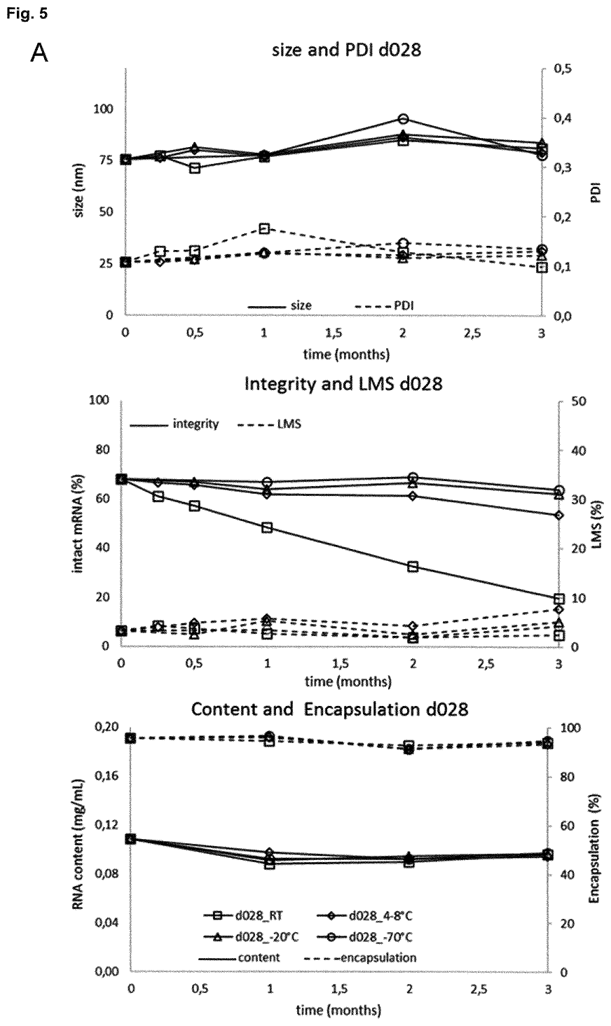

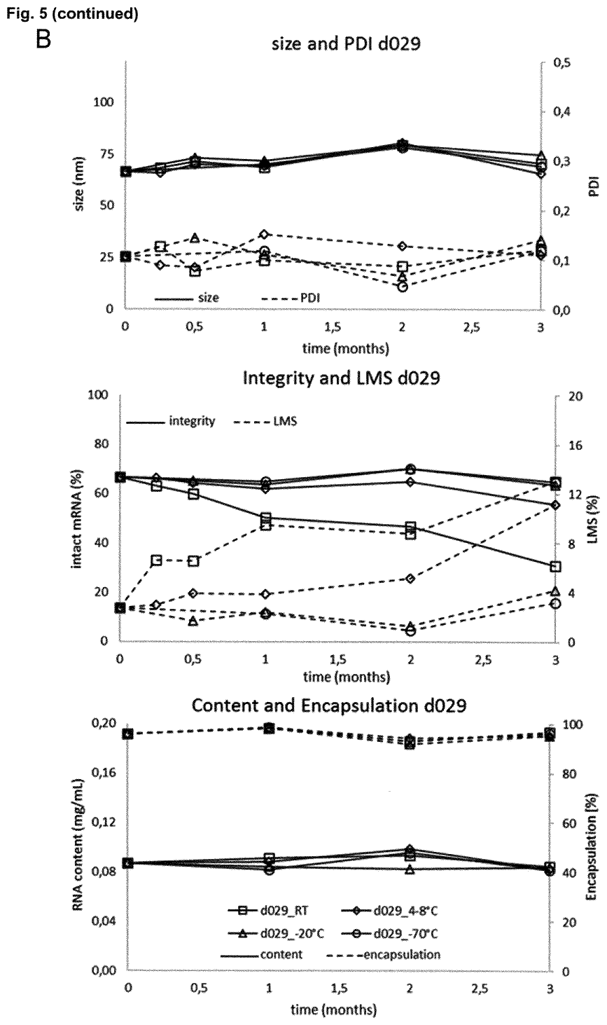

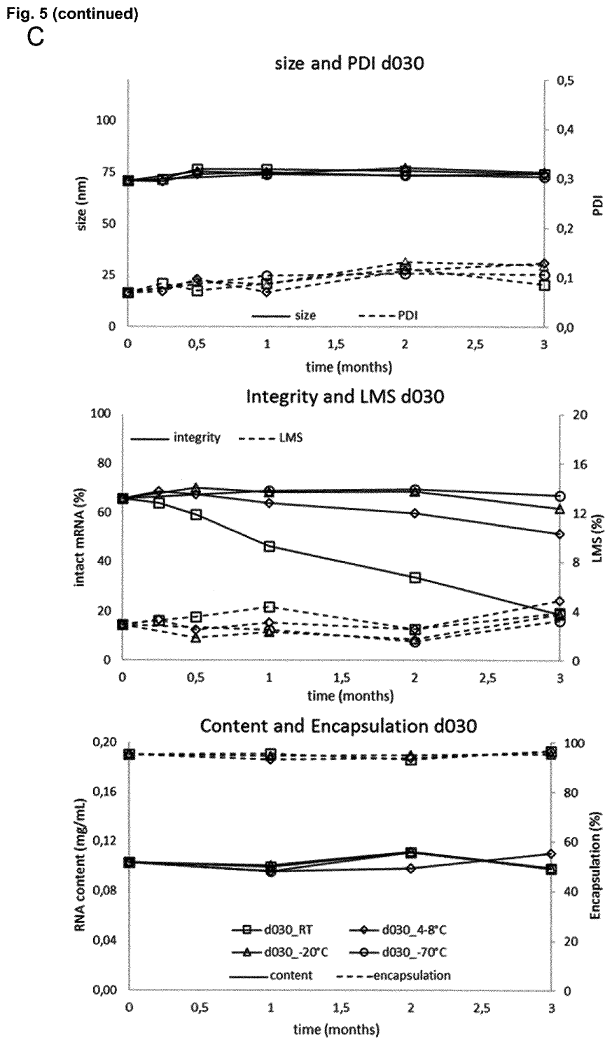

As one objective of the present disclosure is the development of RNA LNP compositions having improved stability the critical quality attributes relating to stability were analyzed. Samples from the RNA LNP compositions D028, D029 and D030 were kept at temperatures ranging between −70° C. and room temperature and characterized with regard to their physicochemical properties and activity in an IVE assay. The results thereof are shown in FIG. 5.

FIG. 5A demonstrates colloidal stability of the RNA LNP composition D028 at all temperature levels. This includes the physical stability of the LNP structure and the overall RNA content of the material. The integrity of the RNA decays in a temperature dependent fashion, as expected but is still within the specification after 12 weeks. Of note, the formation of the highly stable folded form of RNA is essentially absent in this RNA LNP composition even when exposed to room temperature over the entire period of 12 weeks.

The RNA LNP compositions D029 and D030 feature a similar pattern of stability. None of the storage conditions affects the colloidal stability, physical integrity or content of the materials. The RNA integrity remains within the limits of the specification when exposed to +5° C. over 12 weeks; cf., FIGS. 5B and SC.

In summary, RNA LNP compositions employing a buffer system comprising Tris and acetate are comparable in terms of morphology, mouse immunogenicity and for physicochemical properties at release and during stability.

### Example 4

This Example analyzes the effect of (i) the buffer concentration and (ii) the counter anion for Tris as the buffer substance on the colloidal stability and RNA integrity of RNA LNP compositions.

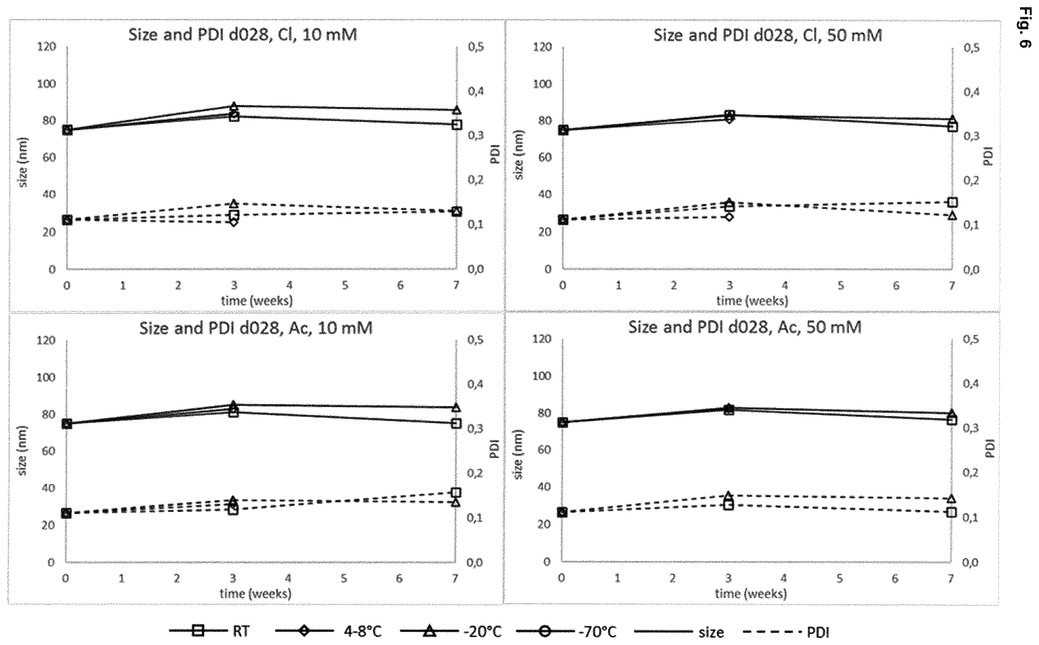

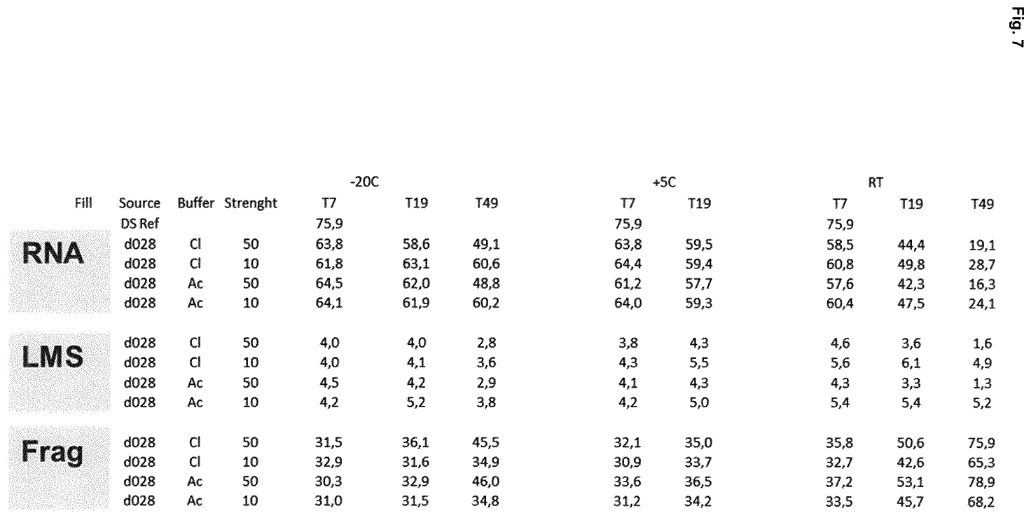

To this end, RNA LNP formulations were generated based on D028, dialyzed against Tris:acetate 10 mM and 50 mM as well as Tris:HCl 10 mM and 50 mM and the resulting RNA LNP compositions were analyzed with regard to their colloidal and RNA stability. In particular, RNA LNP compositions were incubated for up to 49 days in liquid or frozen form. Data at 5° C. were only collected for 19 days, but adhere to the results at room temperature as expected from results presented in Example 2. The results are shown in FIGS. 6 and 7.

As can be seen from FIG. 6, the particles size of RNA LNP compositions stored at −20° C. started to slightly separate from the liquid samples when stored in buffer having 10 mM strength while 50 mM buffer offered full colloidal stability. It can be concluded that RNA, when formulated in Tris buffer having monovalent anions such as acetate or chloride, is sensitive to the buffer strength of the RNA LNP composition matrix.

As can be seen from FIG. 7, the RNA LNP compositions having a buffer strength of 10 mM were more stable compared to those being formulated in 50 mM buffer. The finding is independent of the anion type. The different degradation rates were observed for the frozen material as well.

In summary, the RNA stability is sensitive to the buffer strength of the of RNA LNP compositions when formulated in Tris buffer having monovalent anions such as acetate or chloride.

1. A composition comprising lipid nanoparticles (LNPs) dispersed in an aqueous phase, wherein the LNPs comprise a cationically ionizable lipid and RNA; the aqueous phase comprises a buffer system comprising a buffer substance and a monovalent anion, the buffer substance being selected from the group consisting of tris(hydroxymethyl)aminomethane (Tris) and its protonated form, bis(2-hydroxyethyl)amino-tris(hydroxymethyl)methane (Bis-Tris-methane) and its protonated form, and triethanolamine (TEA) and its protonated form, and the monovalent anion being selected from the group consisting of chloride, acetate, glycolate, lactate, the anion of morpholinoethanesulfonic acid (MES), the anion of 3-(N-morpholino)propanesulfonic acid (MOPS), and the anion of 2-[4-(2-hydroxyethyl)piperazin-1-yl]ethanesulfonic acid (HEPES); the concentration of the buffer substance in the composition is at most about 25 mM; and the aqueous phase is substantially free of inorganic phosphate anions, substantially free of citrate anions, and substantially free of anions of ethylenediaminetetraacetic acid (EDTA).

2. The composition of claim 1, wherein the buffer substance is Tris and its protonated form.

3. The composition of claim 1 or 2, wherein the concentration of the buffer substance, in particular Tris and its protonated form, in the composition is at most about 20 mM, preferably at most about 15 mM, more preferably at most about 10 mM, such as about 10 mM.

4. The composition of any one of claims 1 to 3, wherein the aqueous phase is substantially free of inorganic sulfate anions and/or carbonate anions and/or dibasic organic acid anions and/or polybasic organic acid anions, in particular substantially free of inorganic sulfate anions, carbonate anions, dibasic organic acid anions and polybasic organic acid anions.

5. The composition of any one of claims 1 to 4, wherein the monovalent anion is selected from the group consisting of chloride, acetate, glycolate, and lactate, and the concentration of the monovalent anion in the composition is at most equal to, preferably less than the concentration of the buffer substance in the composition, such as less than about 9 mM.

6. The composition of any one of claims 1 to 4, wherein the monovalent anion is selected from the group consisting of the anions of MES, MOPS, and HEPES, and the concentration of the monovalent anion in the composition is at least equal to, preferably higher than the concentration of the buffer substance in the composition.

7. The composition of any one of claims 1 to 6, wherein the pH of the composition is between about 6.5 and about 8.0, preferably between about 6.9 and about 7.9, such as between about 7.0 and about 7.8.

8. The composition of any one of claims 1 to 7, wherein water is the main component in the composition and/or the total amount of solvent(s) other than water contained in the composition is less than about 0.5% (v/v).

9. The composition of any one of claims 1 to 8, wherein the osmolality of the composition is at most about 400×10⁻³ osmol/kg.

10. The composition of any one of claims 1 to 9, wherein the concentration of the RNA in the composition is about 5 mg/l to about 150 mg/l, preferably about 10 mg/l to about 130 mg/l, more preferably about 30 mg/l to about 120 mg/l.

11. The composition of any one of claims 1 to 10, wherein the composition comprises a cryoprotectant, preferably in a concentration of at least about 1% w/v, wherein the cryoprotectant preferably comprises one or more compounds selected from the group consisting of carbohydrates and sugar alcohols, more preferably the cryoprotectant is selected from the group consisting of sucrose, glucose, glycerol, sorbitol, and a combination thereof, more preferably the cryoprotectant comprises sucrose and/or glycerol.

12. The composition of any one of claims 1 to 10, wherein the composition is substantially free of a cryoprotectant.

13. The composition of any one of claims 1 to 12, wherein the cationically ionizable lipid comprises a head group which includes at least one nitrogen atom which is capable of being protonated under physiological conditions.

14. The composition of any one of claims 1 to 13, wherein the cationically ionizable lipid has the structure of Formula (I): 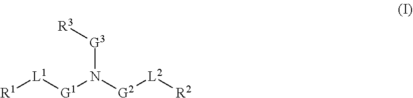
or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NRC(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² am each independently unsubstituted C₁-C₂₄ alkylene or C₂-C₁₂ alkenylene;
G³ is C₁-C₂₄ alkylene, C₂-C₂₄ alkenylene, C₁-C₁₂ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NRIC(═O)R⁴;
R⁴ is C₁-C₁₂ alkyl;
R⁵ is H or C₁-C₆ alkyl; and
x is 0, 1 or 2.

15. The composition of any one of claims 1 to 13, wherein:
(α) the cationically ionizable lipid is selected from the following structures I-1 to I-36:

| No. | Structure |
|---|---|
| I-1 | 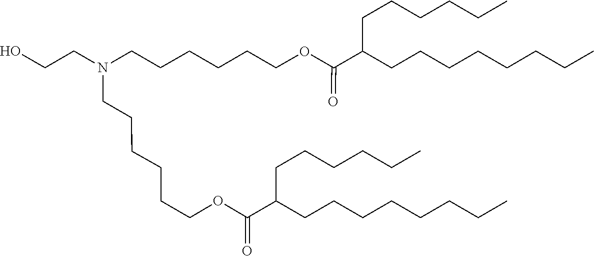 [SMILES: CCCCCCCCC(CCCCCC)C(=O)OCCCCCCN(CCO)CCCCCCOC(=O)C(CCCCCC)CCCCCCCC] |
| I-2 | 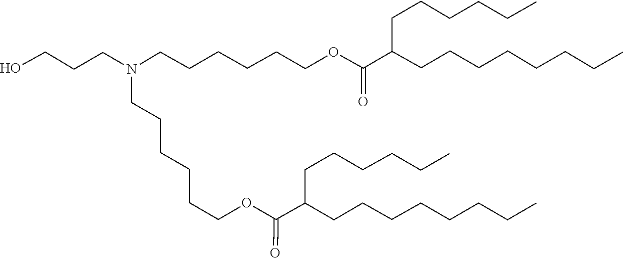 [SMILES: CCCCCCCCC(CCCCCC)C(=O)OCCCCCCN(CCCO)CCCCCCOC(=O)C(CCCCCC)CCCCCCCC] |
| I-3 | 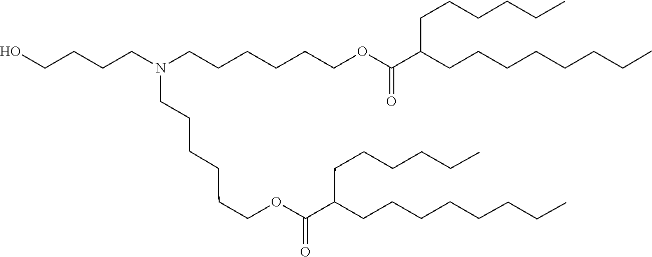 [SMILES: CCCCCCCCC(CCCCCC)C(=O)OCCCCCCN(CCCCO)CCCCCCOC(=O)C(CCCCCC)CCCCCCCC] |
| I-4 | 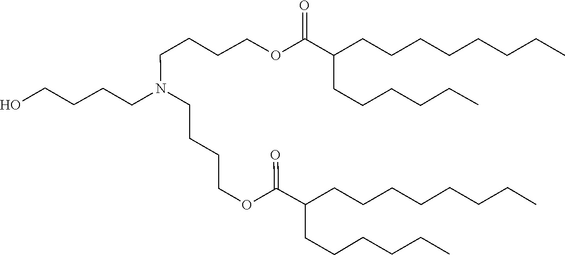 [SMILES: CCCCCCCCC(CCCCCC)C(=O)OCCCCN(CCCCO)CCCCOC(=O)C(CCCCCC)CCCCCCCC] |
| I-5 | 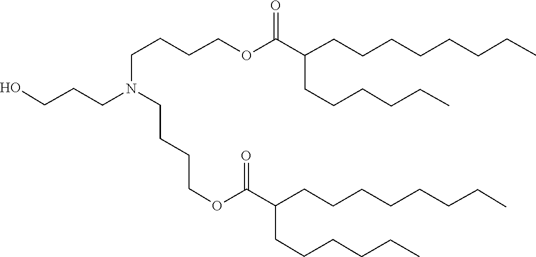 [SMILES: CCCCCCCCC(CCCCCC)C(=O)OCCCCN(CCCO)CCCCOC(=O)C(CCCCCC)CCCCCCCC] |
| I-6 | 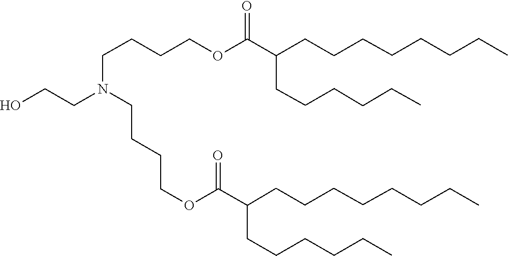 [SMILES: CCCCCCCCC(CCCCCC)C(=O)OCCCCN(CCO)CCCCOC(=O)C(CCCCCC)CCCCCCCC] |
| I-7 | 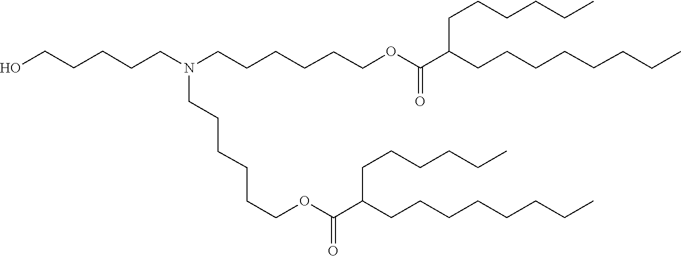 [SMILES: CCCCCCCCC(CCCCCC)C(=O)OCCCCCCN(CCCCCO)CCCCCCOC(=O)C(CCCCCC)CCCCCCCC] |
| I-8 | 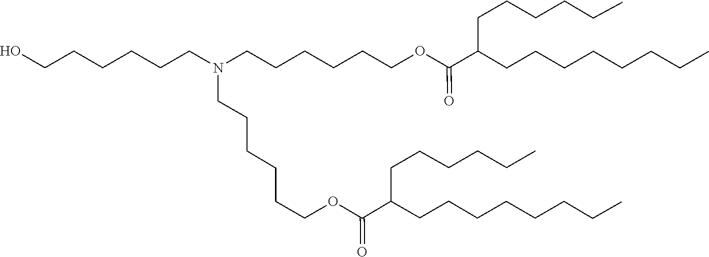 [SMILES: CCCCCCCCC(CCCCCC)C(=O)OCCCCCCN(CCCCCCO)CCCCCCOC(=O)C(CCCCCC)CCCCCCCC] |
| I-9 | 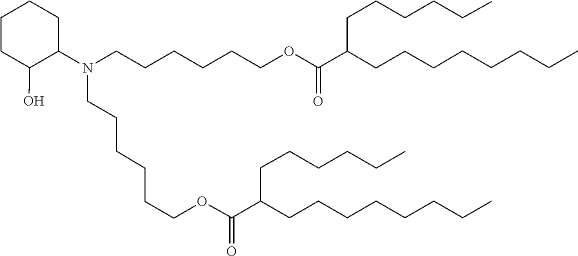 [SMILES: CCCCCCCCC(CCCCCC)C(=O)OCCCCCCN(CCCCCCOC(=O)C(CCCCCC)CCCCCCCC)C1CCCCC1O] |
| I-10 | 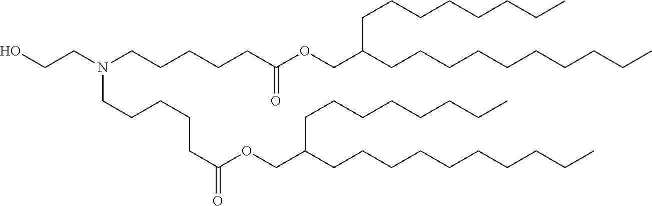 [SMILES: CCCCCCCCCCC(CCCCCCCC)COC(=O)CCCCCN(CCO)CCCCCC(=O)OCC(CCCCCCCC)CCCCCCCCCC] |
| I-11 | 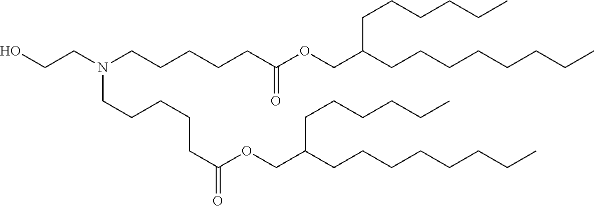 [SMILES: CCCCCCCCC(CCCCCC)COC(=O)CCCCCN(CCO)CCCCCC(=O)OCC(CCCCCC)CCCCCCCC] |
| I-12 | 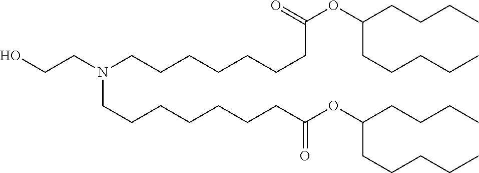 [SMILES: CCCCCC(CCCC)OC(=O)CCCCCCCN(CCO)CCCCCCCC(=O)OC(CCCC)CCCCC] |
| I-13 | 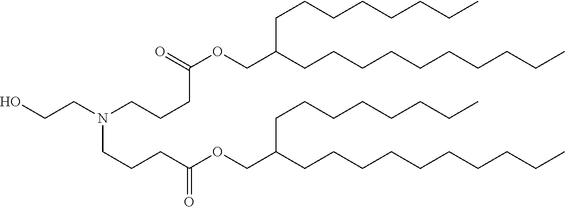 [SMILES: CCCCCCCCCCC(CCCCCCCC)COC(=O)CCCN(CCO)CCCC(=O)OCC(CCCCCCCC)CCCCCCCCCC] |
| 1-14 | 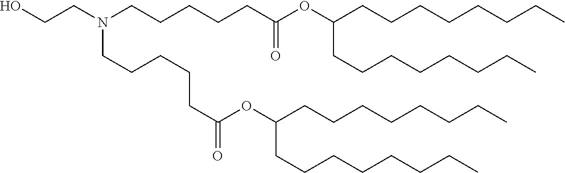 [SMILES: CCCCCCCCC(CCCCCCCC)OC(=O)CCCCCN(CCO)CCCCCC(=O)OC(CCCCCCCC)CCCCCCCC] |
| I-15 | 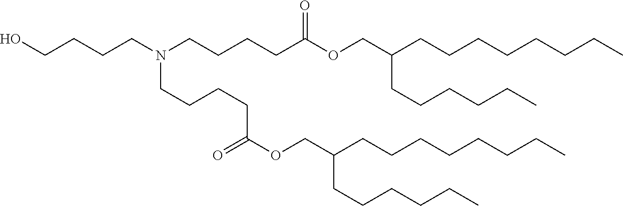 [SMILES: CCCCCCCCC(CCCCCC)COC(=O)CCCCN(CCCCO)CCCCC(=O)OCC(CCCCCC)CCCCCCCC] |
| I-16 | 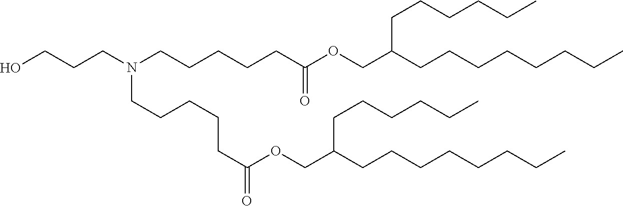 [SMILES: CCCCCCCCC(CCCCCC)COC(=O)CCCCCN(CCCO)CCCCCC(=O)OCC(CCCCCC)CCCCCCCC] |
| I-17 | 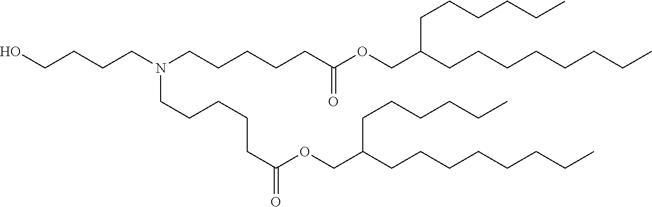 [SMILES: CCCCCCCCC(CCCCCC)COC(=O)CCCCCN(CCCCO)CCCCCC(=O)OCC(CCCCCC)CCCCCCCC] |
| I-18 | 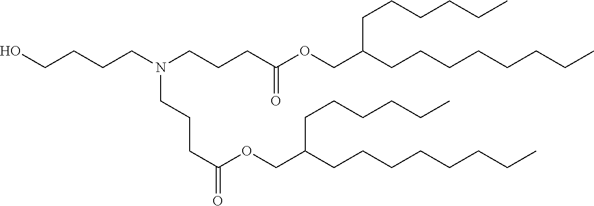 [SMILES: CCCCCCCCC(CCCCCC)COC(=O)CCCN(CCCCO)CCCC(=O)OCC(CCCCCC)CCCCCCCC] |
| I-19 | 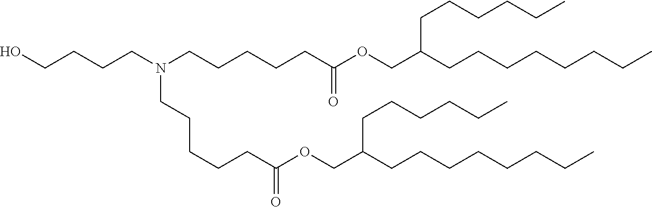 [SMILES: CCCCCCCCC(CCCCCC)COC(=O)CCCCCN(CCCCO)CCCCCC(=O)OCC(CCCCCC)CCCCCCCC] |
| I-20 | 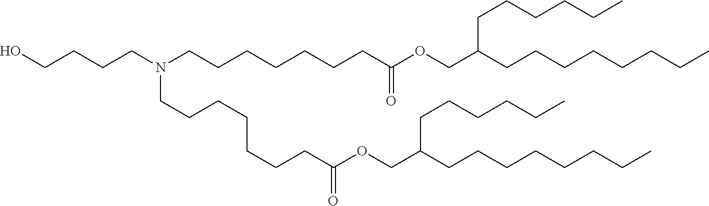 [SMILES: CCCCCCCCC(CCCCCC)COC(=O)CCCCCCCN(CCCCO)CCCCCCCC(=O)OCC(CCCCCC)CCCCCCCC] |
| I-21 | 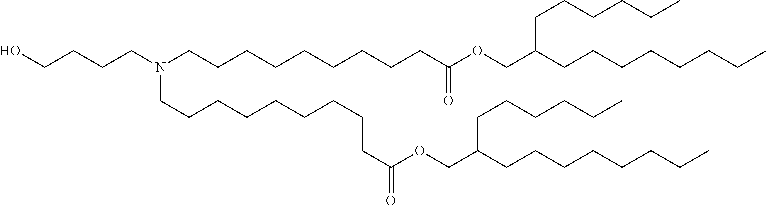 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 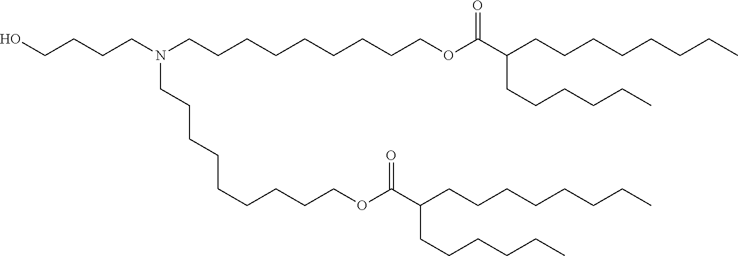 [SMILES: CCCCCCCCC(CCCCCC)C(=O)OCCCCCCCCCN(CCCCO)CCCCCCCCCOC(=O)C(CCCCCC)CCCCCCCC] |
| I-23 | 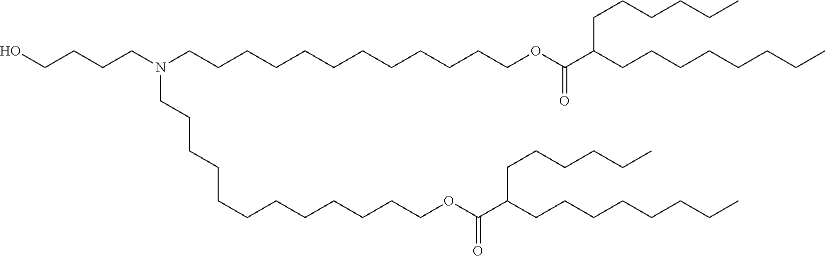 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 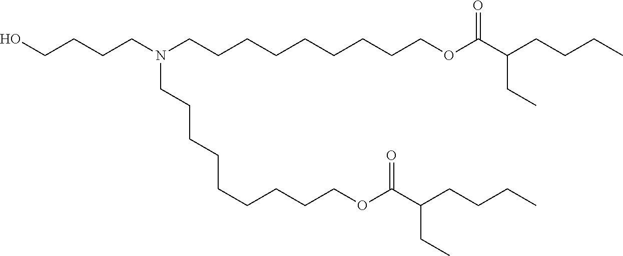 [SMILES: CCCCC(CC)C(=O)OCCCCCCCCCN(CCCCO)CCCCCCCCCOC(=O)C(CC)CCCC] |
| I-25 | 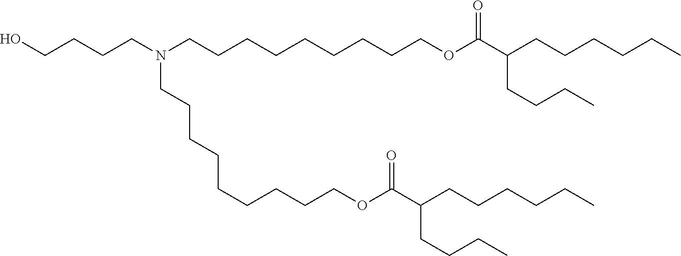 [SMILES: CCCCCCC(CCCC)C(=O)OCCCCCCCCCN(CCCCO)CCCCCCCCCOC(=O)C(CCCC)CCCCCC] |
| I-26 | 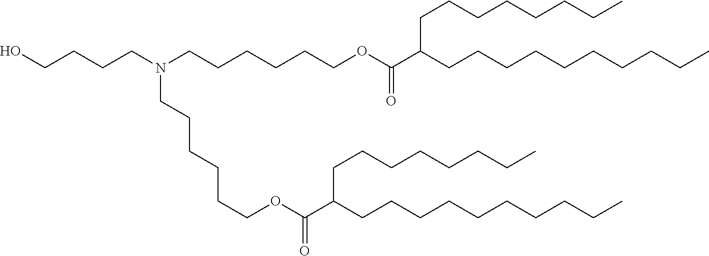 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 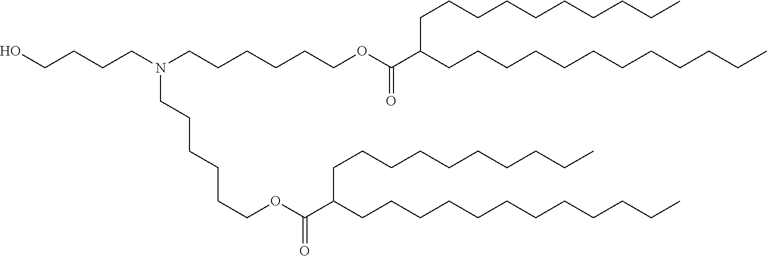 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 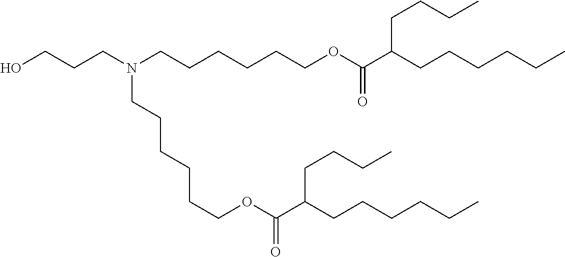 [SMILES: CCCCCCC(CCCC)C(=O)OCCCCCCN(CCCO)CCCCCCOC(=O)C(CCCC)CCCCCC] |
| I-29 | 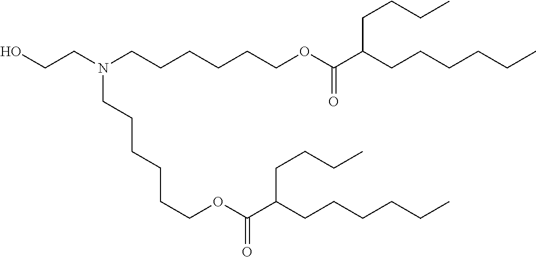 [SMILES: CCCCCCC(CCCC)C(=O)OCCCCCCN(CCO)CCCCCCOC(=O)C(CCCC)CCCCCC] |
| I-30 | 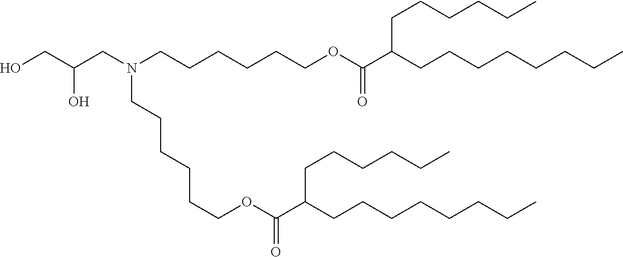 [SMILES: CCCCCCCCC(CCCCCC)C(=O)OCCCCCCN(CCCCCCOC(=O)C(CCCCCC)CCCCCCCC)CC(O)CO] |
| I-31 | 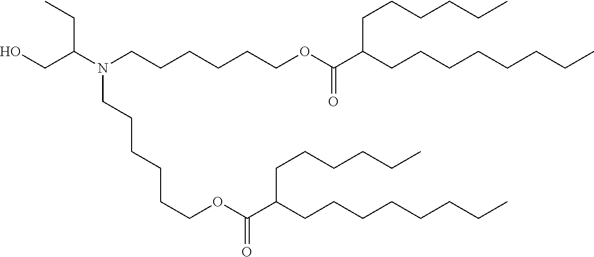 [SMILES: CCCCCCCCC(CCCCCC)C(=O)OCCCCCCN(CCCCCCOC(=O)C(CCCCCC)CCCCCCCC)C(CC)CO] |
| I-32 | 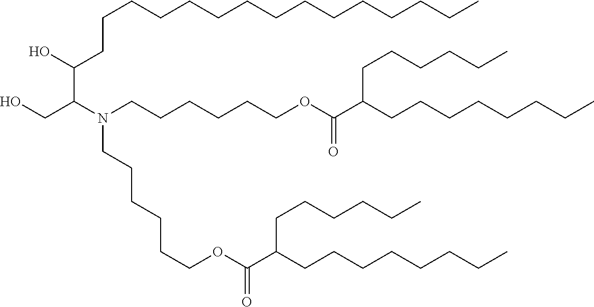 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 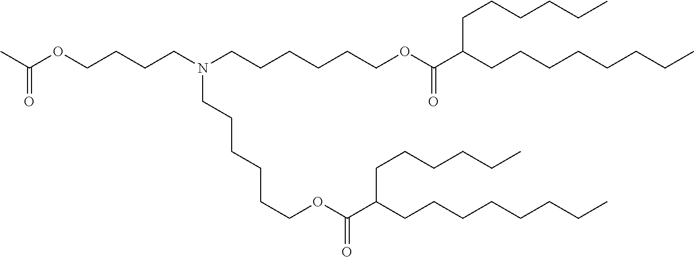 [SMILES: CCCCCCCCC(CCCCCC)C(=O)OCCCCCCN(CCCCCCOC(=O)C(CCCCCC)CCCCCCCC)CCCCOC(C)=O] |
| I-34 | 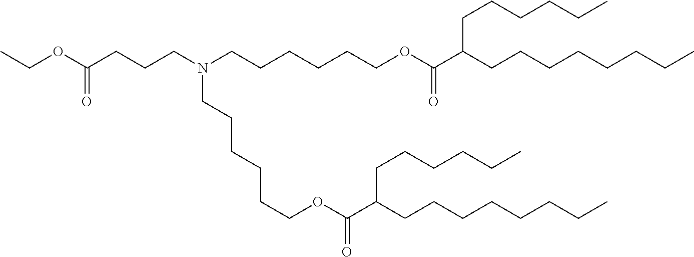 [SMILES: CCCCCCCCC(CCCCCC)C(=O)OCCCCCCN(CCCCCCOC(=O)C(CCCCCC)CCCCCCCC)CCCC(=O)OCC] |
| I-35 | 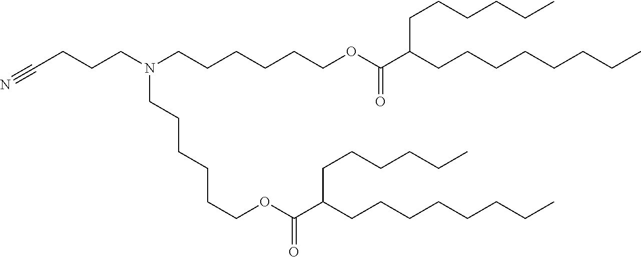 [SMILES: CCCCCCCCC(CCCCCC)C(=O)OCCCCCCN(CCCC#N)CCCCCCOC(=O)C(CCCCCC)CCCCCCCC] |
| I-36 | 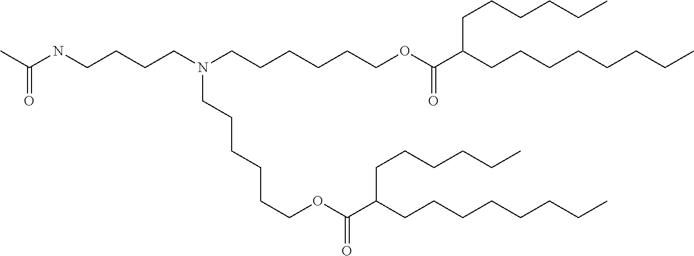 [SMILES: CCCCCCCCC(CCCCCC)C(=O)OCCCCCCN(CCCCCCOC(=O)C(CCCCCC)CCCCCCCC)CCCCNC(C)=O] |

(β) the cationically ionizable lipid is selected from the following structures A to F:

| No. | Structure |
|---|---|
| A | 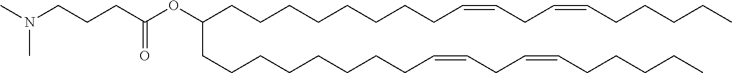 [SMILES: CCCCC/C=C\C/C=C\CCCCCCCCC(CCCCCCCC/C=C\C/C=C\CCCCC)OC(=O)CCCN(C)C] |
| B | 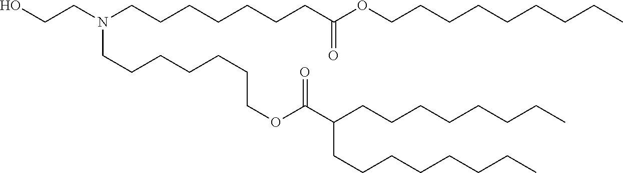 [SMILES: CCCCCCCCCOC(=O)CCCCCCCN(CCO)CCCCCCCOC(=O)C(CCCCCCCC)CCCCCCCC] |
| C | 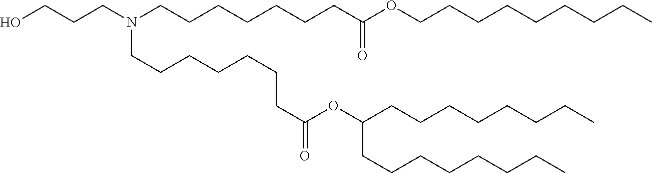 [SMILES: CCCCCCCCCOC(=O)CCCCCCCN(CCCO)CCCCCCCC(=O)OC(CCCCCCCC)CCCCCCCC] |
| D | 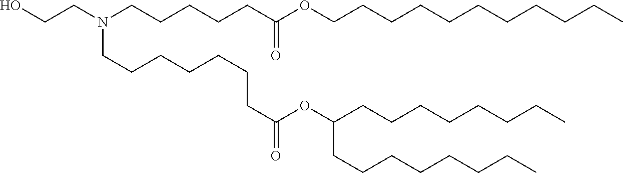 [SMILES: CCCCCCCCCCCOC(=O)CCCCCN(CCO)CCCCCCCC(=O)OC(CCCCCCCC)CCCCCCCC] |
| E | 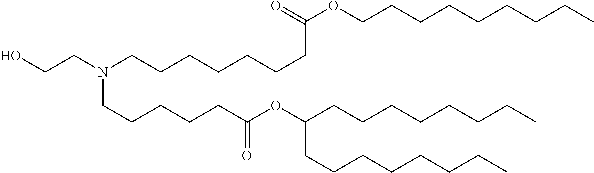 [SMILES: CCCCCCCCCOC(=O)CCCCCCCN(CCO)CCCCCC(=O)OC(CCCCCCCC)CCCCCCCC] |
| F | 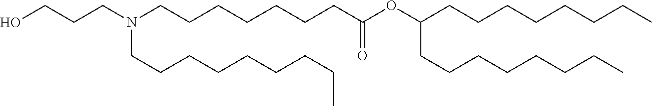 [SMILES: CCCCCCCCCN(CCCO)CCCCCCCC(=O)OC(CCCCCCCC)CCCCCCCC] |

or
(γ) the cationically ionizable lipid is the lipid having the structure I-3.

16. The composition of any one of claims 1 to 15, wherein the LNPs further comprise one or more additional lipids, preferably selected from the group consisting of polymer conjugated lipids, neutral lipids, steroids, and combinations thereof, more preferably the LNPs comprise the cationically ionizable lipid, a polymer conjugated lipid, a neutral lipid, and a steroid.

17. The composition of claim 16, wherein the polymer conjugated lipid comprises a pegylated lipid, wherein the pegylated lipid preferably has the following structure: 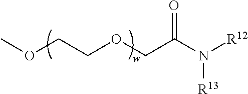
or a pharmaceutically acceptable salt, tautomer or stereoisomer thereof; wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

18. The composition of claim 16, wherein the polymer conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

19. The composition of any one of claims 16 to 18, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

20. The composition of any one of claims 16 to 19, wherein the steroid comprises a sterol such as cholesterol.

21. The composition of any one of claims 1 to 20, wherein the aqueous phase does not comprise a chelating agent.

22. The composition of any one of claims 1 to 21, wherein the LNPs comprise at least about 75%, preferably at least about 80% of the RNA comprised in the composition.

23. The composition of any one of claims 1 to 22, wherein the RNA is encapsulated within or associated with the LNPs.

24. The composition of any one of claims 1 to 23, wherein the RNA comprises a modified nucleoside in place of uridine, wherein the modified nucleoside is preferably selected from pseudouridine (ψ), N1-methyl-pseudouridine (m1ψ), and 5-methyl-uridine (m5U).

25. The composition of any one of claims 1 to 24, wherein the RNA comprises at least one of the following, preferably all of the following: a 5′ cap; a 5′ UTR; a 3′ UTR; and a poly-A sequence.

26. The composition of claim 25, wherein the poly-A sequence comprises at least 100 A nucleotides, wherein the poly-A sequence preferably is an interrupted sequence of A nucleotides.

27. The composition of claim 25 or 26, wherein the 5′ cap is a cap1 or cap2 structure.

28. The composition of any one of claims 1 to 27, wherein the RNA encodes one or more polypeptides, wherein the one or more polypeptides preferably comprise an epitope for inducing an immune response against an antigen in a subject.

29. The composition of claim 28, wherein the RNA comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

30. The composition of claim 28 or 29, wherein the RNA comprises an ORF encoding a full-length SARS-CoV2 S protein variant with proline residue substitutions at positions 986 and 987 of SEQ ID NO: 1.

31. The composition of claim 29 or 30, wherein the SARS-CoV2 S protein variant has at least 80% identity to SEQ ID NO: 7.

32. The composition of any one of claims 1 to 31, wherein the composition is in frozen form.

33. The composition of claim 32, wherein the RNA integrity after thawing the frozen composition is at least 50% compared to the RNA integrity before the composition has been frozen.

34. The composition of claim 32 or 33, wherein the size (Z_{average}) and/or size distribution and/or polydispersity index (PDI) of the LNPs after thawing the frozen composition is equal to the size (Z_{average}) and/or size distribution and/or PDI of the LNPs before the composition has been frozen.

35. The composition of any one of claims 1 to 31, wherein the composition is in liquid form.

36. A method of preparing a composition comprising LNPs dispersed in a final aqueous phase, wherein the LNPs comprise a cationically ionizable lipid and RNA; the final aqueous phase comprises a final buffer system comprising a final buffer substance and a final monovalent anion, the final buffer substance being selected from the group consisting of Tris and its protonated form, Bis-Tris-methane and its protonated form, and TEA and its protonated form, and the final monovalent anion being selected from the group consisting of chloride, acetate, glycolate, lactate, the anion of MES, the anion of MOPS, and the anion of HEPES; the concentration of the final buffer substance in the composition is at most about 25 mM; and the final aqueous phase is substantially free of inorganic phosphate anions, substantially free of citrate anions, and substantially free of anions of EDTA;
wherein the method comprises:
(I) preparing a formulation comprising LNPs dispersed in the final aqueous phase, wherein the LNPs comprise the cationically ionizable lipid and RNA; and
(II) optionally freezing the formulation to about −10° C. or below,
thereby obtaining the composition,
wherein step (1) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing a first intermediate formulation comprising the LNPs dispersed in a first aqueous phase comprising the first buffer system; and
(d) filtrating the first intermediate formulation prepared under (c) using a final aqueous buffer solution comprising the final buffer system,
thereby preparing the formulation comprising the LNPs dispersed in the final aqueous phase.

37. The method of claim 36, wherein step (I) further comprises one or more steps selected from diluting and filtrating.

38. The method of claim 36 or 37, wherein step (I) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f) once or two or more times, wherein the further intermediate formulation comprising the LNPs dispersed in the further aqueous phase comprising the further buffer system obtained after step (f) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f) is absent, or the further intermediate formulation obtained in step (f), if step (f) is present and step (g′) is not present, or the further intermediate formulation obtained after step (g′), if steps (f′) and (g′) are present, using a final aqueous buffer solution comprising the final buffer system and having a pH of at least 6.0; and
(i′) optionally diluting the formulation obtained in step (h′) with a dilution solution;
thereby preparing the formulation comprising the LNPs dispersed in the final aqueous phase.

39. The method of any one of claims 36 to 38, wherein filtrating is tangential flow filtrating or diafiltrating, preferably tangential flow filtrating.

40. The method of any one of claims 36 to 39, which comprises (II) freezing the formulation to about −10° C. or below.

41. The method of any one of claims 36 to 40, wherein the final buffer substance is Tris and its protonated form.

42. The method of any one of claims 36 to 41, wherein the concentration of the final buffer substance, in particular Tris and its protonated form, in the composition is at most about 20 mM, preferably at most about 15 mM, more preferably at most about 10 mM, such as about 10 mM.

43. The method of any one of claims 36 to 42, wherein the final aqueous phase is substantially free of inorganic sulfate anions and/or carbonate anions and/or dibasic organic acid anions and/or polybasic organic acid anions, in particular substantially free of inorganic sulfate anions, carbonate anions dibasic organic acid anions and polybasic organic acid anions.

44. The method of any one of claims 36 to 43, wherein (i) the RNA solution prepared in step (a) further comprises one or more di- and/or polybasic organic acid anions, and step (d) is conducted under conditions which remove the one or more di- and/or polybasic organic acid anions resulting in the formulation comprising the LNPs dispersed in the final aqueous phase with the final aqueous phase being substantially free of the one or more di- and/or polybasic organic acid anions present in the RNA solution prepared in step (a); or (ii) the first aqueous buffer solution and the first aqueous phase comprise one or more di- and/or polybasic organic acid anions and least one of steps (f) to (h′) is conducted under conditions which remove the one or more di- and/or polybasic organic acid anions from the first intermediate formulation and/or from the further intermediate formulation.

45. The method of any one of claims 36 to 44, wherein (i) the RNA solution obtained in step (a) has a pH of below 6.0, preferably at most about 5.0, more preferably at most about 4.5; or (ii) the first aqueous buffer solution has a pH of below 6.0, preferably at most about 5.0, more preferably at most about 4.5.

46. The method of claim 44 or 45, wherein the one or more di- and/or polybasic organic acid anions comprise citrate anions and/or anions of EDTA.

47. The method of any one of claims 36 to 43, wherein (i) the first buffer system used in step (a) comprises the final buffer substance and the final monovalent anion used in step (d), preferably the buffer system and pH of the first buffer system used in step (a) are identical to the buffer system and pH of the final aqueous buffer solution used in step (d); or (ii) each of the first buffer system and every further buffer system used in steps (b′), (f′) and (g′) comprises the final buffer substance and the final monovalent anion used in step (h′), preferably the buffer system and pH of each of the first aqueous buffer solution and of every further aqueous buffer solution used in steps (b′), (f′) and (g′) are identical to the buffer system and pH of the final aqueous buffer solution.

48. The method of any one of claims 36 to 47, wherein the final monovalent anion is selected from the group consisting of chloride, acetate, glycolate, and lactate, and the concentration of the final monovalent anion in the composition is at most equal to, preferably less than the concentration of the final buffer substance in the composition, such as less than about 9 mM.

49. The method of any one of claims 36 to 48, wherein the final monovalent anion is selected from the group consisting of the anions of MES, MOPS, and HEPES, and the concentration of the final monovalent anion in the composition is at least equal to, preferably higher than the concentration of the final buffer substance in the composition.

50. The method of any one of claims 36 to 49, wherein the pH of the composition is between about 6.5 and about 8.0, preferably between about 6.9 and about 7.9, such as between about 7.0 and about 7.8.

51. The method of any one of claims 36 to 50, wherein water is the main component in the formulation and/or composition and/or the total amount of solvent(s) other than water contained in the composition is less than about 0.5% (v/v).

52. The method of any one of claims 36 to 51, wherein the osmolality of the composition is at most about 400×10⁻³ osmol/kg.

53. The method of any one of claims 36 to 52, wherein the concentration of the RNA in the composition is about 5 mg/l to about 150 mg/l, preferably about 10 mg/l to about 130 mg/l, more preferably about 30 mg/l to about 120 mg/l.

54. The method of any one of claims 36 to 53, wherein (i) step (I) further comprises diluting the formulation prepared under (d) with a dilution solution, or step (i′) is present, wherein the dilution solution comprises a cryoprotectant; and/or (ii) the formulation obtained in step (I) and the composition comprise a cryoprotectant, preferably in a concentration of at least about 1% w/v, wherein the cryoprotectant preferably comprises one or more selected from the group consisting of carbohydrates and sugar alcohols, more preferably the cryoprotectant is selected from the group consisting of sucrose, glucose, glycerol, sorbitol, and a combination thereof, more preferably the cryoprotectant comprises sucrose and/or glycerol.

55. The method of any one of claims 36 to 53, wherein the formulation obtained in step (I) and the composition is substantially free of a cryoprotectant.

56. The method of any one of claims 36 to 55, wherein the cationically ionizable lipid comprises a head group which includes at least one nitrogen atom which is capable of being protonated under physiological conditions.

57. The method of any one of claims 36 to 56, wherein the cationically ionizable lipid has the structure of Formula (I): 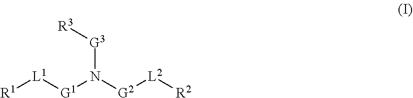
or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or NR^{a}C(═O)O— or a direct bond;
G¹ and G² are each independently unsubstituted C₁-C₁₂ alkylene or C₂-C₁₂ alkenylene;
G³ is C₁-C₂₄ alkylene, C₂-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

58. The method of any one of claims 36 to 56, wherein:
(α) the cationically ionizable lipid is selected from the following structures I-1 to I-36:

| No. | Structure |
|---|---|
| I-1 | 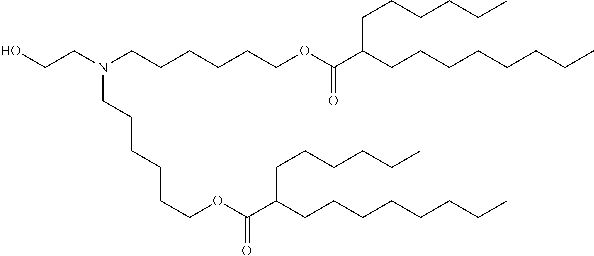 [SMILES: CCCCCCCCC(CCCCCC)C(=O)OCCCCCCN(CCO)CCCCCCOC(=O)C(CCCCCC)CCCCCCCC] |
| I-2 | 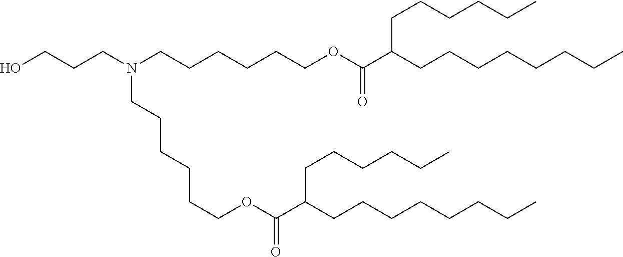 [SMILES: CCCCCCCCC(CCCCCC)C(=O)OCCCCCCN(CCCO)CCCCCCOC(=O)C(CCCCCC)CCCCCCCC] |
| I-3 | 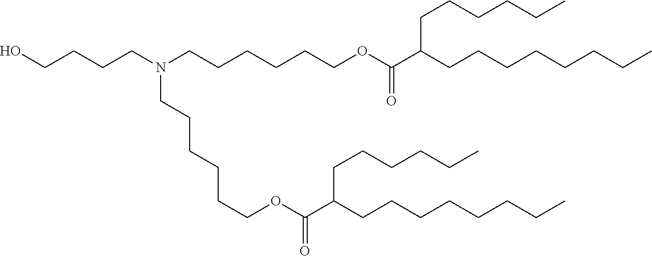 [SMILES: CCCCCCCCC(CCCCCC)C(=O)OCCCCCCN(CCCCO)CCCCCCOC(=O)C(CCCCCC)CCCCCCCC] |
| I-4 | 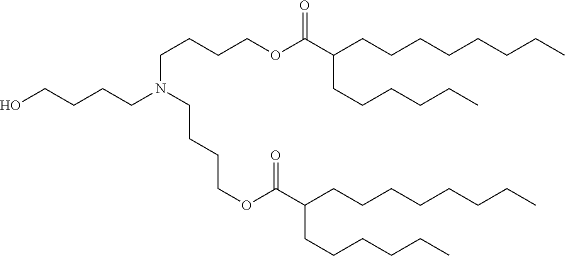 [SMILES: CCCCCCCCC(CCCCCC)C(=O)OCCCCN(CCCCO)CCCCOC(=O)C(CCCCCC)CCCCCCCC] |
| I-5 | 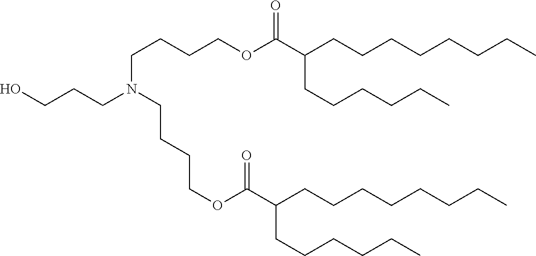 [SMILES: CCCCCCCCC(CCCCCC)C(=O)OCCCCN(CCCO)CCCCOC(=O)C(CCCCCC)CCCCCCCC] |
| I-6 | 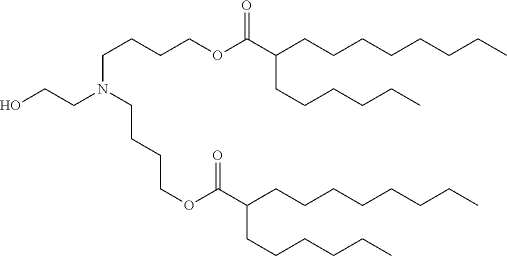 [SMILES: CCCCCCCCC(CCCCCC)C(=O)OCCCCN(CCO)CCCCOC(=O)C(CCCCCC)CCCCCCCC] |
| I-7 | 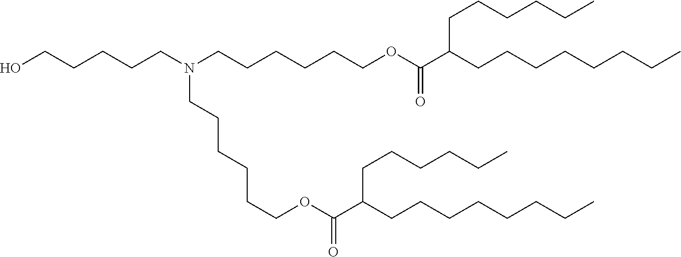 [SMILES: CCCCCCCCC(CCCCCC)C(=O)OCCCCCCN(CCCCCO)CCCCCCOC(=O)C(CCCCCC)CCCCCCCC] |
| I-8 | 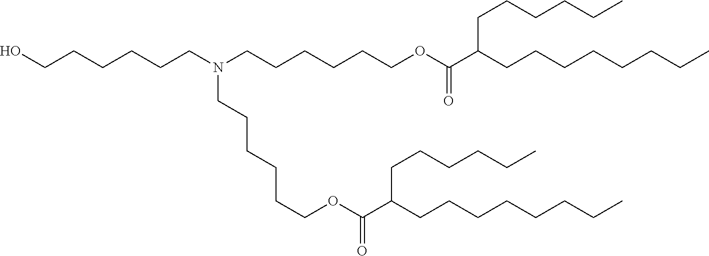 [SMILES: CCCCCCCCC(CCCCCC)C(=O)OCCCCCCN(CCCCCCO)CCCCCCOC(=O)C(CCCCCC)CCCCCCCC] |
| I-9 | 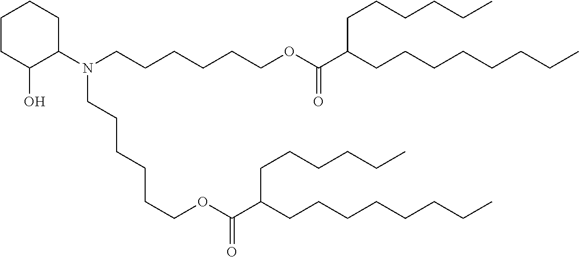 [SMILES: CCCCCCCCC(CCCCCC)C(=O)OCCCCCCN(CCCCCCOC(=O)C(CCCCCC)CCCCCCCC)C1CCCCC1O] |
| I-10 | 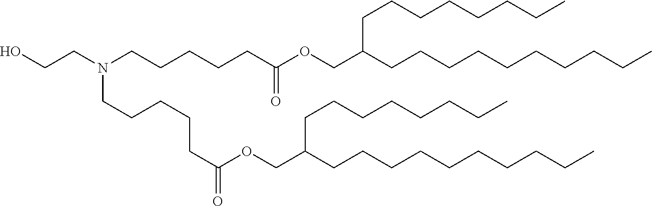 [SMILES: CCCCCCCCCCC(CCCCCCCC)COC(=O)CCCCCN(CCO)CCCCCC(=O)OCC(CCCCCCCC)CCCCCCCCCC] |
| I-11 | 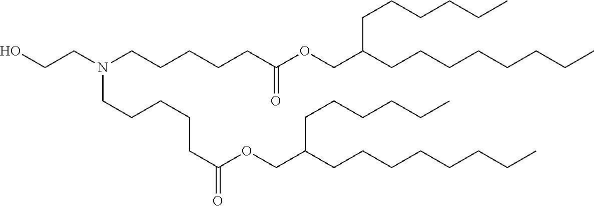 [SMILES: CCCCCCCCC(CCCCCC)COC(=O)CCCCCN(CCO)CCCCCC(=O)OCC(CCCCCC)CCCCCCCC] |
| I-12 | 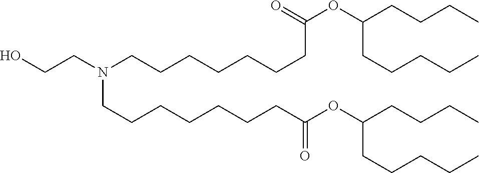 [SMILES: CCCCCC(CCCC)OC(=O)CCCCCCCN(CCO)CCCCCCCC(=O)OC(CCCC)CCCCC] |
| I-13 | 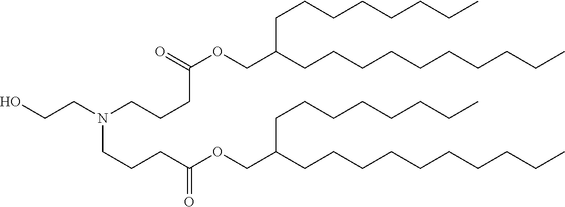 [SMILES: CCCCCCCCCCC(CCCCCCCC)COC(=O)CCCN(CCO)CCCC(=O)OCC(CCCCCCCC)CCCCCCCCCC] |
| I-14 | 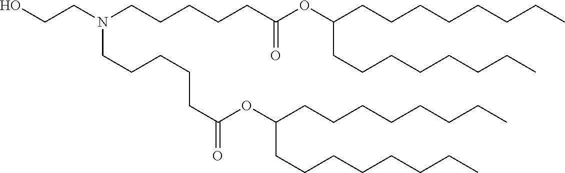 [SMILES: CCCCCCCCC(CCCCCCCC)OC(=O)CCCCCN(CCO)CCCCCC(=O)OC(CCCCCCCC)CCCCCCCC] |
| I-15 | 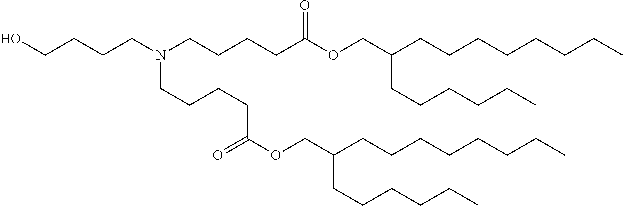 [SMILES: CCCCCCCCC(CCCCCC)COC(=O)CCCCN(CCCCO)CCCCC(=O)OCC(CCCCCC)CCCCCCCC] |
| I-16 | 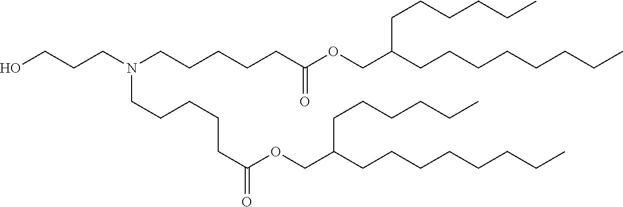 [SMILES: CCCCCCCCC(CCCCCC)COC(=O)CCCCCN(CCCO)CCCCCC(=O)OCC(CCCCCC)CCCCCCCC] |
| I-17 | 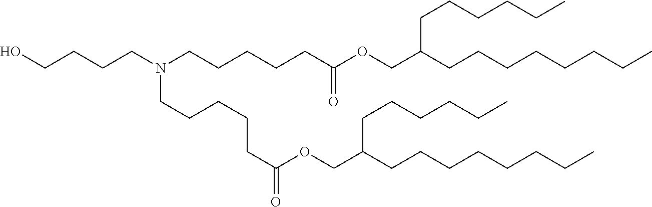 [SMILES: CCCCCCCCC(CCCCCC)COC(=O)CCCCCN(CCCCO)CCCCCC(=O)OCC(CCCCCC)CCCCCCCC] |
| I-18 | 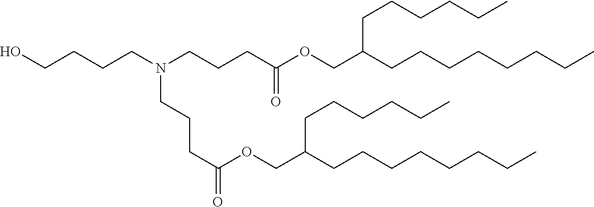 [SMILES: CCCCCCCCC(CCCCCC)COC(=O)CCCN(CCCCO)CCCC(=O)OCC(CCCCCC)CCCCCCCC] |
| I-19 | 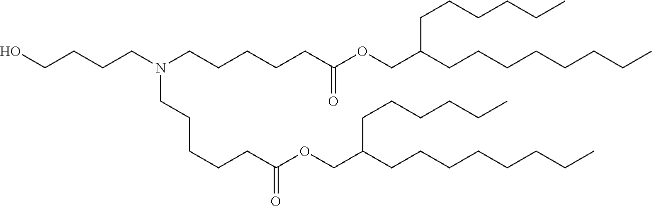 [SMILES: CCCCCCCCC(CCCCCC)COC(=O)CCCCCN(CCCCO)CCCCCC(=O)OCC(CCCCCC)CCCCCCCC] |
| I-20 | 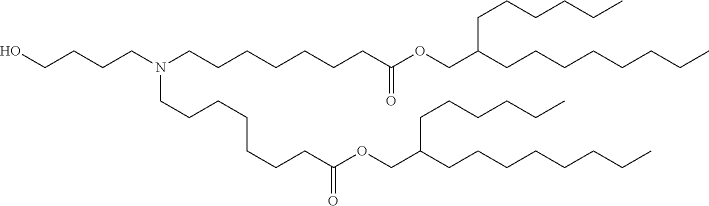 [SMILES: CCCCCCCCC(CCCCCC)COC(=O)CCCCCCCN(CCCCO)CCCCCCCC(=O)OCC(CCCCCC)CCCCCCCC] |
| I-21 | 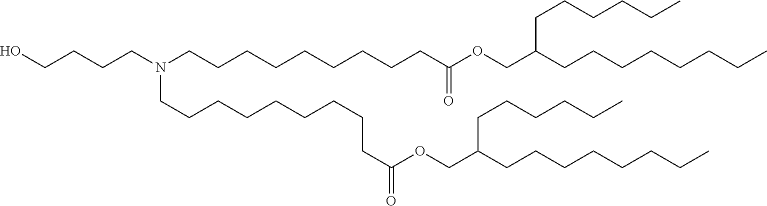 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 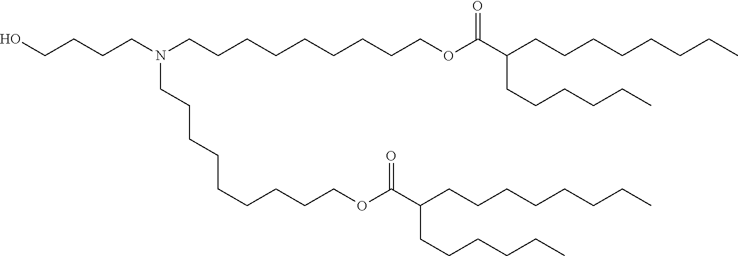 [SMILES: CCCCCCCCC(CCCCCC)C(=O)OCCCCCCCCCN(CCCCO)CCCCCCCCCOC(=O)C(CCCCCC)CCCCCCCC] |
| I-23 | 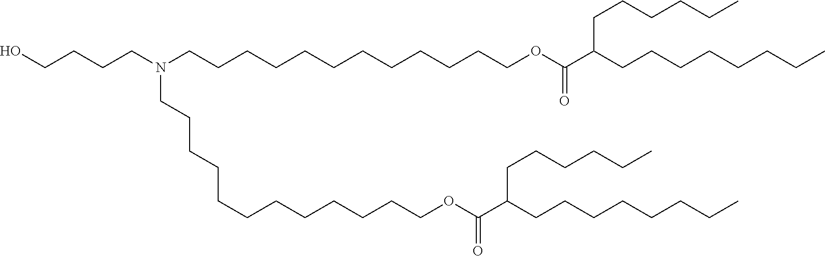 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 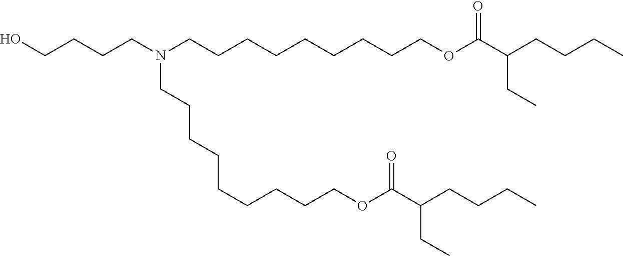 [SMILES: CCCCC(CC)C(=O)OCCCCCCCCCN(CCCCO)CCCCCCCCCOC(=O)C(CC)CCCC] |
| I-25 | 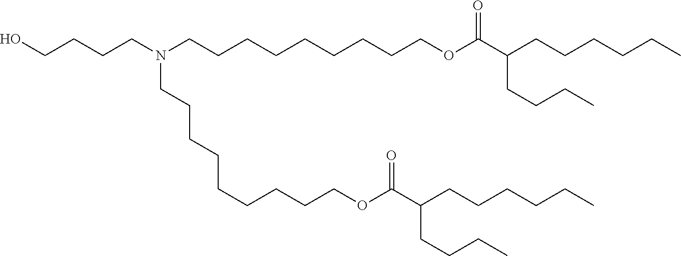 [SMILES: CCCCCCC(CCCC)C(=O)OCCCCCCCCCN(CCCCO)CCCCCCCCCOC(=O)C(CCCC)CCCCCC] |
| I-26 | 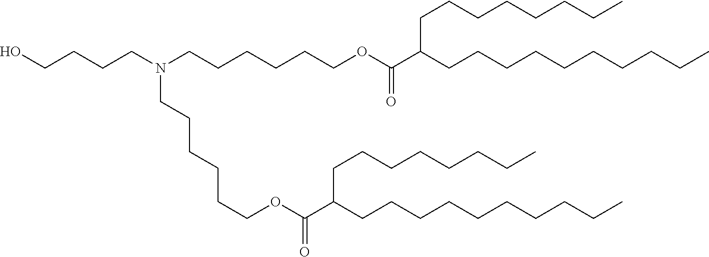 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 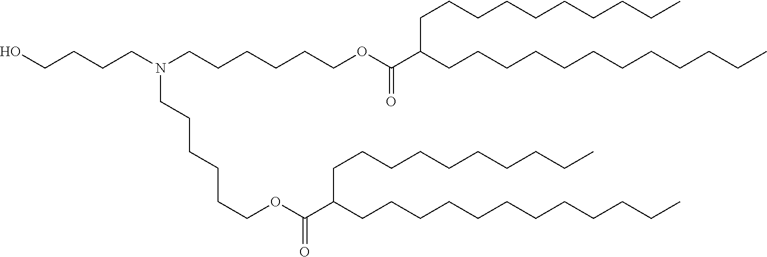 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 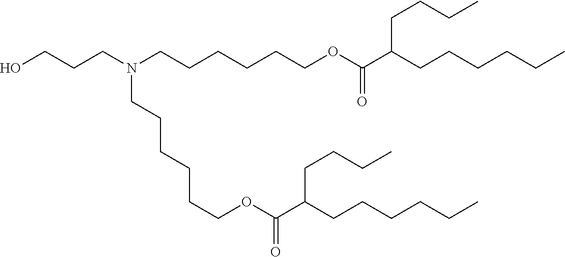 [SMILES: CCCCCCC(CCCC)C(=O)OCCCCCCN(CCCO)CCCCCCOC(=O)C(CCCC)CCCCCC] |
| I-29 | 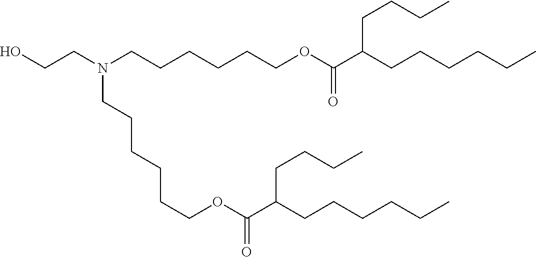 [SMILES: CCCCCCC(CCCC)C(=O)OCCCCCCN(CCO)CCCCCCOC(=O)C(CCCC)CCCCCC] |
| I-30 | 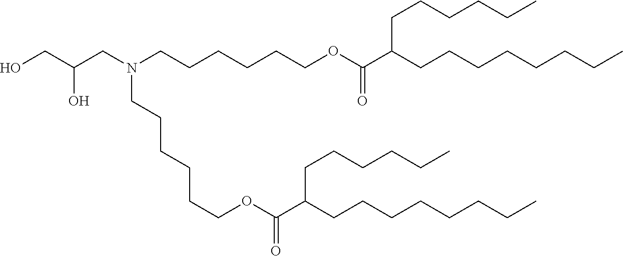 [SMILES: CCCCCCCCC(CCCCCC)C(=O)OCCCCCCN(CCCCCCOC(=O)C(CCCCCC)CCCCCCCC)CC(O)CO] |
| I-31 | 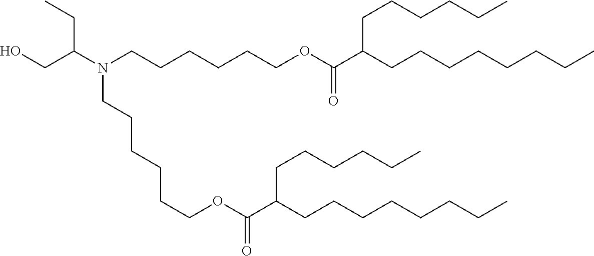 [SMILES: CCCCCCCCC(CCCCCC)C(=O)OCCCCCCN(CCCCCCOC(=O)C(CCCCCC)CCCCCCCC)C(CC)CO] |
| I-32 | 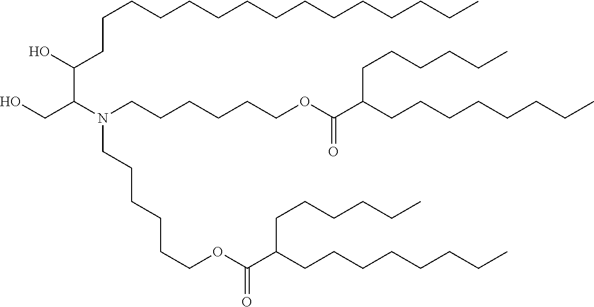 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 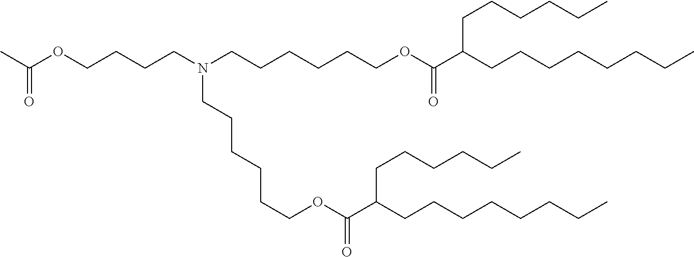 [SMILES: CCCCCCCCC(CCCCCC)C(=O)OCCCCCCN(CCCCCCOC(=O)C(CCCCCC)CCCCCCCC)CCCCOC(C)=O] |
| I-34 | 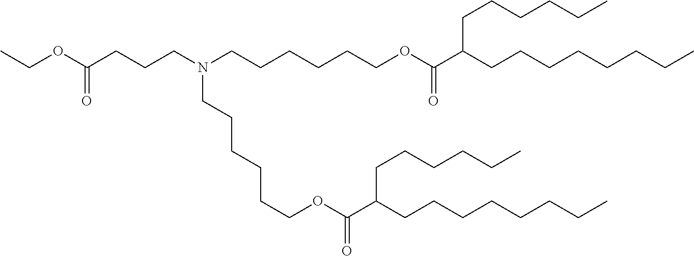 [SMILES: CCCCCCCCC(CCCCCC)C(=O)OCCCCCCN(CCCCCCOC(=O)C(CCCCCC)CCCCCCCC)CCCC(=O)OCC] |
| I-35 | 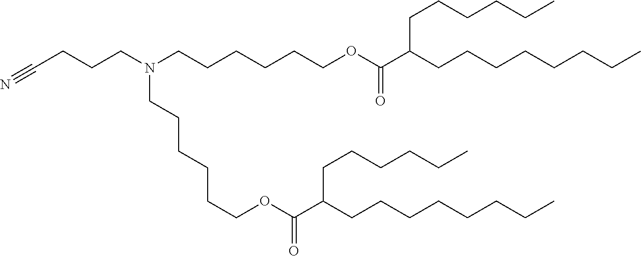 [SMILES: CCCCCCCCC(CCCCCC)C(=O)OCCCCCCN(CCCC#N)CCCCCCOC(=O)C(CCCCCC)CCCCCCCC] |
| I-36 | 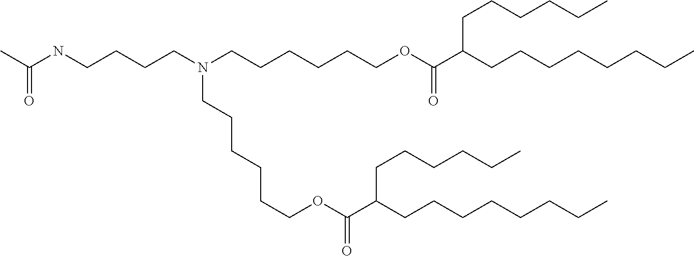 [SMILES: CCCCCCCCC(CCCCCC)C(=O)OCCCCCCN(CCCCCCOC(=O)C(CCCCCC)CCCCCCCC)CCCCNC(C)=O] |

(β) the cationically ionizable lipid is selected from the following structures A to F:

| No. | Structure |
|---|---|
| A | 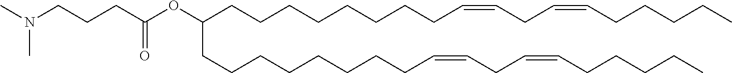 [SMILES: CCCCC/C=C\C/C=C\CCCCCCCCC(CCCCCCCC/C=C\C/C=C\CCCCC)OC(=O)CCCN(C)C] |
| B | 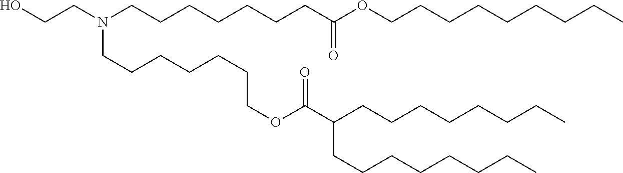 [SMILES: CCCCCCCCCOC(=O)CCCCCCCN(CCO)CCCCCCCOC(=O)C(CCCCCCCC)CCCCCCCC] |
| C | 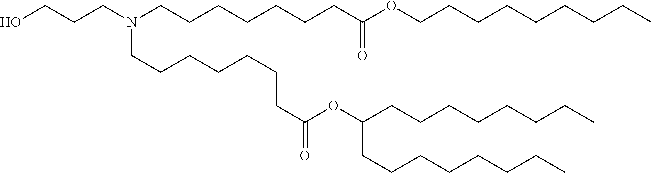 [SMILES: CCCCCCCCCOC(=O)CCCCCCCN(CCCO)CCCCCCCC(=O)OC(CCCCCCCC)CCCCCCCC] |
| D | 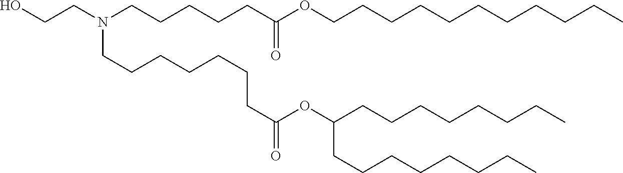 [SMILES: CCCCCCCCCCCOC(=O)CCCCCN(CCO)CCCCCCCC(=O)OC(CCCCCCCC)CCCCCCCC] |
| E | 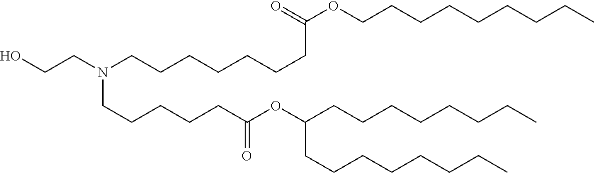 [SMILES: CCCCCCCCCOC(=O)CCCCCCCN(CCO)CCCCCC(=O)OC(CCCCCCCC)CCCCCCCC] |
| F | 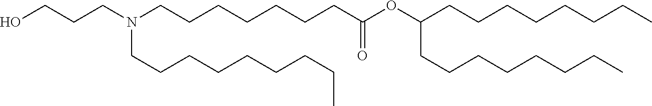 [SMILES: CCCCCCCCCN(CCCO)CCCCCCCC(=O)OC(CCCCCCCC)CCCCCCCC] |

or
(γ) the cationically ionizable lipid is the lipid having the structure I-3.

59. The method of any one of claims 36 to 58, wherein the ethanolic solution prepared in step (b) or (d′) further comprises one or more additional lipids and the LNPs further comprise the one or more additional lipids, wherein the one or more additional lipids are preferably selected from the group consisting of polymer conjugated lipids, neutral lipids, steroids, and combinations thereof, more preferably the one or more additional lipids comprise a polymer conjugated lipid, a neutral lipid, and a steroid.

60. The method of claim 59, wherein the polymer conjugated lipid comprises a pegylated lipid, wherein the pegylated lipid preferably has the following structure: 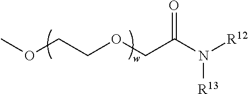
or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

61. The method of claim 59, wherein the polymer conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosne-ceramide conjugate, and a mixture thereof.

62. The method of any one of claims 59 to 61, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

63. The method of any one of claims 59 to 62, wherein the steroid comprises a sterol such as cholesterol.

64. The method of any one of claims 36 to 63, wherein the cationically ionizable lipid, the polymer conjugated lipid, the neutral lipid, and the steroid are present in the ethanolic solution in a molar ratio of 20% to 60% of the cationically ionizable lipid, 0.5% to 15% of the polymer conjugated lipid, 5% to 25% of the neutral lipid, and 25% to 55% of the steroid, preferably in a molar ratio of 45% to 55% of the cationically ionizable lipid, 1.0% to 5% of the polymer conjugated lipid, 8% to 12% of the neutral lipid, and 35% to 45% of the steroid.

65. The method of any one of claims 36 to 64, wherein the final aqueous phase does not comprise a chelating agent.

66. The method of any one of claims 36 to 65, wherein the LNPs comprise at least about 75%, preferably at least about 80% of the RNA comprised in the composition.

67. The method of any one of claims 36 to 66, wherein the RNA is encapsulated within or associated with the LNPs.

68. The method of any one of claims 36 to 67, wherein the RNA comprises a modified nucleoside in place of uridine, wherein the modified nucleoside is preferably selected from pseudouridine (ψ), N1-methyl-pseudouridine (m1ψ), and 5-methyl-uridine (m5U).

69. The method of any one of claims 36 to 68, wherein the RNA comprises at least one of the following, preferably all of the following: a 5′ cap; a 5′ UTR; a 3′ UTR and a poly-A sequence.

70. The method of claim 69, wherein the poly-A sequence comprises at least 100 A nucleotides, wherein the poly-A sequence preferably is an interrupted sequence of A nucleotides.

71. The method of claim 69 or 70, wherein the 5′ cap is a cap1 or cap2 structure.

72. The method of any one of claims 36 to 71, wherein the RNA encodes one or more polypeptides, wherein the one or more polypeptides preferably comprise an epitope for inducing an immune response against an antigen in a subject.

73. The method of claim 72, wherein the RNA comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

74. The method of claim 72 or 73, wherein the RNA comprises an ORF encoding a full-length SARS-CoV2 S protein variant with proline residue substitutions at positions 986 and 987 of SEQ ID NO: 1.

75. The method of claim 73 or 74, wherein the SARS-CoV2 S protein variant has at least 80% identity to SEQ ID NO: 7.

76. The method of any one of claims 36 to 39 and 41 to 75, which does not comprise step (II).

77. A method of storing a composition, comprising preparing a composition according to the method of any one of claims 36 to 75 and storing the composition at a temperature ranging from about −90° C. to about −10° C., such as from about −90° C. to about −40° C. or from about −25° C. to about −10° C.

78. The method of claim 77, wherein storing the composition is for at least 1 week, such as at least 2 weeks, at least 3 weeks, at least 4 weeks, at least 1 month, at least 2 months, at least 3 months, at least 6 months, at least 12 months, at least 24 months, or at least 36 months.

79. A method of storing a composition, comprising preparing a composition according to the method of any one of claims 36 to 78 and storing the composition at a temperature ranging from about 0° C. to about 20° C., such as from about 1° C. to about 15° C., from about 2° C. to about 10° C., or from about 2° C. to about 8° C., or at a temperature of about 5° C.

80. The method of claim 79, wherein storing the composition is for at least 1 week, such as at least 2 weeks, at least 3 weeks, at least 4 weeks, at least 1 month, at least 2 months, at least 3 months, or at least 6 months.

81. A composition preparable by the method of any one of claims 36 to 80.

82. The composition of claim 81, which is in frozen form.

83. The composition of claim 82, wherein the RNA integrity after thawing the frozen composition is at least 50% compared to the RNA integrity of the composition before the composition has been frozen.

84. The composition of claim 82 or 83, wherein the size (Z_{average}) and/or size distribution and/or polydispersity index (PDI) of the LNPs after thawing the frozen composition is equal to the size (Z_{average}) and/or size distribution and/or PDI of the LNPs before the composition has been frozen.

85. The composition of claim 81, which is in liquid form.

86. The composition of claim 85, wherein the RNA integrity after storage of the composition for at least 1 week is at least 50% compared to the RNA integrity before storage.

87. The composition of claim 85 or 86, wherein the size (Z_{average}) and/or size distribution and/or polydispersity index (PDI) of the LNPs after storage of the composition for at least one week is equal to the size (Z_{average}) and/or size distribution and/or PDI of the LNPs before storage.

88. A method for preparing a ready-to-use pharmaceutical composition, the method comprising the steps of providing a frozen composition prepared by the method of any one of claims 36 to 75, 77, and 78, and thawing the frozen composition thereby obtaining the ready-to-use pharmaceutical composition.

89. A method for preparing a ready-to-use pharmaceutical composition, the method comprising the step of providing a liquid composition prepared by the method of any one of claims 36 to 39, 41 to 76, 79, and 80, thereby obtaining the ready-to-use pharmaceutical composition.

90. A ready-to-use pharmaceutical composition preparable by the method of claim 88 or 89.

91. A composition of any one of claims 1 to 35, 81 to 87, and 90 for use in therapy.

92. A composition of any one of claims 1 to 35, 81 to 87, and 90 for use in inducing an immune response in a subject.
